SEQUENCE LISTING (20 sequences)

SEQ ID NO: 1 - protein, 411 aa, synthetic construct
  REGION 1..411: note = Synthetic polypeptide
MAAPDLLDPKSAAQNSKPRLSFSSKPTVLASRVESDSAINVMKWKTVSTIFLVVVLYLIIGATVFKALEQPQEISQRTTIVIQKQTFIAQHACVNSTELDELIQQIVAAINAGIIPLGNSSNQVSHWDLGCSFFFAGTVITTIGFGNISPRTEGGKIFCIIYALLGIPLFGFLLAGVGDQLGTIFGKGIAKVEDTFIKWNVSQTKIRIISTIIFILFGCVLFVALPAVIFKHIEGWSALDAIYFVVITLTTIGFGDYVAGGSDIEYLDFYKPVVWFWILVGLAYFAAVLSMIGDWLRVISKKTKEEVGEFRAHAAEWTANVTAEFKETRRRLSVEIYDKFQRATSVKRKLSAELAGNHNQELTPCRRTLSVNHLTSEREVLPPLLKAESIYLNGLTPHCAGEDIAVIENMK

SEQ ID NO: 2 - protein, 302 aa, synthetic construct
  REGION 1..302: note = Synthetic polypeptide
SFSSKPTVLASRVESDSAINVMKWKTVSTIFLVVVLYLIIGATVFKALEQPQEISQRTTIVIQKQTFIAQHACVNSTELDELIQQIVAAINAGIIPLGNSSNQVSHWDLGCSFFFAGTVITTIGFGNISPRTEGGKIFCIIYALLGIPLFGFLLAGVGDQLGTIFGKGIAKVEDTFIKWNVSQTKIRIISTIIFILFGCVLFVALPAVIFKHIEGWSALDAIYFVVITLTTIGFGDYVAGGSDIEYLDFYKPVVWFWILVGLAYFAAVLSMIGDWLRVISKKTKEEVGEFRAHAAEWTANVT

SEQ ID NO: 3 - protein, 266 aa, synthetic construct
  REGION 1..266: note = Synthetic polypeptide
VSTIFLVVVLYLIIGATVFKALEQPQEISQRTTIVIQKQTFIAQHACVNSTELDELIQQIVAAINAGIIPLGNSSNQVSHWDLGCSFFFAGTVITTIGFGNISPRTEGGKIFCIIYALLGIPLFGFLLAGVGDQLGTIFGKGIAKVEDTFIKWNVSQTKIRIISTIIFILFGCVLFVALPAVIFKHIEGWSALDAIYFVVITLTTIGFGDYVAGGSDIEYLDFYKPVVWFWILVGLAYFAAVLSMIGDWLRVISKKTKEEVGEFRA

SEQ ID NO: 4 - protein, 422 aa, synthetic construct
  REGION 1..422: note = Synthetic polypeptide
MMNPRAKRDFYLAAPDLLDPKSAAQNSKPRLSFSTKPTVLASRVESDTTINVMKWKTVSTIFLVVVLYLIIGATVFKALEQPHEISQRTTIVIQKQTFISQHSCVNSTELDELIQQIVAAINAGIIPLGNTSNQISHWDLGCSFFFAGTVITTIGFGNISPRTEGGKIFCIIYALLGIPLFGFLLAGVGDQLGTIFGKGIAKVEDTFIKWNVSQTKIRIISTIIFILFGCVLFVALPAIIFKHIEGWSALDAIYFVVITLTTIGFGDYVAGGSDIEYLDFYKPVVWFWILVGLAYFAAVLSMIGDWLRVISKKTKEEVGEFRAHAAEWTANVTAEFKETRRRLSVEIYDKFQRATSIKRKLSAELAGNHNQELTPCRRTLSVNHLTSERDVLPPLLKTESIYLNGLTPHCAGEEIAVIENIK

SEQ ID NO: 5 - protein, 538 aa, synthetic construct
  REGION 1..538: note = Synthetic polypeptide
MKFPIETPRKQVNWDPKVAVPAAAPPVCQPKSATNGHHPVPRLSISSRATVVARMEGASQGGLQTVMKWKTVVAIFVVVVVYLVTGGLVFRALEQPFESSQKNTIALEKAEFLRDHICVSPQELETLIQHALDADNAGVSPVGNSSNSSSHWDLGCAFFFAGTVITTIGYGNIAPSTEGGKIFCILYAIFGIPLFGFLLAGIGDQLGTIFGKSIARVEKVFRKKQVSQTKIRVISTILFILAGCIVFVTIPAVIFKYIEGWTALESIYFVVVTLTTVGFGDFVAGGNAGINYREWYKPLVWFWILVGLAYFAAVLSMIGDWLRVLSKKTKEEVGEIKAHAAEWKANVTAEFRETRRRLSVEIHDKLQRAATIRSMERRRLGLDQRAHSLDMLSPEKRSVFAALDTGRFKASSQESINNRPNNLRLKGPEQLTKHGQGASEDNIINKFGSTSKLTKRKNKDLKKTLPEDVQKIYKTFRNYSLDEEKKEDETEKMCNSDNSSTAMLTECIQQQAEMENGMVPTDTKDQGLENNSLLEDRN

SEQ ID NO: 6 - protein, 272 aa, synthetic construct
  REGION 1..272: note = Synthetic polypeptide
QGGLQTVMKWKTVVAIFVVVVVYLVTGGLVFRALEQPFESSQKNTIALEKAEFLRDHICVSPQELETLIQHALDADNAGVSPVGNSSNSSSHWDLGCAFFFAGTVITTIGYGNIAPSTEGGKIFCILYAIFGIPLFGFLLAGIGDQLGTIFGKSIARVEKVFRKKQVSQTKIRVISTILFILAGCIVFVTIPAVIFKYIEGWTALESIYFVVVTLTTVGFGDFVAGGNAGINYREWYKPLVWFWILVGLAYFAAVLSMIGDWLRVLSKKTKE

SEQ ID NO: 7 - protein, 543 aa, synthetic construct
  REGION 1..543: note = Synthetic polypeptide
MKFPIETPRKQVNWDPKVAVPAAAPVCQPKSATNGQPPAPAPTPTPRLSISSRATVVARMEGTSQGGLQTVMKWKTVVAIFVVVVVYLVTGGLVFRALEQPFESSQKNTIALEKAEFLRDHVCVSPQELETLIQHALDADNAGVSPIGNSSNNSSHWDLGCAFFFAGTVITTIGYGNIAPSTEGGKIFCILYAIFGIPLFGFLLAGIGDQLGTIFGKSIARVEKVFRKKQVSQTKIRVISTILFILAGCIVFVTIPAVIFKYIEGWTALESIYFVVVTLTTVGFGDFVAGGNAGINYREWYKPLVWFWILVGLAYFAAVLSMIGDWLRVLSKKTKEEVGEIKAHAAEWKANVTAEFRETRRRLSVEIHDKLQRAATIRSMERRRLGLDQRAHSLDMLSPEKRSVFAALDTGRFKASSQESINNRPNNLRLKGPEQLNKHGQGASEDNIINKFGSTSRLTKRKNKDLKKTLPEDVQKIYKTFRNYSLDEEKKEEETEKMCNSDNSSTAMLTDCIQQHAELENGMIPTDTKDREPENNSLLEDRN

SEQ ID NO: 8 - protein, 274 aa, synthetic construct
  REGION 1..274: note = Synthetic polypeptide
GLQTVMKWKTVVAIFVVVVVYLVTGGLVFRALEQPFESSQKNTIALEKAEFLRDHVCVSPQELETLIQHALDADNAGVSPIGNSSNNSSHWDLGCAFFFAGTVITTIGYGNIAPSTEGGKIFCILYAIFGIPLFGFLLAGIGDQLGTIFGKSIARVEKVFRKKQVSQTKIRVISTILFILAGCIVFVTIPAVIFKYIEGWTALESIYFVVVTLTTVGFGDFVAGGNAGINYREWYKPLVWFWILVGLAYFAAVLSMIGDWLRVLSKKTKEEVGE

SEQ ID NO: 9 - protein, 398 aa, synthetic construct
  REGION 1..398: note = Synthetic polypeptide
MRSTTLLALLALVLLYLVSGALVFQALEQPHEQQAQKKMDHGRDQFLRDHPCVSQKSLEDFIKLLVEALGGGANPETSWTNSSNHSSAWNLGCAFFFSGTIITTIGYGNIVLHTDAGRLFCIFYALVGIPLFGMLLAGVGDRLGSSLRRGIGHIEAIFLKWHVPPGLVRSLSAVLFLLIGCLLFVLTPTFVFSYMESWSKLEAIYFVIVTLTTVGFGDYVPGDGTGQNSPAYQPLVWFWILFGLAYFASVLTTIGNWLRAVSRRTRAEMGGLTAQAASWTGTVTARVTQRTGPSAPPPEKEQPLLPSSLPAPPAVVEPAGRPGSPAPAEKVETPSPPTASALDYPSENLAFIDESSDTQSERGCALPRAPRGRRRPNPSKKPSRPRGPGRLRDKAVPV

SEQ ID NO: 10 - protein, 275 aa, synthetic construct
  REGION 1..275: note = Synthetic polypeptide
MRSTTLLALLALVLLYLVSGALVFQALEQPHEQQAQKKMDHGRDQFLRDHPCVSQKSLEDFIKLLVEALGGGANPETSWTNSSNHSSAWNLGCAFFFSGTIITTIGYGNIVLHTDAGRLFCIFYALVGIPLFGMLLAGVGDRLGSSLRRGIGHIEAIFLKWHVPPGLVRSLSAVLFLLIGCLLFVLTPTFVFSYMESWSKLEAIYFVIVTLTTVGFGDYVPGDGTGQNSPAYQPLVWFWILFGLAYFASVLTTIGNWLRAVSRRTRAEMGGLTAQ

SEQ ID NO: 11 - protein, 393 aa, synthetic construct
  REGION 1..393: note = Synthetic polypeptide
MRSTTLLALLALVLLYLVSGALVFRALEQPHEQQAQRELGEVREKFLRAHPCVSDQELGLLIKEVADALGGGADPETNSTSNSSHSAWDLGCAFFFSGTIITTIGYGNVALRTDAGRLFCIFYALVGIPLFGILLAGVGDRLGSSLRHGIGHIEAIFLKWHVPPELVRVLSAMLFLLIGCLLFVLTPTFVFCYMEDWSKLEAIYFVIVTLTTVGFGDYVAGADPRQDSPAYQPLVWFWILLGLAYFASVLTTIGNWLRVVSRRTRAEMGGLTAQAASWTGTVTARVTQRAGPAAPPPEKEQPLLPPPPCPAQPLGRPRSPSPPEKAQPPSPPTASALDYPSENLAFIDESSDTQSERGCPLPRAPRGRRRPNPPRKPVRPRGPGRPRDKGVPV

SEQ ID NO: 12 - protein, 301 aa, synthetic construct
  REGION 1..301: note = Synthetic polypeptide
MRSTTLLALLALVLLYLVSGALVFRALEQPHEQQAQRELGEVREKFLRAHPCVSDQELGLLIKEVADALGGGADPETNSTSNSSHSAWDLGCAFFFSGTIITTIGYGNVALRTDAGRLFCIFYALVGIPLFGILLAGVGDRLGSSLRHGIGHIEAIFLKWHVPPELVRVLSAMLFLLIGCLLFVLTPTFVFCYMEDWSKLEAIYFVIVTLTTVGFGDYVAGADPRQDSPAYQPLVWFWILLGLAYFASVLTTIGNWLRVVSRRTRAEMGGLTAQAASWTGTVTARVTQRAGPAAPPPEKEQ

SEQ ID NO: 13 - protein, 34 aa, synthetic construct
  REGION 1..34: note = Synthetic polypeptide
PLGNSSNQVSHWDLGSSFFFAGTVITTIGFGNIS

SEQ ID NO: 14 - protein, 34 aa, synthetic construct
  REGION 1..34: note = Synthetic polypeptide
PVGNSSNSSSHWDLGSAFFFAGTVITTIGYGNIA

SEQ ID NO: 15 - protein, 37 aa, synthetic construct
  REGION 1..37: note = Synthetic polypeptide
PETSWTNSSNHSSAWNLGSAFFFSGTIITTIGYGNIV

SEQ ID NO: 16 - protein, 36 aa, synthetic construct
  REGION 1..36: note = Synthetic polypeptide
PETNSTSNSSHSAWDLGSAFFFSGTIITTIGYGNVA

SEQ ID NO: 17 - protein, 34 aa, synthetic construct
  REGION 1..34: note = Synthetic polypeptide
LKPQWLKAPQDWSFLSALFFCCTVFSTVGYGHMY

SEQ ID NO: 18 - protein, 30 aa, synthetic construct
  REGION 1..30: note = Synthetic polypeptide
YFCFVTLTTIGFGDIVLVHPHFFLFFSIYI

SEQ ID NO: 19 - protein, 20 aa, synthetic construct
  REGION 1..20: note = Synthetic polypeptide
AAAGSGGSGGSGGSSGSSGS

SEQ ID NO: 20 - protein, 9 aa, synthetic construct
  REGION 1..9: note = Synthetic polypeptide
YPYDVPDYA

(19) United States
(12) Patent Application Publication
(10) Pub. No.: US 2026/0098008 A1
(43) Pub. Date: Apr. 9, 2026

(54) COVALENT, CHEMOGENETIC ACTIVATORS FOR K2P POTASSIUM CHANNELS AND USES THEREOF

(71) Applicant: THE REGENTS OF THE UNIVERSITY OF CALIFORNIA, Oakland, CA (US)

(72) Inventors: Adam R. Renslo, Oakland, CA (US); Daniel L. Minor, San Francisco, CA (US); Panker E. Deal, Oakland, CA (US); Clifford Bryant, Albuquerque, NM (US); Xochina Cora EiHilali-Pollard, Oakland, CA (US); Marco Gaetano Lorenzo Lolicato, Pavia (IT)

(21) Appl. No.: 19/113,381

(22) PCT Filed: Sep. 22, 2023
(86) PCT No.: PCT/US2023/074948

§ 371 Date: Mar. 19, 2025

Related U.S. Application Data
(60) Provisional application No. 63/376,973, filed on Sep. 23, 2022.
(60) Provisional application No. 63/383,531, filed on Nov. 14, 2022.

(51) Int. Cl. C07C 237/42; A61K 31/167; A61K 31/17; A61K 31/18; A61K 31/196; A61K 31/336; A61K 31/341; A61K 31/36; A61K 31/365; A61K 31/381; A61K 31/397; A61K 31/402; A61K 31/4406; A61K 31/451; A61K 31/472; A61K 31/505; A61K 31/55; A61K 31/553; A61K 31/69; A61K 38/17; A61K 48/00; C07C 233/66; C07C 237/30; C07C 275/40; C07C 275/50; C07D 207/444; C07D 207/452; C07D 207/456; C07D 211/04; C07D 213/57; C07D 217/24; C07D 223/16; C07D 239/42; C07D 267/14; C07D 285/00; C07D 303/36; C07D 307/54; C07D 317/60; C07D 333/24; C07D 493/10; C07F 5/02; C07K 14/705

(52) U.S. Cl. CPC C07C 237/42; A61K 31/167; A61K 31/17; A61K 31/18; A61K 31/196; A61K 31/336; A61K 31/341; A61K 31/36; A61K 31/365; A61K 31/381; A61K 31/397; A61K 31/402; A61K 31/4406; A61K 31/451; A61K 31/472; A61K 31/505; A61K 31/55; A61K 31/553; A61K 31/69; A61K 38/177; A61K 48/005; C07C 233/66; C07C 237/30; C07C 275/40; C07C 275/50; C07D 207/444; C07D 207/452; C07D 207/456; C07D 211/04; C07D 213/57; C07D 217/24; C07D 223/16; C07D 239/42; C07D 267/14; C07D 285/00; C07D 303/36; C07D 307/54; C07D 317/60; C07D 333/24; C07D 493/10; C07F 5/025; C07K 14/705; C07C 2601/02

(57) ABSTRACT

Disclosed herein are, inter alia, activators of K2P potassium channels and pharmaceutical compositions thereof, and methods comprising their use for the treatment of diseases or adverse conditions related to low TREK family protein activity.

### CROSS-REFERENCES TO RELATED APPLICATIONS

This application claims the benefit of U.S. Provisional Application No. 63/376,973, filed Sep. 23, 2022, and U.S. Provisional Application No. 63/383,531, filed Nov. 14, 2022, which are incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with government support under grants no. R01 MH093603, and R01 MH116278 awarded by The National Institutes of Health. The government has certain rights in the invention.

### REFERENCE TO AN ELECTRONIC SEQUENCE LISTING

The contents of the electronic sequence listing (048536-744001WO_Sequence_Listing_ST26.xml; Size: 30,467 bytes; and Date of Creation: Sep. 1, 2023) is hereby incorporated by reference in its entirety.

### BACKGROUND

Background K⁺ currents produced by two-pore domain (K_{2P}) potassium channels play a fundamental role in the stabilization of membrane potential and regulation of cellular excitability (1-3). Consequently, K_{2P}s are important to a variety of physiological processes including action potential propagation (4,5), pain sensation (6-8), sleep (9,10), and intraocular pressure (11), as well as pathological conditions such as migraine (12), depression (13), pulmonary hypertension (14), and lung injury (15). K_{2P}s are highly regulated by both physical gating mechanisms (pH (3,16,17), stretch (18), and temperature (19)) and small molecules (signaling lipids, anesthetics, exogenous chemicals (20-23)). K_{2P} pharmacology, however, remains relatively underdeveloped, with the vast majority of K_{2P} modulators lacking well defined mechanisms of action and structurally defined binding sites. Further, most modulators, particularly activators, possess modest EC50s (μM range) and limited selectivity. Provided herein, for example, are chemogenetic compounds capable of selectively activating K_{2P} channels through the selective engagement of an engineered cysteine residue at the K_{2P} modulator pocket.

### BRIEF SUMMARY

In an aspect provided herein is a compound, or a pharmaceutically acceptable salt thereof, having Formula I.

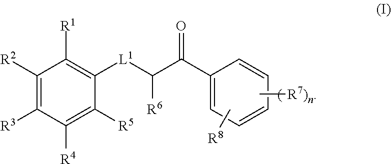

L¹ is a bond, substituted or unsubstituted alkylene or substituted or unsubstituted heteroalkylene.

R¹ is independently is hydrogen, halogen, —CX¹₃, —CHX¹₂, —CH₂X¹, —OCX¹₃, —OCHX¹₂, —OCH₂X¹, —CN, —N₃, —SOᵥ₁R^{1A}, —SOᵥ₁NR^{1B}R^{1C}, —NHNR^{1B}R^{1C}, —ONR^{1B}R^{1C}, —NHC(O)NHNR^{1B}R^{1C}, NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is independently hydrogen, halogen, —CX²₃, —CHX²₂, —CH₂X², —OCX²₃, —OCHX²₂, —OCH₂X², —CN, —N₃, —SOₙ₂R^{2A}, —SOᵥ₂NR^{2B}R^{2C}, —N^{2B}R^{2C}, —ONR^{2B}R^{2C}, —NHC(O)NHNR^{2B}R^{2C}, —NHC(O)NR^{2B}R^{2C}, —N(O)ₘ₂, NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is independently hydrogen, halogen, —CX³₃, —CHX³₂, —CH₂X³, —OCX³₃, —OCHX³₂, —OCH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is independently hydrogen, halogen, —CX⁴₃, —CHX⁴₂, —CH₂X⁴, —OCX⁴₃, —OCHX⁴₂, —OCH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is independently hydrogen, halogen, —CX⁵₃, —CHX⁵₂, —CH₂X⁵, —OCX⁵₃, —OCHX⁵₂, —OCH₂X⁵, —CN, —N₃, —SOₙ₅R^{5A}, —SOᵥ₅NR^{5B}R^{5C}, —N—NR^{SB}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ is independently a substituted or unsubstituted alkyl or substituted or unsubstituted heteroalkyl. R⁶ is optionally joined with L¹ to form a substituted or unsubstituted heterocycloalkyl.

R⁷ is independently halogen, —CX⁷₃, —CHX⁷₂, —CH₂X⁷, —OCX⁷₃, —OCHX⁷₂, —OCH₂X⁷, —CN, —N₃, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, NHC(O)NR^{7B}R^{7C}, —N(O)ₘ₇, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, —NR^{7B}OR^{7D}, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸ is a cysteine binding moiety or a serine binding moiety.

R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{7A}, R^{7B}, R^{7C}, and R^{7D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; and R^{7A} and R^{7B} substituents bonded to the same nitrogen atom may optionally be joined to form a substituted or unsubstituted heterocycloalkyl or substituted or unsubstituted heteroaryl.

X¹, X², X³, X⁴, X⁵, and X⁷ are independently —F, —Cl, —Br, or —I.

The symbols n1, n2, n3, n4, n4, and n7 are independently an integer from 0 to 4. The symbols m1, m2, m3, m4, m5, m7, v1, v2, v3, v4, v5, and v7 are independently 1 or 2. The symbol n is an integer from 0 to 3.

In an aspect is provided a pharmaceutical composition including a compound described herein, or a pharmaceutically acceptable salt thereof, and a pharmaceutically acceptable excipient.

In embodiments, provided herein is a TREK family protein comprising a cysteine residue at an amino position corresponding to position 131 of the TREK-1 protein, or a homolog thereof In embodiments, the TREK family protein is a TREK-1 protein, a TREK-2 protein, a TRAKK protein, or any homolog thereof.

In additional embodiments, provided herein are methods of treating a disease or adverse condition related to low TREK family protein activity in a subject in need thereof. In embodiments, the disclosed methods comprise administering to the subject effective amounts of a nucleic acid encoding a TREK family protein or a homolog thereof, and a TREK family protein agonist. In embodiments, the TREK family protein agonist comprises a cysteine binding moiety. In embodiments, the cysteine binding moiety is capable of covalently binding the TREK family protein or homolog thereof at the cysteine residue.

In embodiments, the disease or adverse condition comprises, but is not limited to, chronic pain, nerve injury, lack of sleep, high intraocular pressure, headache, depression, pulmonary hypertension, lung injury, and decompression sickness. In embodiments, the nerve injury is an injury of the dorsal ganglion nerve.

In embodiments, provided herein are methods of treating a disease or adverse condition related to low TREK family protein activity in a subject in need thereof. In embodiments, the disclosed methods comprise administering to the subject a gene editing system capable of mutating a TREK family protein to comprise a cysteine residue at an amino position corresponding to position 131 of the TREK-1 protein or homolog thereof, and a TREK family protein agonist. In embodiments, the TREK family protein agonist comprises a cysteine binding moiety. In embodiments, the cysteine binding moiety is capable of covalently binding the TREK family protein or homolog thereof at the cysteine residue. In embodiments, the gene editing system is a CRISPR/Cas9 gene editing system.

In embodiments, provided herein are methods of increasing TREK family protein activity in a tissue. In embodiments, the disclosed methods comprise administering to the tissue effective amounts of a nucleic acid encoding a TREK family protein or homolog thereof, and a TREK family protein agonist. In embodiments, the TREK family protein agonist comprises a cysteine binding moiety. In embodiments, the cysteine binding moiety is capable of covalently binding the TREK family protein or homolog thereof at the cysteine residue. In embodiments, the nucleic acid is within a viral particle. Exemplary tissues include, but are not limited to, the brain, the heart, the eye, a smooth muscle tissue, endocrine pancreas, the prostate, and sensory organs.

### BRIEF DESCRIPTION OF THE DRAWINGS

FIGS. 1A-1F show TREK-1 activation with the compound ML336. FIG. 1A: Chemical structures of K_{2P} modulator pocket activators. FIG. 1B: Exemplar 2.9 Å resolution 2Fo-Fc electron density (1σ) showing the K_{2P}2.1 (TREK 1):ML336 covalent complex. Select residues are indicated. K_{2P}2.1 side chains are highlighted. ML336 is indicated. Covalent link is indicated. P1 and M4 helices are labeled. FIG. 1C: Two-electrode voltage clamp (TEVC) traces from Xenopus oocytes expressing K_{2P}2.1 (TREK-1) and activated by ML335 and ML336. FIG. 1D: Timecourse of fold-activation of K_{2P}2.1 (TREK-1) currents at 0 mV following addition of either ML335 or ML336. FIG. 1E: Exemplar TEVC traces from Xenopus oocytes expressing K_{2P}2.1 (TREK-1) S131A and activated by 20 μM ML335 and 20 μM ML336. FIG. 1F: Fold-activation of K_{2P}2.1 (TREK-1) following application and washout of ML335 and ML336 (n=3-5). For each set of 4 bars, the first bar is 20 μM ML335, the second bar is Washout, the third bar is 20 μM ML336, and the fourth bar is Washout. Data are presented as mean±S.E.M.

FIGS. 2A-2L show TREK-1^{CG}* Activation in Oocytes and HEK Cells. FIGS. 2A-2B: Two-electrode voltage clamp (TEVC) traces from Xenopus oocytes expressing K_{2P}2.1 (TREK-1) or TREK-1^{CG}* and activated by 20 μM CAT335. FIG. 2C: Fold-activation of oocytes expressing K_{2P}2.1 (TREK-1) or TREK-1^{CG}* following application of 20 μM ML335 (first bar in each set) or 20 μM CAT335 (second bar in each set) (n=9-10, error bars represent S.E.M.). FIG. 2D: Representative time courses of TREK-1^{CG}* activation in oocytes with 20 μM ML335 and 20 μM CAT335. FIGS. 2E-2F: Representative whole-cell currents from HEK293 cells transfected with K_{2P}2.1 (TREK-1) or TREK-1^{CG}* in the presence of 20 μM ML335 or 20 μM CAT335. FIG. 2G: Fold-activation of HEK293 cells expressing K_{2P}2.1 (TREK-1) or TREK-1^{CG}* following application of 20 μM ML335 (first bar in each set) or 20 M CAT335 (second bar in each set) (n=5-8, error bars represent S.E.M.). FIG. 2H: Representative time course of TREK-1^{CG}* activation in HEK293 cells with 20 μM ML335 and 20 μM CAT335. FIG. 2I: TEVC traces from oocytes expressing TREK-1^{CG}* and activated by 20 μM CAT335a. FIG. 2J: Fold-activation of oocytes expressing TREK-1^{CG}* following application of CAT335a. (n=8, error bars represent S.E.M.) FIG. 2K: TEVC traces from oocytes expressing TREK-1^{CG}* and activated by 20 μM CAT335b. FIG. 2L: Fold-activation of oocytes expressing TREK-1^{CG}* following application of CAT335b (n=6-9, error bars represent S.E.M.).

FIGS. 3A-3E show TREK-1^{CG}* crystal structures.

FIGS. 4A-4F show K_{2P}2.1 (TREK-1) Tandems TEVC. FIG. 4A: Schematic representation of the four tandem constructs tested. FIGS. 4B-4E: Two-electrode voltage clamp (TEVC) traces from Xenopus oocytes expressing tandem K_{2P}2.1 (TREK-1) channels activated by the sequential addition of 20 μM ML335, 20 μM CAT335, and 20 μM BL-1249. Curves from top to bottom: FIG. 4B: BL-1249, Washout (BL-1249), ML335, Washout (ML335), ML337, Washout (ML337), Initial; FIG. 4C: BL-1249, Washout (BL-1249), Washout (ML337), ML337, ML335, Washout (ML335), Initial; FIG. 4D: BL-1249, Washout (BL-1249), Washout (ML337), ML337, ML335, Washout (ML335), Initial; FIG. 4E: BL-1249, Washout (BL-1249), Washout (ML337), ML337, ML335, Washout (ML335), Initial. FIG. 4F: Fold-activation of Xenopus oocytes expressing tandem K_{2P}2.1(TREK-1) channels following the sequential application of 20 μM ML335, 20 μM CAT335, and 20 μM BL-1249 (n=10, error bars are S.E.M.). Significance was measured in GraphPad Prism using Brown-Forsythe and Welch one-way ANOVA with Dunnett's T3 multiple comparisons test (n.s. p>0.1234; * p<0.0332; **p<0.0021; *** p<0.0002; **** p<0.0001).

FIGS. 5A-5R show K_{2P}2.1 (TREK-1) Tandems Single Channel Experiments. Single channel recordings of K_{2P}2.1 (TREK-1) tandem channels from HEK cells in cell-attached mode. FIGS. 5A-5D: Open probability (Po) of tandem channels without activator or after application of 20 μM CAT335 or BL-1249 (n=4-11, error bars S.E.M.). FIGS. 5E-5L: Single channel conductances measured at −100 mV or +50 mV holding potential without activator or 20 μM CAT335 (n=6-13, error bars S.E.M.). FIGS. 5M-5R: Exemplary single channel recordings from tandem channels without activator (control) or after treatment with M CAT335. Significance was measured in GraphPad Prism using Brown-Forsythe and Welch one-way ANOVA with Dunnett's T3 multiple comparisons test (n.s. p>0.1234; * p<0.0332; **p<0.0021; *** p<0.0002; **** p<0.0001).

FIGS. 6A-6E show TEVC with attenuated K_{2P}2.1 (TREK-1) channels. FIG. 6A: Basal currents at 0 mV of K_{2P}2.1 (TREK-1) mutants expressed in Xenopus oocytes. FIG. 6B: Fold-activation of K_{2P} channels measured in Xenopus oocytes at 0 mV in response to the sequential addition of 20 μM ML335 (first bar in each set), 20 μM CAT335 (third bar in each set) and 20 μM BL-1249 (fifth bar in each set) (n=5-8, data represented as mean S.E.M.).

FIG. 6C: Exemplary current traces for K_{2P}2. iv (TREK-1) mutants following the sequential application of 20 μM ML335, 20 μM CAT335, and 20 μM BL-1249. FIG. 6D: Timecourses of K_{2P}2.1 (TREK-1) currents at 0 mV when expressed in Xenopus oocytes and subjected to M ML335, 20 μM CAT335, and 20 μM BL-1249. Activators were applied during the highlighted windows, between which ND96 buffer was perfused. Solid lines represent mean values, dotted lines represent S.E.M. (n=3-6). FIG. 6E: Timecourses of K_{2P}2.1(TREK-1) currents at 0 mV when expressed in Xenopus oocytes and subjected to 20 μM BL-1249, then either 20 μM ML335 or 20 μM CAT335, followed by 20 μM BL-1249. Activators were applied during the highlighted windows, between which ND96 buffer was perfused. Solid lines represent mean values, dotted lines represent S.E.M. (n=3-6).

FIGS. 7A-7C show ChemoKlamp Activation with CAT335 in HEK cells. FIG. 7A: Representative gap-free, I=0 recordings from HEK293 with either no exogenous K_{2P} expression (control), or expressing TREK-1^{CG}* (CG*) or A286F TREK-1^{CG}* (ChemoKlamp). Grey box indicates application of 20 μM CAT335 to cells, white represents perfusion with ND96 buffer, Vk depicts the reversal potential of K⁺. FIG. 7B: Change in Vm before (first set of bars in each set) and after (second set of bars in each set) application of 20 μM CAT335 (n=8-13, error bars are S.E.M). FIG. 7C: Whole-cell currents of HEK293 cells expressing K_{2P}2.1 (TREK-1), TREK-1^{CG}* or ChemoKlamp before (first set of bars in each set) and after application of 20 μM CAT335 (second set of bars in each set) (n=7-13, error bars represent S.E.M.).

FIGS. 8A-8C show K_{2P}10.1 (TREK-2)CG* and K_{2P}4.1(TRAAK)CG*. Comparison of K_{2P}2.1 (TREK-1), K_{2P}10.1(TREK-2) and K_{2P}4.1(TRAAK) sequences at the (FIG. 8A) P1 and (FIG. 8B) M4 face of the K_{2P} modulator pocket. Protein secondary structure is marked above the sequences. CG* mutation site is marked with an arrow. Conserved positions are shaded in grey. Residues involved in direct interactions within the modulator pocket are indicated in light grey text. Sequence and identifiers are as follows: FIG. 8A: K_{2P}2.1 (TREK-1) NP_034737.2; K₂P10.1(TREK-2) NP_001303594.1; K₂P4.1(TRAAK) Mus musculus NP_032457.1; K2P4.1(TRAAK) Homo sapiens NP_001304019.1; K_{2P}18.1(TRESK) Mus musculus NP_997144.1. Sequences shown in FIG. 8A: K_{2P}2.1 (mTREK-1): PLGNSSNQVSHWDLGSSFFFAGTVITTIGFGNIS (SEQ ID NO: 13); K₂P10.1 (mTREK-2): PVGNSSNSSSHWDLGSAFFFAGTVITTIGYGNIA (SEQ ID NO: 14); K₂P4.1 (mTRAAK): PETSWTNSSNHSSAWNLGSAFFFSGTIITTIGYGNIV (SEQ ID NO: 15); K₂P4.1 (hTRAAK): PETNSTSNSSHSAWDLGSAFFFSGTIITTIGYGNVA (SEQ ID NO: 16); K₂P18.1 (mTRESK): LKPQWLKAPQDWSFLSALFFCCTVFSTVGYGHMY (SEQ ID NO: 17). Sequences shown in FIG. 8B: K_{2P}2.1 (mTREK-1): YFVVITLTTIGFGDYVAGGSDIEYLDFYKPVVWFWI (residues 243-278 of SEQ ID NO: 1); K₂P10.1 (mTREK-2): YFVVVTLTTVGFGDFVAGGNAGINYREWYKPLVWFWI (residues 268-304 of SEQ ID NO: 5); K₂P4.1 (mTRAAK): YFVIVTLTTVGFGDYVPGDGTGQNSPAYQPLVWFWI (residues 205-240 of SEQ ID NO: 9); K₂P4.1 (hTRAAK): YFVIVTLTTVGFGDYVAGADPRQDSPAYQPLVWFWI (residues 204-239 of SEQ ID NO: 11); K_{2P}18.1 (mTRESK): YFCFVTLTTIGFGDIVLVHPHFFLFFSIYI (SEQ ID NO: 18). FIG. 8C: Fold-activation of K_{2P} channels measured in Xenopus oocytes at 0 mV in response to 20 μM ML335 (first bar in each set) and 20 μM CAT335 (third bar in each set) (n=9-11, data represented as mean S.E.M.).

FIGS. 9A-9D show ML336 with S131A K_{2P}2.1 (TREK-1) and S131C K_{2P}2.1(TREK-1). FIG. 9A: Exemplary 2.9 Å resolution 2Fo-Fc electron density (1σ) showing the K₂P2.1 (TREK 1):ML336 covalent complex. Select residues are indicated. K_{2P}2.1 sidechains is indicated. ML336 is indicated. Covalent link is indicated. P1 and M4 helices are labeled. FIG. 9B: Comparison of the interactions made by K_{2P}2.1 (TREK-1) S131 hydroxyl with either ML335 or ML336. FIG. 9C: Two-electrode voltage clamp (TEVC) traces from Xenopus oocytes expressing K_{2P}2.1 (TREK-1) S131C and activated with 5 μM ML336 for 2 minutes. FIG. 9D: Fold-activation of wt K_{2P}2.1 (TREK-1) and K_{2P}2.1 (TREK-1) S131C currents at 0 mV following application of 5 μM ML336 for 2 min and after washout (2 minutes buffer) (n=13-16, error bars S.E.M.). Significance measured by two-sided, unpaired, unequal variances t-test where n.s.=p>0.05, *=p<0.05.

FIGS. 10A-10K show TREK-1^{CG}* Characterization. FIG. 10A: EC50 for K_{2P}2.1 (TREK-1) and TREK-1^{CG}*. Currents were measured by TEVC at 0 mV, normalized to initial currents before ML335 application (n=16-19, error bars are S.E.M). FIG. 10B: Temperature response of K_{2P}2.1 (TREK-1) and TREK-1^{CG}* measured by TEVC. Currents were normalized to each channels current at 20° C. (n=10, error bars are S.E.M). FIG. 10C: pH dependence of wt K_{2P}2.1(TREK-1) and TREK-1^{CG}* channel activities at 0 mV measured by TEVC. Wt K_{2P}2.1 (TREK-1) is inhibited at acidic pH (pK=8.3, Hillslope=1.7), while TREK-1^{CG}* was activated at acidic pH (pK=7.44, Hillslope=−2.7). Currents were normalized to the highest mean current for each channel (n=9-21, error bars are S.E.M.).

FIGS. 10D-10K: Representative time courses of K_{2P}2.1 (TREK-1) or TREK-1^{CG}* activation in oocytes with ML335 and CAT335 at 5 M or 20 μM concentrations.

FIGS. 11A-11F show CAT335c and CAT335 Incubation Experiments. FIGS. 11A-11B: Two-electrode voltage clamp (TEVC) traces from Xenopus oocytes expressing K_{2P}2.1 (TREK-1) or TREK-1^{CG}* and activated with 20 μM CAT335c. FIG. 11C: Fold-activation of wt K_{2P}2.1 (TREK-1) and TREK-1^{CG}* currents at 0 mV following application of 20 μM CAT335c for 2 min and after washout (2 minutes buffer) (n=9, error bars are S.E.M.). FIGS. 11D-11F: Currents at 0 mV recorded from oocytes expressing either wild-type K_{2P}2.1 (TREK-1) or TREK-1^{CG}* following 1 hour incubation with covalent activators at varying concentrations. After recording the initial currents following incubation, oocytes were treated with 20 μM ML335 to determine the extent of channel activation. Following ML335 activation, the oocytes were perfused with buffer for 3 minutes to measure the extent of washout.

FIGS. 12A-12D show the ML335 complex (FIG. 12A). FIG. 12B illustrates how both the CAT335 and CAT335a structures showed continuous density that bridged the maleimide moiety and S131C, indicative of the formation of a covalent adduct.

FIGS. 13A-13D show co-application of BL-1249 and ML335/CAT335. Activation of K_{2P}2.1(TREK-1) following co-application of modulator pocket ligands (ML335 or CAT335) and fenestration site ligand BL-1249. FIG. 13A: Two-electrode voltage clamp (TEVC) traces from Xenopus oocytes expressing K_{2P}2.1(TREK-1) mutants lacking the CG* mutation and activated by the simultaneous addition of ML335 and BL-1249. FIG. 13B: TEVC traces from Xenopus oocytes expressing TREK-1^{CG}* mutants and activated by the simultaneous addition of CAT335 and BL-1249. FIG. 13C: Timecourses of K_{2P}2.1 (TREK-1), A286F K_{2P}2.1(TREK-1) and G171F K_{2P}2.1(TREK-1) fold-activation at 0 mV when exposed to ML335 and BL-1249 simultaneously. FIG. 13D: Timecourses of TREK-1^{CG}* A286F TREK-1^{CG}* and G171F TREK-1^{CG}* fold-activation at 0 mV when exposed to CAT335 and BL-1249 simultaneously.

FIGS. 14A-14B show K_{2P} Modulator Pocket Cation-n Effect on CAT335 activation. Fold-activation of Xenopus oocytes expressing K_{2P} channels following application of 20 μM ML335 (first bar in each set) or 20 μM CAT335 (third bar in each set) (n=3-10, data represented as mean S.E.M.).

FIG. 15 shows synthesis of ML336 and CAT335 derivatives.

FIGS. 16A-16H. CATKLAMP activation with CAT335 hyperpolarizes cells. FIG. 16A: Exemplar gap-free, I=0 recordings untransfected HEK293 (control), or HEK293 cells expressing TREK-1^{CG}*, or TREK-1^{CG}* A286F. Grey box indicates application of 20 μM CAT335 to cells, V indicates the K⁺ reversal potential. FIG. 16B: Change in Vm before (first bar in each set) and after (second bar in each set) application of 20 μM CAT335 (n=8-13) for control, TREK-1^{CG}* (CG*), and TREK-1^{CG}* A286F (CG* A286F) cells. FIG. 16C: Whole-cell currents before (first bar in each set) and after application of 20 μM CAT335 (second bar in each set) (n=7-13) for the cells from FIG. 16B. Error bars represent S.E.M. Significance using either paired t-tests (for before and after drug application) or unpaired t-tests (comparing initial currents across different receptors) is indicated. n.s. p>0.12; * p<0.033; **p<0.0021; *** p<0.0002; **** p<0.0001. FIG. 16D: Exemplar mouse primary hippocampal neuron expressing CG* A286F. FIG. 16E: Exemplar neuron responses to current injection to is current steps from −80 pA to 200 pA for wild type and CG* A286F expressing neurons before (top left and bottom left panels, respectively) and after 20 μM CAT335 application (top right and bottom right panels, respectively). FIG. 16F: RMP changes for the indicated neurons showing absolute (left panel) and normalized changes per neuron (right panel). FIG. 16G: Input resistance changes for the indicated neurons showing absolute values (left panel) and normalized changes per neuron (right panel). FIG. 16H: CAT335 effects on neuronal firing frequency in response 1 s current steps for wild type (top panel) and CG* A286F neurons (bottom panel).

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “˜” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

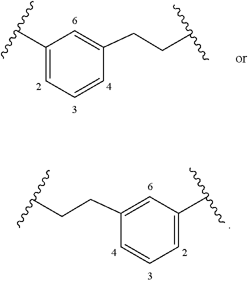 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)=NR″″, —NRC(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₅ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R^{S} may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2}, . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

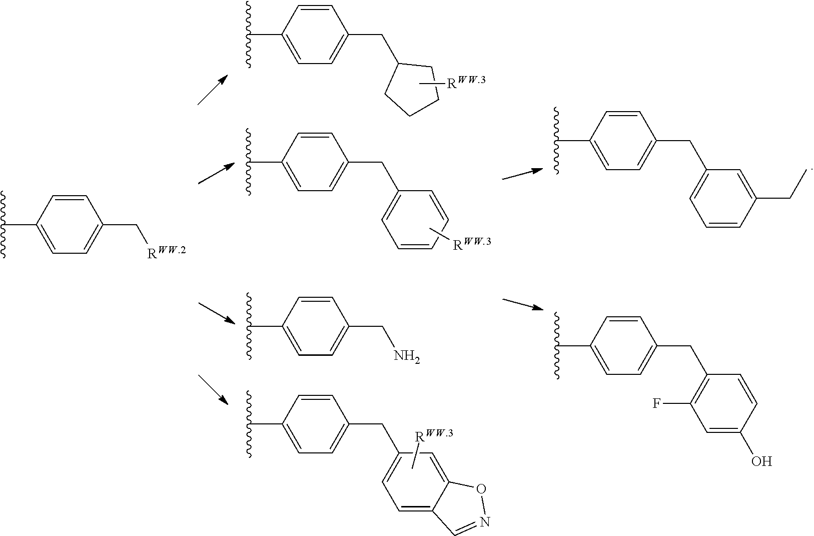

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X i is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂—CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted.

Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is no prophylactic treatment.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, the agonist is administered to a subject with a TREK family protein. In embodiments, the agonist increases expression or activity of a TREK family protein in the subject by at least 10%, 20%, 30%, 40%, 50%, 60%, 70%, 80%, 90%, 95%, 96%, 97%, 98%, or 99% in comparison to the absence of administering a TREK family protein and agonist in a control subject. In embodiments, the agonist increases expression or activity of TREK-1 in the subject by at least 10%, 20%, 30%, 40%, 50%, 60%, 70%, 80%, 90%, 95%, 96%, 97%, 98%, or 99% in comparison to the absence of administering TREK-1 and agonist in a control subject. In embodiments, the agonist increases expression or activity of TREK-2 by at least 10%, 20%, 30%, 40%, 50%, 60%, 70%, 80%, 90%, 95%, 96%, 97%, 98%, or 99% in comparison to the absence of administering TREK-2 and agonist in a control subject. In embodiments, the agonist increases expression or activity of TRAAK by at least 10%, 20%, 30%, 40%, 50%, 60%, 70%, 80%, 90%, 95%, 96%, 97%, 98%, or 99% in comparison to the absence of administering TRAAK and agonist in a control subject.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist. As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is pain.

As used herein, the term “a disease or disorder related to low TREK family protein activity” refers to a disease or disorder related to a TREK family protein activity below the level typically seen in a healthy subject or in subjects without the TREK family disease or disorder.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In embodiments, the TREK family protein and the TREK agonist are administered simultaneously. In embodiments, the TREK family protein and the TREK agonist are administered approximately simultaneously. In embodiments, the TREK family protein and the TREK agonist are administered sequentially.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., pain) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., pain) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid-like compounds that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid-like compounds” refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid-like compounds which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a TREK family protein (or homolog thereof) includes a cysteine corresponding to position 131 of TREK-1 when the selected cysteine residue occupies the same essential spatial or other structural relationship as amino acid position 131 of SEQ ID NO: 1. In embodiments, a TREK family protein (or homolog thereof) includes a cysteine corresponding to position 131 of TREK-1 when the selected cysteine residue occupies the same essential spatial or other structural relationship as amino acid position 111 of SEQ ID NO: 2. In embodiments, a TREK family protein (or homolog thereof) includes a cysteine corresponding to position 131 of TREK-1 when the selected cysteine residue occupies the same essential spatial or other structural relationship as amino acid position 85 of SEQ ID NO: 3. In embodiments, a TREK family protein (or homolog thereof) includes a cysteine corresponding to position 131 of TREK-1 when the selected cysteine residue occupies the same essential spatial or other structural relationship as amino acid position 142 of SEQ ID NO: 4. In embodiments, a TREK family protein (or homolog thereof) includes a cysteine corresponding to position 131 of TREK-1 when the selected cysteine residue occupies the same essential spatial or other structural relationship as amino acid position 156 of SEQ ID NO: 5. In embodiments, a TREK family protein (or homolog thereof) includes a cysteine corresponding to position 131 of TREK-1 when the selected cysteine residue occupies the same essential spatial or other structural relationship as amino acid position 97 of SEQ ID NO: 6. In embodiments, a TREK family protein (or homolog thereof) includes a cysteine corresponding to position 131 of TREK-1 when the selected cysteine residue occupies the same essential spatial or other structural relationship as amino acid position 161 of SEQ ID NO: 7. In embodiments, a TREK family protein (or homolog thereof) includes a cysteine corresponding to position 131 of TREK-1 when the selected cysteine residue occupies the same essential spatial or other structural relationship as amino acid position 95 of SEQ ID NO: 8. In embodiments, a TREK family protein (or homolog thereof) includes a cysteine corresponding to position 131 of TREK-1 when the selected cysteine residue occupies the same essential spatial or other structural relationship as amino acid position 93 of SEQ ID NO: 9. In embodiments, a TREK family protein (or homolog thereof) includes a cysteine corresponding to position 131 of TREK-1 when the selected cysteine residue occupies the same essential spatial or other structural relationship as amino acid position 93 of SEQ ID NO: 10. In embodiments, a TREK family protein (or homolog thereof) includes a cysteine corresponding to position 131 of TREK-1 when the selected cysteine residue occupies the same essential spatial or other structural relationship as amino acid position 92 of SEQ ID NO: 11. In embodiments, a TREK family protein (or homolog thereof) includes a cysteine corresponding to position 131 of TREK-1 when the selected cysteine residue occupies the same essential spatial or other structural relationship as amino acid position 92 of SEQ ID NO: 12.

In some embodiments, where a selected protein is aligned for maximum homology with the TREK-1 protein, the position in the aligned selected protein aligning with position 131 is said to correspond to position 131 of TREK-1. Instead of a primary sequence alignment, a three dimensional structural alignment can also be used, e.g., where the structure of the selected protein is aligned for maximum correspondence with the TREK-1 protein and the overall structures compared. In this case, an amino acid that occupies the same essential position as position 131 in the structural model is said to correspond to the position 131 of TREK-1.

As used herein, the term “cysteine residue” refers to a cysteine located on a protein or polypeptide. Cysteine contains a reactive sulph-hydryl group. In embodiments, the cysteine residue has the ability to react with another cysteine to form a disulfide bond. In embodiments, the disulfide bond confers stability to the protein. In embodiments, a TREK family protein is modified to include a cysteine residue not found a natural TREK family protein. In embodiments, the TREK family protein is a TREK-1 protein, a TREK-2 protein or a TRAAK protein. In embodiments, the TREK family protein is a TREK-1 protein. In embodiments, the TREK family protein is a TREK-2 protein. In embodiments, the TREK family protein is a TRAAK protein.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “selective” or “selectivity” or the like in reference to a compound or agent refers to the compound's or agent's ability to cause an increase or decrease in activity of a particular molecular target, such as a TREK family protein, preferentially over one or more different molecular targets. In embodiments, a “TREK family protein-selective compound” refers to a compound (e.g., compound described herein) having selectivity towards a TREK family protein as provided herein.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target. In embodiments, a TREK family protein agonist comprises a cysteine binding moiety capable of covalently binding a TREK family protein at a cysteine residue corresponding to position 131 of a TREK-1 protein. In embodiments, where the cysteine binding moiety is capable of binding the target TREK family protein, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The terms “patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. In embodiments, the administering includes simultaneous or sequential administration of another active agent in addition to the recited active agents.

As used herein, the term “co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

As used herein, the terms “specific”, “specifically”, “specificity”, or the like of a compound refers to the compound's ability to cause a particular action, such as inhibition, to a particular molecular target with minimal or no action to other proteins in the cell.

“Nucleic acid” refers to deoxyribonucleotides or ribonucleotides and polymers thereof in either single- or double-stranded forms, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

The term “nucleotide” typically refers to a compound containing a nucleoside or a nucleoside analogue and at least one phosphate group or a modified phosphate group linked to it by a covalent bond. Exemplary covalent bonds include, without limitation, an ester bond between the 3′, 2′ or 5′ hydroxyl group of a nucleoside and a phosphate group.

The term “nucleoside” refers to a compound containing a sugar part and a nucleobase, e.g., a pyrimidine or purine base. Exemplary sugars include, without limitation, ribose, 2-deoxyribose, arabinose and the like. Exemplary nucleobases include, without limitation, thymine, uracil, cytosine, adenine, guanine.

The term “nucleoside analogue” may refer to a nucleoside any part of which is replaced by a chemical group of any nature. Exemplary nucleoside analogues include, without limitation, 2′-substituted nucleosides such as 2′-fluoro, 2-deoxy, 2′-O-methyl, 2′-O—P-methoxyethyl, 2′-O-allylriboribonucleosides, 2′-amino, locked nucleic acid (LNA) monomers and the like. The term “nucleoside analogue” may also refer to a nucleoside in which the sugar or base part is modified, e.g., with a non-naturally occurring modification. Exemplary nucleoside analogues in which the sugar part is replaced with another cyclic structure include, without limitation, monomeric units of morpholinos (PMO) and tricyclo-DNA. Exemplary nucleoside analogues in which the sugar part is replaced with an acyclic structure include, without limitation, monomeric units of peptide nucleic acids (PNA) and glycerol nucleic acids (GNA). Suitably, nucleoside analogues may include nucleoside analogues in which the sugar part is replaced by a morpholine ring.

Nucleoside analogues may include deoxyadenosine analogues, adenosine analogues, deoxycytidine analogues, cytidine analogues, deoxyguanosine analogues, guanosine analogues, thymidine analogues, 5-methyluridine analogues, deoxyuridine analogues, or uridine analogues. Examples of deoxyadenosine analogues include didanosine (2′, 3′-dideoxyinosine) and vidarabine (9-D-arabinofuranosyladenine), fludarabine, pentostatin, cladribine. Examples of adenosine analogues include BCX4430 (Immucillin-A). Examples of cytidine analogues include gemcitabine, 5-aza-2′-deoxycytidine, cytarabine. Examples of deoxycytidine analogues include cytarabine, emtricitabine, lamivudine, zalcitabine. Examples of guanosine and deoxyguanosine analogues include abacavir, acyclovir, entecavir. Examples of thymidine and 5-methyluridine analogues include stavudine, telbivudine, zidovudine. Examples of deoxyuridine analogues include idoxuridine and trifluridine.

The terms “purine analogue” or “pyrimdine analogue” refers to modifications, optionally non-naturally occurring modifications, in the nucleobase, for example hypoxanthine, xanthine, 2-aminopurine, 2,6-diaminopurine, 6-azauracil, 5-methylcytosine, 4-fluorouracil, 5-fluoruracil, 5-chlorouracil, 5-bromouracil, 5-iodouracil, 5-trifluoromethyluracil, 5-fluorocytosine, 5-chlorocytosine, 5-bromocytosine, 5-iodocytosine, 5-propynyluracil, 5-propynylcytosine, 7-deazaadenine, 7-deazaguanine, 7-deaza-8-azaadenine, 7-deaza-8-azaguanine, isocytosine, isoguanine, mercaptopurine, thioguanine. Exemplary pyrimidine analogues include, without limitation, 5-position substituted pyrimidines, e.g. substitution with 5-halo, 5′-fluoro. Examples of purine analogues include, without limitation, 6- or 8-position substituted purines, e.g., substitution with 5-halo, 5′-fluoro.

The term “phosphate group” as used herein refers to phosphoric acid H3PO4 wherein any hydrogen atoms are replaced by one, two or three organic radicals to give a phosphoester, phosphodiester, or phosphotriester, respectively. Oligonucleotides may be linked by phosphodiester, phosphorothioate or phosphorodithioate linkages.

In structures of this type, it will be appreciated that the labels 3′ and 5′, as applied to conventional sugar chemistry, apply by analogy.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of nucleic acid molecules may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection”, “transduction”, “transfecting” or “transducing” are used interchangeably throughout and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified (e.g., labeled).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences disclosed herein or individual domains of the polypeptides disclosed herein), when compared and aligned for maximum correspondence over a comparison window, or designated region as measured using one of the following sequence comparison algorithms or by manual alignment and visual inspection. Such sequences are then considered to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Any methods of alignment of sequences for comparison well known in the art are contemplated. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

Example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically or substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

An “antisense nucleic acid” as referred to herein is a nucleic acid (e.g., DNA or RNA molecule) that is complementary to at least a portion of a specific target nucleic acid (e.g., an mRNA translatable into a protein). In embodiments, the antisense nucleic acid is capable of reducing transcription of the target nucleic acid (e.g., mRNA from DNA) or reducing the translation or the amount of the target nucleic acid (e.g. mRNA) or altering transcript splicing (e.g. single stranded morpholino oligo). See, e.g., Weintraub, Scientific American, 262:40 (1990). In embodiments, synthetic antisense nucleic acids (e.g., oligonucleotides) are between 15 and 25 bases in length. In embodiments, the antisense nucleic acids are capable of hybridizing to (e.g., selectively hybridizing to) a target nucleic acid (e.g., target mRNA). In embodiments, the antisense nucleic acid hybridizes to the target nucleic acid sequence (e.g., mRNA) under stringent hybridization conditions. In embodiments, the antisense nucleic acid hybridizes to the target nucleic acid (e.g., mRNA) under moderately stringent hybridization conditions. In embodiments, the antisense nucleic acids may comprise naturally occurring nucleotides or modified nucleotides such as, e.g., phosphorothioate, methylphosphonate, and -anomeric sugar-phosphate, backbonemodified nucleotides. In the cell, the antisense nucleic acids may hybridize to the corresponding mRNA, forming a double-stranded molecule. In embodiments, the antisense nucleic acids interfere with the translation of the mRNA, since the cell will not translate an mRNA that is double-stranded. The use of antisense methods to inhibit the in vitro translation of genes is well known in the art (Marcus-Sakura, Anal. Biochem. 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogues), short hairpin RNAs (shRNA), micro RNAs (miRNA), saRNAs (small activating RNAs) and small nucleolar RNAs (snoRNA) or certain of their derivatives or pre-cursors.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when present in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

A “saRNA,” or “small activating RNA” as provided herein refers to a nucleic acid that forms a double stranded RNA, which double stranded RNA has the ability to increase or activate expression of a gene or target gene when present in the same cell as the gene or target gene. The complementary portions of the nucleic acid that hybridize to form the double stranded molecule typically have substantial or complete identity. In one embodiment, a saRNA is a nucleic acid that has substantial or complete identity to a target gene and forms a double stranded saRNA. Typically, the nucleic acid is at least about 15-50 nucleotides in length (e.g., each complementary sequence of the double stranded saRNA is 15-50 nucleotides in length, and the double stranded saRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

A “shRNA,” “short hairpin RNA,” or “small hairpin RNA” as provided herein refers to an RNA molecule including a hairpin turn that has the ability to reduce or inhibit expression of a target gene or target nucleic acid when expressed in the same cell as the target gene or target nucleic acid. shRNA expression in a cell may be accomplished by delivery of the shRNA the cell using a plasmid or vector. Typically, the shRNA is cleaved by an enzyme (i.e. Dicer) to produce an siRNA product. The siRNA may then associate with RISC, thereby allowing target recognition.

A “PIWI-interacting RNA” or “piRNA” refers to a type of small non-coding RNA (sncRNA), which is 26-31 nucleotides in length and binds to PIWI proteins. In embodiments, piRNAs are independent of the Dicer enzyme and are produced by a single-stranded precursor. In embodiments, piRNA clusters in somatic cells are unidirectional. In embodiments, the majority of germline piRNA clusters are dual-stranded. In embodiments, most mature primary piRNAs contain uridine at the 5′ end, and the 3′ ends of piRNAs are uniquely methylated 2-OH structures. In embodiments, piRNAs are unevenly distributed among various genomic sequences, including exons, introns, and repeat sequences. In embodiments, piRNAs are derived from transposons and from flanking genomic sequences.

In embodiments, piRNAs are not degraded in circulation and are stably expressed in body fluids. PIWI proteins are mainly expressed in the germline and human tumors. The human PIWI protein subfamily consists of PIWIL1, PIWIL2, PIWIL3 and PIWIL4. In embodiments, piRNAs interact with PIWI subfamily proteins, resulting in the development of the piRNA-induced silencing complex (piRISC), which detects and silences complementary sequences at the transcriptional (TGS) and post-transcriptional (PTGS) levels.

A “gapmeR” as provided herein refers to a short DNA antisense oligonucleotide flanked by RNA sequences. In embodiments, the RNA sequences include or are sequences of RNA nucleotide analogs. In embodiments, the RNA nucleotide analog is independently a locked nucleic acid (LNA), 2′-OMe, or 2′-F modified bases. In embodiments, LNA sequences are RNA analogues “locked” into an ideal Watson-Crick base pairing conformation. In embodiments, LNAs, 2′-OMe, or 2′-F modified bases are chemical analogs of natural RNA nucleic acids and allow for an increase in nuclease resistance, reduced immunogenicity, and a decrease in toxicity. In embodiments, gapmers have a high binding affinity to the target mRNA. In embodiments, this high binding affinity may reduce off-target effects, non-specific binding, and unwanted gene silencing. In embodiments, gapmeRs utilize nucleotides modified with phosphorothioate (PS) groups. In humans, the gapmer DNA-mRNA duplex may be degraded by RNase H. In embodiments, the degradation of the mRNA prevents protein synthesis. In embodiments, gapmeRs are designed to hybridize to a target RNA sequence and silence the gene through the induction of RNase H cleavage. In embodiments, binding of the gapmer to the target has a higher affinity due to the modified RNA flanking regions, as well as resistance to degradation by nucleases.

As used herein the term “small molecule” refers to a low molecular weight organic compound, typically involved in a biological process as a substrate or product. In embodiments, small molecules have a mass range of 50-1500 daltons (Da). In embodiments, organic compounds with low molecular weight are small molecule drugs. In embodiments, small molecule drugs can be administered orally. In embodiments, small molecule drugs can pass through cell membranes to reach intracellular targets. In embodiments, small molecule drugs can pass through the blood-brain barrier.

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In embodiments, the polynucleotide (e.g., gRNA) is a single-stranded ribonucleic acid. In aspects, the polynucleotide (e.g., gRNA) is from about 10 to about 200 nucleic acid residues in length. In aspects, the polynucleotide (e.g., gRNA) is from about 50 to about 150 nucleic acid residues in length. In aspects, the polynucleotide (e.g., gRNA) is from about 80 to about 140 nucleic acid residues in length. In aspects, the polynucleotide (e.g., gRNA) is from about 90 to about 130 nucleic acid residues in length. In aspects, the polynucleotide (e.g., gRNA) is from about 100 to about 120 nucleic acid residues in length. In aspects, the length of the polynucleotide (e.g., gRNA) is about 113 nucleic acid residues in length.

In embodiments, a guide sequence (i.e., a DNA-targeting sequence) is any polynucleotide sequence having sufficient complementarity with a target polynucleotide sequence to hybridize with the target sequence (e.g., a genomic or mitochondrial DNA target sequence) and direct sequence-specific binding of a complex (e.g., CRISPR complex) to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In aspects, the degree of complementarity between a guide sequence and its corresponding target sequence, when optimally aligned using a suitable alignment algorithm, is at least about 80%, 85%, 90%, 95%, or 100%. In aspects, the degree of complementarity is at least 90%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In aspects, a guide sequence is about or more than about 10, 20, 30, 35, 40, 45, 50, 75, or more nucleotides in length. In aspects, a guide sequence is about 10 to about 150, about 15 to about 100 nucleotides in length. In aspects, a guide sequence is less than about 75, 50, 45, 40, 35, 30, 25, 20, 15, 12, or fewer nucleotides in length. In aspects, the guide sequence is about or more than about 20 nucleotides in length. The ability of a guide sequence to direct sequence-specific binding of a complex (e.g., CRISPR complex) to a target sequence may be assessed by any suitable assay. For example, the components of a CRISPR system sufficient to form a complex (e.g., CRISPR complex), including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay known in the art. Similarly, cleavage of a target polynucleotide sequence may be evaluated in a test tube by providing the target sequence, components of a complex (e.g., CRISPR complex), including the guide sequence to be tested and a control guide sequence different from the test guide sequence, and comparing binding or rate of cleavage at the target sequence between the test and control guide sequence reactions. Other assays are possible, and will occur to those skilled in the art. The terms “sgRNA,” “single guide RNA,” and “single guide RNA sequence” are used interchangeably and refer to the polynucleotide sequence including the crRNA sequence and optionally the tracrRNA sequence. The crRNA sequence includes a guide sequence (i.e., “guide” or “spacer”) and a tracr mate sequence (i.e., direct repeat(s)”). The term “guide sequence” refers to the sequence that specifies the target site. In aspects, the two RNA can be encoded separately by a crRNA and tracrRNA as 2 RNA molecules which then form an RNA/RNA complex due to complementary base pairing between the crRNA and tracrRNA (i.e., before being competent to bind to nuclease-deficient RNA-guided DNA endonuclease enzyme). In aspects, a first nucleic acid includes a tracrRNA sequence, and a separate second nucleic acid includes a gRNA sequence lacking a tracrRNA sequence. In aspects, the first nucleic acid including the tracrRNA sequence and the second nucleic acid including the gRNA sequence interact with one another, and optionally are included in a complex (e.g., CRISPR complex).

In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) comprises the tracr mate sequence hybridized to the tracr sequence. In general, degree of complementarity is with reference to the optimal alignment of the tracr mate sequence and tracrRNA sequence, along the length of the shorter of the two sequences. Optimal alignment may be determined by any suitable alignment algorithm, and may further account for secondary structures, such as self-complementarity within either the tracrRNA sequence or tracr mate sequence. In aspects, the degree of complementarity between the tracrRNA sequence and tracr mate sequence along the length of the shorter of the two when optimally aligned is about or more than about 25%, 30%, 40%, 50%, 60%, 70%, 80%, 90%, 95%, 97.5%, 99%, or higher. In aspects, the degree of complementarity is about or at least about 80%, 90%, 95%, or 100%. In aspects, the tracrRNA sequence is about or more than about 5, 10, 15, 20, 30, 40, 50, or more nucleotides in length. In aspects, the tracrRNA sequence and tracr mate sequence are contained within a single transcript, such that hybridization between the two produces a transcript having a secondary structure, such as a hairpin.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system.

The term “nuclease-deficient RNA-guided DNA endonuclease enzyme” and the like refer, in the usual and customary sense, to an RNA-guided DNA endonuclease (e.g., a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., single guide RNA (sgRNA)), but is incapable of cleaving the target phosphodiester bond to a significant degree (e.g., there is no measurable cleavage of the phosphodiester bond under physiological conditions). A nuclease-deficient RNA-guided DNA endonuclease thus retains DNA-binding ability (e.g., specific binding to a target sequence) when complexed with a polynucleotide (e.g., sgRNA), but lacks significant endonuclease activity (e.g., any amount of detectable endonuclease activity). In aspects, the nuclease-deficient RNA-guided DNA endonuclease enzyme is a CRISPR-associated protein. In aspects, the nuclease-deficient RNA-guided DNA endonuclease enzyme is dCas9, dCas12a, dCpf1, ddCpf1, Cas-phi, a nuclease-deficient Cas9 variant, a nuclease-deficient Class II CRISPR endonuclease, a leucine zipper domain, a winged helix domain, a helix-turn-helix motif, a helix-loop-helix domain, an H1 MB-box domain, a Wor3 domain, an OB-fold domain, an immunoglobulin domain, or a B3 domain.

The term “CRISPR-associated protein” or “CRISPR protein” refers to any CRISPR protein that functions as a nuclease-deficient RNA-guided DNA endonuclease enzyme, i.e., a CRISPR protein in which catalytic sites for endonuclease activity are defective or lack activity. Exemplary CRISPR proteins include dCas9, dCpf1, ddCpf1, dCas12, ddCas12, dCas12a Cas-phi, a nuclease-deficient Cas9 variant, a nuclease-deficient Class II CRISPR endonuclease, and the like.

The term “nuclease-deficient DNA endonuclease enzyme” refers to a DNA endonuclease (e.g., a mutated form of a naturally occurring DNA endonuclease) that targets a specific phosphodiester bond within a DNA polynucleotide, but that does not require an RNA guide. In embodiments, the “nuclease-deficient DNA endonuclease enzyme” is a zinc finger domain or a transcription activator-like effector (TALE).

In embodiments, the nuclease-deficient DNA endonuclease enzyme is a “zinc finger domain.” The term “zinc finger domain” or “zinc finger binding domain” or “zinc finger DNA binding domain” are used interchangeably and refer to a protein, or a domain within a larger protein, that binds DNA in a sequence-specific manner through one or more zinc fingers, which are regions of amino acid sequence within the binding domain whose structure is stabilized through coordination of a zinc ion. In embodiments, the zinc finger domain is non-naturally occurring in that it is engineered to bind to a target site of choice. In aspects, the zinc finger binding domain refers to a protein, a domain within a larger protein, or a nuclease-deficient RNA-guided DNA endonuclease enzyme that is capable of binding to any zinc finger known in the art, such as the C2H2 type, the CCHC type, the PHD type, or the RING type of zinc fingers.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g., within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

In embodiments, the nuclease-deficient RNA-guided DNA endonuclease enzyme is “ddCpf1” or “ddCas12a”. The terms “DNAse-dead Cpf1” or “ddCpf1” refer to mutated Acidaminococcus sp. Cpf1 (AsCpf1) resulting in the inactivation of Cpf1 DNAse activity.

In embodiments, the nuclease-deficient RNA-guided DNA endonuclease enzyme is dLbCpf1. The term “dLbCpf1: refers to mutated Cpf1 from Lachnospiraceae bacterium ND2006 (LbCpf1) that lacks DNAse activity. In aspects, dLbCpf1 includes a D832A mutation. In aspects, the dLbCpf1 has substantially no detectable endonuclease (e.g., endodeoxyribo-nuclease) activity.

In embodiments, the nuclease-deficient RNA-guided DNA endonuclease enzyme is dFnCpf1. The term “dFnCpf1” refers to mutated Cpf1 from Francisella novicida U112 (FnCpf1) that lacks DNAse activity. In aspects, dFnCpf1 includes a D917A mutation. In aspects, the dFnCpf1 has substantially no detectable endonuclease (e.g., endodeoxyribo-nuclease) activity.

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g., within at least 50%, 80%, 90%, 95%, 96%, 97%, 98%, 99% or 100% activity compared to Cpf1). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Cpf1 protein.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof Non-limiting examples of antibody variants include single-domain antibodies or nanobodies, monospecific Fab2, bispecific Fab2, trispecific Fab3, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable light chain provided herein includes in N-terminal to C-terminal direction a CDR L1, a CDR L2 and a CDR L3.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

“Selective” or “selectivity” or the like of a compound refers to the compound's ability to discriminate between molecular targets.

“Specific”, “specifically”, “specificity”, or the like of a compound refers to the compound's ability to cause a particular action, such as inhibition, to a particular molecular target with minimal or no action to other proteins in the cell.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “expression” includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g. through ionic bond(s), Van Der Waal's bond(s)/interactions, hydrogen bond(s), polar bond(s), or combinations or mixtures thereof).

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. An RNA that is the predominant species present in a preparation is substantially purified.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. In embodiments, a biological sample is a tissue.

In embodiments, a biological sample is blood. In embodiments, a biological sample is a serum sample (e.g., the fluid and solute component of blood without the clotting factors). In embodiments, a biological sample is a plasma sample (e.g, the liquid portion of blood). In embodiments, a biological sample is cell-free RNA obtained from blood.

“Liquid biological sample” refers to liquid materials obtained or derived from a subject or patient. Liquid biological samples include bodily fluids such as blood and blood fractions or products (e.g., serum, plasma, platelets, red blood cells, and the like), sputum, urine, synovial fluid, and the like. In embodiments, a liquid biological sample is a blood sample.

The term “prevent” is used in accordance with its plain and ordinary meaning and refers to a decrease in the occurrence of disease symptoms in a patient. The prevention may be complete (no detectable symptoms) or partial, such that fewer symptoms are observed than would likely occur absent treatment.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

As used herein, the term “Trek Family Protein” refers to a protein belonging to the TRK subgroup of a family of K_{2P} channels. The family contains subgroups of K2P channels which include, but are not limited to, TWIK (tandem of P-domains in a weak inward rectifying K+ channel); TREK, TWIK-related K+ channel; TASK (two-pore domain, acid-sensitive K+ channel); TRAAK (two-pore domain related arachidonic acid activated K+ channel); THIK (two-pore domain halothane inhibited K+ channel); TALK (two-pore domain alkaline activated K+ channel); and TRESK (TWIK-related spinal cord potassium channel). In embodiments, K_{2P} channels are potassium channels characterized by the presence of two pore domains. In embodiments, K_{2P} channels assemble as dimers of four transmembrane segments (M1-M4) and two-pore domain (P1 and P2). In embodiments, K_{2P} channels have an extended M1-P1 extracellular loop and cytosolic N- and C-termini. In embodiments, K_{2P} channels comprise the sequence Gly-Tyr(Phe)-Gly in the first pore (P1) and Gly-Leu(Phe)-Gly in the second pore (P2). In embodiments, TREK family proteins are found on nerve calls. In embodiments, TREK family proteins are found on heart cells. In embodiments, TREK family proteins are found on smooth muscle cells. In embodiments, TREK family proteins are found on pancreas cells. In embodiments, TREK family proteins are found on prostate cells.

As used herein, the term “TREK-i” refers to a K_{2P} channel protein that controls cell excitability and maintains the membrane potential below the threshold of depolarization. In embodiments, TREK1 channels are expressed in dorsal root ganglion (DRG) neurons. In embodiments, TREK1 regulates the excitability of somatosensory nociceptive neurons. In embodiments, TREK-1 regulates pain perception. In embodiments, TREK-1 regulates depression. In embodiments, TREK-1 regulates the response to anesthesia. In embodiments, TREK-1 is expressed in the brain. In embodiments, TREK-1 is expressed in the heart. In embodiments, TREK-1 is expressed in smooth muscle cells. In embodiments, TREK-1 is expressed in the pancreas. In embodiments, TREK-1 is expressed in the prostate. In embodiments, the TREK-1 protein encoded by the KCNK2 gene has the amino acid sequence set forth in or corresponding to Entrez 3776, UniProt 095069, RefSeq (protein) NP_001017424.1, RefSeq (protein) NP_001017425.2, or RefSeq (protein) NP_055032.1. In embodiments, the amino acid sequence sequence is the sequence known at the time of filing of the present application.

As used herein, the term “TREK-2” refers to a K_{2P} channel protein expressed mainly in the cerebellum, spleen and testis. In embodiments, TREK-2 channels are expressed in small DRG neurons. In embodiments, TREK-2 controls the resting membrane potential. In embodiments, TREK-2 channels are mechanosensitive. In embodiments, TREK-2 channels are activated by heat. In embodiments, TREK-2 channels limit spontaneous pain. In embodiments, TREK-2 channels limit neuropathic pain. In embodiments, TREK-2 channels limit hyperalgesia. In embodiments, TREK-2 protects epithelial cells against pressure-induced apoptosis. In embodiments, TREK-2 is expressed in renal epithelial cells. In embodiments, TREK-2 activation prevents kidney damage under conditions of increased blood pressure. In embodiments, TREK-2 activation preserves kidney function under high blood pressure. In embodiments, TREK-2 activation protects against neuropathic pain. In embodiments, the TREK-2 protein encoded by the KCNK10 gene has the amino acid sequence set forth in or corresponding to Entrez 54207, UniProt P57789, RefSeq (protein) NP_066984.1, RefSeq (protein) NP_612190.1, or RefSeq (protein) NP_612191.1. In embodiments, the amino acid sequence sequence is the sequence known at the time of filing of the present application.

As used herein, the term “TRAAK” refers to members of the two pore domain K₂P channel family. In embodiments, TRAAK proteins are found in the nervous system. In embodiments, TRAAK channels are mechanically gated. In embodiments, TRAAK is found in the brain. In embodiments, TRAAK is found in the spinal cord. In embodiments, TRAAK is found in the retina. In embodiments, TRAAK regulates neuronal excitability. In embodiments, TRAAK is expressed in the olfactory system. In embodiments, TRAAK is expressed in the cerebral cortex. In embodiments, TRAAK is expressed in the hippocampal formation. In embodiments, TRAAK is expressed in the habenula. In embodiments, TRAAK is expressed in the basal ganglia. In embodiments, TRAAK is expressed in the cerebellum. In embodiments, TRAAK channels regulate maintenance of the resting membrane potential in excitable cell types. In embodiments, abnormal TRAAK expression may cause facial dysmorphism. In embodiments, abnormal TRAAK expression may cause hypertrichosis. In embodiments, abnormal TRAAK expression may cause epilepsy. In embodiments, abnormal TRAAK expression may cause developmental delay. In embodiments, abnormal TRAAK expression may cause gingival overgrowth. In embodiments, abnormal TRAAK expression may cause neuronal dysplasia. In embodiments, abnormal TRAAK expression may cause cerebral ischemia following a stroke. In embodiments, TRAAK is activated by arachidonic acid. In embodiments, the TRAAK protein encoded by the KCNK4 gene has the amino acid sequence set forth in or corresponding to Entrez 50801, UniProt Q9NYG8, RefSeq (protein) NP_001304019.1, or RefSeq (protein) NP_201567.1. In embodiments, the amino acid sequence sequence is the sequence known at the time of filing of the present application.

As used herein, the term “THIK-1” refers to a K_{2P} channel protein (including homologs, isoforms, and functional fragments thereof). In embodiments, the THIK-1 protein encoded by the KCNK13 gene has the amino acid sequence set forth in or corresponding to Entrez 56659, UniProt Q9HB14, or RefSeq (protein) NP_071337.2. In embodiments, the amino acid sequence sequence is the sequence known at the time of filing of the present application.

As used herein, the term “THIK-2” refers to a K_{2P} channel protein (including homologs, isoforms, and functional fragments thereof). In embodiments, the THIK-2 protein encoded by the KCNK12 gene has the amino acid sequence set forth in or corresponding to Entrez 56660, UniProt Q9HB15, or RefSeq (protein) NP_071338.1. In embodiments, the amino acid sequence sequence is the sequence known at the time of filing of the present application.

As used herein, the term “TWIK-1” refers to a K_{2P} channel protein (including homologs, isoforms, and functional fragments thereof). In embodiments, the TWIK-1 protein encoded by the KCNK1 gene has the amino acid sequence set forth in or corresponding to Entrez 3775, UniProt 000180, or RefSeq (protein) NP_002236.1. In embodiments, the amino acid sequence sequence is the sequence known at the time of filing of the present application.

As used herein, the term “TWIK-2” refers to a K_{2P} channel protein (including homologs, isoforms, and functional fragments thereof). In embodiments, the TWIK-2 protein encoded by the KCNK6 gene has the amino acid sequence set forth in or corresponding to Entrez 9424, UniProt Q9Y257, or RefSeq (protein) NP_004814.1. In embodiments, the amino acid sequence sequence is the sequence known at the time of filing of the present application.

As used herein, the term “KCNK7” refers to a K_{2P} channel protein (including homologs, isoforms, and functional fragments thereof). In embodiments, the KCNK7 protein encoded by the KCNK7 gene has the amino acid sequence set forth in or corresponding to Entrez 10089, UniProt Q9Y2U2, RefSeq (protein) NP_005705.1, RefSeq (protein) NP_203133.1, RefSeq (protein) NP_203134.1, or RefSeq (protein) NP_258416.1. In embodiments, the amino acid sequence sequence is the sequence known at the time of filing of the present application.

As used herein, the term “TRESK” refers to a K_{2P} channel protein (including homologs, isoforms, and functional fragments thereof). In embodiments, the TRESK protein encoded by the KCNK18 gene has the amino acid sequence set forth in or corresponding to Entrez 338567, UniProt Q7Z418, or RefSeq (protein) NP_862823.1. In embodiments, the amino acid sequence sequence is the sequence known at the time of filing of the present application.

As used herein, the term “TASK-i” refers to a K_{2P} channel protein (including homologs, isoforms, and functional fragments thereof). In embodiments, the TASK-1 protein encoded by the KCNK3 gene has the amino acid sequence set forth in or corresponding to Entrez 3777, UniProt 014649, or RefSeq (protein) NP_002237.1. In embodiments, the amino acid sequence sequence is the sequence known at the time of filing of the present application.

As used herein, the term “TASK-3” refers to a K_{2P} channel protein (including homologs, isoforms, and functional fragments thereof). In embodiments, the TASK-3 protein encoded by the KCNK9 gene has the amino acid sequence set forth in or corresponding to Entrez 51305, UniProt Q9NPC2, or RefSeq (protein) NP_001269463.1. In embodiments, the amino acid sequence sequence is the sequence known at the time of filing of the present application.

As used herein, the term “TASK-5” refers to a K_{2P} channel protein (including homologs, isoforms, and functional fragments thereof). In embodiments, the TASK-5 protein encoded by the KCNK15 gene has the amino acid sequence set forth in or corresponding to Entrez 60598, UniProt Q9H427, or RefSeq (protein) NP_071753.2. In embodiments, the amino acid sequence sequence is the sequence known at the time of filing of the present application.

As used herein, the term “TALK-i” refers to a K_{2P} channel protein (including homologs, isoforms, and functional fragments thereof). In embodiments, the TALK-1 protein encoded by the KCNK16 gene has the amino acid sequence set forth in or corresponding to Entrez 83795, UniProt Q96T55, RefSeq (protein) NP_001128577.1, RefSeq (protein) NP_001128578.1, RefSeq (protein) NP_001128579.1, or RefSeq (protein) NP_115491.1. In embodiments, the amino acid sequence sequence is the sequence known at the time of filing of the present application.

As used herein, the term “TALK-2” refers to a K_{2P} channel protein (including homologs, isoforms, and functional fragments thereof). In embodiments, the TALK-2 protein encoded by the KCNK17 gene has the amino acid sequence set forth in or corresponding to Entrez 89822, UniProt Q96T54, RefSeq (protein) NP_001128583.1, or RefSeq (protein) NP_113648.2. In embodiments, the amino acid sequence sequence is the sequence known at the time of filing of the present application.

As used herein, the term “TASK-2” refers to a K_{2P} channel protein (including homologs, isoforms, and functional fragments thereof). In embodiments, the TASK-2 protein encoded by the KCNK5 gene has the amino acid sequence set forth in or corresponding to Entrez 8645, UniProt 095279, or RefSeq (protein) NP_003731.1. In embodiments, the amino acid sequence sequence is the sequence known at the time of filing of the present application.

### II. Compounds

In an aspect is provided a compound, or a pharmaceutically acceptable salt thereof, having the formula:

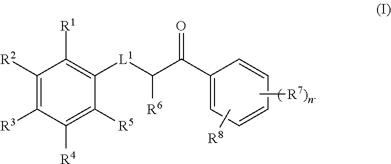

L¹ is a bond, substituted or unsubstituted alkylene (e.g., C₁-C₈, C₁-C₆, C₁-C₄, or C₁-C₂) or substituted or unsubstituted heteroalkylene (e.g., 2 to 8 membered, 2 to 6 membered, 4 to 6 membered, 2 to 3 membered, or 4 to 5 membered).

R¹ is hydrogen, halogen, —CX¹₃, —CHX¹₂, —CH₂X¹, —OCX¹₃, —OCHX¹₂, —OCH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² is hydrogen, halogen, —CX²³, —CHX²₂, —CH₂X², —OCX²₃, —OCHX²₂, —OCH₂X², —CN, —N₃, —SOₙ₂R^{2A}, —SOᵥ₂NR^{2B}, R^{2C}, —NHNR^{2B}, R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is hydrogen, halogen, —CX³₃, —CHX³₂, —CH₂X³, —OCX³₃, —OCHX³₂, —OCH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C} —OR^{3A}, —NR^{3B}SO₂R^{3A}, —NR^{3B}C(O)R^{3D}, —NR^{3B}C(O)OR^{3D}, —NR^{3B}OR^{3D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁴ is hydrogen, halogen, —CX⁴₃, —CHX⁴₂, —CH₂X⁴, —OCX⁴₃, —OCHX⁴₂, —OCH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is hydrogen, halogen, —CX⁵₃, —CHX⁵₂, —CH₂X⁵, —OCX⁵₃, —OCHX⁵₂, —OCH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁶ is hydrogen, a substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered); wherein R⁶ is optionally joined with L¹ to form a substituted or unsubstituted heterocycloalkyl (e.g., 3 to 8 membered, 3 to 6 membered, 4 to 6 membered, 4 to 5 membered, or 5 to 6 membered); or wherein R⁶ is optionally joined with R⁷ to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

R⁷ is independently halogen, —CX⁷₃, —CHX⁷₂, —CH₂X⁷, —OCX⁷₃, —OCHX⁷₂, —OCH₂X⁷, —CN, —N₃, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —N(O)ₘ₇, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, —NR^{7B}OR^{7D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R⁷ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁸ is a cysteine binding moiety or a serine binding moiety.

R^{1A}, R^{1B}, Ric, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{7A}, R^{7B}, R^{7C}, and R^{7D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); and R^{7A} and R^{7B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹, X², X³, X⁴, X⁵, and X⁷ are independently —F, —Cl, —Br, or —I.

The symbols n1, n2, n3, n4, n4, and n7 are independently an integer from 0 to 4.

The symbols m1, m2, m3, m4, m5, m7, v1, v2, v3, v4, v5, and v7 are independently 1 or 2.

The symbol n is an integer from 0 to 3.

In an aspect is provided a compound, or a pharmaceutically acceptable salt thereof, having the formula:

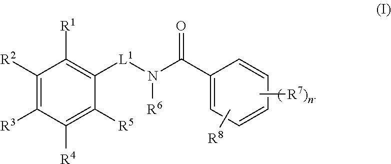

L¹ is a bond, substituted or unsubstituted alkylene or substituted or unsubstituted heteroalkylene. R¹ is independently is hydrogen, halogen, —CX¹₃, —CHX¹₂, —CH₂X¹, —OCX¹₃, —OCHX¹₂, —OCH₂X¹, —CN, —N₃, —SOᵥ₁R^{1A}—SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R² is independently hydrogen, halogen, —CX²₃, —CHX²₂, —CH₂X², —OCX²₃, —OCHX²₂, —OCH₂X², —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R³ is independently hydrogen, halogen, —CX³₃, —CHX³₂, —CH₂X³, —OCX³₃, —OCHX³₂, —OCH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R⁴ is independently hydrogen, halogen, —CX⁴₃, —CHX⁴₂, —CH₂X⁴, —OCX⁴₃, —OCHX⁴₂, —OCH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R⁴ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R⁵ is independently hydrogen, halogen, —CX⁵₃, —CHX⁵₂, —CH₂X⁵, —OCX⁵₃, —OCHX⁵₂, —OCH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R⁵ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R⁶ is independently a substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered); wherein R⁶ is optionally joined with L¹ to form a substituted or unsubstituted heterocycloalkyl (e.g., 3 to 8 membered, 3 to 6 membered, 4 to 6 membered, 4 to 5 membered, or 5 to 6 membered). R⁷ is independently halogen, —CX⁷₃, —CHX⁷₂, —CH₂X⁷, —OCX⁷₃, —OCHX⁷₂, —OCH₂X⁷, —CN, —N₃, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —N(O)ₘ₇, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, —NR^{7B}OR^{7D}, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R⁷ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R⁸ is a cysteine binding moiety or a serine binding moiety. R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{7A}, R^{7B}, R^{7C}, and R^{7D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2A}, and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); and R^{7A} and R^{7B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered). X¹, X², X³, X⁴, X⁵, and X⁷ are independently —F, —Cl, —Br, or —I. The symbols n1, n2, n3, n4, n4, and n7 are an integer from 0 to 4. The symbols m1, m2, m3, m4, m5, m7, v1, v2, v3, v4, v5, and v7 are independently 1 or 2. The symbol n is an integer from 0 to 3.

In embodiments, a substituted L¹(e.g., substituted alkylene or substituted heteroalkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond, substituted or unsubstituted C₁-C₆ alkylene, or substituted or unsubstituted 2 to 6 membered heteroalkylene. In embodiments, L¹ is a bond. In embodiments, L¹ is substituted or unsubstituted C₁-C₆ alkylene. In embodiments, L¹ is substituted or unsubstituted 2 to 6 membered heteroalkylene.

In embodiments, L¹ is unsubstituted C₁-C₆ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene. In embodiments, L¹ is unsubstituted butylene. In embodiments, L¹ is unsubstituted n-butylene. In embodiments, L¹ is unsubstituted isobutylene. In embodiments, L¹ is unsubstituted tert-butylene.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently hydrogen or halogen. In embodiments, R¹ is independently hydrogen. In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl.

In embodiments, R^{1A} is hydrogen. In embodiments, R^{1A} is unsubstituted C₁-C₄ alkyl. In embodiments, R^{1A} is unsubstituted methyl. In embodiments, R^{1A} is unsubstituted ethyl. In embodiments, R^{1A} is unsubstituted propyl. In embodiments, R^{1A} is unsubstituted n-propyl. In embodiments, R^{1A} is unsubstituted isopropyl. In embodiments, R^{1A} is unsubstituted butyl. In embodiments, R^{1A} is unsubstituted n-butyl. In embodiments, R^{1A} is unsubstituted isobutyl. In embodiments, R^{1A} is unsubstituted tert-butyl.

In embodiments, R^{1B} is hydrogen. In embodiments, R^{1B} is unsubstituted C₁-C₄ alkyl. In embodiments, R^{1B} is unsubstituted methyl. In embodiments, R^{1B} is unsubstituted ethyl. In embodiments, R^{1B} is unsubstituted propyl. In embodiments, R^{1B} is unsubstituted n-propyl. In embodiments, R^{1B} is unsubstituted isopropyl. In embodiments, R^{1B} is unsubstituted butyl. In embodiments, R^{1B} is unsubstituted n-butyl. In embodiments, R^{1B} is unsubstituted isobutyl. In embodiments, R^{1B} is unsubstituted tert-butyl.

In embodiments, R^{1C} is hydrogen. In embodiments, R^{1C} is unsubstituted C₁-C₄ alkyl. In embodiments, R^{1C} is unsubstituted methyl. In embodiments, R^{1C} is unsubstituted ethyl. In embodiments, R^{1C} is unsubstituted propyl. In embodiments, R^{1C} is unsubstituted n-propyl. In embodiments, R^{1C} is unsubstituted isopropyl. In embodiments, R^{1C} is unsubstituted butyl. In embodiments, R^{1C} is unsubstituted n-butyl. In embodiments, R^{1C} is unsubstituted isobutyl. In embodiments, R^{1C} is unsubstituted tert-butyl.

In embodiments, R^{1D} is hydrogen. In embodiments, R^{1D} is unsubstituted C₁-C₄ alkyl. In embodiments, R^{1D} is unsubstituted methyl. In embodiments, R^{1D} is unsubstituted ethyl. In embodiments, R^{1D} is unsubstituted propyl. In embodiments, R^{1D} is unsubstituted n-propyl. In embodiments, R^{1D} is unsubstituted isopropyl. In embodiments, R^{1D} is unsubstituted butyl. In embodiments, R^{1D} is unsubstituted n-butyl. In embodiments, R^{1D} is unsubstituted isobutyl. In embodiments, R^{1D} is unsubstituted tert-butyl.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently hydrogen or halogen. In embodiments, R² is independently hydrogen. In embodiments, R² is independently halogen. In embodiments, R² is independently —F. In embodiments, R² is independently —Cl. In embodiments, R² is independently —Br. In embodiments, R² is independently —I. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl.

In embodiments, R^{2A} is hydrogen. In embodiments, R^{2A} is unsubstituted C₁-C₄ alkyl. In embodiments, R^{2A} is unsubstituted methyl. In embodiments, R^{2A} is unsubstituted ethyl. In embodiments, R^{2A} is unsubstituted propyl. In embodiments, R^{2A} is unsubstituted n-propyl. In embodiments, R^{2A} is unsubstituted isopropyl. In embodiments, R^{2A} is unsubstituted butyl. In embodiments, R^{2A} is unsubstituted n-butyl. In embodiments, R^{2A} is unsubstituted isobutyl. In embodiments, R^{2A} is unsubstituted tert-butyl.

In embodiments, R^{2B} is hydrogen. In embodiments, R^{2B} is unsubstituted C₁-C₄ alkyl. In embodiments, R^{2B} is unsubstituted methyl. In embodiments, R^{2B} is unsubstituted ethyl. In embodiments, R^{2B} is unsubstituted propyl. In embodiments, R^{2B} is unsubstituted n-propyl. In embodiments, R^{2B} is unsubstituted isopropyl. In embodiments, R^{2B} is unsubstituted butyl. In embodiments, R^{2B} is unsubstituted n-butyl. In embodiments, R^{2B} is unsubstituted isobutyl. In embodiments, R^{2B} is unsubstituted tert-butyl.

In embodiments, R^{2C} is hydrogen. In embodiments, R^{2C} is unsubstituted C₁-C₄ alkyl. In embodiments, R^{2C} is unsubstituted methyl. In embodiments, R^{2C} is unsubstituted ethyl. In embodiments, R^{2C} is unsubstituted propyl. In embodiments, R^{2C} is unsubstituted n-propyl. In embodiments, R^{2C} is unsubstituted isopropyl. In embodiments, R^{2C} is unsubstituted butyl. In embodiments, R^{2C} is unsubstituted n-butyl. In embodiments, R^{2C} is unsubstituted isobutyl. In embodiments, R^{2C} is unsubstituted tert-butyl.

In embodiments, R^{2D} is hydrogen. In embodiments, R^{2D} is unsubstituted C₁-C₄ alkyl. In embodiments, R^{2D} is unsubstituted methyl. In embodiments, R^{2D} is unsubstituted ethyl. In embodiments, R^{2D} is unsubstituted propyl. In embodiments, R^{2D} is unsubstituted n-propyl. In embodiments, R^{2D} is unsubstituted isopropyl. In embodiments, R^{2D} is unsubstituted butyl. In embodiments, R^{2D} is unsubstituted n-butyl. In embodiments, R^{2D} is unsubstituted isobutyl. In embodiments, R^{2D} is unsubstituted tert-butyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A}, and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is independently halogen or substituted or unsubstituted C₁-C₄ alkynyl. In embodiments, R³ is independently halogen. In embodiments, R³ is independently —F. In embodiments, R³ is independently —Cl. In embodiments, R³ is independently —Br. In embodiments, R³ is independently —I. In embodiments, R³ is independently substituted or unsubstituted C₁-C₄ alkynyl. In embodiments, R³ is unsubstituted methyne (e.g., methynyl).

In embodiments, R³ is unsubstituted ethyne (e.g., ethynyl). In embodiments, R³ is unsubstituted propyne (e.g., propynyl). In embodiments, R³ is unsubstituted n-propyne (e.g., n-propynyl). In embodiments, R³ is unsubstituted isopropyne (e.g., isopropynyl). In embodiments, R³ is unsubstituted butyne (e.g., butynyl). In embodiments, R³ is unsubstituted n-butyne (e.g., n-butynyl). In embodiments, R³ is unsubstituted isobutyne (e.g., isobutynyl).

In embodiments, R³ is unsubstituted tert-butyne (e.g., tert-butynyl). In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl.

In embodiments, R^{3A} is hydrogen. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl.

In embodiments, R^{3B} is hydrogen. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl.

In embodiments, R^{3C} is hydrogen. In embodiments, R^{3C} is unsubstituted C₁-C₄ alkyl. In embodiments, R^{3C} is unsubstituted methyl. In embodiments, R^{3C} is unsubstituted ethyl. In embodiments, R^{3C} is unsubstituted propyl. In embodiments, R^{3C} is unsubstituted n-propyl. In embodiments, R^{3C} is unsubstituted isopropyl. In embodiments, R^{3C} is unsubstituted butyl. In embodiments, R^{3C} is unsubstituted n-butyl. In embodiments, R^{3C} is unsubstituted isobutyl. In embodiments, R^{3C} is unsubstituted tert-butyl.

In embodiments, R^{3D} is hydrogen. In embodiments, R^{3D} is unsubstituted C₁-C₄ alkyl. In embodiments, R^{3D} is unsubstituted methyl. In embodiments, R^{3D} is unsubstituted ethyl. In embodiments, R^{3D} is unsubstituted propyl. In embodiments, R^{3D} is unsubstituted n-propyl. In embodiments, R^{3D} is unsubstituted isopropyl. In embodiments, R^{3D} is unsubstituted butyl. In embodiments, R^{3D} is unsubstituted n-butyl. In embodiments, R^{3D} is unsubstituted isobutyl. In embodiments, R^{3D} is unsubstituted tert-butyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A}, and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D}, (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D}, is substituted, it is substituted with at least one size-limited substituent group. In embodiments, when R^{4D}, is substituted, it is substituted with at least one lower substituent group.

In embodiments, R⁴ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen. In embodiments, R⁴ is halogen. In embodiments, R⁴ is —F. In embodiments, R⁴ is —Cl. In embodiments, R⁴ is —Br. In embodiments, R⁴ is —I.

In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl.

In embodiments, R^{4A} is hydrogen. In embodiments, R^{4A} is unsubstituted C₁-C₄ alkyl. In embodiments, R^{4A} is unsubstituted methyl. In embodiments, R^{4A} is unsubstituted ethyl. In embodiments, R^{4A} is unsubstituted propyl. In embodiments, R^{4A} is unsubstituted n-propyl. In embodiments, R^{4A} is unsubstituted isopropyl. In embodiments, R^{4A} is unsubstituted butyl. In embodiments, R^{4A} is unsubstituted n-butyl. In embodiments, R^{4A} is unsubstituted isobutyl. In embodiments, R^{4A} is unsubstituted tert-butyl.

In embodiments, R^{4B} is hydrogen. In embodiments, R^{4B} is unsubstituted C₁-C₄ alkyl. In embodiments, R^{4B} is unsubstituted methyl. In embodiments, R^{4B} is unsubstituted ethyl. In embodiments, R^{4B} is unsubstituted propyl. In embodiments, R^{4B} is unsubstituted n-propyl. In embodiments, R^{4B} is unsubstituted isopropyl. In embodiments, R^{4B} is unsubstituted butyl. In embodiments, R^{4B} is unsubstituted n-butyl. In embodiments, R^{4B} is unsubstituted isobutyl. In embodiments, R^{4B} is unsubstituted tert-butyl.

In embodiments, R^{4C} is hydrogen. In embodiments, R^{4C} is unsubstituted C₁-C₄ alkyl. In embodiments, R^{4C} is unsubstituted methyl. In embodiments, R^{4C} is unsubstituted ethyl. In embodiments, R^{4C} is unsubstituted propyl. In embodiments, R^{4C} is unsubstituted n-propyl. In embodiments, R^{4C} is unsubstituted isopropyl. In embodiments, R^{4C} is unsubstituted butyl. In embodiments, R^{4C} is unsubstituted n-butyl. In embodiments, R^{4C} is unsubstituted isobutyl. In embodiments, R^{4C} is unsubstituted tert-butyl.

In embodiments, R^{4D} is hydrogen. In embodiments, R^{4D}, is unsubstituted C₁-C₄ alkyl. In embodiments, R^{4D}, is unsubstituted methyl. In embodiments, R^{4D}, is unsubstituted ethyl. In embodiments, R^{4D}, is unsubstituted propyl. In embodiments, R^{4D}, is unsubstituted n-propyl. In embodiments, R^{4D}, is unsubstituted isopropyl. In embodiments, R^{4D}, is unsubstituted butyl. In embodiments, R^{4D}, is unsubstituted n-butyl. In embodiments, R^{4D}, is unsubstituted isobutyl. In embodiments, R^{4D}, is unsubstituted tert-butyl.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R^{S} is substituted, it is substituted with at least one substituent group. In embodiments, when R^{S} is substituted, it is substituted with at least one size-limited substituent group. In embodiments, when R^{S} is substituted, it is substituted with at least one lower substituent group.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when R^{5A} and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R⁵ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen. In embodiments, R⁵ is halogen. In embodiments, R⁵ is —F. In embodiments, R⁵ is —Cl. In embodiments, R⁵ is —Br. In embodiments, R⁵ is —I.

In embodiments, R⁵ is unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl.

In embodiments, R^{5A} is hydrogen. In embodiments, R^{5A} is unsubstituted C₁-C₄ alkyl. In embodiments, R^{5A} is unsubstituted methyl. In embodiments, R^{5A} is unsubstituted ethyl. In embodiments, R^{5A} is unsubstituted propyl. In embodiments, R^{5A} is unsubstituted n-propyl. In embodiments, R^{5A} is unsubstituted isopropyl. In embodiments, R^{5A} is unsubstituted butyl. In embodiments, R^{5A} is unsubstituted n-butyl. In embodiments, R^{5A} is unsubstituted isobutyl. In embodiments, R^{5A} is unsubstituted tert-butyl.

In embodiments, R^{5B} is hydrogen. In embodiments, R^{5B} is unsubstituted C₁-C₄ alkyl. In embodiments, R^{5B} is unsubstituted methyl. In embodiments, R^{5B} is unsubstituted ethyl. In embodiments, R^{5B} is unsubstituted propyl. In embodiments, R^{5B} is unsubstituted n-propyl. In embodiments, R^{5B} is unsubstituted isopropyl. In embodiments, R^{5B} is unsubstituted butyl. In embodiments, R^{5B} is unsubstituted n-butyl. In embodiments, R^{5B} is unsubstituted isobutyl. In embodiments, R^{5B} is unsubstituted tert-butyl.

In embodiments, R^{5C} is hydrogen. In embodiments, R^{5C} is unsubstituted C₁-C₄ alkyl. In embodiments, R^{5C} is unsubstituted methyl. In embodiments, R^{5C} is unsubstituted ethyl. In embodiments, R^{5C} is unsubstituted propyl. In embodiments, R^{5C} is unsubstituted n-propyl. In embodiments, R^{5C} is unsubstituted isopropyl. In embodiments, R^{5C} is unsubstituted butyl. In embodiments, R^{5C} is unsubstituted n-butyl. In embodiments, R^{5C} is unsubstituted isobutyl. In embodiments, R^{5C} is unsubstituted tert-butyl.

In embodiments, R^{5D} is hydrogen. In embodiments, R^{5D} is unsubstituted C₁-C₄ alkyl. In embodiments, R^{5D} is unsubstituted methyl. In embodiments, R^{5D} is unsubstituted ethyl. In embodiments, R^{5D} is unsubstituted propyl. In embodiments, R^{5D} is unsubstituted n-propyl. In embodiments, R^{5D} is unsubstituted isopropyl. In embodiments, R^{5D} is unsubstituted butyl. In embodiments, R^{5D} is unsubstituted n-butyl. In embodiments, R^{5D} is unsubstituted isobutyl. In embodiments, R^{5D} is unsubstituted tert-butyl.

In embodiments, a substituted R⁶ (e.g., substituted alkyl and/or substituted heteroalkyl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, a substituted ring formed when R⁶ and L¹ substituents are joined (e.g., substituted heterocycloalkyl) is substituted with at least one substituent group, size-limited substituent group, or lower substituent group; wherein if the substituted ring formed when R⁶ and L¹ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁶ and L¹ substituents are joined is substituted, it is substituted with at least one substituent group. In embodiments, when the substituted ring formed when R⁶ and L¹ substituents are joined is substituted, it is substituted with at least one size-limited substituent group. In embodiments, when the substituted ring formed when R⁶ and L¹ substituents are joined is substituted, it is substituted with at least one lower substituent group.

In embodiments, a substituted ring formed when R⁶ and R⁷ substituents are joined (e.g., substituted heterocycloalkyl and/or substituted heteroaryl) is substituted with at least one substituent group, size-limited substituent group, or lower substituent group; wherein if the substituted ring formed when R⁶ and R⁷ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁶ and R⁷ substituents are joined is substituted, it is substituted with at least one substituent group. In embodiments, when the substituted ring formed when R⁶ and R⁷ substituents are joined is substituted, it is substituted with at least one size-limited substituent group. In embodiments, when the substituted ring formed when R⁶ and R⁷ substituents are joined is substituted, it is substituted with at least one lower substituent group.

In embodiments, R⁶ is independently hydrogen, substituted or unsubstituted C₁-C₄ alkyl or R⁶ and L¹ are joined together to form a substituted or unsubstituted 5 to 7 membered heterocycloalkyl.

In embodiments, R⁶ is independently hydrogen. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ and L¹ are joined together to form a substituted or unsubstituted 5 to 7 membered heterocycloalkyl. In embodiments, R⁶ and L¹ are joined together to form an unsubstituted pyrrolidine. In embodiments, R⁶ and L¹ are joined together to form an unsubstituted pyrazolidine. In embodiments, R⁶ and L¹ are joined together to form an unsubstituted imidazolidine. In embodiments, R⁶ and L¹ are joined together to form an unsubstituted tetrahydrofuran. In embodiments, R⁶ and L¹ are joined together to form an unsubstituted 1,3-dioxolane. In embodiments, R⁶ and L¹ are joined together to form an unsubstituted tetrahydrothiophene. In embodiments, R⁶ and L¹ are joined together to form an unsubstituted sulfolane. In embodiments, R⁶ and L¹ are joined together to form an unsubstituted 2,4-thiazolidinedione. In embodiments, R⁶ and L¹ are joined together to form an unsubstituted succinimide. In embodiments, R⁶ and L¹ are joined together to form an unsubstituted 2-oxazolidone. In embodiments, R⁶ and L¹ are joined together to form an unsubstituted hydantoin. In embodiments, R⁶ and L¹ are joined together to form an unsubstituted piperidine. In embodiments, R⁶ and L¹ are joined together to form an unsubstituted piperazine. In embodiments, R⁶ and L¹ are joined together to form an unsubstituted tetrahydropyran. In embodiments, R⁶ and L¹ are joined together to form an unsubstituted thiane. In embodiments, R⁶ and L¹ are joined together to form an unsubstituted dithiane. In embodiments, R⁶ and L¹ are joined together to form an unsubstituted trithiane.

In embodiments, R⁶ and L¹ are joined together to form an unsubstituted morpholine. In embodiments, R⁶ and L¹ are joined together to form an unsubstituted thiomorpholine. In embodiments, R⁶ and L¹ are joined together to form an unsubstituted dioxine. In embodiments, R⁶ and L¹ are joined together to form an unsubstituted thiomorpholine dioxide.

In embodiments, R⁶ and L¹ are joined together to form an unsubstituted oxepane. In embodiments, R⁶ and L¹ are joined together to form an unsubstituted azepane. In embodiments, R⁶ and L¹ are joined together to form an unsubstituted thiopane. In embodiments, R⁶ and L¹ are joined together to form an unsubstituted azepan-2-one. In embodiments, R⁶ and L¹ are joined together to form an unsubstituted azepan-5-one. In embodiments, R⁶ and L¹ are joined together to form an unsubstituted piperidin-2-one.

In embodiments, R⁶ and R⁷ are joined to form a substituted or unsubstituted 3 to 8 membered heterocycloalkyl or substituted or unsubstituted 5 to 10 membered heteroaryl. In embodiments, R⁶ and R⁷ are joined to form a substituted 3 to 8 membered heterocycloalkyl.

In embodiments, R⁶ and R⁷ are joined to form a substituted oxazepanyl. In embodiments, R⁶, and R⁷ are joined to form a substituted oxo-substituted oxazepanyl. In embodiments, R⁶ and R⁷ are joined to form a substituted azepanyl. In embodiments, R⁶ and R⁷ are joined to form an oxo-substituted azepanyl. In embodiments, R⁶ and R⁷ are joined to form a substituted piperidinyl. In embodiments, R⁶ and R⁷ are joined to form a substituted oxo-substituted piperidinyl.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, a substituted ring formed when two R⁷ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R⁷ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R⁷ substituents are joined is substituted, it is substituted with at least one substituent group. In embodiments, when the substituted ring formed when two R⁷ substituents are joined is substituted, it is substituted with at least one size-limited substituent group. In embodiments, when the substituted ring formed when two R⁷ substituents are joined is substituted, it is substituted with at least one lower substituent group.

In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B} is substituted, it is substituted with at least one lower substituent group.

In embodiments, a substituted ring formed when R^{7A} and R^{7B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{7A} and R^{7B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{7A} and R^{7B} substituents bonded to the same nitrogen atom are joined is substituted, it is substituted with at least one substituent group. In embodiments, when the substituted ring formed when R^{7A}, and R^{7B} substituents bonded to the same nitrogen atom are joined is substituted, it is substituted with at least one size-limited substituent group. In embodiments, when the substituted ring formed when R^{7A} and R^{7B} substituents bonded to the same nitrogen atom are joined is substituted, it is substituted with at least one lower substituent group.

In embodiments, a substituted R^{7C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7C} is substituted with a plurality of groups selected from substituent groups, size-limited substituent groups, and lower substituent groups; each substituent group, size-limited substituent group, and/or lower substituent group may optionally be different. In embodiments, when R^{7C} is substituted, it is substituted with at least one substituent group. In embodiments, when R^{7C} is substituted, it is substituted with at least one size-limited substituent group. In embodiments, when R^{7C} is substituted, it is substituted with at least one lower substituent group.

In embodiments, a substituted R^{7D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7D} is substituted with a plurality of groups selected from substituent groups, size-limited substituent groups, and lower substituent groups; each substituent group, size-limited substituent group, and/or lower substituent group may optionally be different. In embodiments, when R^{7D} is substituted, it is substituted with at least one substituent group. In embodiments, when R^{7D} is substituted, it is substituted with at least one size-limited substituent group. In embodiments, when R^{7D} is substituted, it is substituted with at least one lower substituent group.

In embodiments, R⁷ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is a substituted or unsubstituted C₁-C₄ alkyl, substituted or unsubstituted 2 to 4 membered heteroalkyl, substituted or unsubstituted C₃-C₅ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

In embodiments, R⁷ is an unsubstituted C₁-C₄ alkyl, unsubstituted 2 to 4 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl.

In embodiments, R⁷ is an unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl.

In embodiments, R⁷ is an unsubstituted cyclopropyl. In embodiments, R⁷ is an unsubstituted cyclobutyl. In embodiments, R⁷ is an unsubstituted cyclopentyl. In embodiments, R⁷ is an unsubstituted cycohexyl. In embodiments, R⁷ is an unsubstituted cycloheptyl. In embodiments, R⁷ is an unsubstituted cyclooctyl.

In embodiments, R⁷ is an unsubstituted aziridine. In embodiments, R⁷ is an unsubstituted oxirane. In embodiments, R⁷ is an unsubstituted thiirane. In embodiments, R⁷ is an unsubstituted azetidine. In embodiments, R⁷ is an unsubstituted 1,3-diazetidine. In embodiments, R⁷ is an unsubstituted oxetane. In embodiments, R⁷ is an unsubstituted thietane. In embodiments, R⁷ is an unsubstituted pyrrolidine. In embodiments, R⁷ is an unsubstituted pyrazolidine. In embodiments, R⁷ is an unsubstituted imidazolidine. In embodiments, R⁷ is an unsubstituted tetrahydrofuran. In embodiments, R⁷ is an unsubstituted 1,3-dioxolane. In embodiments, R⁷ is an unsubstituted tetrahydrothiophene. In embodiments, R⁷ and L¹ is an unsubstituted sulfolane. In embodiments, R⁷ is an unsubstituted 2,4-thiazolidibedione. In embodiments, R⁷ is an unsubstituted succinimide. In embodiments, R⁷ is an unsubstituted 2-oxazolidone. In embodiments, R⁷ is an unsubstituted hydantoin. In embodiments, R⁷ is an unsubstituted piperidine. In embodiments, R⁷ is an unsubstituted piperazine. In embodiments, R⁷ is an unsubstituted tetrahydropyran. In embodiments, R⁷ is an unsubstituted thiane. In embodiments, R⁷ is an unsubstituted dithiane.

In embodiments, R⁷ is an unsubstituted trithiane. In embodiments, R⁷ is an unsubstituted morpholine. In embodiments, R⁷ is an unsubstituted thiomorpholine. In embodiments, R⁷ is an unsubstituted dioxine. In embodiments, R⁷ is an unsubstituted thiomorpholine dioxide. In embodiments, R⁷ is an unsubstituted oxepane. In embodiments, R⁷ is an unsubstituted azepane. In embodiments, R⁷ is an unsubstituted thiopane. In embodiments, R⁷ is an unsubstituted azocane. In embodiments, R⁷ is an unsubstituted thiocane.

In embodiments, R⁷ is an unsubstituted phenyl.

In embodiments, R⁷ is an unsubstituted pyridine. In embodiments, R⁷ is an unsubstituted pyrudazine. In embodiments, R⁷ is an unsubstituted pyrimidine. In embodiments, R⁷ is an unsubstituted pyrazine. In embodiments, R⁷ is an unsubstituted triazine. In embodiments, R⁷ is an unsubstituted pyran. In embodiments, R⁷ is an unsubstituted 1,4-dioxine. In embodiments, R⁷ is an unsubstituted thiopyran. In embodiments, R⁷ is an unsubstituted oxazine. In embodiments, R⁷ is an unsubstituted thiazine. In embodiments, R⁷ is an unsubstituted cytosine. In embodiments, R⁷ is an unsubstituted thymine. In embodiments, R⁷ is an unsubstituted uracil. In embodiments, R⁷ is an unsubstituted 1,4,5,6-tetrahydrocyclopenta[b]pyrrole. In embodiments, R⁷ is an unsubstituted tetrahydropyrrolo[3,2-b]pyrrole. In embodiments, R⁷ is an unsubstituted dihydropyrrolo[3,2-b]pyrrole. In embodiments, R⁷ is an unsubstituted furo[2,3-b]pyrrole. In embodiments, R⁷ is an unsubstituted thieno[2,3-b]pyrrole. In embodiments, R⁷ is an unsubstituted indole. In embodiments, R⁷ is an unsubstituted isoindole. In embodiments, R⁷ is an unsubstituted dihydro-1H-indene. In embodiments, R⁷ is an unsubstituted indene. In embodiments, R⁷ is an unsubstituted indolene. In embodiments, R⁷ is an unsubstituted indolizine. In embodiments, R⁷ is an unsubstituted 1H-indazole. In embodiments, R⁷ is an unsubstituted benzimidazole. In embodiments, R⁷ is an unsubstituted azaindole. In embodiments, R⁷ is an unsubstituted azaindazole. In embodiments, R⁷ is an unsubstituted pyrazolo[1,5-a]pyrimidine. In embodiments, R⁷ is an unsubstituted purine. In embodiments, R⁷ is an unsubstituted benzofuran. In embodiments, R⁷ is an unsubstituted isobenzofuran. In embodiments, R⁷ is an unsubstituted benzo[c]thiophene. In embodiments, R⁷ is an unsubstituted benzizoxazole. In embodiments, R⁷ is an unsubstituted benzisothiazole. In embodiments, R⁷ is an unsubstituted benzoxazole. In embodiments, R⁷ is an unsubstituted benzthiazole. In embodiments, R⁷ is an unsubstituted benzo[c][1,2,5]thiadiazole. In embodiments, R⁷ is an unsubstituted adenine. In embodiments, R⁷ is an unsubstituted guanine. In embodiments, R⁷ is an unsubstituted quinolone. In embodiments, R⁷ is an unsubstituted isoquinoline. In embodiments, R⁷ is an unsubstituted dihydroquinooline. In embodiments, R⁷ is an unsubstituted tetrahydroquinoline. In embodiments, R⁷ is an unsubstituted quinolizine. In embodiments, R⁷ is an unsubstituted quinoxaline. In embodiments, R⁷ is an unsubstituted quinizolilne. In embodiments, R⁷ is an unsubstituted cinnoline. In embodiments, R⁷ is an unsubstituted phthalazine. In embodiments, R⁷ is an unsubstituted pyridopyrimidine. In embodiments, R⁷ is an unsubstituted pyridopyrazine. In embodiments, R⁷ is an unsubstituted pteridine. In embodiments, R⁷ is an unsubstituted benzooxazine. In embodiments, R⁷ is an unsubstituted quinolinone. In embodiments, R⁷ is an unsubstituted isoquinolinone. In embodiments, R⁷ is an unsubstituted azepine. In embodiments, R⁷ is an unsubstituted diazepine. In embodiments, R⁷ is an unsubstituted thiepine. In embodiments, R⁷ is an unsubstituted thiazepine. In embodiments, R⁷ is an unsubstituted azocine. In embodiments, R⁷ is an unsubstituted azecine.

In embodiments, R^{7A} is hydrogen. In embodiments, R^{7A} is unsubstituted C₁-C₄ alkyl. In embodiments, R^{7A} is unsubstituted methyl. In embodiments, R^{7A} is unsubstituted ethyl. In embodiments, R^{7A} is unsubstituted propyl. In embodiments, R^{7A} is unsubstituted n-propyl. In embodiments, R^{7A} is unsubstituted isopropyl. In embodiments, R^{7A} is unsubstituted butyl. In embodiments, R^{7A} is unsubstituted n-butyl. In embodiments, R^{7A} is unsubstituted isobutyl. In embodiments, R^{7A} is unsubstituted tert-butyl.

In embodiments, R^{7B} is hydrogen. In embodiments, R^{7B} is unsubstituted C₁-C₄ alkyl. In embodiments, R^{7B} is unsubstituted methyl. In embodiments, R^{7B} is unsubstituted ethyl. In embodiments, R^{7B} is unsubstituted propyl. In embodiments, R^{7B} is unsubstituted n-propyl. In embodiments, R^{7B} is unsubstituted isopropyl. In embodiments, R^{7B} is unsubstituted butyl. In embodiments, R^{7B} is unsubstituted n-butyl. In embodiments, R^{7B} is unsubstituted isobutyl. In embodiments, R^{7B} is unsubstituted tert-butyl.

In embodiments, R^{7C} is hydrogen. In embodiments, R^{7C} is unsubstituted C₁-C₄ alkyl. In embodiments, R^{7C} is unsubstituted methyl. In embodiments, R^{7C} is unsubstituted ethyl. In embodiments, R^{7C} is unsubstituted propyl. In embodiments, R^{7C} is unsubstituted n-propyl. In embodiments, R^{7C} is unsubstituted isopropyl. In embodiments, R^{7C} is unsubstituted butyl. In embodiments, R^{7C} is unsubstituted n-butyl. In embodiments, R^{7C} is unsubstituted isobutyl. In embodiments, R^{7C} is unsubstituted tert-butyl.

In embodiments, R^{7D} is hydrogen. In embodiments, R^{7D} is unsubstituted C₁-C₄ alkyl. In embodiments, R^{7D} is unsubstituted methyl. In embodiments, R^{7D} is unsubstituted ethyl. In embodiments, R^{7D} is unsubstituted propyl. In embodiments, R^{7D} is unsubstituted n-propyl. In embodiments, R^{7D} is unsubstituted isopropyl. In embodiments, R^{7D} is unsubstituted butyl. In embodiments, R^{7D} is unsubstituted n-butyl. In embodiments, R^{7D} is unsubstituted isobutyl. In embodiments, R^{7D} is unsubstituted tert-butyl.

In embodiments, n is 0. In embodiments, n is 1. In embodiments, n is 2. In embodiments, n is 3.

In embodiments, R⁸ is a cysteine binding moiety. In embodiments, R⁸ is a serine binding moiety.

In embodiments, the cysteine binding moiety is:

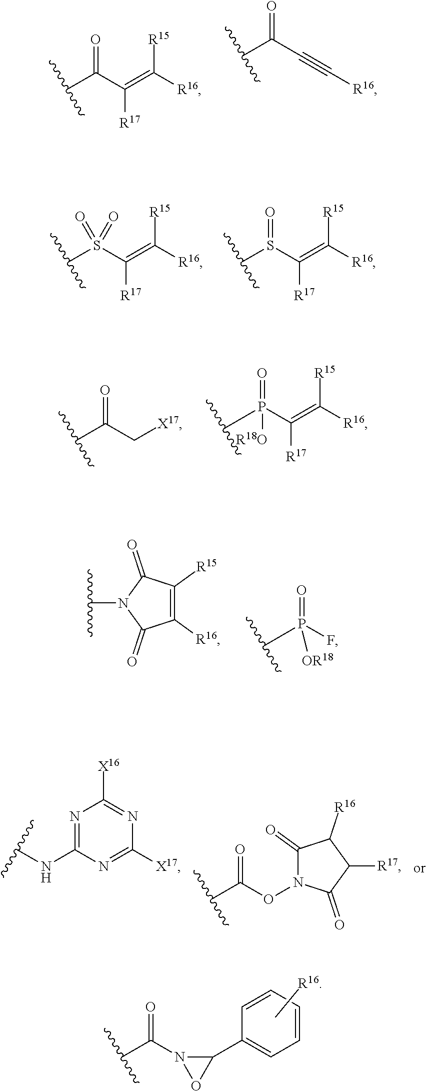

R¹⁵ is independently hydrogen, halogen, —CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁶ is independently hydrogen, halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, N(O)ₘ₁₆, —NR^{16A}R^{16B}, —C(O)R^{16C}, —C(O)—OR^{16C}, —C(O)NR^{16A}R^{16B}, —OR^{16D}, NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁷ is independently hydrogen, halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, N(O)ₘ₁₇, —NR^{17A}R^{17B}, —C(O)R^{17C}, —C(O)—OR^{17C}, —C(O)NR^{17A}R^{17B}, —OR^{17D}, NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, and R^{18C} are independently hydrogen, —CX₃, —CN, —COOH, —CONH₂, —CHX₂, —CH₂X, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{15A} and R^{15B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{16A} and R^{16B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{17A} and R^{17B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{18A} and R^{18B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X, X¹⁵, X¹⁶, X¹⁷, and X¹⁸ are independently —F, —Cl, —Br, or —I.

The symbols n15, n16, and n17 are independently an integer from 0 to 4.

The symbols m15, m16, m17, v15, v16, and v17 are independently an integer from 1 to 2.

In embodiments, the cysteine binding moiety is:

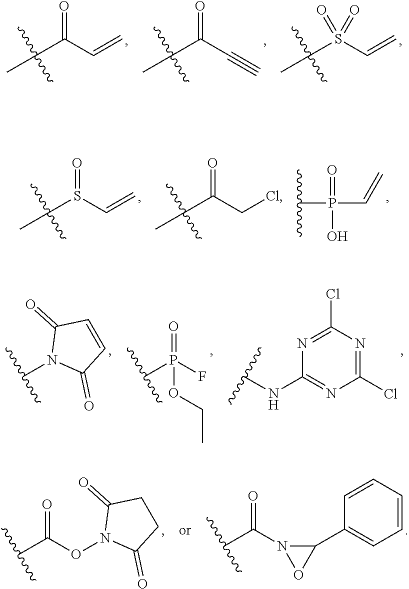

In embodiments, the cysteine binding moiety is

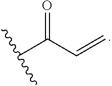 [SMILES: *C(=O)C=C]

In embodiments, the cysteine binding moiety is

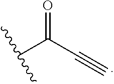 [SMILES: *C(=O)C#C]

In embodiments, the cysteine binding moiety is

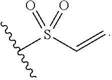 [SMILES: *S(=O)(=O)C=C]

In embodiments, the cysteine binding moiety is

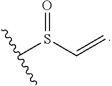 [SMILES: *S(=O)C=C]

In embodiments, the cysteine binding moiety is

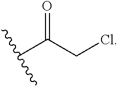 [SMILES: *C(=O)CCl]

In embodiments, the cysteine binding moiety is OH

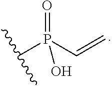 [SMILES: *P(=O)(O)C=C]

In embodiments, the cysteine binding moiety is

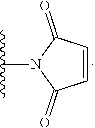 [SMILES: *N1C(=O)C=CC1=O]

In embodiments, the cysteine binding moiety is

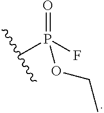 [SMILES: *P(=O)(F)OCC]

In embodiments, the cysteine binding moiety is

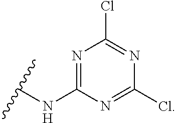 [SMILES: *Nc1nc(Cl)nc(Cl)n1]

In embodiments, the cysteine binding moiety is

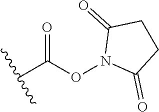 [SMILES: *C(=O)ON1C(=O)CCC1=O]

In embodiments, the cysteine binding moiety is

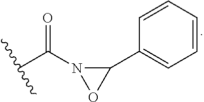 [SMILES: *C(=O)N1OC1c1ccccc1]

In embodiments, a substituted R¹⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁵ is substituted with a plurality of groups selected from substituent groups, size-limited substituent groups, and lower substituent groups; each substituent group, size-limited substituent group, and/or lower substituent group may optionally be different. In embodiments, when R¹⁵ is substituted, it is substituted with at least one substituent group. In embodiments, when R¹⁵ is substituted, it is substituted with at least one size-limited substituent group. In embodiments, when R¹⁵ is substituted, it is substituted with at least one lower substituent group.

In embodiments, a substituted R^{15A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15A} is substituted with a plurality of groups selected from substituent groups, size-limited substituent groups, and lower substituent groups; each substituent group, size-limited substituent group, and/or lower substituent group may optionally be different. In embodiments, when R^{15A} is substituted, it is substituted with at least one substituent group. In embodiments, when R^{15A} is substituted, it is substituted with at least one size-limited substituent group. In embodiments, when R^{15A} is substituted, it is substituted with at least one lower substituent group.

In embodiments, a substituted R^{15B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15B} is substituted with a plurality of groups selected from substituent groups, size-limited substituent groups, and lower substituent groups; each substituent group, size-limited substituent group, and/or lower substituent group may optionally be different. In embodiments, when R^{15B} is substituted, it is substituted with at least one substituent group. In embodiments, when R^{15B} is substituted, it is substituted with at least one size-limited substituent group. In embodiments, when R^{15B} is substituted, it is substituted with at least one lower substituent group.

In embodiments, a substituted ring formed when R^{15A} and R^{15B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{15A} and R^{15B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{15A} and R^{15B} substituents bonded to the same nitrogen atom are joined is substituted, it is substituted with at least one substituent group. In embodiments, when the substituted ring formed when R^{15A} and R^{15B} substituents bonded to the same nitrogen atom are joined is substituted, it is substituted with at least one size-limited substituent group. In embodiments, when the substituted ring formed when R^{15A} and R^{15B} substituents bonded to the same nitrogen atom are joined is substituted, it is substituted with at least one lower substituent group.

In embodiments, a substituted R^{15C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15C} is substituted with a plurality of groups selected from substituent groups, size-limited substituent groups, and lower substituent groups; each substituent group, size-limited substituent group, and/or lower substituent group may optionally be different. In embodiments, when R^{15C} is substituted, it is substituted with at least one substituent group. In embodiments, when R^{15C} is substituted, it is substituted with at least one size-limited substituent group. In embodiments, when R^{15C} is substituted, it is substituted with at least one lower substituent group.

In embodiments, a substituted R^{15D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15D} is substituted with a plurality of groups selected from substituent groups, size-limited substituent groups, and lower substituent groups; each substituent group, size-limited substituent group, and/or lower substituent group may optionally be different. In embodiments, when R^{15D} is substituted, it is substituted with at least one substituent group. In embodiments, when R^{15D} is substituted, it is substituted with at least one size-limited substituent group. In embodiments, when R^{5D} is substituted, it is substituted with at least one lower substituent group.

In embodiments, R¹⁵ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁵ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁵ is independently hydrogen, substituted or unsubstituted C₁-C₄ alkyl or substituted or unsubstituted 2 to 4 membered heteroalkyl. In embodiments, R¹⁵ is hydrogen. In embodiments, R¹⁵ is unsubstituted methyl. In embodiments, R¹⁵ is unsubstituted ethyl. In embodiments, R¹⁵ is unsubstituted propyl. In embodiments, R¹⁵ is unsubstituted n-propyl. In embodiments, R¹⁵ is unsubstituted isopropyl. In embodiments, R¹⁵ is unsubstituted butyl. In embodiments, R¹⁵ is unsubstituted n-butyl. In embodiments, R¹⁵ is unsubstituted isobutyl. In embodiments, R¹⁵ is unsubstituted tert-butyl.

In embodiments, R^{15A} is hydrogen. In embodiments, R^{15A} is unsubstituted C₁-C₄ alkyl. In embodiments, R^{15A} is unsubstituted methyl. In embodiments, R^{15A} is unsubstituted ethyl. In embodiments, R^{15A} is unsubstituted propyl. In embodiments, R^{15A} is unsubstituted n-propyl. In embodiments, R^{15A} is unsubstituted isopropyl. In embodiments, R^{15A} is unsubstituted butyl. In embodiments, R^{15A} is unsubstituted n-butyl. In embodiments, R^{15A} is unsubstituted isobutyl. In embodiments, R^{15A} is unsubstituted tert-butyl.

In embodiments, R^{15B} is hydrogen. In embodiments, R^{15B} is unsubstituted C₁-C₄ alkyl. In embodiments, R^{15B} is unsubstituted methyl. In embodiments, R^{15B} is unsubstituted ethyl. In embodiments, R^{15B} is unsubstituted propyl. In embodiments, R^{15B} is unsubstituted n-propyl. In embodiments, R^{15B} is unsubstituted isopropyl. In embodiments, R^{15B} is unsubstituted butyl. In embodiments, R^{15B} is unsubstituted n-butyl. In embodiments, R^{15B} is unsubstituted isobutyl. In embodiments, R^{15B} is unsubstituted tert-butyl.

In embodiments, R^{15C} is hydrogen. In embodiments, R^{15C} is unsubstituted C₁-C₄ alkyl. In embodiments, R^{15C} is unsubstituted methyl. In embodiments, R^{15C} is unsubstituted ethyl. In embodiments, R^{15C} is unsubstituted propyl. In embodiments, R^{15C} is unsubstituted n-propyl. In embodiments, R^{15C} is unsubstituted isopropyl. In embodiments, R^{15C} is unsubstituted butyl. In embodiments, R^{15C} is unsubstituted n-butyl. In embodiments, R^{15C} is unsubstituted isobutyl. In embodiments, R^{15C} is unsubstituted tert-butyl.

In embodiments, R^{15D} is hydrogen. In embodiments, R^{15D} is unsubstituted C₁-C₄ alkyl. In embodiments, R^{15D} is unsubstituted methyl. In embodiments, R^{15D} is unsubstituted ethyl. In embodiments, R^{15D} is unsubstituted propyl. In embodiments, R^{15D} is unsubstituted n-propyl. In embodiments, R^{15D} is unsubstituted isopropyl. In embodiments, R^{15D} is unsubstituted butyl. In embodiments, R^{15D} is unsubstituted n-butyl. In embodiments, R^{15D} is unsubstituted isobutyl. In embodiments, R^{15D} is unsubstituted tert-butyl.

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, a substituted R^{16A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16A} is substituted with a plurality of groups selected from substituent groups, size-limited substituent groups, and lower substituent groups; each substituent group, size-limited substituent group, and/or lower substituent group may optionally be different. In embodiments, when R^{16A} is substituted, it is substituted with at least one substituent group. In embodiments, when R^{16A} is substituted, it is substituted with at least one size-limited substituent group. In embodiments, when R^{16A} is substituted, it is substituted with at least one lower substituent group.

In embodiments, a substituted R^{16B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16B} is substituted with a plurality of groups selected from substituent groups, size-limited substituent groups, and lower substituent groups; each substituent group, size-limited substituent group, and/or lower substituent group may optionally be different. In embodiments, when R^{16B} is substituted, it is substituted with at least one substituent group. In embodiments, when R^{16B} is substituted, it is substituted with at least one size-limited substituent group. In embodiments, when R^{16B} is substituted, it is substituted with at least one lower substituent group.

In embodiments, a substituted ring formed when R^{16A} and R^{16B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{16A} and R^{16B}, substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{16A} and R^{16B} substituents bonded to the same nitrogen atom are joined is substituted, it is substituted with at least one substituent group. In embodiments, when the substituted ring formed when R^{16A} and R^{16B} substituents bonded to the same nitrogen atom are joined is substituted, it is substituted with at least one size-limited substituent group. In embodiments, when the substituted ring formed when R^{16A} and R^{16B} substituents bonded to the same nitrogen atom are joined is substituted, it is substituted with at least one lower substituent group.

In embodiments, a substituted R^{16C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16C} is substituted with a plurality of groups selected from substituent groups, size-limited substituent groups, and lower substituent groups; each substituent group, size-limited substituent group, and/or lower substituent group may optionally be different. In embodiments, when R^{16C} is substituted, it is substituted with at least one substituent group. In embodiments, when R^{16C} is substituted, it is substituted with at least one size-limited substituent group. In embodiments, when R^{16C} is substituted, it is substituted with at least one lower substituent group.

In embodiments, a substituted R^{16D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16D} is substituted with a plurality of groups selected from substituent groups, size-limited substituent groups, and lower substituent groups; each substituent group, size-limited substituent group, and/or lower substituent group may optionally be different. In embodiments, when R^{16D} is substituted, it is substituted with at least one substituent group. In embodiments, when R^{16D} is substituted, it is substituted with at least one size-limited substituent group. In embodiments, when R^{16D} is substituted, it is substituted with at least one lower substituent group.

In embodiments, R¹⁶ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁶ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁶ is independently hydrogen, substituted or unsubstituted C₁-C₄ alkyl or substituted or unsubstituted 2 to 4 membered heteroalkyl. In embodiments, R¹⁶ is hydrogen. In embodiments, R¹⁶ is unsubstituted methyl. In embodiments, R¹⁶ is unsubstituted ethyl. In embodiments, R¹⁶ is unsubstituted propyl. In embodiments, R¹⁶ is unsubstituted n-propyl. In embodiments, R¹⁶ is unsubstituted isopropyl. In embodiments, R¹⁶ is unsubstituted butyl. In embodiments, R¹⁶ is unsubstituted n-butyl. In embodiments, R¹⁶ is unsubstituted isobutyl. In embodiments, R¹⁶ is unsubstituted tert-butyl. In embodiments, R¹⁶ is unsubstituted C₃-C₈ cycloalkyl. In embodiments, R¹⁶ is unsubstituted cyclopropyl. In embodiments, R¹⁶ is unsubstituted cyclobutyl. In embodiments, R¹⁶ is unsubstituted cyclopentyl. In embodiments, R¹⁶ is unsubstituted cyclohexyl. In embodiments, R¹⁶ is unsubstituted cycloheptyl. In embodiments, R¹⁶ is unsubstituted cyclooctyl. In embodiments, R¹⁶ is unsubstituted 5 to 6 membered heteroaryl. In embodiments, R¹⁶ is unsubstituted thienyl. In embodiments, R¹⁶ is unsubstituted 1-thienyl.

In embodiments, R¹⁶ is unsubstituted 2-thienyl. In embodiments, R¹⁶ is unsubstituted furanyl.

In embodiments, R¹⁶ is unsubstituted 1-furanyl. In embodiments, R¹⁶ is unsubstituted 2-furanyl. In embodiments, R¹⁶ is substituted or unsubstituted phenyl. In embodiments, R¹⁶ is substituted phenyl. In embodiments, R¹⁶ is unsubstituted phenyl. In embodiments, R¹⁶ is unsubstituted fused ring aryl. In embodiments, R¹⁶ is

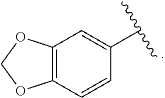 [SMILES: *c1ccc2c(c1)OCO2]

In embodiments, R^{16A} is hydrogen. In embodiments, R^{16A} is unsubstituted C₁-C₄ alkyl. In embodiments, R^{16A} is unsubstituted methyl. In embodiments, R^{16A} is unsubstituted ethyl. In embodiments, R^{16A} is unsubstituted propyl. In embodiments, R^{16A} is unsubstituted n-propyl. In embodiments, R^{16A} is unsubstituted isopropyl. In embodiments, R^{16A} is unsubstituted butyl. In embodiments, R^{16A} is unsubstituted n-butyl. In embodiments, R^{16A} is unsubstituted isobutyl. In embodiments, R^{16A} is unsubstituted tert-butyl.

In embodiments, R^{16B} is hydrogen. In embodiments, R^{16B} is unsubstituted C₁-C₄ alkyl. In embodiments, R^{16B} is unsubstituted methyl. In embodiments, R^{16B} is unsubstituted ethyl. In embodiments, R^{16B} is unsubstituted propyl. In embodiments, R^{16B} is unsubstituted n-propyl. In embodiments, R^{16B} is unsubstituted isopropyl. In embodiments, R^{16B} is unsubstituted butyl. In embodiments, R^{16B} is unsubstituted n-butyl. In embodiments, R^{16B} is unsubstituted isobutyl. In embodiments, R^{16B} is unsubstituted tert-butyl.

In embodiments, R^{16C} is hydrogen. In embodiments, R^{16C} is unsubstituted C₁-C₄ alkyl. In embodiments, R^{16C} is unsubstituted methyl. In embodiments, R^{16C} is unsubstituted ethyl. In embodiments, R^{16C} is unsubstituted propyl. In embodiments, R^{16C} is unsubstituted n-propyl. In embodiments, R^{16C} is unsubstituted isopropyl. In embodiments, R^{16C} is unsubstituted butyl. In embodiments, R^{16C} is unsubstituted n-butyl. In embodiments, R^{16C} is unsubstituted isobutyl. In embodiments, R^{16C} is unsubstituted tert-butyl.

In embodiments, R^{16D} is hydrogen. In embodiments, R^{16D} is unsubstituted C₁-C₄ alkyl. In embodiments, R^{16D} is unsubstituted methyl. In embodiments, R^{16D} is unsubstituted ethyl. In embodiments, R^{16D} is unsubstituted propyl. In embodiments, R^{16D} is unsubstituted n-propyl. In embodiments, R^{16D} is unsubstituted isopropyl. In embodiments, R^{16D} is unsubstituted butyl. In embodiments, R^{16D} is unsubstituted n-butyl. In embodiments, R^{16D} is unsubstituted isobutyl. In embodiments, R^{16D} is unsubstituted tert-butyl.

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, a substituted R^{17A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17A} is substituted with a plurality of groups selected from substituent groups, size-limited substituent groups, and lower substituent groups; each substituent group, size-limited substituent group, and/or lower substituent group may optionally be different. In embodiments, when R^{17A} is substituted, it is substituted with at least one substituent group. In embodiments, when R^{17A} is substituted, it is substituted with at least one size-limited substituent group. In embodiments, when R^{17A} is substituted, it is substituted with at least one lower substituent group.

In embodiments, a substituted R^{17B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17B} is substituted with a plurality of groups selected from substituent groups, size-limited substituent groups, and lower substituent groups; each substituent group, size-limited substituent group, and/or lower substituent group may optionally be different. In embodiments, when R^{17B} is substituted, it is substituted with at least one substituent group. In embodiments, when R^{17B} is substituted, it is substituted with at least one size-limited substituent group. In embodiments, when R^{17B} is substituted, it is substituted with at least one lower substituent group.

In embodiments, a substituted ring formed when R^{17A} and R^{17B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{17A} and R^{17B}, substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{17A} and R^{17B} substituents bonded to the same nitrogen atom are joined is substituted, it is substituted with at least one substituent group. In embodiments, when the substituted ring formed when R^{17A} and R^{17B} substituents bonded to the same nitrogen atom are joined is substituted, it is substituted with at least one size-limited substituent group. In embodiments, when the substituted ring formed when R^{17A} and R^{17B} substituents bonded to the same nitrogen atom are joined is substituted, it is substituted with at least one lower substituent group.

In embodiments, a substituted R^{17C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17C} is substituted with a plurality of groups selected from substituent groups, size-limited substituent groups, and lower substituent groups; each substituent group, size-limited substituent group, and/or lower substituent group may optionally be different. In embodiments, when R^{17C} is substituted, it is substituted with at least one substituent group. In embodiments, when R^{17C} is substituted, it is substituted with at least one size-limited substituent group. In embodiments, when R^{17C} is substituted, it is substituted with at least one lower substituent group.

In embodiments, a substituted R^{17D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17D} is substituted with a plurality of groups selected from substituent groups, size-limited substituent groups, and lower substituent groups; each substituent group, size-limited substituent group, and/or lower substituent group may optionally be different. In embodiments, when R^{17D} is substituted, it is substituted with at least one substituent group. In embodiments, when R^{17D} is substituted, it is substituted with at least one size-limited substituent group. In embodiments, when R^{17D} is substituted, it is substituted with at least one lower substituent group.

In embodiments, R¹⁷ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁷ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁷ is independently hydrogen, substituted or unsubstituted C₁-C₄ alkyl or substituted or unsubstituted 2 to 4 membered heteroalkyl. In embodiments, R¹⁷ is hydrogen. In embodiments, R¹⁷ is unsubstituted methyl. In embodiments, R¹⁷ is unsubstituted ethyl. In embodiments, R¹⁷ is unsubstituted propyl. In embodiments, R¹⁷ is unsubstituted n-propyl. In embodiments, R¹⁷ is unsubstituted isopropyl. In embodiments, R¹⁷ is unsubstituted butyl. In embodiments, R¹⁷ is unsubstituted n-butyl. In embodiments, R¹⁷ is unsubstituted isobutyl. In embodiments, R¹⁷ is unsubstituted tert-butyl. In embodiments, R¹⁷ is —CN.

In embodiments, R^{17A} is hydrogen. In embodiments, R^{17A} is unsubstituted C₁-C₄ alkyl. In embodiments, R^{17A} is unsubstituted methyl. In embodiments, R^{17A} is unsubstituted ethyl. In embodiments, R^{17A} is unsubstituted propyl. In embodiments, R^{17A} is unsubstituted n-propyl. In embodiments, R^{17A} is unsubstituted isopropyl. In embodiments, R^{17A} is unsubstituted butyl. In embodiments, R^{17A} is unsubstituted n-butyl. In embodiments, R^{17A} is unsubstituted isobutyl. In embodiments, R^{17A} is unsubstituted tert-butyl.

In embodiments, R^{17B} is hydrogen. In embodiments, R^{17B} is unsubstituted C₁-C₄ alkyl. In embodiments, R^{17B} is unsubstituted methyl. In embodiments, R^{17B} is unsubstituted ethyl. In embodiments, R^{17B} is unsubstituted propyl. In embodiments, R^{17B} is unsubstituted n-propyl. In embodiments, R^{17B} is unsubstituted isopropyl. In embodiments, R^{17B} is unsubstituted butyl. In embodiments, R^{17B} is unsubstituted n-butyl. In embodiments, R^{17B} is unsubstituted isobutyl. In embodiments, R^{17B} is unsubstituted tert-butyl.

In embodiments, R^{17C} is hydrogen. In embodiments, R^{17C} is unsubstituted C₁-C₄ alkyl. In embodiments, R^{17C} is unsubstituted methyl. In embodiments, R^{17C} is unsubstituted ethyl. In embodiments, R^{17C} is unsubstituted propyl. In embodiments, R^{17C} is unsubstituted n-propyl. In embodiments, R^{17C} is unsubstituted isopropyl. In embodiments, R^{17C} is unsubstituted butyl. In embodiments, R^{17C} is unsubstituted n-butyl. In embodiments, R^{17C} is unsubstituted isobutyl. In embodiments, R^{17C} is unsubstituted tert-butyl.

In embodiments, R^{17D} is hydrogen. In embodiments, R^{17D} is unsubstituted C₁-C₄ alkyl. In embodiments, R^{17D} is unsubstituted methyl. In embodiments, R^{17D} is unsubstituted ethyl. In embodiments, R^{17D} is unsubstituted propyl. In embodiments, R^{17D} is unsubstituted n-propyl. In embodiments, R^{17D} is unsubstituted isopropyl. In embodiments, R^{17D} is unsubstituted butyl. In embodiments, R^{17D} is unsubstituted n-butyl. In embodiments, R^{17D} is unsubstituted isobutyl. In embodiments, R^{17D} is unsubstituted tert-butyl.

In embodiments, a substituted R¹⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

In embodiments, a substituted R^{18A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18A} is substituted with a plurality of groups selected from substituent groups, size-limited substituent groups, and lower substituent groups; each substituent group, size-limited substituent group, and/or lower substituent group may optionally be different. In embodiments, when R^{18A} is substituted, it is substituted with at least one substituent group. In embodiments, when R^{18A} is substituted, it is substituted with at least one size-limited substituent group. In embodiments, when R^{18A} is substituted, it is substituted with at least one lower substituent group.

In embodiments, a substituted R^{18B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18B} is substituted with a plurality of groups selected from substituent groups, size-limited substituent groups, and lower substituent groups; each substituent group, size-limited substituent group, and/or lower substituent group may optionally be different. In embodiments, when R^{18B} is substituted, it is substituted with at least one substituent group. In embodiments, when R^{18B} is substituted, it is substituted with at least one size-limited substituent group. In embodiments, when R⁸B is substituted, it is substituted with at least one lower substituent group.

In embodiments, a substituted ring formed when R^{18A} and R^{18B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{18A} and R^{18B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{18A} and R^{18B} substituents bonded to the same nitrogen atom are joined is substituted, it is substituted with at least one substituent group. In embodiments, when the substituted ring formed when R^{18A} and R^{18B} substituents bonded to the same nitrogen atom are joined is substituted, it is substituted with at least one size-limited substituent group. In embodiments, when the substituted ring formed when R^{18A} and R^{18B} substituents bonded to the same nitrogen atom are joined is substituted, it is substituted with at least one lower substituent group.

In embodiments, a substituted R^{18C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18C} is substituted with a plurality of groups selected from substituent groups, size-limited substituent groups, and lower substituent groups; each substituent group, size-limited substituent group, and/or lower substituent group may optionally be different. In embodiments, when R^{18C} is substituted, it is substituted with at least one substituent group. In embodiments, when R^{18C} is substituted, it is substituted with at least one size-limited substituent group. In embodiments, when R^{18C} is substituted, it is substituted with at least one lower substituent group.

In embodiments, R¹⁸ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁸ is independently hydrogen, substituted or unsubstituted C₁-C₄ alkyl or substituted or unsubstituted 2 to 4 membered heteroalkyl. In embodiments, R¹⁸ is hydrogen. In embodiments, R¹⁸ is unsubstituted methyl. In embodiments, R¹⁸ is unsubstituted ethyl. In embodiments, R¹⁸ is unsubstituted propyl. In embodiments, R¹⁸ is unsubstituted n-propyl. In embodiments, R¹⁸ is unsubstituted isopropyl. In embodiments, R¹⁸ is unsubstituted butyl. In embodiments, R¹⁸ is unsubstituted n-butyl. In embodiments, R¹⁸ is unsubstituted isobutyl. In embodiments, R¹⁸ is unsubstituted tert-butyl.

In embodiments, R^{18A} is hydrogen. In embodiments, R^{18A} is unsubstituted C₁-C₄ alkyl. In embodiments, R^{18A} is unsubstituted methyl. In embodiments, R^{18A} is unsubstituted ethyl. In embodiments, R^{18A} is unsubstituted propyl. In embodiments, R^{18A} is unsubstituted n-propyl. In embodiments, R^{18A} is unsubstituted isopropyl. In embodiments, R^{18A} is unsubstituted butyl. In embodiments, R^{18A} is unsubstituted n-butyl. In embodiments, R^{18A} is unsubstituted isobutyl. In embodiments, R^{18A} is unsubstituted tert-butyl.

In embodiments, R^{18B} is hydrogen. In embodiments, R^{18B} is unsubstituted C₁-C₄ alkyl. In embodiments, R^{18B} is unsubstituted methyl. In embodiments, R^{18B} is unsubstituted ethyl. In embodiments, R^{18B} is unsubstituted propyl. In embodiments, R^{18B} is unsubstituted n-propyl. In embodiments, R^{18B} is unsubstituted isopropyl. In embodiments, R′″ is unsubstituted butyl. In embodiments, R^{18B} is unsubstituted n-butyl. In embodiments, R′″ is unsubstituted isobutyl. In embodiments, R^{18B} is unsubstituted tert-butyl.

In embodiments, R^{18C} is hydrogen. In embodiments, R^{18C} is unsubstituted C₁-C₄ alkyl. In embodiments, R^{18C} is unsubstituted methyl. In embodiments, R^{18C} is unsubstituted ethyl. In embodiments, R^{18C} is unsubstituted propyl. In embodiments, R^{18C} is unsubstituted n-propyl. In embodiments, R^{18C} is unsubstituted isopropyl. In embodiments, R^{18C} is unsubstituted butyl. In embodiments, R^{18C} is unsubstituted n-butyl. In embodiments, R^{18C} is unsubstituted isobutyl. In embodiments, R^{18C} is unsubstituted tert-butyl.

In embodiments, X¹⁶ is halogen. In embodiments, X¹⁶ is —F. In embodiments, X¹⁶ is —Cl. In embodiments, X¹⁶ is —Br. In embodiments, X¹⁶ is —I.

In embodiments, X¹⁷ is halogen. In embodiments, X¹⁷ is —F. In embodiments, X¹⁷ is —Cl. In embodiments, X¹⁷ is —Br. In embodiments, X¹⁷ is —I.

In embodiments, the serine binding moiety is:

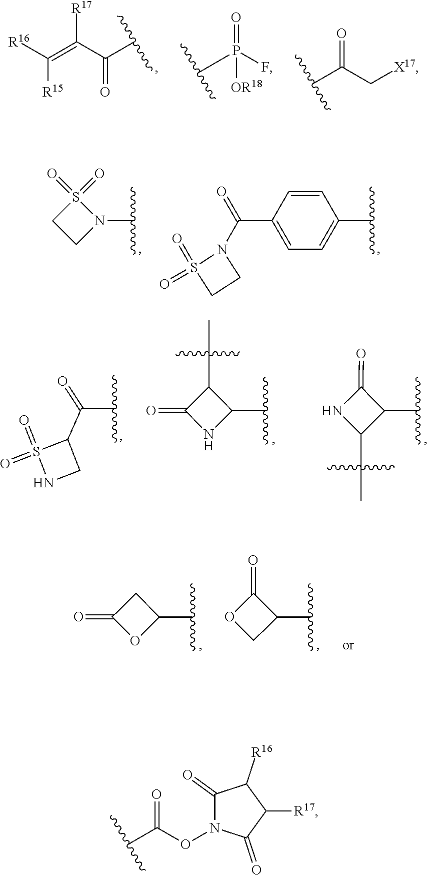

wherein R¹⁵, R¹⁶, R¹⁷, R¹⁸, and X¹⁷ are as described herein, including embodiments.

In embodiments, the serine binding moiety is

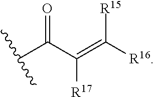

In embodiments, the serine binding moiety is

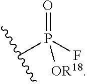

In embodiments, the serine binding moiety is

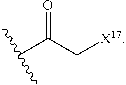

In embodiments, the serine binding moiety is

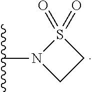 [SMILES: *N1CCS1(=O)=O]

In embodiments, the serine binding moiety is

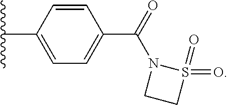 [SMILES: *c1ccc(C(=O)N2CCS2(=O)=O)cc1]

In embodiments, the serine binding moiety is

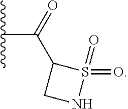 [SMILES: *C(=O)C1CNS1(=O)=O]

In embodiments, the serine binding moiety is

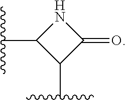 [SMILES: *C1NC(=O)C1*]

In embodiments, the serine binding moiety is

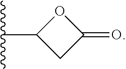 [SMILES: *C1CC(=O)O1]

In embodiments, the serine binding moiety is

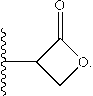 [SMILES: *C1COC1=O]

In embodiments, the serine binding moiety is

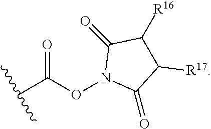

In embodiments, the serine binding moiety is:

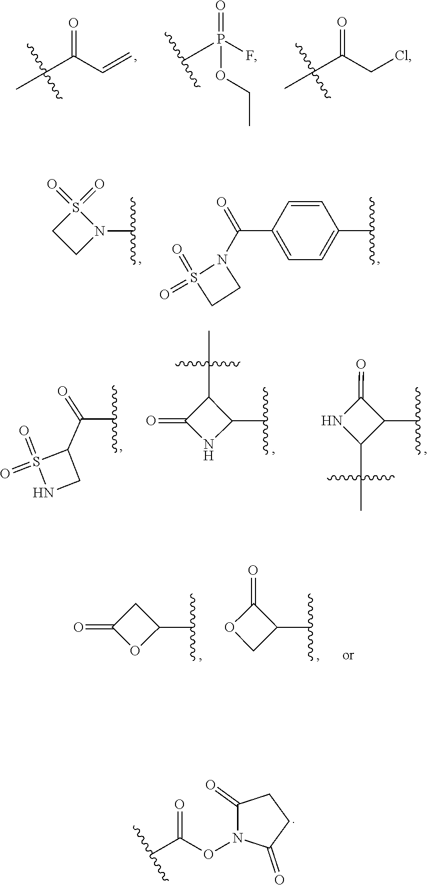

In embodiments, the serine binding moiety is

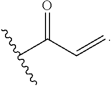 [SMILES: *C(=O)C=C]

In embodiments, the serine binding moiety is

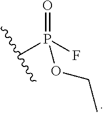 [SMILES: *P(=O)(F)OCC]

In embodiments, the serine binding moiety is

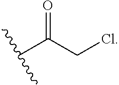 [SMILES: *C(=O)CCl]

In embodiments, the serine binding moiety is

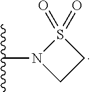 [SMILES: *N1CCS1(=O)=O]

In embodiments, the serine binding moiety is

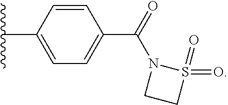 [SMILES: *c1ccc(C(=O)N2CCS2(=O)=O)cc1]

In embodiments, the serine binding moiety is

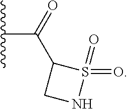 [SMILES: *C(=O)C1CNS1(=O)=O]

In embodiments, the serine binding moiety is

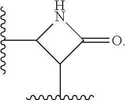 [SMILES: *C1NC(=O)C1*]

In embodiments, the serine binding moiety is

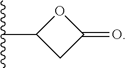 [SMILES: *C1CC(=O)O1]

In embodiments, the serine binding moiety is

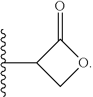 [SMILES: *C1COC1=O]

In embodiments, the serine binding moiety is

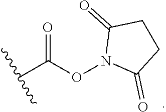 [SMILES: *C(=O)ON1C(=O)CCC1=O]

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, when R¹ is substituted, R¹ is substituted with one or more first substituent groups denoted by R^{1.1} as explained in the definitions section above in the description of “first substituent group(s)”. In embodiments, when an R¹ substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, Ric, Rici, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to Ric, Rici, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A}1 as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{2C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R⁴B is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D}, is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R⁶ and L¹ substituents bonded to the same nitrogen atom are optionally joined to form a moiety that is substituted (e.g., a substituted heterocycloalkyl), the moiety is substituted with one or more first substituent groups denoted by R^{6.1} as explained in the definitions section above in the description of “first substituent group(s)”. In embodiments, when an R⁶¹ substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R^{6.1}, R^{6.2}, and R⁶³ have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6.1}, R^{6.2}, and R^{6.3}, respectively.

In embodiments, when R⁶ and L¹ substituents bonded to the same nitrogen atom are optionally joined to form a moiety that is substituted (e.g., a substituted heterocycloalkyl), the moiety is substituted with one or more first substituent groups denoted by L¹ as explained in the definitions section above in the description of “first substituent group(s)”. In embodiments, when an L^{1.1} substituent group is substituted, the L^{1.1} substituent group is substituted with one or more second substituent groups denoted by L^{1.2} as explained in the definitions section above in the description of “first substituent group(s)”. In embodiments, when an L^{1.2} substituent group is substituted, the L^{1.2} substituent group is substituted with one or more third substituent groups denoted by L^{1.3} as explained in the definitions section above in the description of “first substituent group(s)”. In the above embodiments, L^{1.1}, L^{1.2}, and L^{1.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to L^{1.1}, L^{1.2}, and L^{1.3}, respectively.

In embodiments, when R⁶ and R⁷ substituents bonded to the same nitrogen atom are optionally joined to form a moiety that is substituted (e.g., a substituted heterocycloalkyl or substituted heteroaryl), the moiety is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R^{6.1}, R^{6.2}, and R^{6.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6.1}, R^{6.2}, and R^{6.3}, respectively.

In embodiments, when R⁶ and R⁷ substituents bonded to the same nitrogen atom are optionally joined to form a moiety that is substituted (e.g., a substituted heterocycloalkyl or substituted heteroaryl), the moiety is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R^{7.1}, R^{7.2}, and R^{7.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7.1}, R^{7.2}, and R^{7.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when two adjacent R⁷ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when R^{7A} is substituted, R^{7A} is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A}, R^{7A.1}, R^{7A.2}, and R^{7A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7A}, R⁷¹, R^{7A.2}, and R^{7A.3}, respectively.

In embodiments, when R^{7B} is substituted, R^{7B} is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B}, R^{7B.1}, R^{7B.2}, and R^{7B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7B}, R^{7B.1}, R^{7B.2}, and R^{7B.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A.1}, R^{7A.2}, and R^{7A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7A.1}, R^{7A.2}, and R^{7A.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B.1}, R^{7B.2}, and R^{7B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7B.1}, R^{7B.2}, and R^{7B.3}, respectively.

In embodiments, when R^{7C} is substituted, R^{7C} is substituted with one or more first substituent groups denoted by R^{7C.1} as explained in the definitions section above in the description of “first substituent group(s)”. In embodiments, when an R^{7C.1} substituent group is substituted, the R^{7C.1} substituent group is substituted with one or more second substituent groups denoted by R^{7C.2} as explained in the definitions section above in the description of “first substituent group(s)”. In embodiments, when an R^{7C.2} substituent group is substituted, the R^{7C.2} substituent group is substituted with one or more third substituent groups denoted by R^{7C.3} as explained in the definitions section above in the description of “first substituent group(s)”. In the above embodiments, R^{7C}, R^{7C.1}, R^{7C.2}, and R^{7C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7C}, R^{7C.1}, R^{7C.2}, and R^{7C.3}, respectively.

In embodiments, when R^{7D} is substituted, R^{7D} is substituted with one or more first substituent groups denoted by R^{7D.1} as explained in the definitions section above in the description of “first substituent group(s)”. In embodiments, when an R^{7D.1} substituent group is substituted, the R^{7D.1} substituent group is substituted with one or more second substituent groups denoted by R^{7D.2} as explained in the definitions section above in the description of “first substituent group(s)”. In embodiments, when an R^{7D.2} substituent group is substituted, the R^{7D.2} substituent group is substituted with one or more third substituent groups denoted by R^{7D.3} as explained in the definitions section above in the description of “first substituent group(s)”. In the above embodiments, R^{7D}, R^{7D.1}, R^{7D.2}, and R^{7D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7D}, R^{7D.1}, R^{7D.2}, and R^{7D.3}, respectively.

In embodiments, when R¹⁵ is substituted, R¹⁵ is substituted with one or more first substituent groups denoted by R^{15.1} as explained in the definitions section above in the description of “first substituent group(s)”. In embodiments, when an R^{15.1} substituent group is substituted, the R^{15.1} substituent group is substituted with one or more second substituent groups denoted by R^{15.2} as explained in the definitions section above in the description of “first substituent group(s)”. In embodiments, when an R^{15.2} substituent group is substituted, the R^{15.2} substituent group is substituted with one or more third substituent groups denoted by R^{15.3} as explained in the definitions section above in the description of “first substituent group(s)”. In the above embodiments, R¹⁵, R^{15.1}, R^{15.2}, and R^{15.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁵, R^{15.1}, R^{15.2}, and R^{15.3}, respectively.

In embodiments, when R^{15A} is substituted, R^{15A} is substituted with one or more first substituent groups denoted by R^{15A.1} as explained in the definitions section above in the description of “first substituent group(s)”. In embodiments, when an R^{15A.1} substituent group is substituted, the R^{15A.1} substituent group is substituted with one or more second substituent groups denoted by R^{15A.2} as explained in the definitions section above in the description of “first substituent group(s)”. In embodiments, when an R^{15A.2} substituent group is substituted, the R^{15A.2} substituent group is substituted with one or more third substituent groups denoted by R^{15A.3} as explained in the definitions section above in the description of “first substituent group(s)”. In the above embodiments, R^{15A}, R^{15A.1}, R^{15A.2}, and R^{15A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{15A}, R^{15A.1}, R^{15A.2}, and R^{15A.3}, respectively.

In embodiments, when R^{15B} is substituted, R^{15B} is substituted with one or more first substituent groups denoted by R^{15B.1} as explained in the definitions section above in the description of “first substituent group(s)”. In embodiments, when an R^{15B.1} substituent group is substituted, the R^{15B.1} substituent group is substituted with one or more second substituent groups denoted by R^{15B.2} as explained in the definitions section above in the description of “first substituent group(s)”. In embodiments, when an R^{15B.2} substituent group is substituted, the R^{15B.2} substituent group is substituted with one or more third substituent groups denoted by R^{15B.3} as explained in the definitions section above in the description of “first substituent group(s)”. In the above embodiments, R^{15B}, R^{15B.1}, R^{15B.2}, and R^{15B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{15B}, R^{15B.1}, R^{15B.2} and R^{15.3}, respectively.

In embodiments, when R^{15A} and R^{15B} substituents bonded to the same nitrogen atom are optionally joined to form a moiety that is substituted (e.g., a substituted heterocycloalkyl or substituted heteroaryl), the moiety is substituted with one or more first substituent groups denoted by R^{15A.1} as explained in the definitions section above in the description of “first substituent group(s)”. In embodiments, when an R^{15A.1} substituent group is substituted, the R^{15A.1} substituent group is substituted with one or more second substituent groups denoted by R^{15A.2} as explained in the definitions section above in the description of “first substituent group(s)”. In embodiments, when an R^{15A.2} substituent group is substituted, the R^{15A.2} substituent group is substituted with one or more third substituent groups denoted by R^{15A.3} as explained in the definitions section above in the description of “first substituent group(s)”. In the above embodiments, R^{15A.1}, R^{15A.2}, and R^{15A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{15A.1}, R^{15A.2}, and R^{15A.3}, respectively.

In embodiments, when R^{15A} and R^{15B} substituents bonded to the same nitrogen atom are optionally joined to form a moiety that is substituted (e.g., a substituted heterocycloalkyl or substituted heteroaryl), the moiety is substituted with one or more first substituent groups denoted by R^{15B.1} as explained in the definitions section above in the description of “first substituent group(s)”. In embodiments, when an R^{15B.1} substituent group is substituted, the R^{15B.1} substituent group is substituted with one or more second substituent groups denoted by R^{15B.2} as explained in the definitions section above in the description of “first substituent group(s)”. In embodiments, when an R^{15B.2} substituent group is substituted, the R^{15B.2} substituent group is substituted with one or more third substituent groups denoted by R^{15B.3} as explained in the definitions section above in the description of “first substituent group(s)”. In the above embodiments, R^{15B.1}, R^{15B.2}, and R^{15B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{15B.1}, R^{15B.2}, and R^{15B.3}, respectively.

In embodiments, when R^{15C} is substituted, R^{15C} is substituted with one or more first substituent groups denoted by R^{15C.1} as explained in the definitions section above in the description of “first substituent group(s)”. In embodiments, when an R^{15C.1} substituent group is substituted, the R^{15C.1} substituent group is substituted with one or more second substituent groups denoted by R^{15C.2} as explained in the definitions section above in the description of “first substituent group(s)”. In embodiments, when an R^{15C.2} substituent group is substituted, the R^{15C.2} substituent group is substituted with one or more third substituent groups denoted by R^{15C.3} as explained in the definitions section above in the description of “first substituent group(s)”. In the above embodiments, R^{15C}, R^{15C.1}, R^{15C.2}, and R^{15C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{15C}, R^{15C.1}, R^{15C.2}, and R^{15C.3}, respectively.

In embodiments, when R^{15D} is substituted, R^{15D} is substituted with one or more first substituent groups denoted by R^{15D.1} as explained in the definitions section above in the description of “first substituent group(s)”. In embodiments, when an R^{15D.1} substituent group is substituted, the R^{15D.1} substituent group is substituted with one or more second substituent groups denoted by R^{15D.2} as explained in the definitions section above in the description of “first substituent group(s)”. In embodiments, when an R^{15D.2} substituent group is substituted, the R^{15D.2} substituent group is substituted with one or more third substituent groups denoted by R^{15D.3} as explained in the definitions section above in the description of “first substituent group(s)”. In the above embodiments, R^{15D}, R^{15D.1}, R^{15D.2}, and R^{15D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{15D}, R^{15D.1}, R^{15D.2} and R^{15D.3}, respectively.

In embodiments, when R¹⁶ is substituted, R¹⁶ is substituted with one or more first substituent groups denoted by R^{16.1} as explained in the definitions section above in the description of “first substituent group(s)”. In embodiments, when an R^{16.1} substituent group is substituted, the R^{16.1} substituent group is substituted with one or more second substituent groups denoted by R^{16.2} as explained in the definitions section above in the description of “first substituent group(s)”. In embodiments, when an R^{16.2} substituent group is substituted, the R^{16.2} substituent group is substituted with one or more third substituent groups denoted by R¹⁶³ as explained in the definitions section above in the description of “first substituent group(s)”. In the above embodiments, R¹⁶, R^{16.1}, R^{16.2}, and R^{16.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁶, R^{16.1}, R^{16.2}, and R^{16.3}, respectively.

In embodiments, when R^{16A} is substituted, R^{16A} is substituted with one or more first substituent groups denoted by R^{16A.1} as explained in the definitions section above in the description of “first substituent group(s)”. In embodiments, when an R^{16A.1} substituent group is substituted, the R^{16A.1} substituent group is substituted with one or more second substituent groups denoted by R^{16A.2} as explained in the definitions section above in the description of “first substituent group(s)”. In embodiments, when an R^{16A.2} substituent group is substituted, the R^{16A.2} substituent group is substituted with one or more third substituent groups denoted by R^{16A.3} as explained in the definitions section above in the description of “first substituent group(s)”. In the above embodiments, R^{16A}, R^{16A.1}, R^{16A.2}, and R^{16A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{16A}, R^{16A.1}, R^{16A.2}, and R^{16A.3}, respectively.

In embodiments, when R^{16B} is substituted, R^{16B} is substituted with one or more first substituent groups denoted by R^{16B.1} as explained in the definitions section above in the description of “first substituent group(s)”. In embodiments, when an R^{16B.1} substituent group is substituted, the R^{16B.1} substituent group is substituted with one or more second substituent groups denoted by R^{16B.2} as explained in the definitions section above in the description of “first substituent group(s)”. In embodiments, when an R^{16B.2} substituent group is substituted, the R^{16B.2} substituent group is substituted with one or more third substituent groups denoted by R^{16B.3} as explained in the definitions section above in the description of “first substituent group(s)”. In the above embodiments, R^{16B}, R^{16B.1}, R^{16B.2}, and R^{16B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{16B}, R^{16B.1}, R^{16B.2} and R^{16.3}, respectively.

In embodiments, when R^{16A} and R^{16B} substituents bonded to the same nitrogen atom are optionally joined to form a moiety that is substituted (e.g., a substituted heterocycloalkyl or substituted heteroaryl), the moiety is substituted with one or more first substituent groups denoted by R^{16A.1} as explained in the definitions section above in the description of “first substituent group(s)”. In embodiments, when an R^{16A.1} substituent group is substituted, the R^{16A.1} substituent group is substituted with one or more second substituent groups denoted by R^{6A.2} as explained in the definitions section above in the description of “first substituent group(s)”. In embodiments, when an R^{16A.2} substituent group is substituted, the R^{16A.2} substituent group is substituted with one or more third substituent groups denoted by R^{16A.3} as explained in the definitions section above in the description of “first substituent group(s)”. In the above embodiments, R^{16A.1}, R^{16A.2}, and R^{16A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{16A.1}, R^{16A.2}, and R^{16A.3}, respectively.

In embodiments, when R^{16A} and R^{16B} substituents bonded to the same nitrogen atom are optionally joined to form a moiety that is substituted (e.g., a substituted heterocycloalkyl or substituted heteroaryl), the moiety is substituted with one or more first substituent groups denoted by R^{16B.1} as explained in the definitions section above in the description of “first substituent group(s)”. In embodiments, when an R^{16B.1} substituent group is substituted, the R^{16B.1} substituent group is substituted with one or more second substituent groups denoted by R^{16B.2} as explained in the definitions section above in the description of “first substituent group(s)”. In embodiments, when an R^{16B.2} substituent group is substituted, the R^{16B.2} substituent group is substituted with one or more third substituent groups denoted by R^{16B.3} as explained in the definitions section above in the description of “first substituent group(s)”. In the above embodiments, R^{16B.1}, R^{16B.2}, and R^{16B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{16B.1}, R^{16B.2}, and R^{16B.3}, respectively.

In embodiments, when R^{16C} is substituted, R^{16C} is substituted with one or more first substituent groups denoted by R^{16C.1} as explained in the definitions section above in the description of “first substituent group(s)”. In embodiments, when an R^{16C.1} substituent group is substituted, the R^{16C.1} substituent group is substituted with one or more second substituent groups denoted by R^{16C.2} as explained in the definitions section above in the description of “first substituent group(s)”. In embodiments, when an R^{16C.2} substituent group is substituted, the R^{16C.2} substituent group is substituted with one or more third substituent groups denoted by R^{16C.3} as explained in the definitions section above in the description of “first substituent group(s)”. In the above embodiments, R^{16C}, R^{16C.1}, R^{16C.2}, and R^{16C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{16C}, R^{16C.1}, R^{16C.2} and R^{16C.3}, respectively.

In embodiments, when R^{16D} is substituted, R^{16D} is substituted with one or more first substituent groups denoted by R^{16D.1} as explained in the definitions section above in the description of “first substituent group(s)”. In embodiments, when an R^{16D.1} substituent group is substituted, the R^{16D.1} substituent group is substituted with one or more second substituent groups denoted by R^{16D.2} as explained in the definitions section above in the description of “first substituent group(s)”. In embodiments, when an R^{16D.2} substituent group is substituted, the R^{16D.2} substituent group is substituted with one or more third substituent groups denoted by R^{16D.3} as explained in the definitions section above in the description of “first substituent group(s)”. In the above embodiments, R^{16D}, R^{16D.1}, R^{16D.2}, and R^{16D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{16D}, R^{16D.1}, R^{16D.2}, and R^{16D.3}, respectively.

In embodiments, when R¹⁷ is substituted, R¹⁷ is substituted with one or more first substituent groups denoted by R^{17.1} as explained in the definitions section above in the description of “first substituent group(s)”. In embodiments, when an R^{17.1} substituent group is substituted, the R^{17.1} substituent group is substituted with one or more second substituent groups denoted by R^{17.2} as explained in the definitions section above in the description of “first substituent group(s)”. In embodiments, when an R^{17.2} substituent group is substituted, the R^{17.2} substituent group is substituted with one or more third substituent groups denoted by R^{17.3} as explained in the definitions section above in the description of “first substituent group(s)”. In the above embodiments, R¹⁷, R^{17.1}, R^{17.2}, and R^{17.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁷, R^{17.1}, R^{17.2}, and R^{17.3}, respectively.

In embodiments, when R^{17A} is substituted, R^{17A} is substituted with one or more first substituent groups denoted by R^{17A.1} as explained in the definitions section above in the description of “first substituent group(s)”. In embodiments, when an R^{17A.1} substituent group is substituted, the R^{17A.1} substituent group is substituted with one or more second substituent groups denoted by R^{17A.2} as explained in the definitions section above in the description of “first substituent group(s)”. In embodiments, when an R^{17A.2} substituent group is substituted, the R^{17A.2} substituent group is substituted with one or more third substituent groups denoted by R^{17A.3} as explained in the definitions section above in the description of “first substituent group(s)”. In the above embodiments, R^{17A}, R^{17A.1}, R^{17A.2}, and R^{17A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{17A}, R^{17A.1}, R^{17A.2}, and R^{17A.3}, respectively.

In embodiments, when R^{17B} is substituted, R^{17B} is substituted with one or more first substituent groups denoted by R^{17B.1} as explained in the definitions section above in the description of “first substituent group(s)”. In embodiments, when an R^{17B.1} substituent group is substituted, the R^{17B.1} substituent group is substituted with one or more second substituent groups denoted by R^{17B.2} as explained in the definitions section above in the description of “first substituent group(s)”. In embodiments, when an R^{17B.2} substituent group is substituted, the R^{17B.2} substituent group is substituted with one or more third substituent groups denoted by R^{17B.3} as explained in the definitions section above in the description of “first substituent group(s)”. In the above embodiments, R^{17B}, R^{17B.1}, R^{17B.2}, and R^{17B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{17B}, R^{17B.1}, R^{17B.2}, and R^{17B.3}, respectively.

In embodiments, when R^{17A} and R^{17B} substituents bonded to the same nitrogen atom are optionally joined to form a moiety that is substituted (e.g., a substituted heterocycloalkyl or substituted heteroaryl), the moiety is substituted with one or more first substituent groups denoted by R^{17A.1} as explained in the definitions section above in the description of “first substituent group(s)”. In embodiments, when an R^{17A.1} substituent group is substituted, the R^{17A.1} substituent group is substituted with one or more second substituent groups denoted by R^{17A.2} as explained in the definitions section above in the description of “first substituent group(s)”. In embodiments, when an R^{17A.2} substituent group is substituted, the R^{17A.2} substituent group is substituted with one or more third substituent groups denoted by R^{17A.3} as explained in the definitions section above in the description of “first substituent group(s)”. In the above embodiments, R^{17A.1}, R^{17A.2}, and R^{17A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{17A.1}, R^{17A.2}, and R^{17A.3}, respectively.

In embodiments, when R^{17A} and R^{17B} substituents bonded to the same nitrogen atom are optionally joined to form a moiety that is substituted (e.g., a substituted heterocycloalkyl or substituted heteroaryl), the moiety is substituted with one or more first substituent groups denoted by R^{17B.1} as explained in the definitions section above in the description of “first substituent group(s)”. In embodiments, when an R^{17B.1} substituent group is substituted, the R^{17B.1} substituent group is substituted with one or more second substituent groups denoted by R^{17B.2} as explained in the definitions section above in the description of “first substituent group(s)”. In embodiments, when an R^{17B.2} substituent group is substituted, the R^{17B.2} substituent group is substituted with one or more third substituent groups denoted by R^{17B.3} as explained in the definitions section above in the description of “first substituent group(s)”. In the above embodiments, R^{17B.1}, R^{17B.2}, and R^{17B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{17B.1}, R^{17B.2}, and R^{17B.3}, respectively.

In embodiments, when R^{17C} is substituted, R^{17C} is substituted with one or more first substituent groups denoted by R^{17C.1} as explained in the definitions section above in the description of “first substituent group(s)”. In embodiments, when an R^{17C.1} substituent group is substituted, the R^{17C.1} substituent group is substituted with one or more second substituent groups denoted by R^{17C.2} as explained in the definitions section above in the description of “first substituent group(s)”. In embodiments, when an R^{17C.2} substituent group is substituted, the R^{17C.2} substituent group is substituted with one or more third substituent groups denoted by R^{17C.3} as explained in the definitions section above in the description of “first substituent group(s)”. In the above embodiments, R^{17C}, R^{17C.1}, R^{17C.2}, and R^{17C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{17C}, R^{17C.1}, R^{17C.2} and R^{17C.3}, respectively.

In embodiments, when R^{17D} is substituted, R^{17D} is substituted with one or more first substituent groups denoted by R^{17D.1} as explained in the definitions section above in the description of “first substituent group(s)”. In embodiments, when an R^{17D.1} substituent group is substituted, the R^{17D.1} substituent group is substituted with one or more second substituent groups denoted by R^{17D.2} as explained in the definitions section above in the description of “first substituent group(s)”. In embodiments, when an R^{17D.2} substituent group is substituted, the R^{17D.2} substituent group is substituted with one or more third substituent groups denoted by R^{17D.3} as explained in the definitions section above in the description of “first substituent group(s)”. In the above embodiments, R^{17D}, R^{17D.1}, R^{17D.2}, and R^{17D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{17D}, R^{17D.1}, R^{17D.2} and R^{17D.3}, respectively.

In embodiments, when R¹⁸ is substituted, R¹⁸ is substituted with one or more first substituent groups denoted by R^{18.1} as explained in the definitions section above in the description of “first substituent group(s)”. In embodiments, when an R^{18.1} substituent group is substituted, the R^{18.1} substituent group is substituted with one or more second substituent groups denoted by R^{18.2} as explained in the definitions section above in the description of “first substituent group(s)”. In embodiments, when an R^{18.2} substituent group is substituted, the R^{18.2} substituent group is substituted with one or more third substituent groups denoted by R^{18.3} as explained in the definitions section above in the description of “first substituent group(s)”. In the above embodiments, R¹⁸, R^{18.1}, R^{18.2}, and R^{18.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁸, R^{18.1}, R^{18.2}, and R^{18.3}, respectively.

In embodiments, when R^{18A} is substituted, R^{18A} is substituted with one or more first substituent groups denoted by R^{18A.1} as explained in the definitions section above in the description of “first substituent group(s)”. In embodiments, when an R^{18A.1} substituent group is substituted, the R^{18A.1} substituent group is substituted with one or more second substituent groups denoted by R^{18A.2} as explained in the definitions section above in the description of “first substituent group(s)”. In embodiments, when an R^{18A.2} substituent group is substituted, the R^{18A.2} substituent group is substituted with one or more third substituent groups denoted by R^{18A.3} as explained in the definitions section above in the description of “first substituent group(s)”. In the above embodiments, R^{18A}, R^{18A.1}, R^{18A.2}, and R^{18A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{18A}, R^{18A.1}, R^{18A.2} and R^{18A.3}, respectively.

In embodiments, when R^{18B} is substituted, R^{18B} is substituted with one or more first substituent groups denoted by R^{18B.1} as explained in the definitions section above in the description of “first substituent group(s)”. In embodiments, when an R^{18B.1} substituent group is substituted, the R^{18B.1} substituent group is substituted with one or more second substituent groups denoted by R^{18B.2} as explained in the definitions section above in the description of “first substituent group(s)”. In embodiments, when an R^{18B.2} substituent group is substituted, the R^{18B.2} substituent group is substituted with one or more third substituent groups denoted by R^{18B.3} as explained in the definitions section above in the description of “first substituent group(s)”. In the above embodiments, R^{18B}, R^{18B.1}, R^{18B.2}, and R^{18B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{18B}, R^{18B.1}, R^{18B.2}, and R^{18B.3}, respectively.

In embodiments, when R^{18A} and R^{18B} substituents bonded to the same nitrogen atom are optionally joined to form a moiety that is substituted (e.g., a substituted heterocycloalkyl or substituted heteroaryl), the moiety is substituted with one or more first substituent groups denoted by R^{18A.1} as explained in the definitions section above in the description of “first substituent group(s)”. In embodiments, when an R^{18A.1} substituent group is substituted, the R^{18A.1} substituent group is substituted with one or more second substituent groups denoted by R^{18A.2} as explained in the definitions section above in the description of “first substituent group(s)”. In embodiments, when an R^{18A.2} substituent group is substituted, the R^{18A.2} substituent group is substituted with one or more third substituent groups denoted by R^{18A.3} as explained in the definitions section above in the description of “first substituent group(s)”. In the above embodiments, R^{18A.1}, R^{18A.2}, and R^{18A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{18A.1}, R^{18A.2}, and R^{18A.3}, respectively.

In embodiments, when R^{18A} and R^{18B} substituents bonded to the same nitrogen atom are optionally joined to form a moiety that is substituted (e.g., a substituted heterocycloalkyl or substituted heteroaryl), the moiety is substituted with one or more first substituent groups denoted by R^{18B.1} as explained in the definitions section above in the description of “first substituent group(s)”. In embodiments, when an R^{18B.1} substituent group is substituted, the R^{18B.1} substituent group is substituted with one or more second substituent groups denoted by R^{18B.2} as explained in the definitions section above in the description of “first substituent group(s)”. In embodiments, when an R^{18B.2} substituent group is substituted, the R^{18B.2} substituent group is substituted with one or more third substituent groups denoted by R^{18B.3} as explained in the definitions section above in the description of “first substituent group(s)”. In the above embodiments, R^{18B.1}, R^{18B.2}, and R^{18B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{18B.1}, R^{18B.2}, and R^{18B.3}, respectively.

In embodiments, when R^{18C} is substituted, R^{18C} is substituted with one or more first substituent groups denoted by R^{18C.1} as explained in the definitions section above in the description of “first substituent group(s)”. In embodiments, when an R^{18C.1} substituent group is substituted, the R^{18C.1} substituent group is substituted with one or more second substituent groups denoted by R^{18C.2} as explained in the definitions section above in the description of “first substituent group(s)”. In embodiments, when an R^{18C.2} substituent group is substituted, the R^{18C.2} substituent group is substituted with one or more third substituent groups denoted by R^{18C.3} as explained in the definitions section above in the description of “first substituent group(s)”. In the above embodiments, R^{18C}, R^{18C.1}, R^{18C.2}, and R^{18C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{18C}, R^{18C.1}, R^{18C.2}, and R^{18C.3}, respectively.

In embodiments, the compound has the formula:

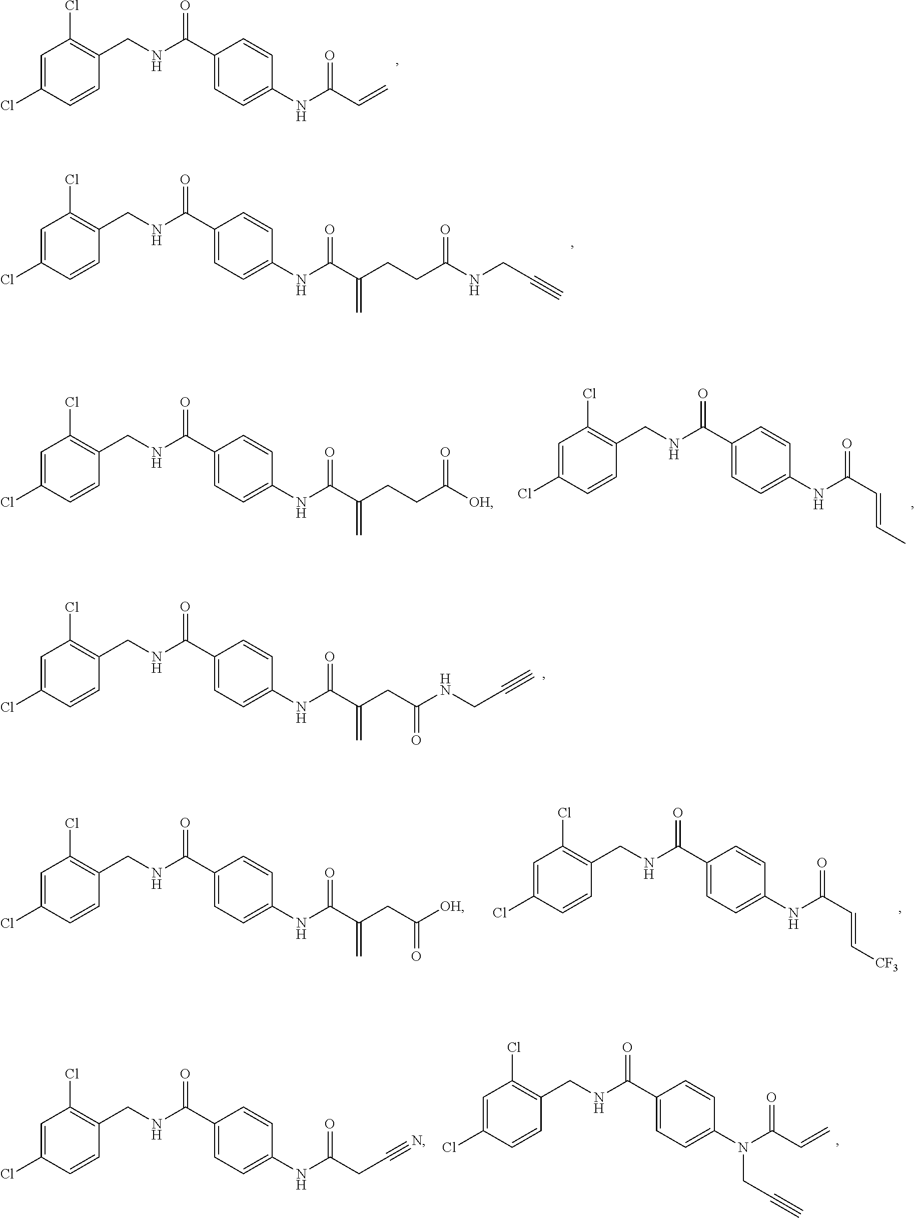

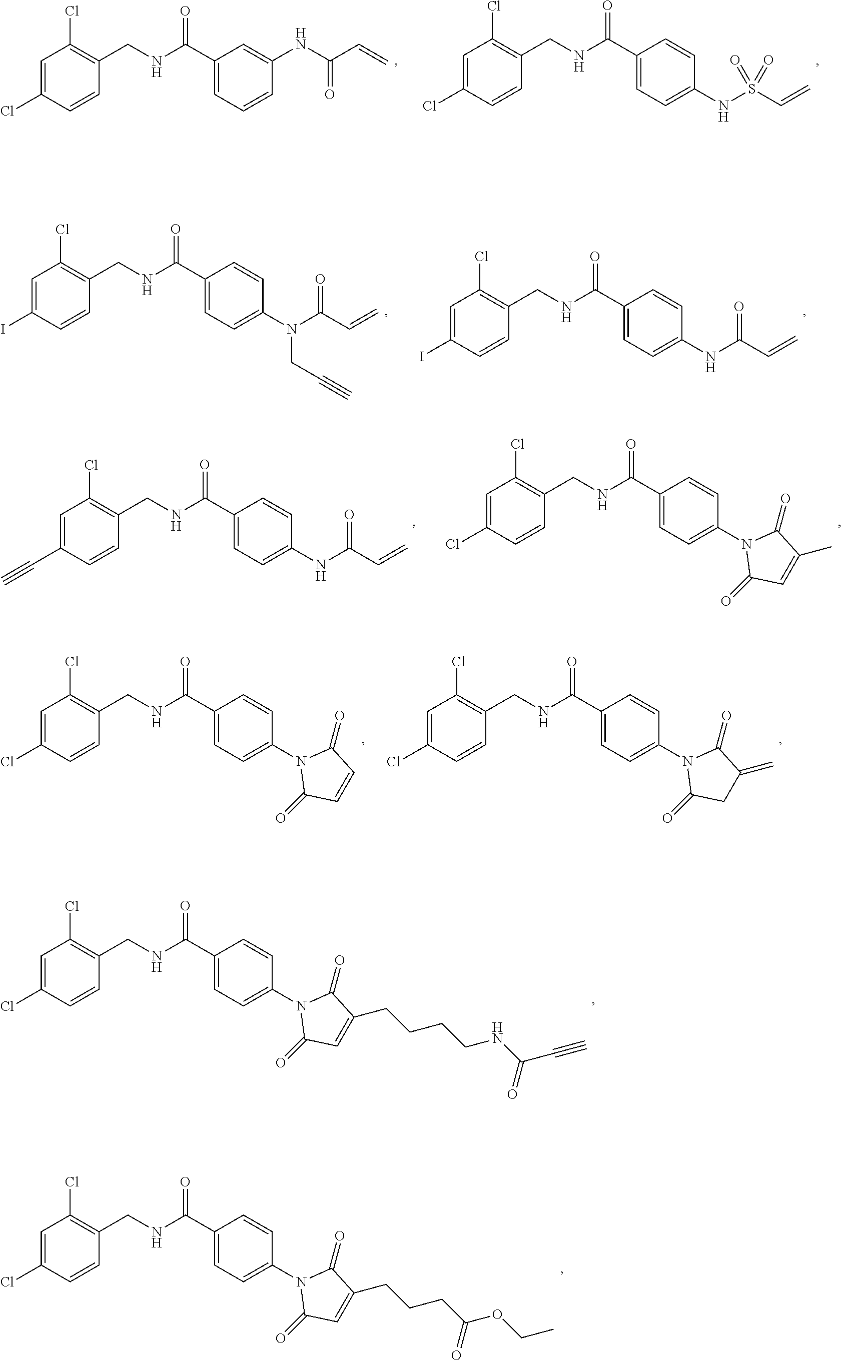

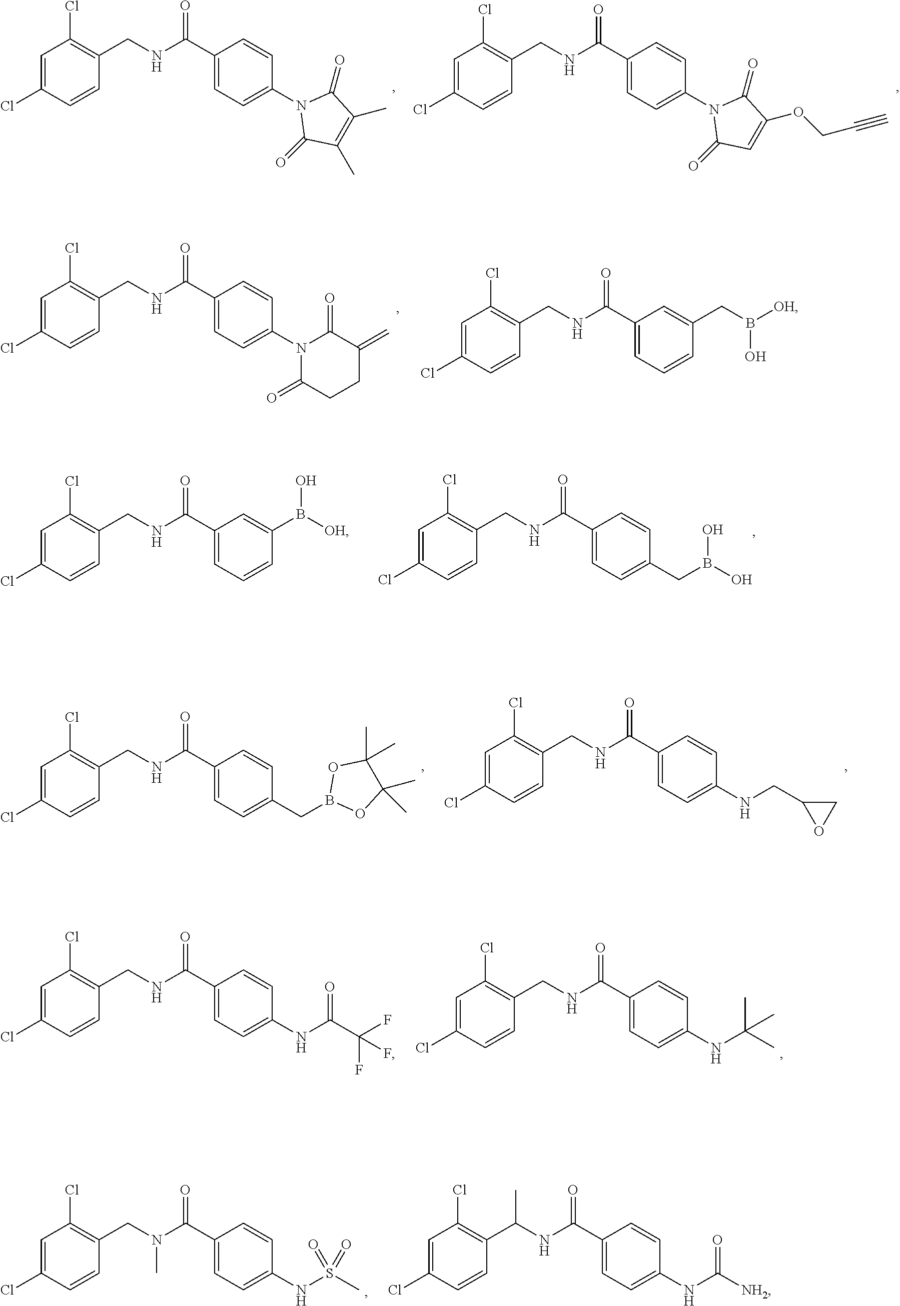

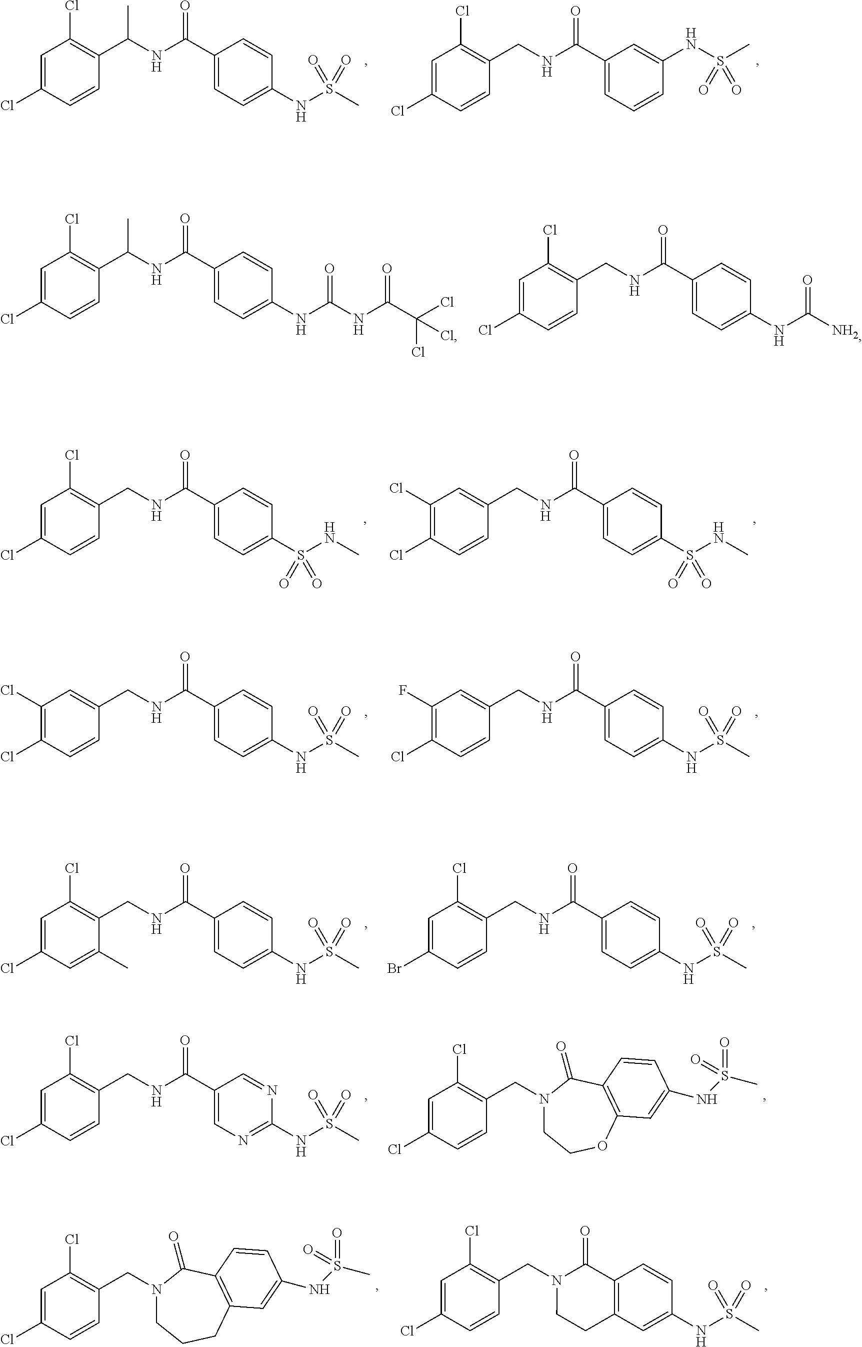

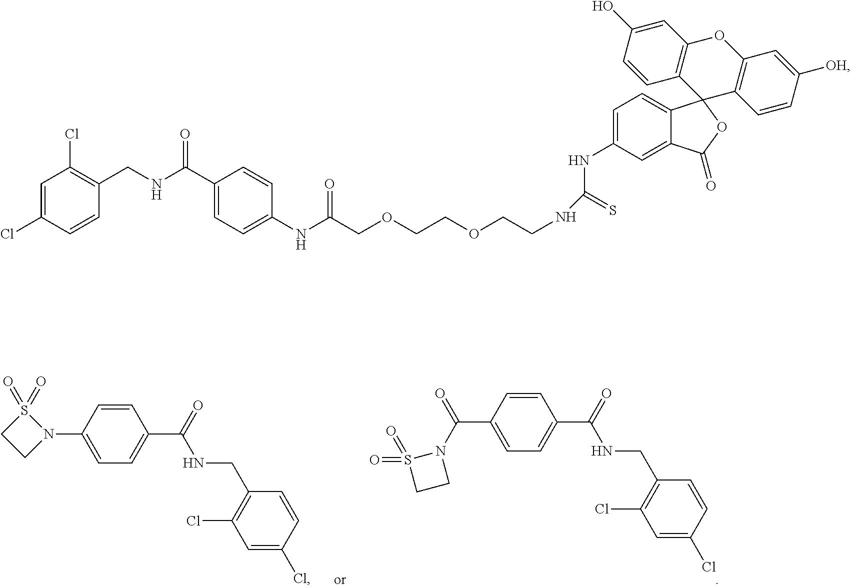 [SMILES: O=C(COCCOCCNC(=S)Nc1ccc2c(c1)C(=O)OC21c2ccc(O)cc2Oc2cc(O)ccc21)Nc1ccc(C(=O)NCc2ccc(Cl)cc2Cl)cc1 ; O=C(NCc1ccc(Cl)cc1Cl)c1ccc(N2CCS2(=O)=O)cc1 ; O=C(NCc1ccc(Cl)cc1Cl)c1ccc(C(=O)N2CCS2(=O)=O)cc1]

In embodiments, the compound has the formula:

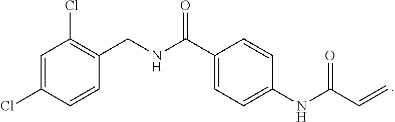 [SMILES: C=CC(=O)Nc1ccc(C(=O)NCc2ccc(Cl)cc2Cl)cc1]

In embodiments, the compound has the formula:

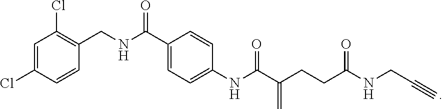 [SMILES: C#CCNC(=O)CCC(=C)C(=O)Nc1ccc(C(=O)NCc2ccc(Cl)cc2Cl)cc1]

In embodiments, the compound has the formula

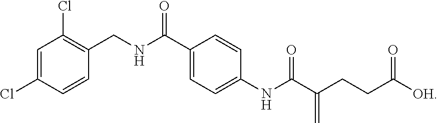 [SMILES: C=C(CCC(=O)O)C(=O)Nc1ccc(C(=O)NCc2ccc(Cl)cc2Cl)cc1]

In embodiments, the compound has the formula:

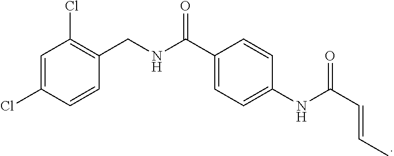 [SMILES: C/C=C/C(=O)Nc1ccc(C(=O)NCc2ccc(Cl)cc2Cl)cc1]

In embodiments, the compound has the formula

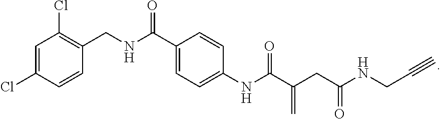 [SMILES: C#CCNC(=O)CC(=C)C(=O)Nc1ccc(C(=O)NCc2ccc(Cl)cc2Cl)cc1]

In embodiments, the compound has the formula:

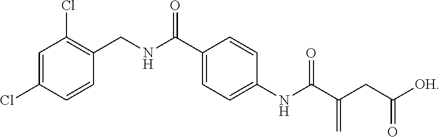 [SMILES: C=C(CC(=O)O)C(=O)Nc1ccc(C(=O)NCc2ccc(Cl)cc2Cl)cc1]

In embodiments, the compound has the formula:

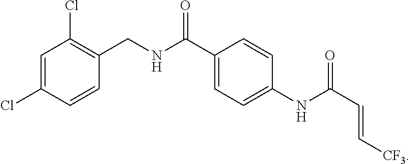 [SMILES: O=C(/C=C/C(F)(F)F)Nc1ccc(C(=O)NCc2ccc(Cl)cc2Cl)cc1]

In embodiments, the compound has the formula:

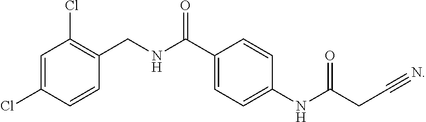 [SMILES: N#CCC(=O)Nc1ccc(C(=O)NCc2ccc(Cl)cc2Cl)cc1]

In embodiments, the compound has the formula:

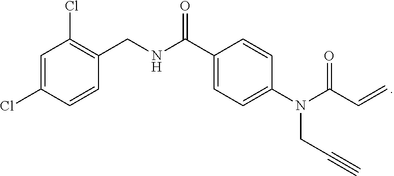 [SMILES: C#CCN(C(=O)C=C)c1ccc(C(=O)NCc2ccc(Cl)cc2Cl)cc1]

In embodiments, the compound has the formula:

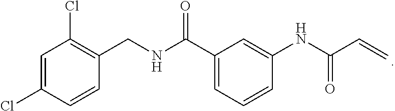 [SMILES: C=CC(=O)Nc1cccc(C(=O)NCc2ccc(Cl)cc2Cl)c1]

In embodiments, the compound has the formula:

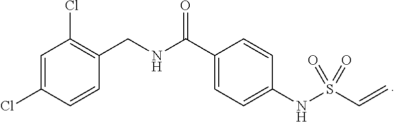 [SMILES: C=CS(=O)(=O)Nc1ccc(C(=O)NCc2ccc(Cl)cc2Cl)cc1]

In embodiments, the compound has the formula:

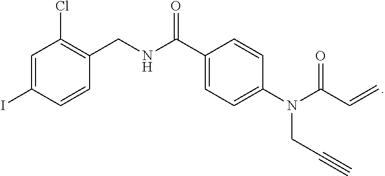 [SMILES: C#CCN(C(=O)C=C)c1ccc(C(=O)NCc2ccc(I)cc2Cl)cc1]

In embodiments, the compound has the formula:

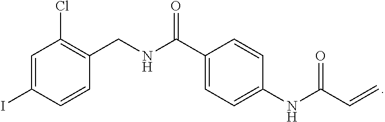 [SMILES: C=CC(=O)Nc1ccc(C(=O)NCc2ccc(I)cc2Cl)cc1]

In embodiments, the compound has the formula:

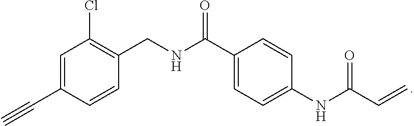 [SMILES: C#Cc1ccc(CNC(=O)c2ccc(NC(=O)C=C)cc2)c(Cl)c1]

In embodiments, the compound has the formula:

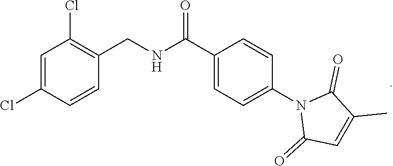 [SMILES: CC1=CC(=O)N(c2ccc(C(=O)NCc3ccc(Cl)cc3Cl)cc2)C1=O]

In embodiments, the compound has the formula:

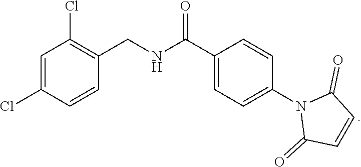 [SMILES: O=C(NCc1ccc(Cl)cc1Cl)c1ccc(N2C(=O)C=CC2=O)cc1]

In embodiments, the compound has the formula:

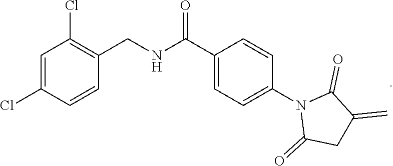 [SMILES: C=C1CC(=O)N(c2ccc(C(=O)NCc3ccc(Cl)cc3Cl)cc2)C1=O]

In embodiments, the compound has the formula:

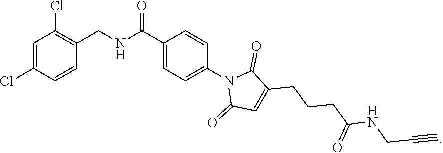 [SMILES: C#CCNC(=O)CCCC1=CC(=O)N(c2ccc(C(=O)NCc3ccc(Cl)cc3Cl)cc2)C1=O]

In embodiments, the compound has the formula:

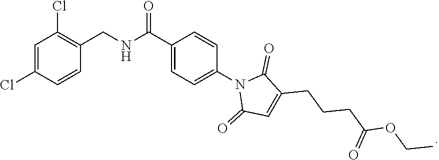 [SMILES: CCOC(=O)CCCC1=CC(=O)N(c2ccc(C(=O)NCc3ccc(Cl)cc3Cl)cc2)C1=O]

In embodiments, the compound has the formula:

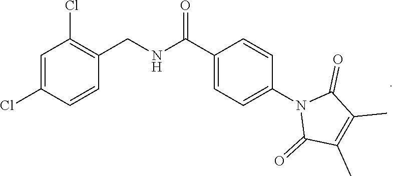 [SMILES: CC1=C(C)C(=O)N(c2ccc(C(=O)NCc3ccc(Cl)cc3Cl)cc2)C1=O]

In embodiments, the compound has the formula:

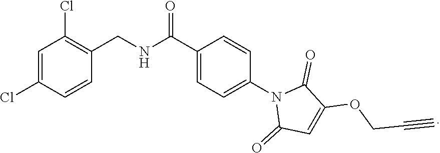 [SMILES: C#CCOC1=CC(=O)N(c2ccc(C(=O)NCc3ccc(Cl)cc3Cl)cc2)C1=O]

In embodiments, the compound has the formula

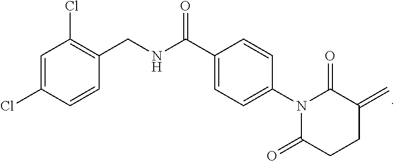 [SMILES: C=C1CCC(=O)N(c2ccc(C(=O)NCc3ccc(Cl)cc3Cl)cc2)C1=O]

In embodiments, the compound has the formula:

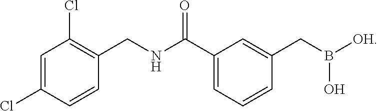 [SMILES: O=C(NCc1ccc(Cl)cc1Cl)c1cccc(CB(O)O)c1]

In embodiments, the compound has the formula:

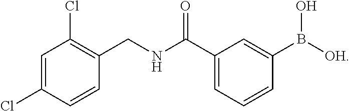 [SMILES: O=C(NCc1ccc(Cl)cc1Cl)c1cccc(B(O)O)c1]

In embodiments, the compound has the formula:

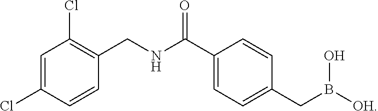 [SMILES: O=C(NCc1ccc(Cl)cc1Cl)c1ccc(CB(O)O)cc1]

In embodiments, the compound has the formula:

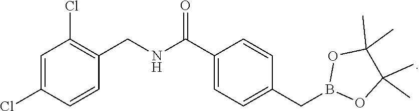 [SMILES: CC1(C)OB(Cc2ccc(C(=O)NCc3ccc(Cl)cc3Cl)cc2)OC1(C)C]

In embodiments, the compound has the formula:

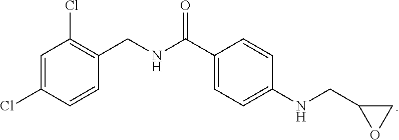 [SMILES: O=C(NCc1ccc(Cl)cc1Cl)c1ccc(NCC2CO2)cc1]

In embodiments, the compound has the formula:

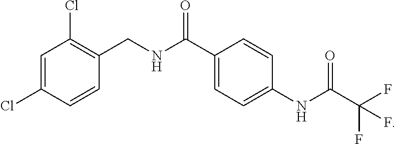 [SMILES: O=C(NCc1ccc(Cl)cc1Cl)c1ccc(NC(=O)C(F)(F)F)cc1]

In embodiments, the compound has the formula:

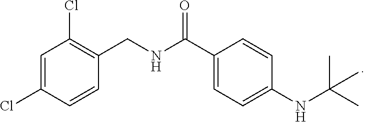 [SMILES: CC(C)(C)Nc1ccc(C(=O)NCc2ccc(Cl)cc2Cl)cc1]

In embodiments, the compound has the formula:

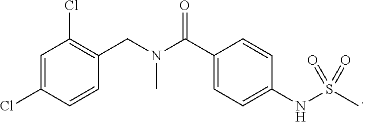 [SMILES: CN(Cc1ccc(Cl)cc1Cl)C(=O)c1ccc(NS(C)(=O)=O)cc1]

In embodiments, the compound has the formula:

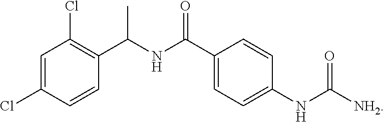 [SMILES: CC(NC(=O)c1ccc(NC(N)=O)cc1)c1ccc(Cl)cc1Cl]

In embodiments, the compound has the formula:

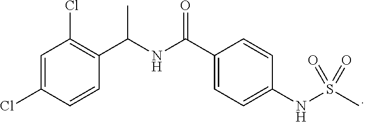 [SMILES: CC(NC(=O)c1ccc(NS(C)(=O)=O)cc1)c1ccc(Cl)cc1Cl]

In embodiments, the compound has the formula:

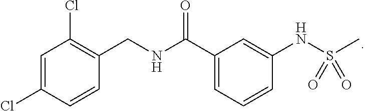 [SMILES: CS(=O)(=O)Nc1cccc(C(=O)NCc2ccc(Cl)cc2Cl)c1]

In embodiments, the compound has the formula:

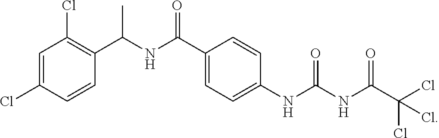 [SMILES: CC(NC(=O)c1ccc(NC(=O)NC(=O)C(Cl)(Cl)Cl)cc1)c1ccc(Cl)cc1Cl]

In embodiments, the compound has the formula:

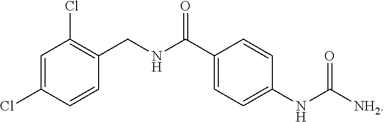 [SMILES: NC(=O)Nc1ccc(C(=O)NCc2ccc(Cl)cc2Cl)cc1]

In embodiments, the compound has the formula:

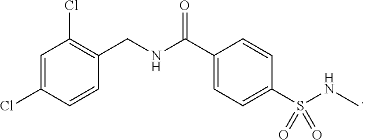 [SMILES: CNS(=O)(=O)c1ccc(C(=O)NCc2ccc(Cl)cc2Cl)cc1]

In embodiments, the compound has the formula:

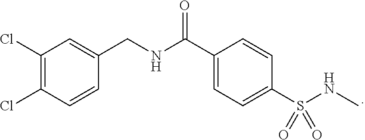 [SMILES: CNS(=O)(=O)c1ccc(C(=O)NCc2ccc(Cl)c(Cl)c2)cc1]

In embodiments, the compound has the formula:

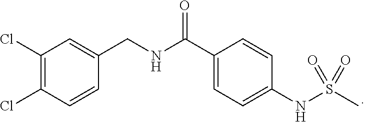 [SMILES: CS(=O)(=O)Nc1ccc(C(=O)NCc2ccc(Cl)c(Cl)c2)cc1]

In embodiments, the compound has the formula:

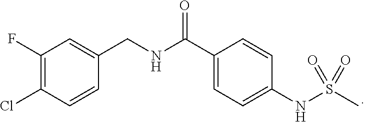 [SMILES: CS(=O)(=O)Nc1ccc(C(=O)NCc2ccc(Cl)c(F)c2)cc1]

In embodiments, the compound has the formula:

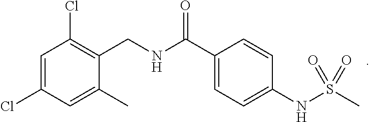 [SMILES: Cc1cc(Cl)cc(Cl)c1CNC(=O)c1ccc(NS(C)(=O)=O)cc1]

In embodiments, the compound has the formula:

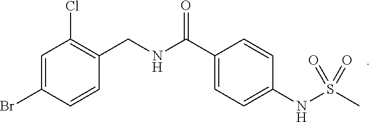 [SMILES: CS(=O)(=O)Nc1ccc(C(=O)NCc2ccc(Br)cc2Cl)cc1]

In embodiments, the compound has the formula:

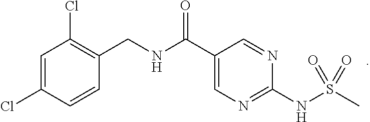 [SMILES: CS(=O)(=O)Nc1ncc(C(=O)NCc2ccc(Cl)cc2Cl)cn1]

In embodiments, the compound has the formula:

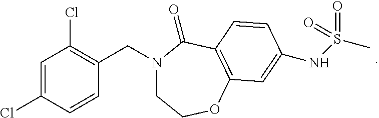 [SMILES: CS(=O)(=O)Nc1ccc2c(c1)OCCN(Cc1ccc(Cl)cc1Cl)C2=O]

In embodiments, the compound has the formula:

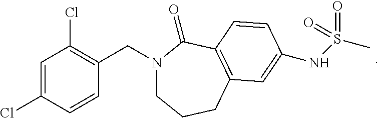 [SMILES: CS(=O)(=O)Nc1ccc2c(c1)CCCN(Cc1ccc(Cl)cc1Cl)C2=O]

In embodiments, the compound has the formula:

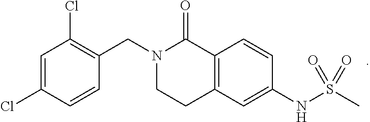 [SMILES: CS(=O)(=O)Nc1ccc2c(c1)CCN(Cc1ccc(Cl)cc1Cl)C2=O]

In embodiments, the compound has the formula:

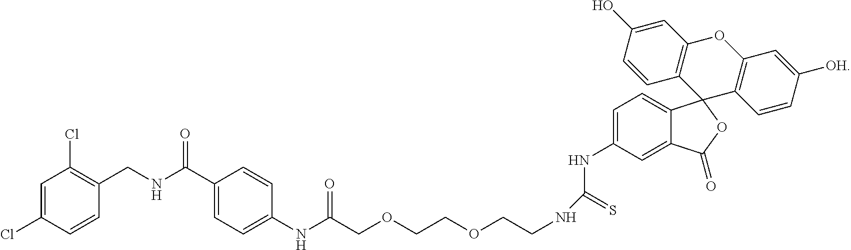 [SMILES: O=C(COCCOCCNC(=S)Nc1ccc2c(c1)C(=O)OC21c2ccc(O)cc2Oc2cc(O)ccc21)Nc1ccc(C(=O)NCc2ccc(Cl)cc2Cl)cc1]

In embodiments, the compound has the formula:

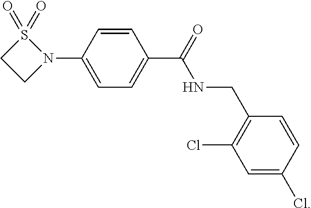 [SMILES: O=C(NCc1ccc(Cl)cc1Cl)c1ccc(N2CCS2(=O)=O)cc1]

In embodiments, the compound has the formula:

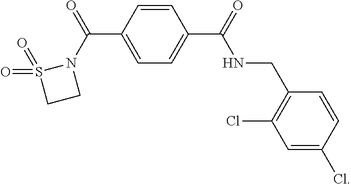 [SMILES: O=C(NCc1ccc(Cl)cc1Cl)c1ccc(C(=O)N2CCS2(=O)=O)cc1]

In embodiments, the compound has the formula:

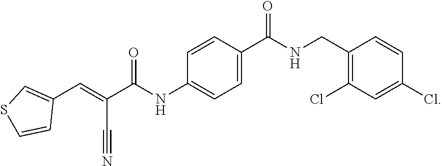 [SMILES: N#C/C(=C\c1ccsc1)C(=O)Nc1ccc(C(=O)NCc2ccc(Cl)cc2Cl)cc1]

In embodiments, the compound has the formula:

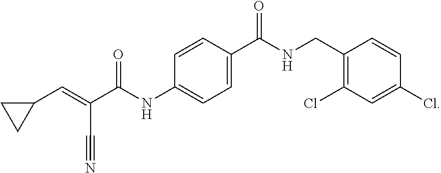 [SMILES: N#C/C(=C\C1CC1)C(=O)Nc1ccc(C(=O)NCc2ccc(Cl)cc2Cl)cc1]

In embodiments, the compound has the formula:

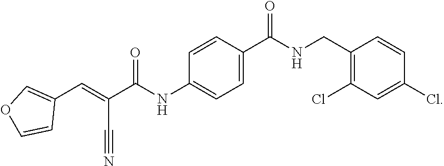 [SMILES: N#C/C(=C\c1ccoc1)C(=O)Nc1ccc(C(=O)NCc2ccc(Cl)cc2Cl)cc1]

In embodiments, the compound has the formula:

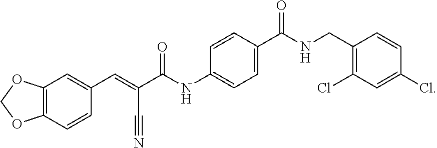 [SMILES: N#C/C(=C\c1ccc2c(c1)OCO2)C(=O)Nc1ccc(C(=O)NCc2ccc(Cl)cc2Cl)cc1]

In embodiments, the compound has the formula:

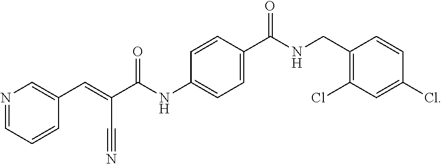 [SMILES: N#C/C(=C\c1cccnc1)C(=O)Nc1ccc(C(=O)NCc2ccc(Cl)cc2Cl)cc1]

As used herein, the labels “CAT335” and “ML337” are used interchangeably, and refer to the structure having the formula:

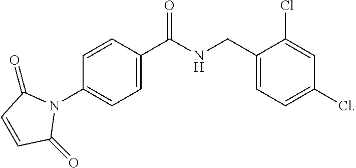 [SMILES: O=C(NCc1ccc(Cl)cc1Cl)c1ccc(N2C(=O)C=CC2=O)cc1]

As used herein, the labels “CAT335a” and “ML337a” are used interchangeably, and refer to the structure having the formula:

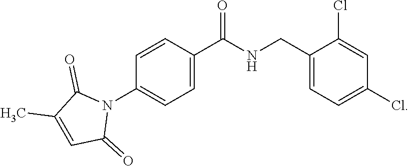 [SMILES: CC1=CC(=O)N(c2ccc(C(=O)NCc3ccc(Cl)cc3Cl)cc2)C1=O]

As used herein, the labels “CAT335b” and “ML337b” are used interchangeably, and refer to the structure having the formula:

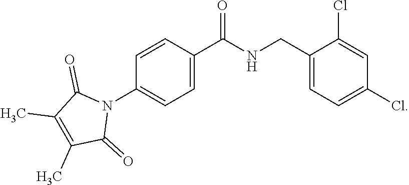 [SMILES: CC1=C(C)C(=O)N(c2ccc(C(=O)NCc3ccc(Cl)cc3Cl)cc2)C1=O]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Modified Proteins

In an aspect is provided a K_{2P} family protein or homolog thereof including a cysteine residue at an amino position corresponding to position 131 of TREK-1. In embodiments is provided a K_{2P} family protein including a cysteine residue at an amino position corresponding to position 131 of TREK-1. In embodiments is provided a K2P family protein including a cysteine residue at an amino position corresponding to position 131 of TREK-1 and a phenylalanine at an amino acid position corresponding to position 286 of TREK-1. In embodiments is provided a K2P family protein including a cysteine residue at an amino position corresponding to position 131 of TREK-1 and no other mutations relative to the wild type sequence of said K2P family protein. In embodiments is provided a K2P family protein including a cysteine residue at an amino position corresponding to position 131 of TREK-1 and a phenylalanine at an amino acid position corresponding to position 286 of TREK-1 and no other mutations relative to the wild type sequence of said K2P family protein.

In embodiments, the K_{2P} family protein is a TREK family protein. In embodiments, the TREK family protein is a TREK-1 protein, a TREK-2 protein, or a TRAAK protein. In embodiments, the K_{2P} family protein is a THIK-1 protein or a THIK-2 protein. In embodiments, the K_{2P} family protein is a TWIK-1 protein, a TWIK-2 protein, or a KCNK7 protein. In embodiments, the K_{2P} family protein is a TRESK protein. In embodiments, the K_{2P} family protein is a TASK-1 protein, a TASK-3 protein, or a TASK-5 protein. In embodiments, the K_{2P} family protein is a TALK-1 protein, a TALK-2 protein, or a TASK-2 protein.

In embodiments, provided herein are modified TREK family proteins comprising a cysteine residue. In embodiments, the cysteine residue is not found in a natural TREK family protein. In embodiments, the cysteine residue is positioned at an amino position corresponding to position 131 of TREK-1. In embodiments is provided a TREK family protein including a cysteine residue at an amino position corresponding to position 131 of TREK-1. In embodiments is provided aTREK family protein including a cysteine residue at an amino position corresponding to position 131 of TREK-1 and a phenylalanine at an amino acid position corresponding to position 286 of TREK-1. In embodiments is provided a TREK family protein including a cysteine residue at an amino position corresponding to position 131 of TREK-1 and no other mutations relative to the wild type sequence of said TREK family protein. In embodiments is provided a TREK family protein including a cysteine residue at an amino position corresponding to position 131 of TREK-1 and a phenylalanine at an amino acid position corresponding to position 286 of TREK-1 and no other mutations relative to the wild type sequence of said TREK family protein. In embodiments, the TREK family protein is a TREK-1 protein, a TREK-2 protein or a TRAAK protein. In embodiments, the TREK family protein is a TREK-1 protein. In embodiments, the TREK family protein is a TREK-2 protein. In embodiments, the TREK family protein is a TRAAK protein.

In embodiments, the TREK-1 protein comprises SEQ ID NO: 1. In embodiments, the TREK-1 protein comprises a 100 amino acid contiguous sequence of SEQ ID NO: 1. In embodiments, the TREK-1 protein comprises an amino acid sequence that has 80% identity to SEQ ID NO: 1. In embodiments, the TREK-1 protein comprises an amino acid sequence that has 80% identity to a 100 amino acid contiguous sequence of SEQ ID NO: 1. In embodiments, the TREK-1 protein comprises an amino acid sequence that has 85% identity to SEQ ID NO: 1. In embodiments, the TREK-1 protein comprises an amino acid sequence that has 85% identity to a 100 amino acid contiguous sequence of SEQ ID NO: 1. In embodiments, the TREK-1 protein comprises an amino acid sequence that has 90% identity to SEQ ID NO: 1. In embodiments, the TREK-1 protein comprises an amino acid sequence that has 90% identity to a 100 amino acid contiguous sequence of SEQ ID NO: 1. In embodiments, the TREK-1 protein comprises an amino acid sequence that has 95% identity to SEQ ID NO: 1. In embodiments, the TREK-1 protein comprises an amino acid sequence that has 95% identity to a 100 amino acid contiguous sequence of SEQ ID NO: 1. In embodiments, the TREK-1 protein comprises an amino acid sequence that has 96% identity to SEQ ID NO: 1. In embodiments, the TREK-1 protein comprises an amino acid sequence that has 96% identity to a 100 amino acid contiguous sequence of SEQ ID NO: 1. In embodiments, the TREK-1 protein comprises an amino acid sequence that has 97% identity to SEQ ID NO: 1. In embodiments, the TREK-1 protein comprises an amino acid sequence that has 97% identity to a 100 amino acid contiguous sequence of SEQ ID NO: 1. In embodiments, the TREK-1 protein comprises an amino acid sequence that has 98% identity to SEQ ID NO: 1. In embodiments, the TREK-1 protein comprises an amino acid sequence that has 98% identity to a 100 amino acid contiguous sequence of SEQ ID NO: 1. In embodiments, the TREK-1 protein comprises an amino acid sequence that has 99% identity to SEQ ID NO: 1. In embodiments, the TREK-1 protein comprises an amino acid sequence that has 99% identity to a 100 amino acid contiguous sequence of SEQ ID NO: 1.

In embodiments, the TREK-1 protein comprises SEQ ID NO: 2. In embodiments, the TREK-1 protein comprises a 100 amino acid contiguous sequence of SEQ ID NO: 2. In embodiments, the TREK-1 protein comprises an amino acid sequence that has 80% identity to SEQ ID NO: 2. In embodiments, the TREK-1 protein comprises an amino acid sequence that has 80% identity to a 100 amino acid contiguous sequence of SEQ ID NO: 2. In embodiments, the TREK-1 protein comprises an amino acid sequence that has 85% identity to SEQ ID NO: 2. In embodiments, the TREK-1 protein comprises an amino acid sequence that has 85% identity to a 100 amino acid contiguous sequence of SEQ ID NO: 2. In embodiments, the TREK-1 protein comprises an amino acid sequence that has 90% identity to SEQ ID NO: 2. In embodiments, the TREK-1 protein comprises an amino acid sequence that has 90% identity to a 100 amino acid contiguous sequence of SEQ ID NO: 2. In embodiments, the TREK-1 protein comprises an amino acid sequence that has 95% identity to SEQ ID NO: 2. In embodiments, the TREK-1 protein comprises an amino acid sequence that has 95% identity to a 100 amino acid contiguous sequence of SEQ ID NO: 2. In embodiments, the TREK-1 protein comprises an amino acid sequence that has 96% identity to SEQ ID NO: 2. In embodiments, the TREK-1 protein comprises an amino acid sequence that has 96% identity to a 100 amino acid contiguous sequence of SEQ ID NO: 2. In embodiments, the TREK-1 protein comprises an amino acid sequence that has 97% identity to SEQ ID NO: 2. In embodiments, the TREK-1 protein comprises an amino acid sequence that has 97% identity to a 100 amino acid contiguous sequence of SEQ ID NO: 2. In embodiments, the TREK-1 protein comprises an amino acid sequence that has 98% identity to SEQ ID NO: 2. In embodiments, the TREK-1 protein comprises an amino acid sequence that has 98% identity to a 100 amino acid contiguous sequence of SEQ ID NO: 2. In embodiments, the TREK-1 protein comprises an amino acid sequence that has 99% identity to SEQ ID NO: 2. In embodiments, the TREK-1 protein comprises an amino acid sequence that has 99% identity to a 100 amino acid contiguous sequence of SEQ ID NO: 2.

In embodiments, the TREK-1 protein comprises SEQ ID NO: 3. In embodiments, the TREK-1 protein comprises a 100 amino acid contiguous sequence of SEQ ID NO: 3. In embodiments, the TREK-1 protein comprises an amino acid sequence that has 80% identity to SEQ ID NO: 3. In embodiments, the TREK-1 protein comprises an amino acid sequence that has 80% identity to a 100 amino acid contiguous sequence of SEQ ID NO: 3. In embodiments, the TREK-1 protein comprises an amino acid sequence that has 85% identity to SEQ ID NO: 3. In embodiments, the TREK-1 protein comprises an amino acid sequence that has 85% identity to a 100 amino acid contiguous sequence of SEQ ID NO: 3. In embodiments, the TREK-1 protein comprises an amino acid sequence that has 90% identity to SEQ ID NO: 3. In embodiments, the TREK-1 protein comprises an amino acid sequence that has 90% identity to a 100 amino acid contiguous sequence of SEQ ID NO: 3. In embodiments, the TREK-1 protein comprises an amino acid sequence that has 95% identity to SEQ ID NO: 3. In embodiments, the TREK-1 protein comprises an amino acid sequence that has 95% identity to a 100 amino acid contiguous sequence of SEQ ID NO: 3. In embodiments, the TREK-1 protein comprises an amino acid sequence that has 96% identity to SEQ ID NO: 3. In embodiments, the TREK-1 protein comprises an amino acid sequence that has 96% identity to a 100 amino acid contiguous sequence of SEQ ID NO: 3. In embodiments, the TREK-1 protein comprises an amino acid sequence that has 97% identity to SEQ ID NO: 3. In embodiments, the TREK-1 protein comprises an amino acid sequence that has 97% identity to a 100 amino acid contiguous sequence of SEQ ID NO: 3. In embodiments, the TREK-1 protein comprises an amino acid sequence that has 98% identity to SEQ ID NO: 3. In embodiments, the TREK-1 protein comprises an amino acid sequence that has 98% identity to a 100 amino acid contiguous sequence of SEQ ID NO: 3. In embodiments, the TREK-1 protein comprises an amino acid sequence that has 99% identity to SEQ ID NO: 3. In embodiments, the TREK-1 protein comprises an amino acid sequence that has 99% identity to a 100 amino acid contiguous sequence of SEQ ID NO: 3.

In embodiments, the TREK-1 protein comprises SEQ ID NO: 4. In embodiments, the TREK-1 protein comprises a 100 amino acid contiguous sequence of SEQ ID NO: 4. In embodiments, the TREK-1 protein comprises an amino acid sequence that has 80% identity to SEQ ID NO: 4. In embodiments, the TREK-1 protein comprises an amino acid sequence that has 80% identity to a 100 amino acid contiguous sequence of SEQ ID NO: 4. In embodiments, the TREK-1 protein comprises an amino acid sequence that has 85% identity to SEQ ID NO: 4. In embodiments, the TREK-1 protein comprises an amino acid sequence that has 85% identity to a 100 amino acid contiguous sequence of SEQ ID NO: 4. In embodiments, the TREK-1 protein comprises an amino acid sequence that has 90% identity to SEQ ID NO: 4. In embodiments, the TREK-1 protein comprises an amino acid sequence that has 90% identity to a 100 amino acid contiguous sequence of SEQ ID NO: 4. In embodiments, the TREK-1 protein comprises an amino acid sequence that has 95% identity to SEQ ID NO: 4. In embodiments, the TREK-1 protein comprises an amino acid sequence that has 95% identity to a 100 amino acid contiguous sequence of SEQ ID NO: 4. In embodiments, the TREK-1 protein comprises an amino acid sequence that has 96% identity to SEQ ID NO: 4. In embodiments, the TREK-1 protein comprises an amino acid sequence that has 96% identity to a 100 amino acid contiguous sequence of SEQ ID NO: 4. In embodiments, the TREK-1 protein comprises an amino acid sequence that has 97% identity to SEQ ID NO: 4. In embodiments, the TREK-1 protein comprises an amino acid sequence that has 97% identity to a 100 amino acid contiguous sequence of SEQ ID NO: 4. In embodiments, the TREK-1 protein comprises an amino acid sequence that has 98% identity to SEQ ID NO: 4. In embodiments, the TREK-1 protein comprises an amino acid sequence that has 98% identity to a 100 amino acid contiguous sequence of SEQ ID NO: 4. In embodiments, the TREK-1 protein comprises an amino acid sequence that has 99% identity to SEQ ID NO: 4. In embodiments, the TREK-1 protein comprises an amino acid sequence that has 99% identity to a 100 amino acid contiguous sequence of SEQ ID NO: 4.

In embodiments, the TREK-2 protein comprises SEQ ID NO: 5. In embodiments, the TREK-2 protein comprises a 100 amino acid contiguous sequence of SEQ ID NO: 5. In embodiments, the TREK-2 protein comprises an amino acid sequence that has 80% identity to SEQ ID NO: 5. In embodiments, the TREK-2 protein comprises an amino acid sequence that has 80% identity to a 100 amino acid contiguous sequence of SEQ ID NO: 5. In embodiments, the TREK-2 protein comprises an amino acid sequence that has 85% identity to SEQ ID NO: 5. In embodiments, the TREK-2 protein comprises an amino acid sequence that has 85% identity to a 100 amino acid contiguous sequence of SEQ ID NO: 5. In embodiments, the TREK-2 protein comprises an amino acid sequence that has 90% identity to SEQ ID NO: 5. In embodiments, the TREK-2 protein comprises an amino acid sequence that has 90% identity to a 100 amino acid contiguous sequence of SEQ ID NO: 5. In embodiments, the TREK-2 protein comprises an amino acid sequence that has 95% identity to SEQ ID NO: 5. In embodiments, the TREK-2 protein comprises an amino acid sequence that has 95% identity to a 100 amino acid contiguous sequence of SEQ ID NO: 5. In embodiments, the TREK-2 protein comprises an amino acid sequence that has 96% identity to SEQ ID NO: 5. In embodiments, the TREK-2 protein comprises an amino acid sequence that has 96% identity to a 100 amino acid contiguous sequence of SEQ ID NO: 5. In embodiments, the TREK-2 protein comprises an amino acid sequence that has 97% identity to SEQ ID NO: 5. In embodiments, the TREK-2 protein comprises an amino acid sequence that has 97% identity to a 100 amino acid contiguous sequence of SEQ ID NO: 5. In embodiments, the TREK-2 protein comprises an amino acid sequence that has 98% identity to SEQ ID NO: 5. In embodiments, the TREK-2 protein comprises an amino acid sequence that has 98% identity to a 100 amino acid contiguous sequence of SEQ ID NO: 5. In embodiments, the TREK-2 protein comprises an amino acid sequence that has 99% identity to SEQ ID NO: 5. In embodiments, the TREK-2 protein comprises an amino acid sequence that has 99% identity to a 100 amino acid contiguous sequence of SEQ ID NO: 5.

In embodiments, the TREK-2 protein comprises SEQ ID NO: 6. In embodiments, the TREK-2 protein comprises a 100 amino acid contiguous sequence of SEQ ID NO: 6. In embodiments, the TREK-2 protein comprises an amino acid sequence that has 80% identity to SEQ ID NO: 6. In embodiments, the TREK-2 protein comprises an amino acid sequence that has 80% identity to a 100 amino acid contiguous sequence of SEQ ID NO: 6. In embodiments, the TREK-2 protein comprises an amino acid sequence that has 85% identity to SEQ ID NO: 6. In embodiments, the TREK-2 protein comprises an amino acid sequence that has 85% identity to a 100 amino acid contiguous sequence of SEQ ID NO: 6. In embodiments, the TREK-2 protein comprises an amino acid sequence that has 90% identity to SEQ ID NO: 6. In embodiments, the TREK-2 protein comprises an amino acid sequence that has 90% identity to a 100 amino acid contiguous sequence of SEQ ID NO: 6. In embodiments, the TREK-2 protein comprises an amino acid sequence that has 95% identity to SEQ ID NO: 6. In embodiments, the TREK-2 protein comprises an amino acid sequence that has 95% identity to a 100 amino acid contiguous sequence of SEQ ID NO: 6. In embodiments, the TREK-2 protein comprises an amino acid sequence that has 96% identity to SEQ ID NO: 6. In embodiments, the TREK-2 protein comprises an amino acid sequence that has 96% identity to a 100 amino acid contiguous sequence of SEQ ID NO: 6. In embodiments, the TREK-2 protein comprises an amino acid sequence that has 97% identity to SEQ ID NO: 6. In embodiments, the TREK-2 protein comprises an amino acid sequence that has 97% identity to a 100 amino acid contiguous sequence of SEQ ID NO: 6. In embodiments, the TREK-2 protein comprises an amino acid sequence that has 98% identity to SEQ ID NO: 6. In embodiments, the TREK-2 protein comprises an amino acid sequence that has 98% identity to a 100 amino acid contiguous sequence of SEQ ID NO: 6. In embodiments, the TREK-2 protein comprises an amino acid sequence that has 99% identity to SEQ ID NO: 6. In embodiments, the TREK-2 protein comprises an amino acid sequence that has 99% identity to a 100 amino acid contiguous sequence of SEQ ID NO: 6.

In embodiments, the TREK-2 protein comprises SEQ ID NO: 7. In embodiments, the TREK-2 protein comprises a 100 amino acid contiguous sequence of SEQ ID NO: 7. In embodiments, the TREK-2 protein comprises an amino acid sequence that has 80% identity to SEQ ID NO: 7. In embodiments, the TREK-2 protein comprises an amino acid sequence that has 80% identity to a 100 amino acid contiguous sequence of SEQ ID NO: 7. In embodiments, the TREK-2 protein comprises an amino acid sequence that has 85% identity to SEQ ID NO: 7. In embodiments, the TREK-2 protein comprises an amino acid sequence that has 85% identity to a 100 amino acid contiguous sequence of SEQ ID NO: 7. In embodiments, the TREK-2 protein comprises an amino acid sequence that has 90% identity to SEQ ID NO: 7. In embodiments, the TREK-2 protein comprises an amino acid sequence that has 90% identity to a 100 amino acid contiguous sequence of SEQ ID NO: 7. In embodiments, the TREK-2 protein comprises an amino acid sequence that has 95% identity to SEQ ID NO: 7. In embodiments, the TREK-2 protein comprises an amino acid sequence that has 95% identity to a 100 amino acid contiguous sequence of SEQ ID NO: 7. In embodiments, the TREK-2 protein comprises an amino acid sequence that has 96% identity to SEQ ID NO: 7. In embodiments, the TREK-2 protein comprises an amino acid sequence that has 96% identity to a 100 amino acid contiguous sequence of SEQ ID NO: 7. In embodiments, the TREK-2 protein comprises an amino acid sequence that has 97% identity to SEQ ID NO: 7. In embodiments, the TREK-2 protein comprises an amino acid sequence that has 97% identity to a 100 amino acid contiguous sequence of SEQ ID NO: 7. In embodiments, the TREK-2 protein comprises an amino acid sequence that has 98% identity to SEQ ID NO: 7. In embodiments, the TREK-2 protein comprises an amino acid sequence that has 98% identity to a 100 amino acid contiguous sequence of SEQ ID NO: 7. In embodiments, the TREK-2 protein comprises an amino acid sequence that has 99% identity to SEQ ID NO: 7. In embodiments, the TREK-2 protein comprises an amino acid sequence that has 99% identity to a 100 amino acid contiguous sequence of SEQ ID NO: 7.

In embodiments, the TREK-2 protein comprises SEQ ID NO: 8. In embodiments, the TREK-2 protein comprises a 100 amino acid contiguous sequence of SEQ ID NO: 8. In embodiments, the TREK-2 protein comprises an amino acid sequence that has 80% identity to SEQ ID NO: 8. In embodiments, the TREK-2 protein comprises an amino acid sequence that has 80% identity to a 100 amino acid contiguous sequence of SEQ ID NO: 8. In embodiments, the TREK-2 protein comprises an amino acid sequence that has 85% identity to SEQ ID NO: 8. In embodiments, the TREK-2 protein comprises an amino acid sequence that has 85% identity to a 100 amino acid contiguous sequence of SEQ ID NO: 8. In embodiments, the TREK-2 protein comprises an amino acid sequence that has 90% identity to SEQ ID NO: 8. In embodiments, the TREK-2 protein comprises an amino acid sequence that has 90% identity to a 100 amino acid contiguous sequence of SEQ ID NO: 8. In embodiments, the TREK-2 protein comprises an amino acid sequence that has 95% identity to SEQ ID NO: 8. In embodiments, the TREK-2 protein comprises an amino acid sequence that has 95% identity to a 100 amino acid contiguous sequence of SEQ ID NO: 8. In embodiments, the TREK-2 protein comprises an amino acid sequence that has 96% identity to SEQ ID NO: 8. In embodiments, the TREK-2 protein comprises an amino acid sequence that has 96% identity to a 100 amino acid contiguous sequence of SEQ ID NO: 8. In embodiments, the TREK-2 protein comprises an amino acid sequence that has 97% identity to SEQ ID NO: 8. In embodiments, the TREK-2 protein comprises an amino acid sequence that has 97% identity to a 100 amino acid contiguous sequence of SEQ ID NO: 8. In embodiments, the TREK-2 protein comprises an amino acid sequence that has 98% identity to SEQ ID NO: 8. In embodiments, the TREK-2 protein comprises an amino acid sequence that has 98% identity to a 100 amino acid contiguous sequence of SEQ ID NO: 8. In embodiments, the TREK-2 protein comprises an amino acid sequence that has 99% identity to SEQ ID NO: 8. In embodiments, the TREK-2 protein comprises an amino acid sequence that has 99% identity to a 100 amino acid contiguous sequence of SEQ ID NO: 8.

In embodiments, the TRAAK protein comprises SEQ ID NO: 9. In embodiments, the TRAAK protein comprises a 100 amino acid contiguous sequence of SEQ ID NO: 9. In embodiments, the TRAAK protein comprises an amino acid sequence that has 80% identity to SEQ ID NO: 9. In embodiments, the TRAAK protein comprises an amino acid sequence that has 80% identity to a 100 amino acid contiguous sequence of SEQ ID NO: 9. In embodiments, the TRAAK protein comprises an amino acid sequence that has 85% identity to SEQ ID NO: 9. In embodiments, the TRAAK protein comprises an amino acid sequence that has 85% identity to a 100 amino acid contiguous sequence of SEQ ID NO: 9. In embodiments, the TRAAK protein comprises an amino acid sequence that has 90% identity to SEQ ID NO: 9. In embodiments, the TRAAK protein comprises an amino acid sequence that has 90% identity to a 100 amino acid contiguous sequence of SEQ ID NO: 9. In embodiments, the TRAAK protein comprises an amino acid sequence that has 95% identity to SEQ ID NO: 9. In embodiments, the TRAAK protein comprises an amino acid sequence that has 95% identity to a 100 amino acid contiguous sequence of SEQ ID NO: 9. In embodiments, the TRAAK protein comprises an amino acid sequence that has 96% identity to SEQ ID NO: 9. In embodiments, the TRAAK protein comprises an amino acid sequence that has 96% identity to a 100 amino acid contiguous sequence of SEQ ID NO: 9. In embodiments, the TRAAK protein comprises an amino acid sequence that has 97% identity to SEQ ID NO: 9. In embodiments, the TRAAK protein comprises an amino acid sequence that has 97% identity to a 100 amino acid contiguous sequence of SEQ ID NO: 9. In embodiments, the TRAAK protein comprises an amino acid sequence that has 98% identity to SEQ ID NO: 9. In embodiments, the TRAAK protein comprises an amino acid sequence that has 98% identity to a 100 amino acid contiguous sequence of SEQ ID NO: 9. In embodiments, the TRAAK protein comprises an amino acid sequence that has 99% identity to SEQ ID NO: 9. In embodiments, the TRAAK protein comprises an amino acid sequence that has 99% identity to a 100 amino acid contiguous sequence of SEQ ID NO: 9.

In embodiments, the TRAAK protein comprises SEQ ID NO: 10. In embodiments, the TRAAK protein comprises a 100 amino acid contiguous sequence of SEQ ID NO: 10. In embodiments, the TRAAK protein comprises an amino acid sequence that has 80% identity to SEQ ID NO: 10. In embodiments, the TRAAK protein comprises an amino acid sequence that has 80% identity to a 100 amino acid contiguous sequence of SEQ ID NO: 10. In embodiments, the TRAAK protein comprises an amino acid sequence that has 85% identity to SEQ ID NO: 10. In embodiments, the TRAAK protein comprises an amino acid sequence that has 85% identity to a 100 amino acid contiguous sequence of SEQ ID NO: 10. In embodiments, the TRAAK protein comprises an amino acid sequence that has 90% identity to SEQ ID NO: 10. In embodiments, the TRAAK protein comprises an amino acid sequence that has 90% identity to a 100 amino acid contiguous sequence of SEQ ID NO: 10. In embodiments, the TRAAK protein comprises an amino acid sequence that has 95% identity to SEQ ID NO: 10. In embodiments, the TRAAK protein comprises an amino acid sequence that has 95% identity to a 100 amino acid contiguous sequence of SEQ ID NO: 10. In embodiments, the TRAAK protein comprises an amino acid sequence that has 96% identity to SEQ ID NO: 10. In embodiments, the TRAAK protein comprises an amino acid sequence that has 96% identity to a 100 amino acid contiguous sequence of SEQ ID NO: 10. In embodiments, the TRAAK protein comprises an amino acid sequence that has 97% identity to SEQ ID NO: 10. In embodiments, the TRAAK protein comprises an amino acid sequence that has 97% identity to a 100 amino acid contiguous sequence of SEQ ID NO: 10. In embodiments, the TRAAK protein comprises an amino acid sequence that has 98% identity to SEQ ID NO: 10. In embodiments, the TRAAK protein comprises an amino acid sequence that has 98% identity to a 100 amino acid contiguous sequence of SEQ ID NO: 10. In embodiments, the TRAAK protein comprises an amino acid sequence that has 99% identity to SEQ ID NO: 10. In embodiments, the TRAAK protein comprises an amino acid sequence that has 99% identity to a 100 amino acid contiguous sequence of SEQ ID NO: 10.

In embodiments, the TRAAK protein comprises SEQ ID NO: 11. In embodiments, the TRAAK protein comprises a 100 amino acid contiguous sequence of SEQ ID NO: 11. In embodiments, the TRAAK protein comprises an amino acid sequence that has 80% identity to SEQ ID NO: 11. In embodiments, the TRAAK protein comprises an amino acid sequence that has 80% identity to a 100 amino acid contiguous sequence of SEQ ID NO: 11. In embodiments, the TRAAK protein comprises an amino acid sequence that has 85% identity to SEQ ID NO: 11. In embodiments, the TRAAK protein comprises an amino acid sequence that has 85% identity to a 100 amino acid contiguous sequence of SEQ ID NO: 11. In embodiments, the TRAAK protein comprises an amino acid sequence that has 90% identity to SEQ ID NO: 11. In embodiments, the TRAAK protein comprises an amino acid sequence that has 90% identity to a 100 amino acid contiguous sequence of SEQ ID NO: 11. In embodiments, the TRAAK protein comprises an amino acid sequence that has 95% identity to SEQ ID NO: 11. In embodiments, the TRAAK protein comprises an amino acid sequence that has 95% identity to a 100 amino acid contiguous sequence of SEQ ID NO: 11. In embodiments, the TRAAK protein comprises an amino acid sequence that has 96% identity to SEQ ID NO: 11. In embodiments, the TRAAK protein comprises an amino acid sequence that has 96% identity to a 100 amino acid contiguous sequence of SEQ ID NO: 11. In embodiments, the TRAAK protein comprises an amino acid sequence that has 97% identity to SEQ ID NO: 11. In embodiments, the TRAAK protein comprises an amino acid sequence that has 97% identity to a 100 amino acid contiguous sequence of SEQ ID NO: 11. In embodiments, the TRAAK protein comprises an amino acid sequence that has 98% identity to SEQ ID NO: 11. In embodiments, the TRAAK protein comprises an amino acid sequence that has 98% identity to a 100 amino acid contiguous sequence of SEQ ID NO: 11. In embodiments, the TRAAK protein comprises an amino acid sequence that has 99% identity to SEQ ID NO: 11. In embodiments, the TRAAK protein comprises an amino acid sequence that has 99% identity to a 100 amino acid contiguous sequence of SEQ ID NO: 11.

In embodiments, the TRAAK protein comprises SEQ ID NO: 12. In embodiments, the TRAAK protein comprises a 100 amino acid contiguous sequence of SEQ ID NO: 12. In embodiments, the TRAAK protein comprises an amino acid sequence that has 80% identity to SEQ ID NO: 12. In embodiments, the TRAAK protein comprises an amino acid sequence that has 80% identity to a 100 amino acid contiguous sequence of SEQ ID NO: 12. In embodiments, the TRAAK protein comprises an amino acid sequence that has 85% identity to SEQ ID NO: 12. In embodiments, the TRAAK protein comprises an amino acid sequence that has 85% identity to a 100 amino acid contiguous sequence of SEQ ID NO: 12. In embodiments, the TRAAK protein comprises an amino acid sequence that has 90% identity to SEQ ID NO: 12. In embodiments, the TRAAK protein comprises an amino acid sequence that has 90% identity to a 100 amino acid contiguous sequence of SEQ ID NO: 12. In embodiments, the TRAAK protein comprises an amino acid sequence that has 95% identity to SEQ ID NO: 12. In embodiments, the TRAAK protein comprises an amino acid sequence that has 95% identity to a 100 amino acid contiguous sequence of SEQ ID NO: 12. In embodiments, the TRAAK protein comprises an amino acid sequence that has 96% identity to SEQ ID NO: 12. In embodiments, the TRAAK protein comprises an amino acid sequence that has 96% identity to a 100 amino acid contiguous sequence of SEQ ID NO: 12. In embodiments, the TRAAK protein comprises an amino acid sequence that has 97% identity to SEQ ID NO: 12. In embodiments, the TRAAK protein comprises an amino acid sequence that has 97% identity to a 100 amino acid contiguous sequence of SEQ ID NO: 12. In embodiments, the TRAAK protein comprises an amino acid sequence that has 98% identity to SEQ ID NO: 12. In embodiments, the TRAAK protein comprises an amino acid sequence that has 98% identity to a 100 amino acid contiguous sequence of SEQ ID NO: 12. In embodiments, the TRAAK protein comprises an amino acid sequence that has 99% identity to SEQ ID NO: 12. In embodiments, the TRAAK protein comprises an amino acid sequence that has 99% identity to a 100 amino acid contiguous sequence of SEQ ID NO: 12.

### IV. Methods of Treatment

Provided herein, inter alia, are methods for treating a disease or adverse condition related to low TREK family protein activity in a subject in need thereof. The disclosed methods comprise administering to the subject a nucleic acid encoding a TREK family protein as provided herein (e.g. TREK-1, TREK-2 or TRAAK protein) and a TREK family protein agonist as provided herein. In embodiments, the TREK family protein agonist comprises a cysteine binding moiety capable of covalently binding the TREK family protein at the modified cysteine residue. In embodiments, the nucleic acid encoding a TREK family protein as provided herein and the TREK family protein agonist are provided in an effective amount, also referred to herein as a combined effective amount. In embodiments, the nucleic acid encoding a TREK family protein as provided herein and the TREK family protein agonist are provided in a therapeutically effective amount, also referred to herein as a combined therapeutically effective amount. In embodiments, the nucleic acid encoding a TREK family protein as provided herein is provided in an effective amount (e.g. a therapeutically effective amount). In embodiments, the TREK family protein agonist as provided herein is provided in an effective amount (e.g. a therapeutically effective amount).

In embodiments, the nucleic acid is within a viral particle. In embodiments, the viral particle is an inactivated or genetically modified human papillomavirus, rhinovirus, hepatitis B virus, or herpesvirus. In embodiments, the viral particle is an inactivated or genetically modified human papillomavirus. In embodiments, the viral particle is an inactivated or genetically modified rhinovirus. In embodiments, the viral particle is an inactivated or genetically modified hepatitis B virus. In embodiments, the viral particle is an inactivated or genetically modified herpesvirus. In embodiments, the viral protein encoding the nucleic acid encoding a TREK family protein as provided herein and a TREK family protein agonist is provided in an effective amount (e.g. a therapeutically effective amount).

In embodiments, the TREK family protein agonist is a short-hairpin RNA (shRNA), a small interference RNA (siRNA), a piwi-interacting RNA (piRNA), a microRNA (miRNA), an antisense oligonucleotide, such as a GapmeR or a morpholinooligonucleotide, a CRISPR Cas guide RNA (gRNA), a small molecule compound, or an antibody.

In embodiments, the TREK family protein agonist is a short-hairpin RNA (shRNA).

In embodiments, the TREK family protein agonist is a small interference RNA (siRNA).

In embodiments, the TREK family protein agonist is a piwi-interacting RNA (piRNA).

In embodiments, the TREK family protein agonist is a microRNA (miRNA).

In embodiments, the TREK family protein agonist is an antisense oligonucleotide.

In embodiments, the TREK family protein agonist is a GapmeR.

In embodiments, the TREK family protein agonist is a morpholinooligonucleotide.

In embodiments, the TREK family protein agonist is a CRISPR Cas guide RNA (gRNA). In embodiments, the CRISPR Cas guide RNA is CRISPR Cas 12 guide RNA. In embodiments, the CRISPR Cas guide RNA is CRISPR Cas 9 guide RNA.

In embodiments, the TREK family protein agonist is an antibody.

In embodiments, the TREK family protein agonist is a small molecule compound of the structural Formula (I), or a pharmaceutically acceptable salt thereof, as described herein.

In embodiments, the methods provided herein can be used to treat or prevent a disorder, disease, or condition, such as a disease, disorder or condition related to low TREK family protein activity, in a subject in need thereof. Exemplary diseases, disorders and conditions include, but are not limited to, chronic pain, nerve injury, insomnia, lack of sleep, high intraocular pressure, headache, depression, pulmonary hypertension, lung injury, and decompression sickness. In embodiments, the nerve injury is an injury of the dorsal ganglion nerve.

In embodiments, provided herein are methods of treating a disease or adverse condition related to low TREK family protein activity in a subject in need thereof The disclosed methods comprise administering to the subject a gene editing system capable of mutating a TREK family protein, by inserting a cysteine residue at an amino position corresponding to position 131 of TREK-1, and a TREK family protein agonist. In embodiments, the TREK family protein agonist comprises a cysteine binding moiety that is capable of covalently binding the TREK family protein at the modified cysteine residue.

In embodiments, the gene editing system is a CRISPR Cas guide RNA (gRNA), a transcription activator-like effector nuclease (TALEN), or a zinc-finger nuclease (ZFN). In embodiments, the gene editing system is a CRISPR Cas guide RNA (gRNA). In embodiments, the CRISPR Cas guide RNA is CRISPR Cas 12 guide RNA. In embodiments, the CRISPR Cas guide RNA is CRISPR Cas 9 guide RNA. In embodiments, the gene editing system is a transcription activator-like effector nuclease (TALEN). In embodiments, the gene editing system is a zinc-finger nuclease (ZFN).

In embodiments, the cysteine binding moiety is:

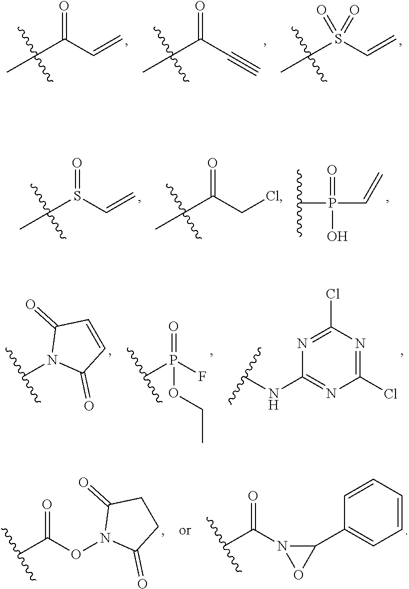

Additionally provided herein are methods for increasing TREK family protein activity in a tissue, such as a mammal or human tissue. The disclosed methods comprise administering to the tissue a nucleic acid encoding a TREK family protein as provided herein and a TREK family protein agonist. In embodiments, the TREK family protein agonist comprises a cysteine binding moiety capable of covalently binding the TREK family protein at the modified cysteine residue.

In embodiments, the tissue is a brain, heart, smooth muscle, nerve, nerve ganglia, eye, endocrine, pancreas, prostate, or sensory organ tissue. In embodiments, the tissue is a brain. In embodiments, the tissue is a heart. In embodiments, the tissue is a smooth muscle. In embodiments, the tissue is a nerve. In embodiments, the tissue is a nerve ganglion. In embodiments, the tissue is a In embodiments, the tissue is an endocrine tissue. In embodiments, the tissue is is a pancreas. In embodiments, the tissue is an eye. In embodiments, the tissue is a prostate. In embodiments, the tissue is a sensory organ tissue.

In embodiments, the TREK family protein agonist is a short-hairpin RNA (shRNA), a small interference RNA (siRNA), a piwi-interacting RNA (piRNA), a microRNA (miRNA), an antisense oligonucleotide, such as a GapmeR or a morpholinooligonucleotide, a CRISPR Cas guide RNA (gRNA), a small molecule compound, or an antibody.

In embodiments, the TREK family protein agonist is a short-hairpin RNA (shRNA).

In embodiments, the TREK family protein agonist is a small interference RNA (siRNA).

In embodiments, the TREK family protein agonist is a piwi-interacting RNA (piRNA).

In embodiments, the TREK family protein agonist is a microRNA (miRNA).

In embodiments, the TREK family protein agonist is an antisense oligonucleotide.

In embodiments, the TREK family protein agonist is a GapmeR.

In embodiments, the TREK family protein agonist is a morpholinooligonucleotide.

In embodiments, the TREK family protein agonist is a CRISPR Cas guide RNA (gRNA). In embodiments, the CRISPR Cas guide RNA is CRISPR Cas 12 guide RNA. In embodiments, the CRISPR Cas guide RNA is CRISPR Cas 9 guide RNA.

In embodiments, the TREK family protein agonist is an antibody.

In embodiments, the TREK family protein agonist is a small molecule compound of the structural Formula (I), or a pharmaceutically acceptable salt thereof, as described herein.

The present invention contemplates the administration of the compounds described herein, and compositions (e.g., pharmaceutical salts, pharmaceutical composition) thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the compounds disclosed herein over a defined period of time. In embodiments, the administration is oral, lingual, sublingual, parenteral, rectal, topical, transdermal or pulmonary administration.

The compounds of the present invention may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the compounds of the present invention may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the compounds contemplated by the present invention may be administered at dosage levels of about 0.01 mg/kg to about 50 mg/kg, or about 1 mg/kg to about 25 mg/kg, of subject body weight per day, one, two, three, four or more times a day, to obtain the desired therapeutic effect. For administration of an oral agent, the compositions can be provided in the form of tablets, capsules and the like containing from 0.05 to 1000 milligrams of the active ingredient, particularly 0.05, 0.1, 0.25, 0.5, 0.75, 1.0, 1.25, 1.5, 1.75, 2.0, 2.5, 5.0, 7.5, 10.0, 15.0, 20.0, 25.0, 50.0, 75.0, 100.0, 125.0, 150.0, 175.0, 200.0, 250.0, 300.0, 400.0, 500.0, 600.0, 750.0, 800.0, 900.0, and 1000.0 milligrams of the active ingredient. A pharmaceutically acceptable carrier(s), diluent(s) and/or excipient(s) may be present in an amount of from about 0.1 g to about 2.0 g.

In embodiments, the dosage of the desired compound is contained in a “unit dosage form”. The phrase “unit dosage form” refers to physically discrete units, each unit including a predetermined amount of the compound, sufficient to produce the desired effect. It will be appreciated that the parameters of a unit dosage form will depend on the particular agent and the effect to be achieved.

In embodiments, the TREK family protein and the TREK family protein agonist are administered in a combined effective amount and/or in a combined therapeutically effective amount to perform the methods described herein.

### V. Pharmaceutical Compositions

Additionally provided herein are pharmaceutical compositions for preventing or treating a disease or adverse condition related to low TREK family protein activity in a subject in need thereof. The disclosed pharmaceutical compositions comprise an effective amount of a nucleic acid encoding a TREK family protein as provided herein, a TREK family protein agonist, and one or more excipients or additives.

In embodiments, the TREK family protein agonist comprises a cysteine binding moiety capable of covalently binding the TREK family protein at the modified cysteine residue.

In embodiments, the cysteine binding moiety is:

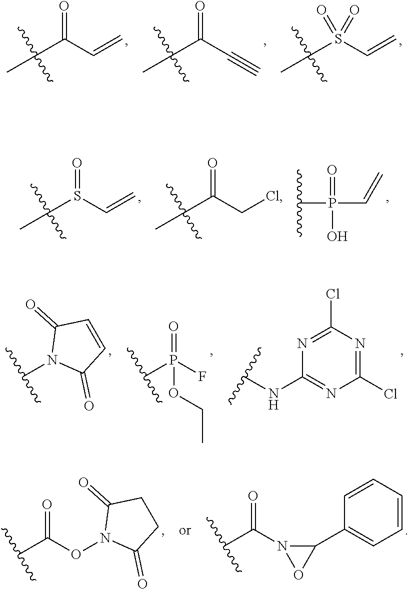

In embodiments, the nucleic acid is within a viral particle. In embodiments, the viral particle is an inactivated or genetically modified human papillomavirus, rhinovirus, hepatitis B virus, or herpesvirus. In embodiments, the viral particle is an inactivated or genetically modified human papillomavirus. In embodiments, the viral particle is an inactivated or genetically modified rhinovirus. In embodiments, the viral particle is an inactivated or genetically modified hepatitis B virus. In embodiments, the viral particle is an inactivated or genetically modified herpesvirus.

In embodiments, the TREK family protein agonist is a short-hairpin RNA (shRNA), a small interference RNA (siRNA), a piwi-interacting RNA (piRNA), a microRNA (miRNA), an antisense oligonucleotide, such as a GapmeR or a morpholinooligonucleotide, a CRISPR Cas guide RNA (gRNA), a small molecule compound, or an antibody.

In embodiments, the TREK family protein agonist is a short-hairpin RNA (shRNA).

In embodiments, the TREK family protein agonist is a small interference RNA (siRNA).

In embodiments, the TREK family protein agonist is a piwi-interacting RNA (piRNA).

In embodiments, the TREK family protein agonist is a microRNA (miRNA).

In embodiments, the TREK family protein agonist is an antisense oligonucleotide.

In embodiments, the TREK family protein agonist is a GapmeR.

In embodiments, the TREK family protein agonist is a morpholinooligonucleotide.

In embodiments, the TREK family protein agonist is a CRISPR Cas guide RNA (gRNA). In embodiments, the CRISPR Cas guide RNA is CRISPR Cas 12 guide RNA. In embodiments, the CRISPR Cas guide RNA is CRISPR Cas 9 guide RNA.

In embodiments, the TREK family protein agonist is a TREK family protein antibody.

In embodiments, the TREK family protein agonist is a small molecule compound of the structural Formula (I), or a pharmaceutically acceptable salt thereof, as described herein.

In embodiments, the pharmaceutical compositions provided herein can be used to treat or prevent a disorder, disease, or condition, such as a disease, disorder or condition related to low TREK family protein activity, in a subject in need thereof Exemplary diseases, disorders and conditions include, but are not limited to, chronic pain, nerve injury, insomnia, lack of sleep, high intraocular pressure, headache, depression, pulmonary hypertension, lung injury, and decompression sickness. In embodiments, the nerve injury is an injury of the dorsal ganglion nerve.

The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

The disclosed pharmaceutical compositions may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients that are suitable for the manufacture thereof. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release.

Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition.

For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a TREK protein family contemplated by the present disclosure, a therapeutically effective amount of a TREK family protein agonist described herein, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder.

Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a Wnt/catenin signaling pathway inhibitor, including implants (e.g., implantable pumps) and catheter systems, slow injection pumps and devices, all of which are well known to the skilled artisan.

Depot injections, which are generally administered subcutaneously or intramuscularly, may also be utilized to release a compound disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein.

The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, NJ) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

The present disclosure contemplates the administration of the compounds described herein in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

In embodiments, the pharmaceutical compositions provided herein are suitable for oral, lingual, sublingual, parenteral, rectal, topical, transdermal or pulmonary administration.

In an aspect is provided a pharmaceutical composition including a compound described herein, or a pharmaceutically acceptable salt thereof, and a pharmaceutically acceptable excipient.

In embodiments, the pharmaceutical composition includes an effective amount of the TREK family protein and an effective amount of a TREK agonist. In embodiments, the pharmaceutical composition includes a therapeutically effective amount of the TREK family protein and a therapeutically effective amount of a TREK agonist.

In embodiments, the TREK agonist is a compound of formula (I).

In embodiments, the pharmaceutical composition further includes a third agent. In embodiments, the third agent is an analgesic (for example, non-steroidal anti-inflammatory drugs, such as ibuprofen, naproxen, diclofenac, celecoxib, mefenamic acid, etoricoxib, indomethacin, and aspirin), opioid (e.g., morphine, fentanyl, hydrocodone, methadone, buprenorphine, oxycodone, codeine, tramadol, or tapendatol), antibiotic, or anticonvulsant, such as Gabapentin. In embodiments, the third agent is an anesthetic. In embodiments, the third agent is a local anesthetic (e.g., bupivacaine). In embodiments, the pharmaceutical composition includes a therapeutically effective amount of the third agent.

### VI. Embodiments

Embodiment P1. A compound having the formula (I):

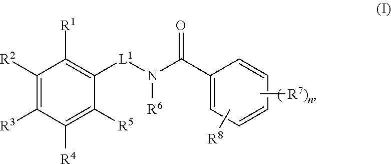

wherein:
L is a bond, substituted or unsubstituted alkylene or substituted or unsubstituted heteroalkylene;
R¹ is independently is hydrogen, halogen, —CX¹₃, —CHX¹₂, —CH₂X¹, —OCX¹₃, —OCHX¹₂, —OCH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, halogen, —CX²₃, —CHX²₂, —CH₂X², —OCX²₃, —OCHX²₂, —OCH₂X², —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently hydrogen, halogen, —CX³₃, —CHX³₂, —CH₂X³, —OCX³₃, —OCHX³₂, —OCH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D} —NR^{3B}C(O)OR^{3D} —NR^{3B}OR^{3D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is independently hydrogen, halogen, —CX⁴₃, —CHX⁴₂, —CH₂X⁴, —OCX⁴₃, —OCHX⁴₂, —OCH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{S} is independently hydrogen, halogen, —CX⁵³, —CHX⁵₂, —CH₂X⁵, —OCX⁵₃, —OCHX⁵₂, —OCH₂X⁵, —CN, —N₃, —SOₙ₅R^{5A}, —SOᵥ₅NR^{5B}R^{5C}, —NHNR^{SB}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is independently a substituted or unsubstituted alkyl or substituted or unsubstituted heteroalkyl; wherein R⁶ is optionally joined with L¹ to form a substituted or unsubstituted heterocycloalkyl;
R⁷ is independently halogen, —CX⁷₃, —CHX⁷₂, —CH₂X⁷, —OCX⁷₃, —OCHX⁷₂, —OCH₂X⁷, —CN, —N₃, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —N(O)ₘ₇, —NR′^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, —NR^{7B}OR^{7D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, Ric, RID, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{7A}, R^{7B}, R^{7C}, and R^{7D} are independently hydrogen, halogen, —CF₃, —Cl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹, X², X³, X⁴, X⁵, and X⁷ are independently halogen;
n is an integer from 0 to 3;
n1, n2, n3, n4, n5, and n7 is independently an integer from 0 to 4;
m1, m2, m3, m4, m5, m7, v1, v2, v3, v4, v5, and v7 are independently 1 or 2; and
R⁸ is a cysteine binding moiety or a serine binding moiety.

Embodiment P2. The compound of embodiment P1, wherein the cysteine binding moiety is:

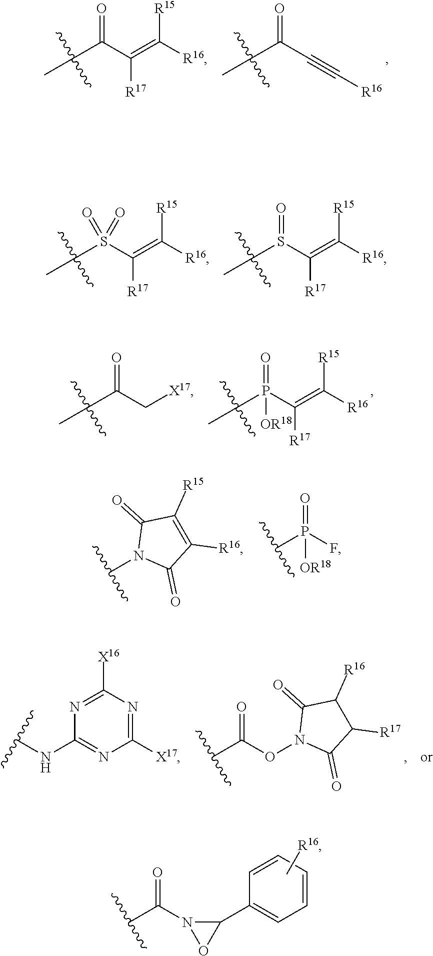

wherein:
R¹⁵, R¹⁶, R¹⁶, and R¹⁸ are independently hydrogen, substituted or unsubstituted C₁-C₄ alkyl or substituted or unsubstituted 2 to 4 membered heteroalkyl; and
X¹⁷ is halogen.

Embodiment P3. The compound of embodiment P2, wherein R¹⁵, R¹⁶, R¹⁶, and R¹⁸ are independently hydrogen or unsubstituted C₁-C₂ alkyl and X¹⁷ is chlorine.

Embodiment P4. The compound of embodiments P2 or P3, wherein the cysteine binding moiety is:

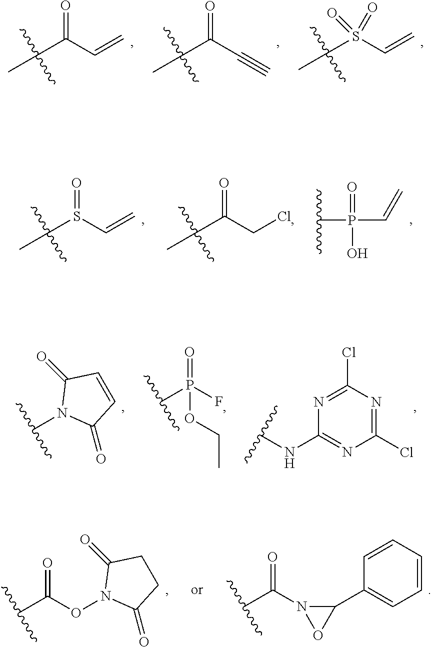

Embodiment P5. The compound of embodiment P1, wherein the serine binding moiety is:

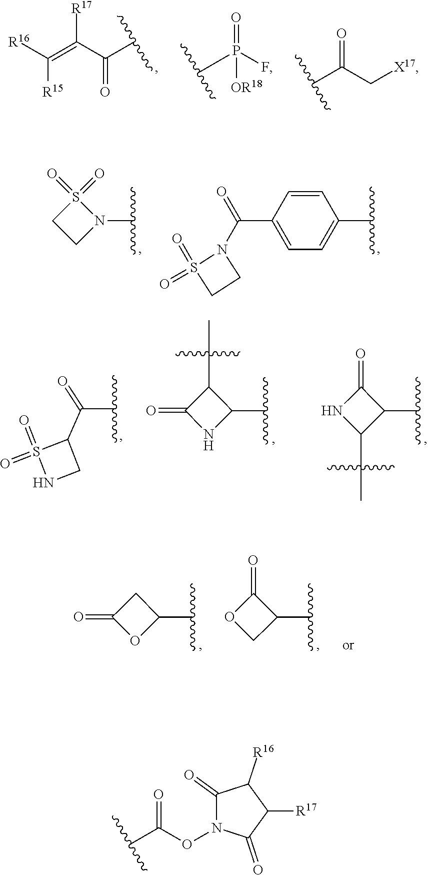

wherein:
R¹⁵, R¹⁶, R¹⁶, and R⁸ are independently hydrogen, substituted or unsubstituted C₁-C₄ alkyl or substituted or unsubstituted 2 to 4 membered heteroalkyl; and
X¹⁷ is halogen.

Embodiment P6. The compound of embodiment P5, wherein R¹⁵, R¹⁶, R¹⁶, and R¹⁸ are independently hydrogen or unsubstituted C₁-C₂ alkyl and X¹⁷ is chlorine.

Embodiment P7. The compound of embodiments P5 or P6, wherein the serine binding moiety is:

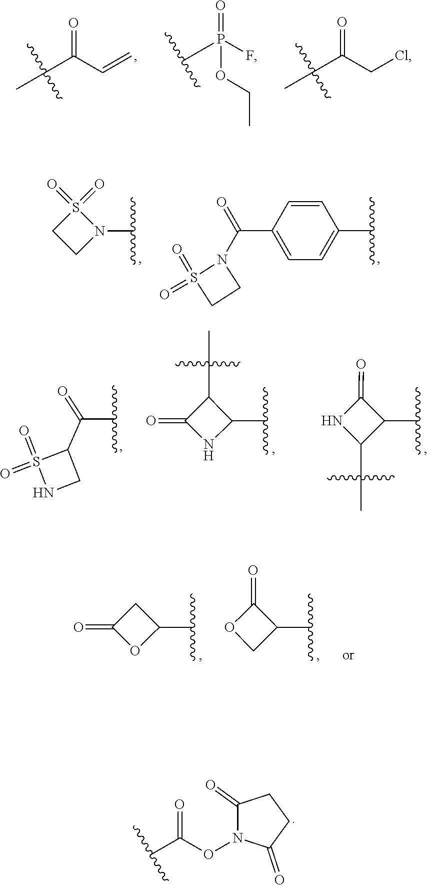

Embodiment P8. The compound of embodiment P1, wherein R¹ is hydrogen or halogen.

Embodiment P9. The compound of embodiment P8, wherein R¹ is —Cl.

Embodiment P10. The compound of embodiment P1, wherein R² is hydrogen or halogen.

Embodiment P11. The compound of embodiment P10, wherein R² is hydrogen.

Embodiment P12. The compound of embodiment P10, wherein R² is —F or —Cl.

Embodiment P13. The compound of embodiment P1, wherein R³ is halogen or C₁-C₄ substituted or unsubstituted alkynyl.

Embodiment P14. The compound of embodiment P13, wherein R³ is halogen.

Embodiment P15. The compound of embodiment P14, wherein R³ is —Cl, —Br, or —I.

Embodiment P16. The compound of embodiment P13, wherein R³ is unsubstituted 1λ³,2λ³-ethyne.

Embodiment P17. The compound of embodiment P1, wherein R⁴ and R⁵ are independently hydrogen.

Embodiment P18. The compound of embodiment P1, wherein R⁶ is hydrogen, substituted or unsubstituted C₁-C₄ alkyl or R⁶ and L¹ are joined together to form a substituted or unsubstituted 5 to 7 membered heterocycloalkyl.

Embodiment P19. The compound of embodiment P18, wherein R⁶ is hydrogen.

Embodiment P20. The compound of embodiment P18, wherein R⁶ is unsubstituted methyl.

Embodiment P21. The compound of embodiment P18, wherein R⁶ is 5 to 7 membered heterocycloalkyl.

Embodiment P22. The compound of embodiment P1, wherein R⁶ is 1λ²-azepan-2-one, 1,4λ²-oxazepan-5-one, or 1λ²-piperidin-2-one.

Embodiment P23. The compound of embodiment P1, wherein R⁷ is a substituted or unsubstituted C₁-C₄ alkyl, substituted or unsubstituted 2 to 4 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P24. The compound of embodiment P23, wherein R⁷ is an unsubstituted C₁-C₄ alkyl, unsubstituted 2 to 4 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl.

Embodiment P25. A TREK family protein or homolog thereof comprising a cysteine residue at an amino position corresponding to position 131 of TREK-1.

Embodiment P26. The TREK family protein or homolog thereof of embodiment P25, wherein the TREK family protein is a TREK-1 protein, a TREK-2 protein or a TRAKK protein.

Embodiment P27. The TREK Family protein of embodiment P26, wherein the TREK family protein comprises any one of SEQ ID NOS: 1-12.

Embodiment P28. The TREK Family protein of embodiment P27, wherein the TREK family protein comprises SEQ ID NO: 1.

Embodiment P29. The TREK Family protein of embodiment P27, wherein the TREK family protein comprises SEQ ID NO: 2.

Embodiment P30. The TREK Family protein of embodiment P27, wherein the TREK family protein comprises SEQ ID NO: 3.

Embodiment P31. The TREK Family protein of embodiment P27, wherein the TREK family protein comprises SEQ ID NO: 4.

Embodiment P32. The TREK Family protein of embodiment P27, wherein the TREK family protein comprises SEQ ID NO: 5.

Embodiment P33. The TREK Family protein of embodiment P27, wherein the TREK family protein comprises SEQ ID NO: 6.

Embodiment P34. The TREK Family protein of embodiment P27, wherein the TREK family protein comprises SEQ ID NO: 7.

Embodiment P35. The TREK Family protein of embodiment P27, wherein the TREK family protein comprises SEQ ID NO: 8.

Embodiment P36. The TREK Family protein of embodiment P27, wherein the TREK family protein comprises SEQ ID NO: 9.

Embodiment P37. The TREK Family protein of embodiment P27, wherein the TREK family protein comprises SEQ ID NO: 10.

Embodiment P38. The TREK Family protein of embodiment P27, wherein the TREK family protein comprises SEQ ID NO: 11.

Embodiment P39. The TREK Family protein of embodiment P27, wherein the TREK family protein comprises SEQ ID NO: 12.

Embodiment P40. A method of treating a disease or adverse condition related to low TREK family protein activity in a subject in need thereof, the method comprising administering to the subject an effective amount of a nucleic acid encoding the TREK family protein of embodiment P25 and a therapeutically effective amount of a TREK family protein agonist, wherein said TREK family protein agonist comprises a cysteine binding moiety capable of covalently binding said TREK family protein at said cysteine residue.

Embodiment P41. The method of embodiment P40, wherein the TREK family protein is a TREK-1 protein, a TREK-2 protein or a TRAAK protein.

Embodiment P42. The method of embodiment P40, wherein said nucleic acid is within a viral particle.

Embodiment P43. The method of embodiment P42, wherein the viral particle is an inactivated or genetically modified human papillomavirus, rhinovirus, hepatitis B virus, or herpesvirus.

Embodiment P44. The method of embodiment P40, wherein said TREK family protein agonist is a short-hairpin RNA (shRNA), a small interference RNA (siRNA), a piwi-interacting RNA (piRNA), a microRNA (miRNA), an antisense oligonucleotide, a morpholinooligonucleotide, a CRISPR Cas guide RNA (gRNA), a small molecule compound, an antibody, or a nucleic acid.

Embodiment P45. The method of embodiment P44, wherein said TREK family protein agonist is a short-hairpin RNA (shRNA).

Embodiment P46. The method of embodiment P44, wherein said TREK family protein agonist is a small interference RNA (siRNA).

Embodiment P47. The method of embodiment P44, wherein said TREK family protein agonist is a piwi-interacting RNA (piRNA).

Embodiment P48. The method of embodiment P44, wherein said TREK family protein agonist is a microRNA (miRNA).

Embodiment P49. The method of embodiment P44, wherein said TREK family protein agonist is an antisense oligonucleotide.

Embodiment P50. The method of embodiment P44, wherein said TREK family protein agonist is a GapmeR.

Embodiment P51. The method of embodiment P44, wherein said TREK family protein agonist is a morpholinooligonucleotide.

Embodiment P52. The method of embodiment P44, wherein said TREK family protein agonist is a CRISPR Cas guide RNA (gRNA).

Embodiment P53. The method of embodiment P52, wherein said CRISPR Cas guide RNA is CRISPR Cas 12 guide RNA.

Embodiment P54. The method of embodiment P52, wherein said CRISPR Cas guide RNA is CRISPR Cas 9 guide RNA.

Embodiment P55. The method of embodiment P44, wherein said TREK family protein agonist is aTREK family protein antibody.

Embodiment P56. The method of embodiment P44, wherein said TREK family protein agonist is a nucleic acid binding said cysteine residue.

Embodiment P57. The method of embodiment P44, wherein said TREK family protein agonist is the compound of any one of embodiments P1-P24.

Embodiment P58. The method of any one of embodiments P40-P57, wherein the cysteine residue is at an amino position corresponding to position 131 of TREK-1.

Embodiment P59. The method of any one of embodiments P40-P58, wherein said disease or adverse condition is chronic pain, nerve injury, lack of sleep, high intraocular pressure, headache, depression, pulmonary hypertension, lung injury, or decompression sickness.

Embodiment P60. The method of embodiment P59, wherein the nerve injury is an injury of the dorsal ganglion nerve.

Embodiment P61. A method of treating a disease or adverse condition related to low TREK family protein activity in a subject in need thereof, the method comprising administering to said subject a gene editing system capable of mutating a TREK family protein to comprise a cysteine residue at an amino position corresponding to position 131 of TREK-1, and a TREK family protein agonist, wherein said TREK family protein agonist comprises a cysteine binding moiety that is capable of covalently binding said TREK family protein at said cysteine residue.

Embodiment P62. The method of embodiment P61, wherein the TREK family protein is a TREK-1 protein, a TREK-2 protein or a TRAAK protein.

Embodiment P63. The method of embodiment P61, wherein said gene editing system is a CRISPR Cas guide RNA (gRNA), a transcription activator-like effector nuclease (TALEN), or a zinc-finger nuclease (ZFN).

Embodiment P64. The method of embodiment P63, wherein said CRISPR Cas guide RNA is CRISPR Cas 12 guide RNA.

Embodiment P65. The method of embodiment P63, wherein said CRISPR Cas guide RNA is CRISPR Cas 9 guide RNA.

Embodiment P66. A method of increasing TREK family protein activity in a tissue, said method comprising administering to said tissue a nucleic acid encoding the TREK family protein of embodiment P25 and a TREK family protein agonist, wherein said TREK family protein agonist comprises a cysteine binding moiety capable of covalently binding said TREK family protein at said cysteine residue.

Embodiment P67. The method of embodiment P66, wherein the TREK family protein is a TREK-1 protein, a TREK-2 protein or a TRAAK protein.

Embodiment P68. The method of embodiment P66, wherein said TREK family protein agonist is a short-hairpin RNA (shRNA), a small interference RNA (siRNA), a piwi-interacting RNA (piRNA), a microRNA (miRNA), an antisense oligonucleotide, a morpholinooligonucleotide, a CRISPR Cas guide RNA (gRNA), a small molecule compound, an antibody, or a nucleic acid.

Embodiment P69. The method of embodiment P68, wherein said TREK family protein agonist is a short-hairpin RNA (shRNA).

Embodiment P70. The method of embodiment P68, wherein said TREK family protein agonist is a small interference RNA (siRNA).

Embodiment P71. The method of embodiment P68, wherein said TREK family protein agonist is a piwi-interacting RNA (piRNA).

Embodiment P72. The method of embodiment P68, wherein said TREK family protein agonist is a microRNA (miRNA).

Embodiment P73. The method of embodiment P68, wherein said TREK family protein agonist is an antisense oligonucleotide.

Embodiment P74. The method of embodiment P68, wherein said TREK family protein agonist is a GapmeR.

Embodiment P75. The method of embodiment P68, wherein said TREK family protein agonist is a morpholinooligonucleotide.

Embodiment P76. The method of embodiment P68, wherein said TREK family protein agonist is a CRISPR Cas guide RNA (gRNA).

Embodiment P77. The method of embodiment P76, wherein said CRISPR Cas guide RNA is CRISPR Cas 12 guide RNA.

Embodiment P78. The method of embodiment P76, wherein said CRISPR Cas guide RNA is CRISPR Cas 9 guide RNA.

Embodiment P79. The method of embodiment P68, wherein said TREK family protein agonist is aTREK family protein antibody.

Embodiment P80. The method of embodiment P61, wherein said TREK family protein agonist is a nucleic acid binding said cysteine residue.

Embodiment P81. The method of embodiment P61, wherein said TREK family protein agonist is the compound of any one of embodiments P1-P24.

Embodiment P82. The method of any one of embodiments P61-P81, wherein the cysteine residue is at an amino position corresponding to position 131 of TREK-1.

Embodiment P83. The method of embodiment P61, wherein said tissue is a brain, heart, nerve, nerve ganglia, eye, smooth muscle, endocrine, pancreas, prostate, or sensory organ tissue.

### VII. ADDITIONAL EMBODIMENTS

Embodiment 1. A compound, or a pharmaceutically acceptable salt thereof, having the formula (I):

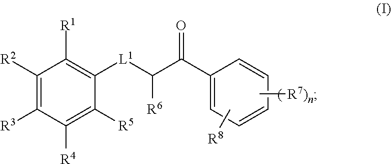

wherein:
L¹ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCHX¹₂, —OCH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCHX²₂, —OCH₂X², —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCHX³₂, —OCH₂X³, —CN, —N₃, —SOₙ₃R^{3A}, —SOᵥ₃NR^{3B}R^{3C}, —NH—R^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D} —NR^{3B}OR^{3D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCHX⁴₂, —OCH₂X⁴, —CN, —N₃, —SOₙ₄R^{4A}, —SOᵥ₄NR^{4B}R^{4C}, —NR^{4B}R^{4C}, —ONR^{4B}R^{4C}, —NHC(O)NHNR^{4B}R^{4C}, —NHC(O)NR^{4B}R^{4C}, N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{S} is hydrogen, halogen, —CX⁵₃, —CHX⁵², —CH₂X⁵, —OCX⁵₃, —OCHX⁵₂, —OCH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, substituted or unsubstituted alkyl, or substituted or unsubstituted heteroalkyl; or R⁶ is optionally joined with L¹ to form a substituted or unsubstituted heterocycloalkyl;
R⁷ is independently halogen, —CX⁷₃, —CHX⁷₂, —CH₂X⁷, —OCX⁷₃, —OCHX⁷₂, —OCH₂X⁷, —CN, —N₃, —SOₙ₇R^{7A}, —SOᵥ₇NR^{7B}R^{7C}, —, NR^{7B}R^{7C}, —ONR^{7B}R^{7C}, —NHC(O)NHNR^{7B}R^{7C}, NHC(O)NR^{7B}R^{7C}, —N(O)ₘ₇, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, —NR^{7B}OR^{7D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, Ric, RID, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{7A}, R^{7B}, R^{7C}, and R^{7D} are independently hydrogen, halogen, —CF₃, —Cl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each X¹, X², X³, X⁴, X⁵, and X⁷ is independently halogen;
n is an integer from 0 to 3;
n1, n2, n3, n4, n5, and n7 are independently an integer from 0 to 4;
m1, m2, m3, m4, m5, m7, v1, v2, v3, v4, v5, and v7 are independently 1 or 2; and
R⁸ is a cysteine binding moiety or a serine binding moiety.

Embodiment 2. The compound of embodiment 1, wherein the cysteine binding moiety is:

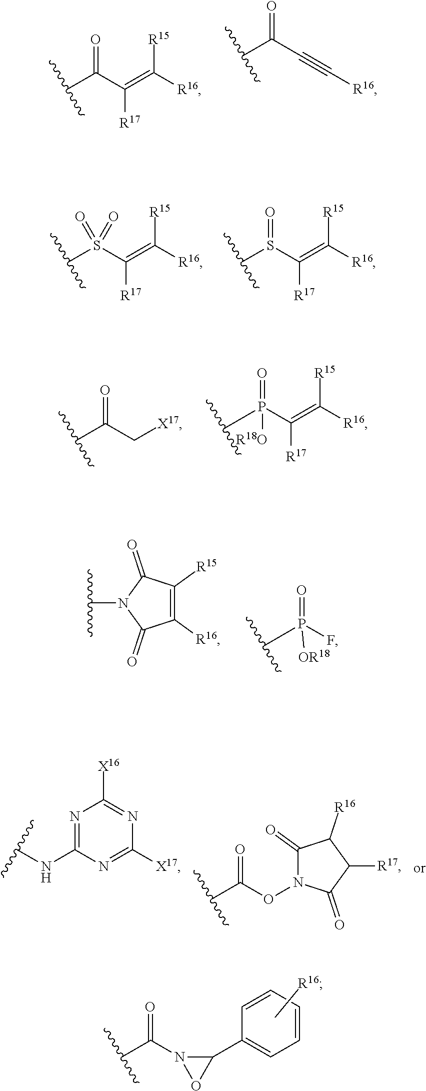

wherein:
R¹⁵ is hydrogen, halogen, —CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁶ is hydrogen, halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B} C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁷ is hydrogen, halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁸ is hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, and R^{18C} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;
X, X¹⁵, X¹⁶, X¹⁷, and X¹⁸ are independently —F, —Cl, —Br, or —I;
n15, n16, and n17 are independently an integer from 0 to 4; and
m15, m16, m17, v15, v16, and v17 are independently 1 or 2.

Embodiment 3. The compound of embodiment 2, wherein R¹⁵, R¹⁶, R¹⁷, and R¹⁸ are independently hydrogen, substituted or unsubstituted C₁-C₄ alkyl, or substituted or unsubstituted 2 to 4 membered heteroalkyl.

Embodiment 4. The compound of embodiment 2, wherein R¹⁵, R¹⁶, R¹⁷, and R¹⁸ are independently hydrogen or unsubstituted C₁-C₂ alkyl and X¹⁷ is —Cl.

Embodiment 5. The compound of one of embodiments 1 to 4, wherein the cysteine binding moiety is:

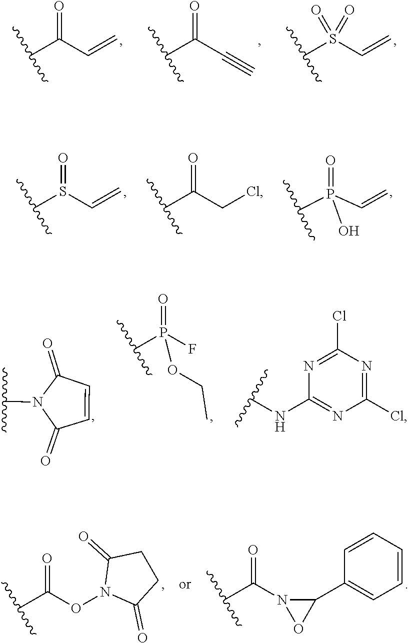

Embodiment 6. The compound of embodiment 1, wherein the serine binding moiety is:

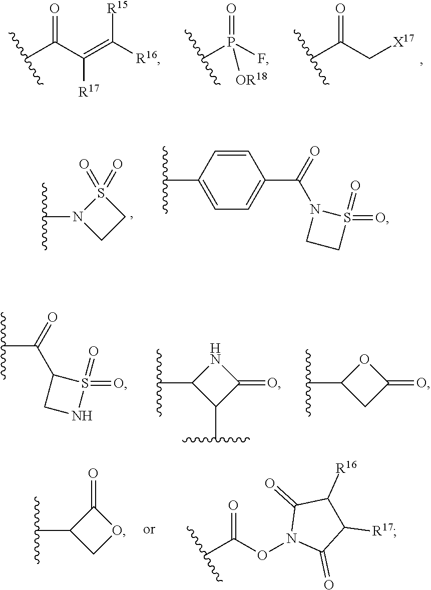

wherein:
R¹⁵, R¹⁶, R¹⁶, and R¹⁸ are independently hydrogen, substituted or unsubstituted C₁-C₄ alkyl, or substituted or unsubstituted 2 to 4 membered heteroalkyl; and X¹⁷ is halogen.

Embodiment 7. The compound of embodiment 6, wherein R¹⁵, R¹⁶, R¹⁷, and R¹⁸ are independently hydrogen or unsubstituted C₁-C₂ alkyl and X¹⁷ is —Cl.

Embodiment 8. The compound of embodiment 6 or embodiment 7, wherein the serine binding moiety is:

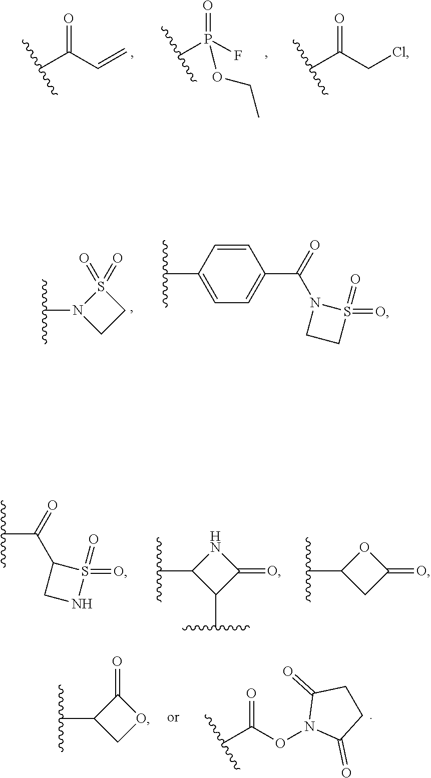

Embodiment 9. The compound of one of embodiments 1 to 8, wherein R¹ is hydrogen or halogen.

Embodiment 10. The compound of one of embodiments 1 to 8, wherein R¹ is —Cl.

Embodiment 11. The compound of one of embodiments 1 to 10, wherein R² is hydrogen or halogen.

Embodiment 12. The compound of one of embodiments 1 to 10, wherein R² is hydrogen.

Embodiment 13. The compound of one of embodiments 1 to 10, wherein R² is —F or —Cl.

Embodiment 14. The compound of one of embodiments 1 to 13, wherein R³ is halogen or C₁-C₄ substituted or unsubstituted alkynyl.

Embodiment 15. The compound of one of embodiments 1 to 13, wherein R³ is halogen.

Embodiment 16. The compound of one of embodiments 1 to 13, wherein R³ is —Cl, —Br, or —I.

Embodiment 17. The compound of one of embodiments 1 to 13, wherein R³ is unsubstituted 1λ³,2λ²-ethyne.

Embodiment 18. The compound of one of embodiments 1 to 17, wherein R⁴ and R⁵ are hydrogen.

Embodiment 19. The compound of one of embodiments 1 to 18, wherein R⁶ is hydrogen, substituted or unsubstituted C₁-C₄ alkyl; or R⁶ and L¹ are joined together to form a substituted or unsubstituted 5 to 7 membered heterocycloalkyl.

Embodiment 20. The compound of one of embodiments 1 to 18, wherein R⁶ is hydrogen.

Embodiment 21. The compound of one of embodiments 1 to 18, wherein R⁶ is unsubstituted methyl.

Embodiment 22. The compound of one of embodiments 1 to 18, wherein R⁶ and L¹ are joined together to form a substituted or unsubstituted 5 to 7 membered heterocycloalkyl.

Embodiment 23. The compound of one of embodiments 1 to 18, wherein R⁶ and L¹ are joined together to form a substituted or unsubstituted 1λ²-azepan-2-one, substituted or unsubstituted 1,4λ²-oxazepan-5-one, or substituted or unsubstituted 1λ²-piperidin-2-one.

Embodiment 24. The compound of one of embodiments 1 to 23, wherein R⁷ is independently a substituted or unsubstituted C₁-C₄ alkyl, substituted or unsubstituted 2 to 4 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 25. The compound of one of embodiments 1 to 23, wherein R⁷ is an unsubstituted C₁-C₄ alkyl, unsubstituted 2 to 4 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl.

Embodiment 26. A TREK family protein or homolog thereof comprising a cysteine residue at an amino position corresponding to position 131 of TREK-1.

Embodiment 27. The TREK family protein or homolog thereof of embodiment 26, wherein the TREK family protein is a TREK-1 protein, a TREK-2 protein, or a TRAKK protein.

Embodiment 28. The TREK family protein or homolog thereof of embodiment 26 or embodiment 27, wherein the TREK family protein comprises any one of SEQ ID NOS: 1-12.

Embodiment 29. The TREK family protein or homolog thereof of one of embodiments 26 to 28, wherein the TREK family protein comprises SEQ ID NO: 1.

Embodiment 30. The TREK family protein or homolog thereof of one of embodiments 26 to 28, wherein the TREK family protein comprises SEQ ID NO: 2.

Embodiment 31. The TREK family protein or homolog thereof of one of embodiments 26 to 28, wherein the TREK family protein comprises SEQ ID NO: 3.

Embodiment 32. The TREK family protein or homolog thereof of one of embodiments 26 to 28, wherein the TREK family protein comprises SEQ ID NO: 4.

Embodiment 33. The TREK family protein or homolog thereof of one of embodiments 26 to 28, wherein the TREK family protein comprises SEQ ID NO: 5.

Embodiment 34. The TREK family protein or homolog thereof of one of embodiments 26 to 28, wherein the TREK family protein comprises SEQ ID NO: 6.

Embodiment 35. The TREK family protein or homolog thereof of one of embodiments 26 to 28, wherein the TREK family protein comprises SEQ ID NO: 7.

Embodiment 36. The TREK family protein or homolog thereof of one of embodiments 26 to 28, wherein the TREK family protein comprises SEQ ID NO: 8.

Embodiment 37. The TREK family protein or homolog thereof of one of embodiments 26 to 28, wherein the TREK family protein comprises SEQ ID NO: 9.

Embodiment 38. The TREK family protein or homolog thereof of one of embodiments 26 to 28, wherein the TREK family protein comprises SEQ ID NO: 10.

Embodiment 39. The TREK family protein or homolog thereof of one of embodiments 26 to 28, wherein the TREK family protein comprises SEQ ID NO: 11.

Embodiment 40. The TREK family protein or homolog thereof of one of embodiments 26 to 28, wherein the TREK family protein comprises SEQ ID NO: 12.

Embodiment 41. A nucleic acid encoding the TREK family protein or homolog thereof of one of embodiments 26 to 40.

Embodiment 42. A viral particle comprising the nucleic acid of embodiment 41.

Embodiment 43. A method of treating a disease or adverse condition related to low TREK family protein activity in a subject in need thereof, the method comprising administering to the subject an effective amount of a nucleic acid encoding the TREK family protein of one of embodiments 26 to 40 and a therapeutically effective amount of a TREK family protein agonist, wherein said TREK family protein agonist comprises a cysteine binding moiety capable of covalently binding said TREK family protein at said cysteine residue.

Embodiment 44. The method of embodiment 43, wherein the TREK family protein is a TREK-1 protein, a TREK-2 protein, or a TRAAK protein.

Embodiment 45. The method of embodiment 43, wherein said nucleic acid is within a viral particle.

Embodiment 46. The method of embodiment 45, wherein the viral particle is an inactivated or genetically modified human papillomavirus, rhinovirus, hepatitis B virus, or herpesvirus.

Embodiment 47. The method of embodiment 43, wherein said TREK family protein agonist is a short-hairpin RNA (shRNA), a small interference RNA (siRNA), a piwi-interacting RNA (piRNA), a microRNA (miRNA), an antisense oligonucleotide, a morpholinooligonucleotide, a CRISPR Cas guide RNA (gRNA), a small molecule compound, an antibody, or a nucleic acid.

Embodiment 48. The method of embodiment 47, wherein said TREK family protein agonist is a short-hairpin RNA (shRNA).

Embodiment 49. The method of embodiment 47, wherein said TREK family protein agonist is a small interference RNA (siRNA).

Embodiment 50. The method of embodiment 47, wherein said TREK family protein agonist is a piwi-interacting RNA (piRNA).

Embodiment 51. The method of embodiment 47, wherein said TREK family protein agonist is a microRNA (miRNA).

Embodiment 52. The method of embodiment 47, wherein said TREK family protein agonist is an antisense oligonucleotide.

Embodiment 53. The method of embodiment 47, wherein said TREK family protein agonist is a GapmeR.

Embodiment 54. The method of embodiment 47, wherein said TREK family protein agonist is a morpholinooligonucleotide.

Embodiment 55. The method of embodiment 47, wherein said TREK family protein agonist is a CRISPR Cas guide RNA (gRNA).

Embodiment 56. The method of embodiment 55, wherein said CRISPR Cas guide RNA is CRISPR Cas 12 guide RNA.

Embodiment 57. The method of embodiment 55, wherein said CRISPR Cas guide RNA is CRISPR Cas 9 guide RNA.

Embodiment 58. The method of embodiment 47, wherein said TREK family protein agonist is aTREK family protein antibody.

Embodiment 59. The method of embodiment 47, wherein said TREK family protein agonist is a nucleic acid binding said cysteine residue.

Embodiment 60. The method of embodiment 47, wherein said TREK family protein agonist is the compound of any one of embodiments 1 to 25.

Embodiment 61. The method of any one of embodiments 43 to 60, wherein the cysteine residue is at an amino position corresponding to position 131 of TREK-1.

Embodiment 62. The method of any one of embodiments 43 to 61, wherein said disease or adverse condition is chronic pain, nerve injury, lack of sleep, high intraocular pressure, headache, depression, pulmonary hypertension, lung injury, or decompression sickness.

Embodiment 63. The method of embodiment 62, wherein the nerve injury is an injury of the dorsal ganglion nerve.

Embodiment 64. A method of treating a disease or adverse condition related to low TREK family protein activity in a subject in need thereof, the method comprising administering to said subject a gene editing system capable of mutating a TREK family protein to comprise a cysteine residue at an amino position corresponding to position 131 of TREK-1, and a TREK family protein agonist, wherein said TREK family protein agonist comprises a cysteine binding moiety that is capable of covalently binding said TREK family protein at said cysteine residue.

Embodiment 65. The method of embodiment 64, wherein the TREK family protein is a TREK-1 protein, a TREK-2 protein, or a TRAAK protein.

Embodiment 66. The method of embodiment 64, wherein said gene editing system is a CRISPR Cas guide RNA (gRNA), a transcription activator-like effector nuclease (TALEN), or a zinc-finger nuclease (ZFN).

Embodiment 67. The method of embodiment 66, wherein said CRISPR Cas guide RNA is CRISPR Cas 12 guide RNA.

Embodiment 68. The method of embodiment 66, wherein said CRISPR Cas guide RNA is CRISPR Cas 9 guide RNA.

Embodiment 69. A method of increasing TREK family protein activity in a tissue, said method comprising administering to said tissue a nucleic acid encoding the TREK family protein of one of embodiments 26 to 40 and a TREK family protein agonist, wherein said TREK family protein agonist comprises a cysteine binding moiety capable of covalently binding said TREK family protein at said cysteine residue.

Embodiment 70. The method of embodiment 69, wherein the TREK family protein is a TREK-1 protein, a TREK-2 protein, or a TRAAK protein.

Embodiment 71. The method of embodiment 69, wherein said TREK family protein agonist is a short-hairpin RNA (shRNA), a small interference RNA (siRNA), a piwi-interacting RNA (piRNA), a microRNA (miRNA), an antisense oligonucleotide, a morpholinooligonucleotide, a CRISPR Cas guide RNA (gRNA), a small molecule compound, an antibody, or a nucleic acid.

Embodiment 72. The method of embodiment 71, wherein said TREK family protein agonist is a short-hairpin RNA (shRNA).

Embodiment 73. The method of embodiment 71, wherein said TREK family protein agonist is a small interference RNA (siRNA).

Embodiment 74. The method of embodiment 71, wherein said TREK family protein agonist is a piwi-interacting RNA (piRNA).

Embodiment 75. The method of embodiment 71, wherein said TREK family protein agonist is a microRNA (miRNA).

Embodiment 76. The method of embodiment 71, wherein said TREK family protein agonist is an antisense oligonucleotide.

Embodiment 77. The method of embodiment 71, wherein said TREK family protein agonist is a GapmeR.

Embodiment 78. The method of embodiment 71, wherein said TREK family protein agonist is a morpholinooligonucleotide.

Embodiment 79. The method of embodiment 71, wherein said TREK family protein agonist is a CRISPR Cas guide RNA (gRNA).

Embodiment 80. The method of embodiment 79, wherein said CRISPR Cas guide RNA is CRISPR Cas 12 guide RNA.

Embodiment 81. The method of embodiment 79, wherein said CRISPR Cas guide RNA is CRISPR Cas 9 guide RNA.

Embodiment 82. The method of embodiment 71, wherein said TREK family protein agonist is aTREK family protein antibody.

Embodiment 83. The method of embodiment 64 or embodiment 69, wherein said TREK family protein agonist is a nucleic acid binding said cysteine residue.

Embodiment 84. The method of embodiment 64 or embodiment 69, wherein said TREK family protein agonist is the compound of any one of embodiments 1 to 25.

Embodiment 85. The method of any one of embodiments 64 to 84, wherein the cysteine residue is at an amino position corresponding to position 131 of TREK-1.

Embodiment 86. The method of any one of embodiments 69 to 85, wherein said tissue is a brain, heart, nerve, nerve ganglia, eye, smooth muscle, endocrine, pancreas, prostate, or sensory organ tissue.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Introduction

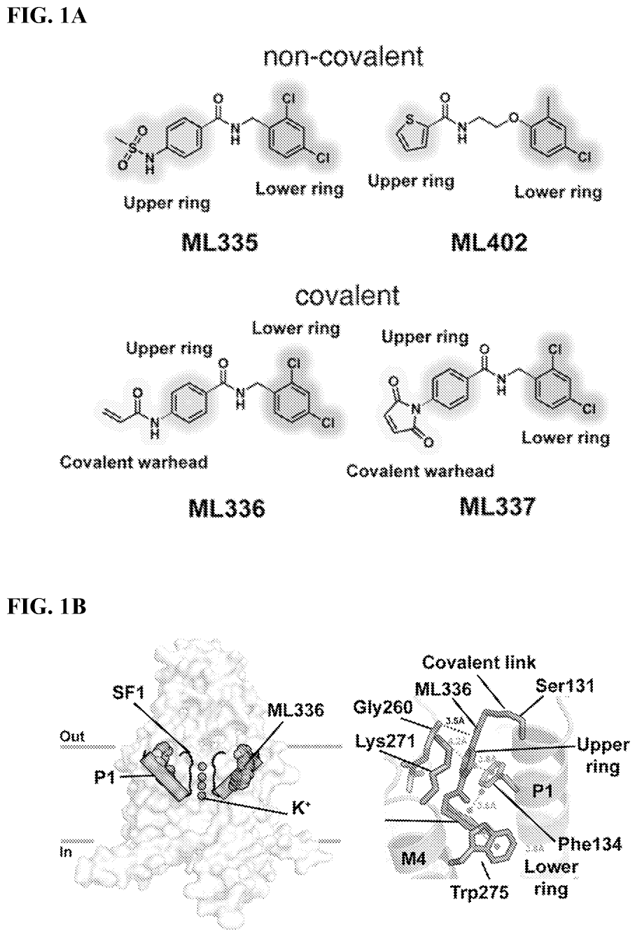

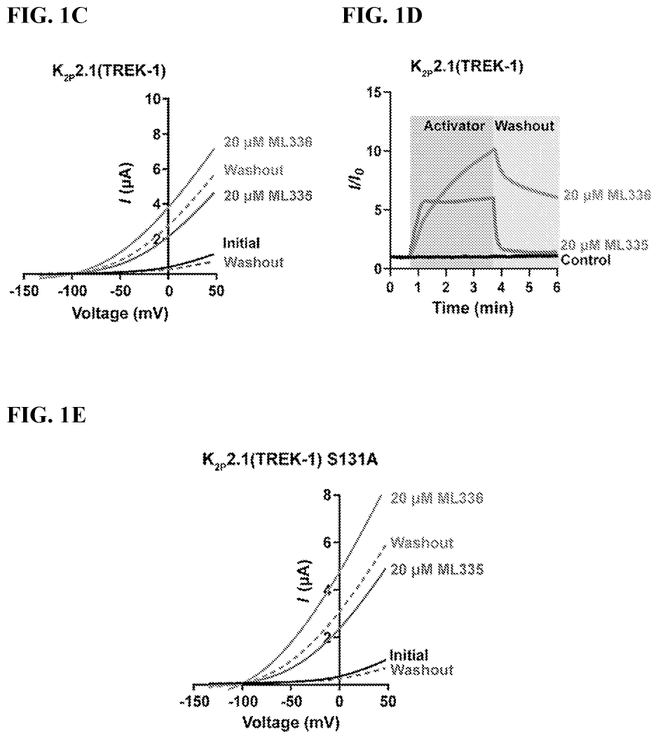

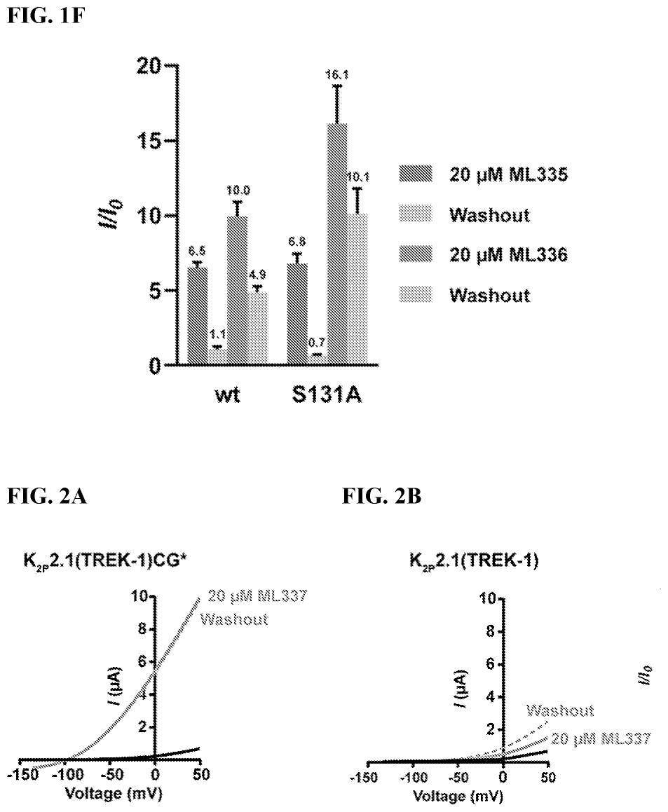

Described herein is the development of a covalent, chemogenetic activator for the TREK subfamily of K_{2P}s. Unlike traditional chemogenetic systems that rely on steric interactions to provide selectivity, we targeted genetically-defined K_{2P}s through covalent engagement using electrophilic ligands. Guided by x-ray crystallography and functional studies, we identified Ser131 of K_{2P}2.1 (TREK-1) as a favorable residue for covalent ligand engagement, due to its proximity to a known K_{2P} modulatory binding site (37). It was found that an acrylamide-functionalized K_{2P} agonist (ML336, FIG. 1A) bound Ser131 of wild-type channel under co-crystallization conditions. To further enhance rate of ligand capture at this site, a cysteine mutation at Ser131 of K_{2P}2.1 (TREK-1) was introduced and sought to identify a cognate ligand that would selectively engage the mutant over the wild-type channel. It was found that a bulkier maleimide warhead produced a ligand (CAT335, FIG. 1A) that selectively and irreversibly activates K_{2P}2.1 (TREK-1)^{CG}* (CG* denoting chemogenetic), but not wild-type K_{2P}2.1(TREK-1). We next used the CAT335/CG* system to study activation of ‘tandem’ K_{2P}2.1 (TREK-1) channels containing 0, 1 or 2 CG* subunits, and found that activation of just one CG* subunit in a heterodimeric channel was sufficient to achieve near-maximal channel activation. It was additionally shown that K_{2P}2.1(TREK-1)^{CG}* mutations can be favorably combined with previously reported inactivating mutations A286F and G171F (38). These attenuated CG* channels possessed significantly reduced basal leak currents, but were still sensitive to activation by CAT335. We then showed that attenuated CG* channels elicit ligand-specific K⁺ currents, hyperpolarizing plasma membranes only in the presence of CAT335. Finally, we demonstrated that the CG* system is amenable to other TREK subfamily members K₂P10.1(TREK-2) and K_{2P}4.1(TRAAK). Together, these results demonstrate the potential of chemogenetic-based tools for studying K_{2P} function.

An optimal chemogenetic system comprises a protein of interest engineered to respond to a cognate ligand, where the same ligand does not modulate endogenous protein function (28). Chemogenetic tools can thus offer precise chemical control of protein function that is orthogonal to native protein-ligand interactions (28).

### Example 1: Covalent modification of the k₂ₚ2.1 (trek-1) modulator pocket

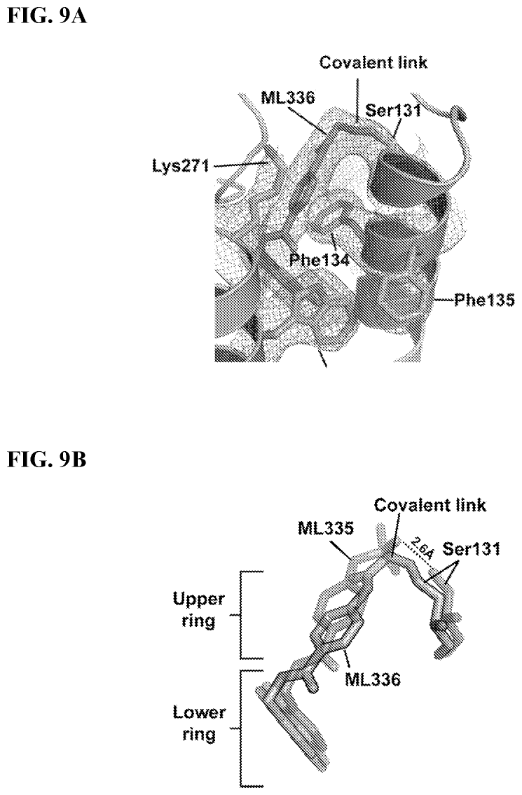

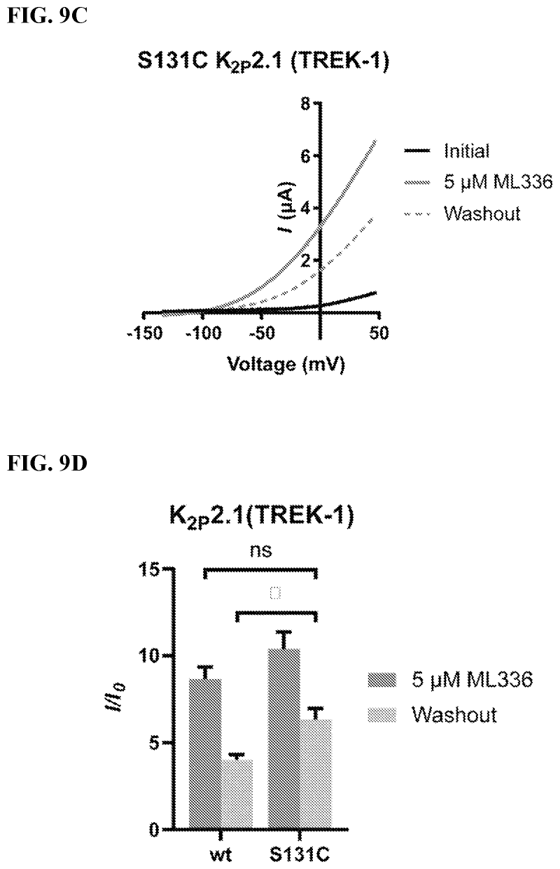

In the course of characterizing electrophilic moiety-bearing derivatives of the TREK subfamily activator ML335, we determined the co-crystal structure of a previously characterized K_{2P}2.1 (TREK-1) construct, K_{2P}2.1 (TREK-1)_{cryst} complexed with an ML335 acrylamide derivative, ML336 (FIG. 1A) at 2.9 Å resolution by X-ray crystallography (FIG. 1B, FIG. 9A). The structure showed ML336 bound to the K_{2P} modulator pocket (FIG. 1B) and revealed the presence of continuous electron density between the acrylamide and the Ser131 sidechain hydroxyl (FIG. 9A) indicating the formation of a covalent bond between ML336 and the channel. The ML336 lower ring is positioned similarly to its counterpart in ML335, while its upper ring is positioned ˜1.5 Å lower in the pocket relative to ML335 (FIG. 9B). As with ML335, ML336 interacts with Phe134, Lys271, and Trp275 (FIG. 1). Comparison with the ML335 structure shows that the ML336 covalent link takes the place of the hydrogen bond between the Ser131 hydroxyl and the ML335 methyl sulfonamide (FIG. 9B) (37). Having observed the covalent link to ML336 in the crystal structure, we turned to functional studies using two-electrode voltage clamp (TEVC) in Xenopus oocytes to assess whether we could find functional evidence for this interaction. Comparison of the effects of 20 μM ML335 and ML336 on oocytes expressing K_{2P}2.1 (TREK-1) showed that ML336 activates K_{2P}2.1(TREK-1) (I_{ML336}/Iᵢₙᵢₜᵢₐₗ=10+1.0, FIG. 1C) more strongly than ML335 (FIG. 1F). Moreover, unlike ML335, ML336 activation was sustained following washout over the course of several minutes (FIG. 1D). To determine if this activation was related to the covalent link observed crystallographically, we examined the effects of ML335 and ML336 on a mutant that should be incapable of forming such a link, S131A, and one that should enhance reactivity to the acrylamide, S131C. Surprisingly, application of ML336 to K_{2P}2.1 (TREK-1) S131 Å activated the channel in nearly identical fashion to K_{2P}2.1(TREK-1) (FIGS. 1E-1F). Additionally, ML336 application to K_{2P}2.1 (TREK-1) S131C showed washout behavior similar to the ML336: K_{2P}2.1 (TREK-1) combination, contrary to the expected reaction enhancement from the cysteine substitution (FIGS. 9C-9D). Thus, while ML336 was able to form a covalent adduct with Ser131 under crystallization conditions, which involve high concentrations of the components and a week long timeframe, we could not establish evidence for formation of this covalent adduct in the minute timescales used for functional studies. Nevertheless, the discovery of the ML336-Ser131 adduct demonstrates the availability of this position for covalent modification by activators that target the K_{2P} modulator pocket.

### Example 2: Development of a Chemogenetic K₂ₚ2.1 (Trek-1) Pair

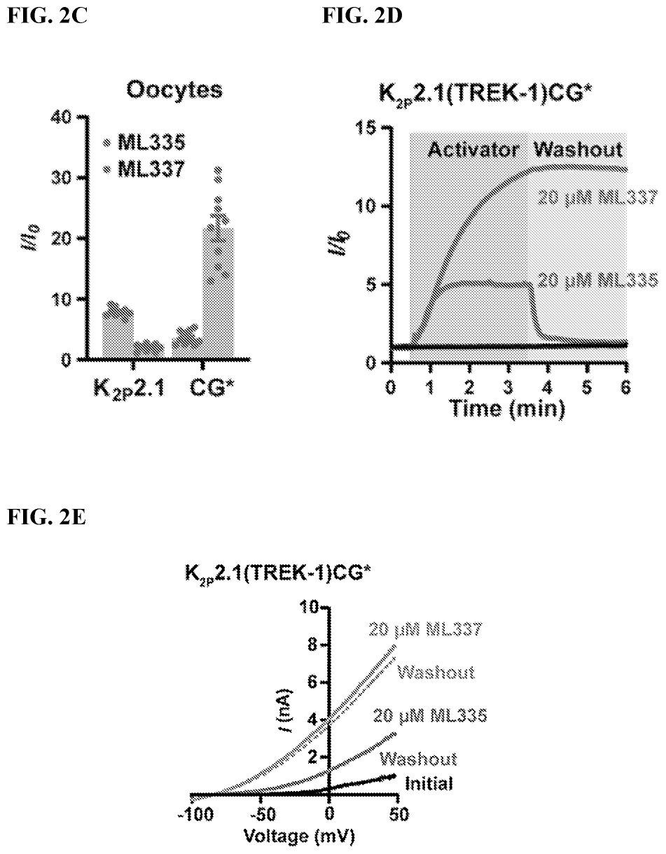

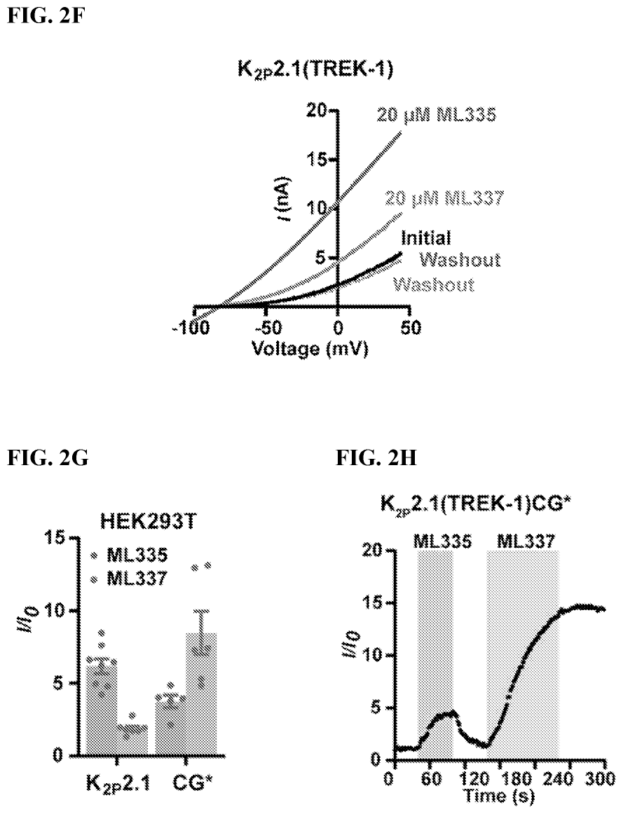

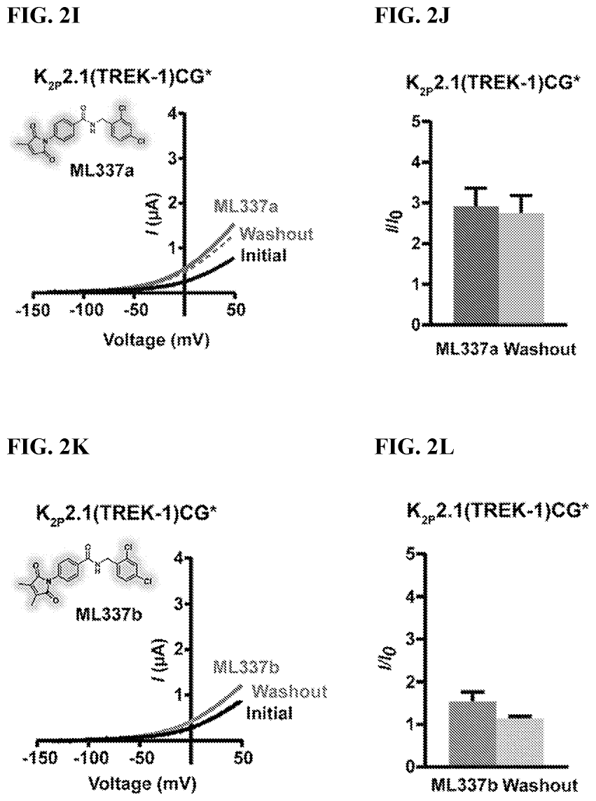

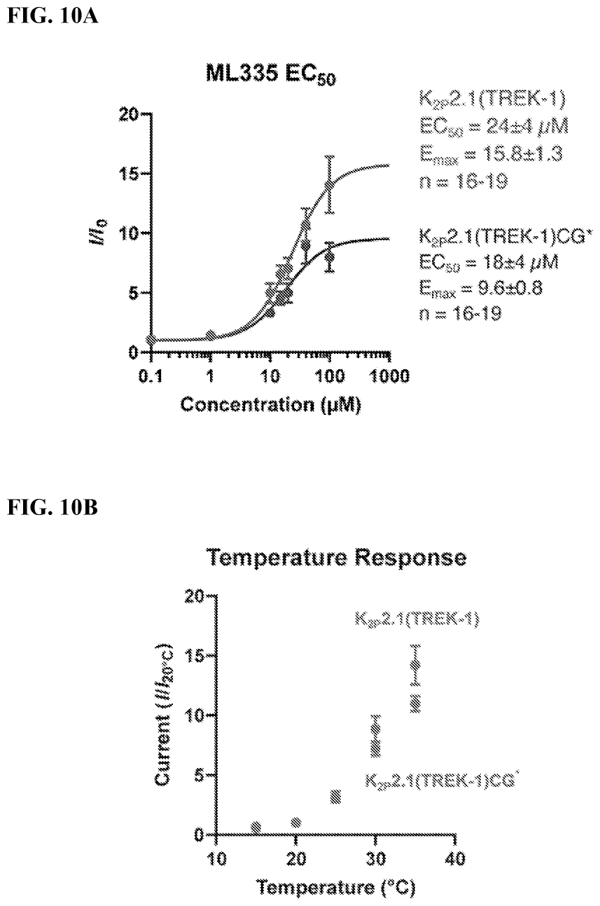

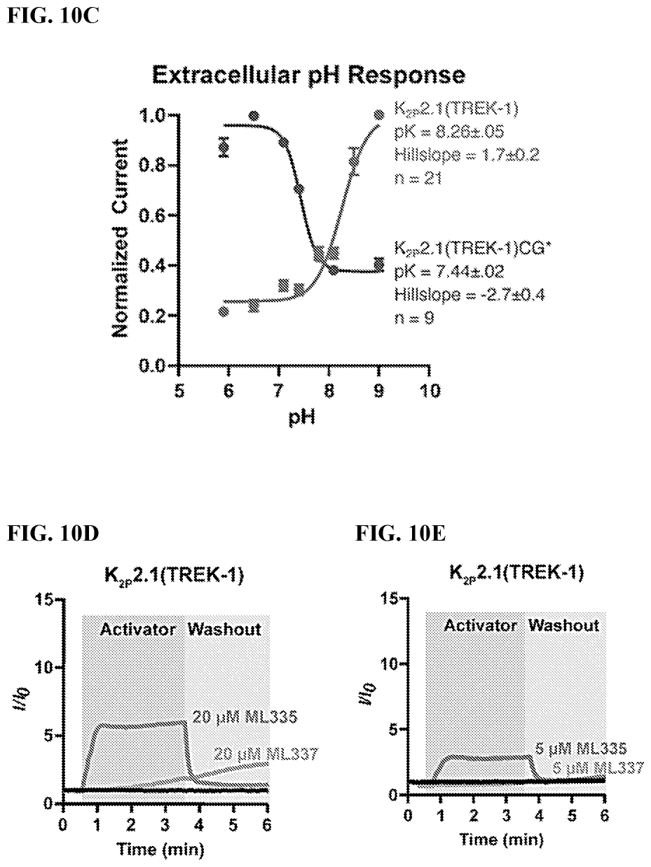

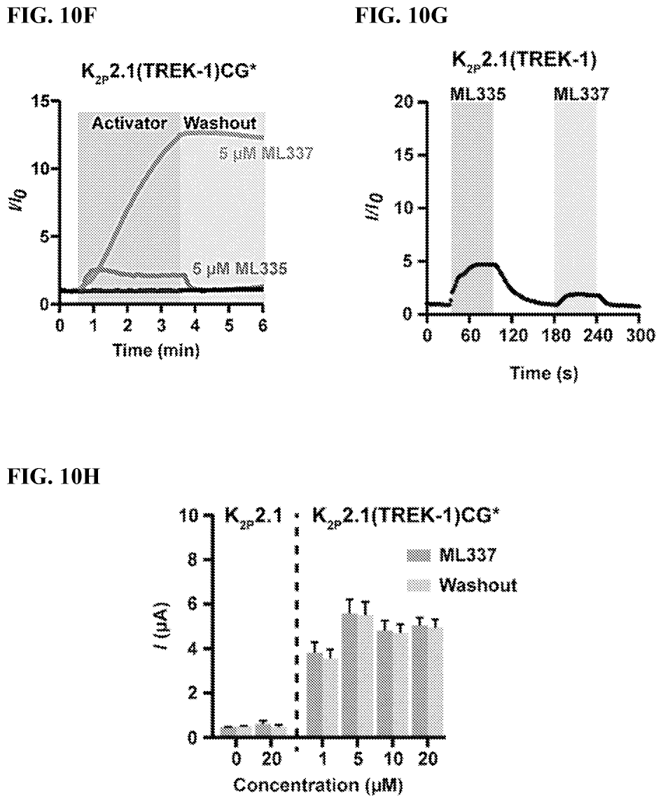

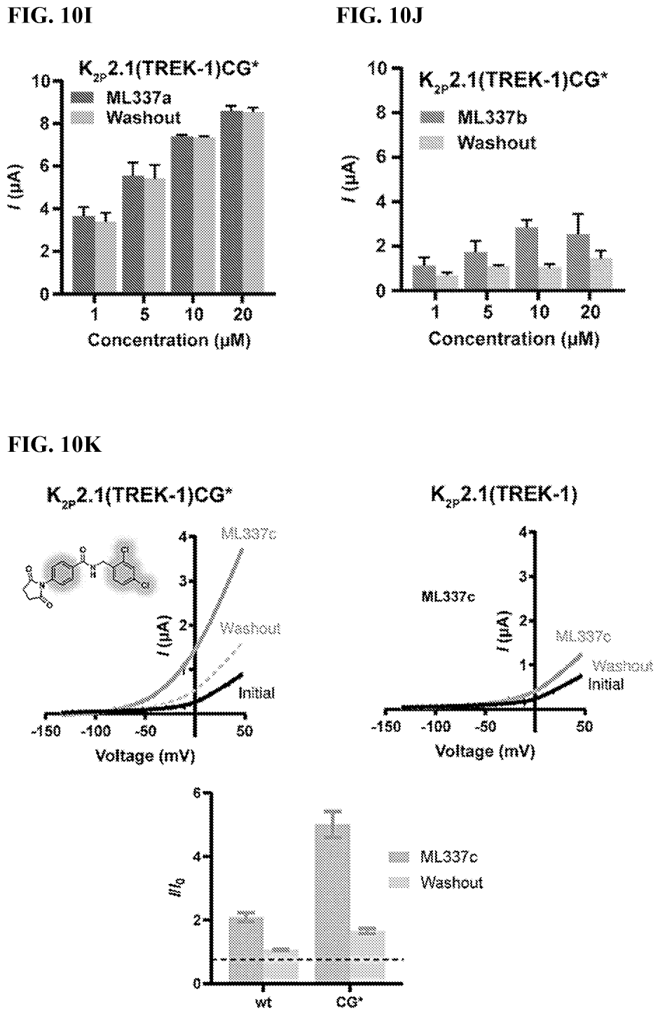

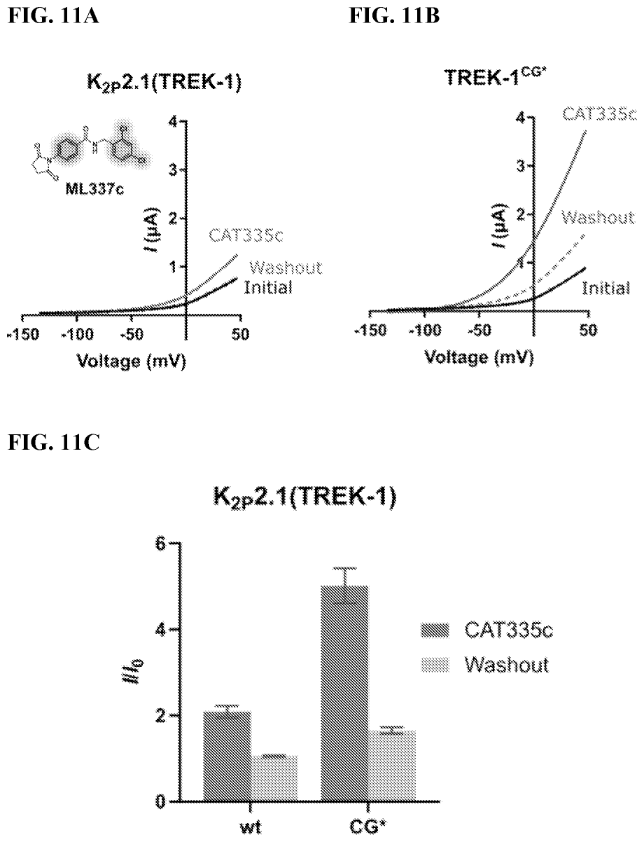

To develop a modulator:channel pair capable of rapid, irreversible activation, we modified the ML336 scaffold to replace the acrylamide group with the more reactive maleimide to afford CAT335 (Covalent Activator of TREK, FIG. 1A, SI Scheme 1), and tested whether this compound could irreversibly activate the S131C mutant (denoted hereafter as TREK-1^{CG}*, for chemogenetic). When applied to Xenopus oocytes expressing TREK-1^{CG}*, 20 μM CAT335 produced a large, irreversible activation (I_{CAT335}/I_{control}=22±2) and by contrast elicited a minimal response from K₂P2.1 (TREK-1) (I_{CAT335}/I_{control}=2.0±0.2, FIGS. 2A-2C). Comparison of the washout time course showed immediate reversal of the effects of the non-covalent activator ML335 on both TREK-1^{CG}* and K_{2P}2.1 (TREK-1) that contrasted the persistent activation of TREK-1^{CG}* by CAT335. Importantly, TREK-1^{CG}* maintained a response to the non-covalent activator ML335 similar to K_{2P}2.1 (TREK-1) (FIG. 10A) (EC₅₀=18±4 μM and 24±4 μM, and Eₘₐₓ I_{ML335}/I_{control}=9.6±0.8 and 15.8±1.3 fold activation, for K_{2P}2.1(TREK-1)CG* and K_{2P}2.1(TREK-1) respectively). Further, TREK-1^{CG}* displayed temperature-dependent activation similar to temperature K_{2P}2.1 (TREK-1) (FIG. 10B), indicating that the S131C change did not dramatically alter channel properties.

K_{2P}2.1 (TREK-1)^{CG}* and wt possess similar responses to K_{2P}2.1 (TREK-1)^{CG}* response to extracellular pH was altered, likely due to the proximity of Cys131 to His126, a key residue for TREK-1 extracellular pH sensing (FIG. 10C) (17).

We noticed a small degree of activation of K_{2P}2.1 (TREK-1) by 20 μM CAT335 following compound washout (I_{CAT335}/I_{control}=4.0+0.6 after washout) (FIG. 2B and FIG. 10D). This was less apparent at 5 μM where CAT335 still potently activated TREK-1^{CG}* (FIGS. 10E-10F). To examine whether this latent activation of K_{2P}2.1 (TREK-1) was general or due to the use of oocytes, we used whole cell patch clamp electrophysiology to characterize the CAT335 responses of TREK-1^{CG}* and K_{2P}2.1(TREK-1) in transfected HEK293 cells. These experiments revealed that, as in oocytes, CAT335 caused a strong, irreversible activation of TREK-1^{CG}* (I_{CAT335}/I_{control}=8.5+1.5), however elicited a much more modest and reversible response from K₂P2.1 (TREK-1) (I_{CAT335}/I_{control}=1.9+0.2), indicating that the observed latent activation was a consequence of the oocyte system and not a general property of CAT335 (FIGS. 2E-2H). As in oocytes, the non-covalent activator ML335 activated both channels but was slightly less effective at stimulating TREK-1^{CG}* (I_{ML335}/I_{control}=6.2+0.5 and 3.7+0.4 at 20 μM, respectively). Most importantly, CAT335 activation was irreversible in TREK-1^{CG}* expressing cells, but was fully reversible in wild-type K_{2P}2.1 (TREK-1) expressing cells (FIGS. 2E-2G), indicating that the observed latent activation was a consequence of the oocyte system and not a general property of CAT335.

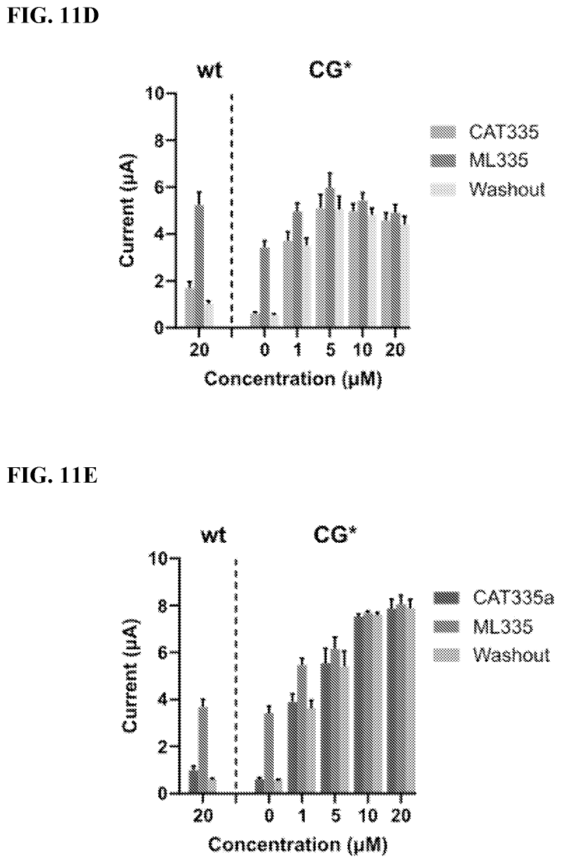

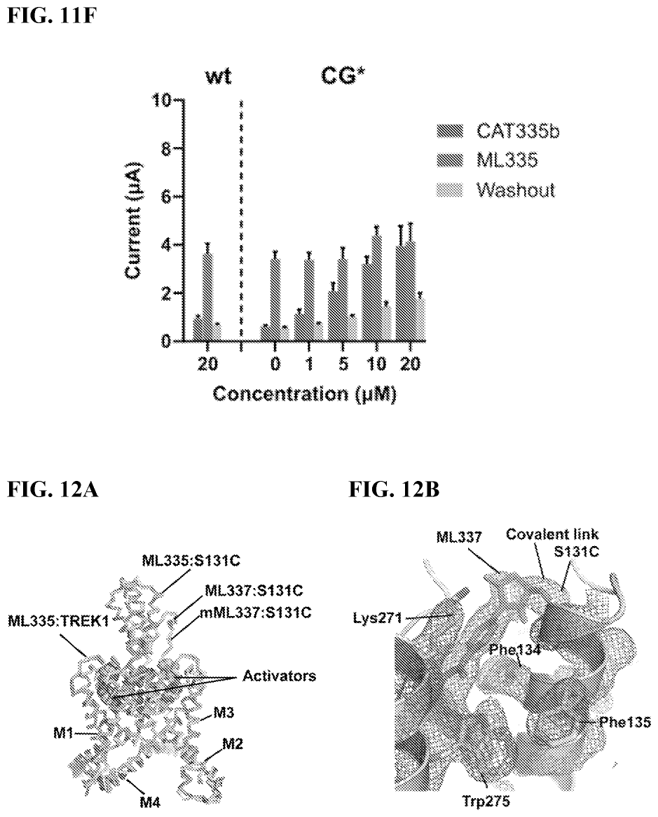

To explore the potential of the maleimide reactive functionality further, we synthesized a set of CAT335 derivatives that included two functionalized maleimides (CAT335a and CAT335b) and a succinimide (CAT335c). Methyl-substituted maleimide CAT335a was able to irreversibly activate TREK-1^{CG}*, however the magnitude of the response was slightly reduced relative to CAT335 for the equivalent amount of time (I_{CAT335a}/I_{control}=7.4±0.7 at 20 μM) (FIGS. 2A and 2I, 2C and 2J). The dimethyl-substituted CAT335b caused a further reduction in TREK-1^{CG}* activation (I_{CAT335b}/I_{control}=4.9±0.6 at 20 μM) that, unlike CAT335 and CAT335a, was partially reversible with washout (FIGS. 2K-2L). The non-electrophilic CAT335c had a minimal effect on K_{2P}2.1 (TREK-1) (I_{CAT335c}/I_{control}=2.09+0.14 at 20 μM) and a slightly larger, but reversible, activation of TREK-1^{CG}* (I_{CAT335c}/I_{contol}=5.0+0.4 at 20 μM) (FIGS. 11A-11C). To test whether the differences in activation were related to the kinetics of channel modification, we applied each of the three maleimide-based activators to wild-type K_{2P}2.1 (TREK-1) and TREK-1^{CG}* expressing oocytes for 1 hour at varying concentrations. We found that these extended incubation times resulted in significantly increased channel activation for all three maleimide-based activators (FIGS. 11D-11F), however CAT335b activation remained reversible, implying the dimethyl substitution of the maleimide prevents covalent engagement of Cys131. We named these chemogenetic pairs CATKLAMP for Covalent Activator of TREK channels whose action clamps the membrane potential near the potassium reversal potential.

### Structures of CATKLAMP Pairs

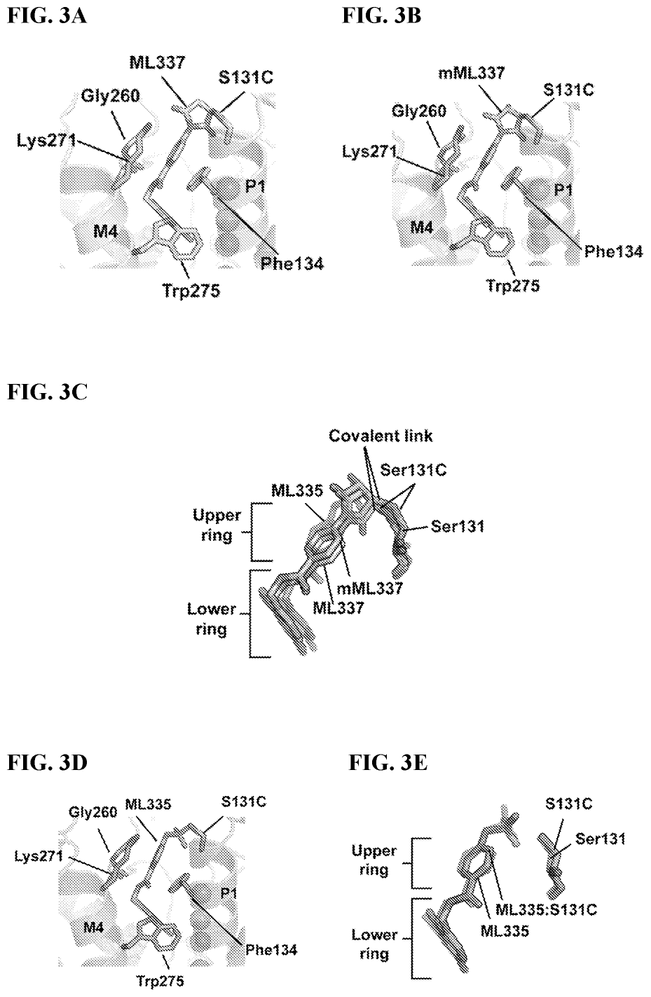

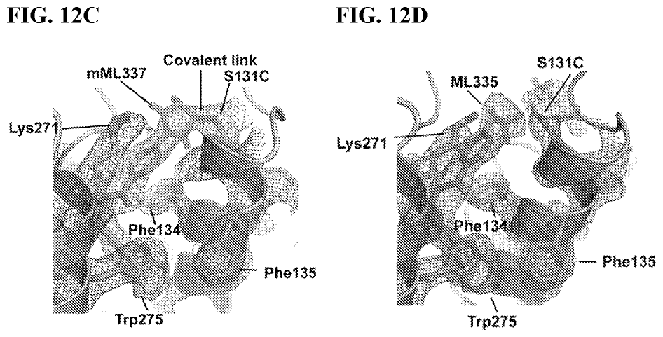

To understand the interactions of the CATKLAMP pairs, we determined the structures of complexes of TREK-1^{CG}* with CAT335, CAT335a, and the non-covalent activator ML335 at resolutions of 3.O each. These structures showed no major global deviations from the previously reported K_{2P}2.1 (TREK-1):ML335 complex (FIG. 12A) (Root mean square deviation (RMSD)Cα=0.446, 0.554, and 0.335 for CAT335, CAT335a, and ML335 complexes versus PDB:6CQ8). Importantly, both the CAT335 and CAT335a structures showed continuous density that bridged the maleimide moiety and S131C, indicative of the formation of a covalent adduct (FIGS. 3A-3B and FIGS. 12B-12C) and in line with the functional studies that indicate that both compounds act on TREK-1^{CG}* in an irreversible manner (FIGS. 2A, 2D, 2E, 2H, 2I, 2J, 10F, 10H, and 10I). The surrounding residues in the K_{2P} modulator pocket (Phe134, Gly260, Lys271, and Trp275) make interactions with both compounds that are similar to those seen in both the K_{2P}2.1 (TREK-1):ML335 structure (FIGS. 3A-3C) and in the complex of ML335 with TREK-1^{CG}* (FIGS. 3D-3E). The positions of both the upper and lower rings largely match those of the non-covalent ML335 complex (FIG. 3C) and indicate that similar to the acrylamide (FIG. 1B), the covalent links formed by the maleimide moiety are compatible with binding of the ML335 core scaffold to the K_{2P} modulator pocket.

### Example 3: Concatenated Tandem Channels to Study Activation of Single Subunits

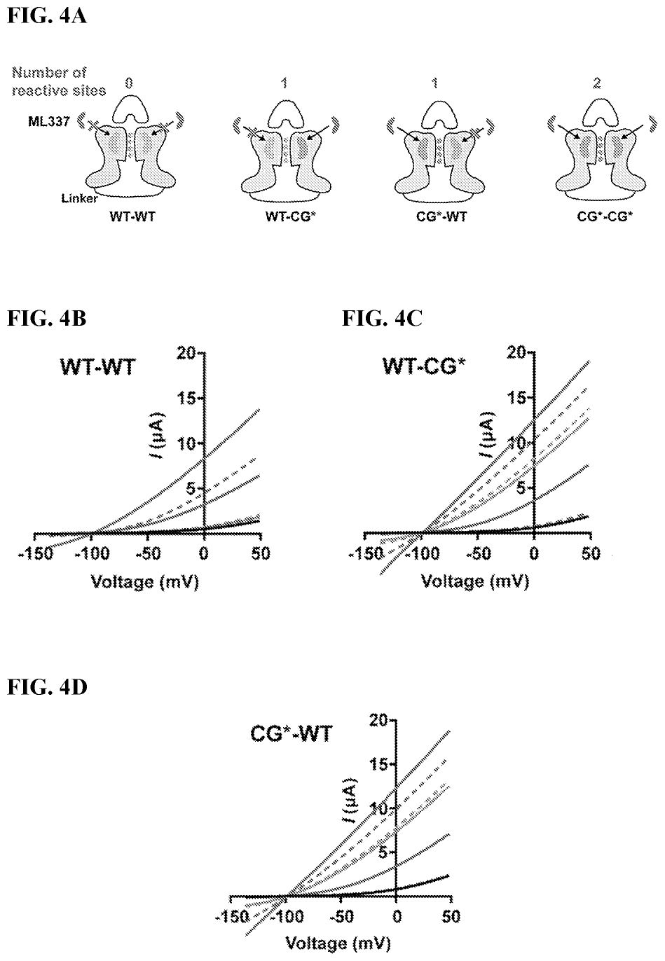

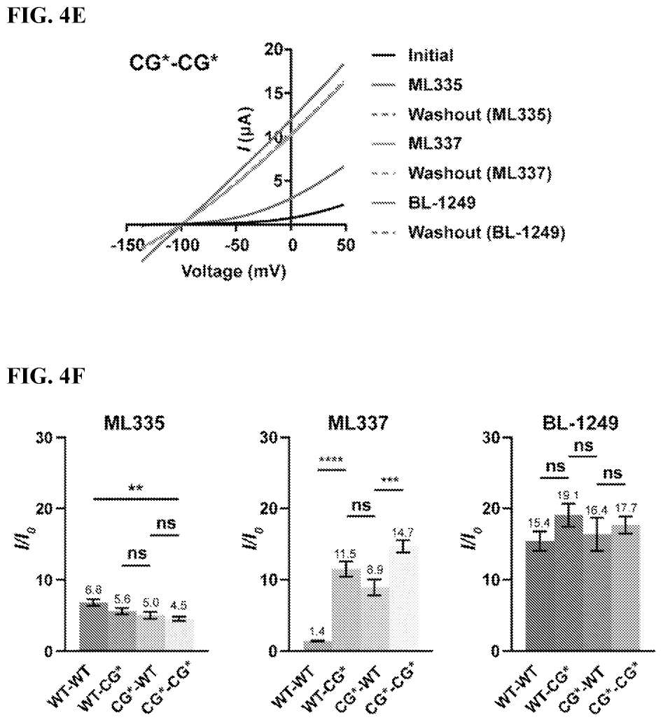

Given the ability of CAT335 to activate TREK-1^{CG}* selectively relative to unmodified K_{2P}2.1(TREK-1) (FIGS. 2C and 2G), we wondered if we could use this chemogenetic pair to probe the details of the C-type gate mechanism central to K_{2P} function. In order to create channels having defined numbers CAT335 reactive sites, we used a linker strategy (39) to generate tandem K_{2P}2.1(TREK-1) constructs bearing two (CG*-CG*), one (CG*-WT and WT-CG*), or zero (WT-WT) chemogenetic K_{2P} modulator sites (FIG. 4A). We first evaluated the effect of tethering using the non-covalent activator ML335, which binds both wild-type and CG* K_{2P} modulator pockets. 20 μM ML335 activated WT-WT (I_{ML335}/I_{control}=6.8+0.5) and CG*-CG* (I_{ML335}/I_{control}=4.5±0.3) tandem channels nearly identically to their untethered counterparts (FIGS. 4D-4E, FIG. 10A, Table 1). 20 μM ML335 also produced similar activations in the tethered heterodimers CG*-WT (I_{ML335}/I_{control}=5.0±0.5) and WT-CG* (I_{ML335}/I_{control}=5.6+0.4), indicating the linkage of monomers has minimal effect on K_{2P} modulator pocket activation (FIGS. 4B-4C, 4E). Treatment of CG*-CG* and WT-WT tandems with 20 μM CAT335 recapitulated the patterns observed with the unlinked homodimers, with CG*-CG* being strongly activated (I_{CAT335}/I_{control}=14.7+0.9) while WT-WT was only weakly activated (I_{CAT335}/I_{control}=1.37+0.07) (FIGS. 4D-4E).

Heterodimeric channels WT-CG* and CG*-WT were activated by CAT335 to a similar degree (I_{CAT335}/I_{control}=11.5+1.1 and 8.9+1.1, respectively), and the fold-activation was 60-80% of the activation of the CG*-CG* homodimer (FIGS. 4B-4C, 4E). We also tested the sensitivity of the four tandem constructs to BL-1249, a K_{2P}2.1(TREK-1) activator which binds to a distinct intracellular binding pocket (40,41), and observed no difference in activation between the four tandem constructs (FIGS. 4B-4E).

TABLE 1
Response of K_{2P} channels to activators

| | ML335 | CAT335 | BL-1249 | ML336 | CAT335a | CAT335b |
|---|---|---|---|---|---|---|
| K_{2P}2.1(TREK-1) | | | | | | |
| Wild-type | 6.5 | 2.0 ± 0.2 | | 10.0 (FIG. 1) | | |
| | (FIG. 1) | (FIG. 2) | | | | |
| S131A | 6.8 | | — | 16.1 (FIG. 1) | — | — |
| | (FIG. 1) | | | | | |
| TREK-1^{CG}* | | 22 ± 2 | | | 7.4 ± 0.7 | 4.9 ± 0.6 |
| | | (FIG. 2) | | | (FIG. 2) | (FIG. 2) |
| TREK-1^{CG}* A286F | | | | — | — | — |
| TREK-1^{CG}* G171F | | | | — | — | — |
| TREK-1 Tandems | | | | | | |
| WT-WT | 6.8 ± 0.5 | 1.37 ± 0.07 | | — | — | — |
| CG*-WT | 5.0 ± 0.5 | 8.9 ± 1.1 | | — | — | — |
| WT-CG* | 5.6 ± 0.4 | 11.5 ± 1.1 | | — | — | — |
| CG*-CG* | 4.5 ± 0.3 | 14.7 ± 0.9 | | — | — | — |
| K_{2P}10.1(TREK-2) | | | | | | |
| Wild-type | | | | — | — | — |
| TREK-2^{CG}* | | | | — | — | — |
| K_{2P}4.1(TRAAK) | | | | | | |
| Wild-type | | | | — | — | — |
| TRAAK^{CG}* | | | | — | — | — |

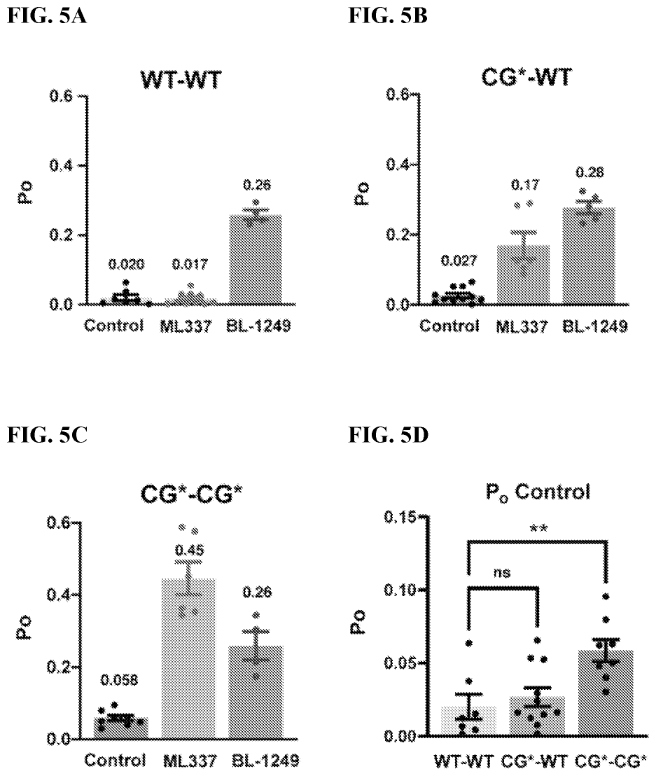

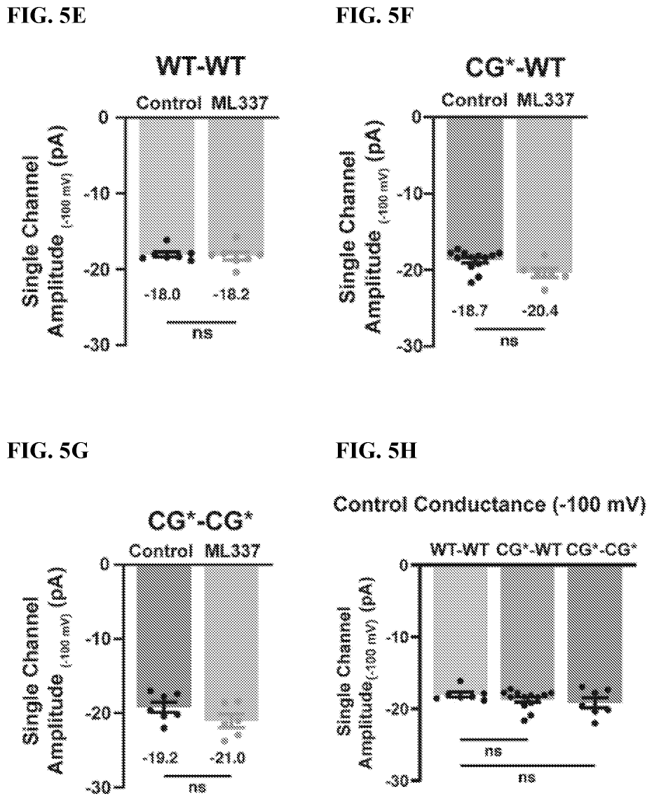

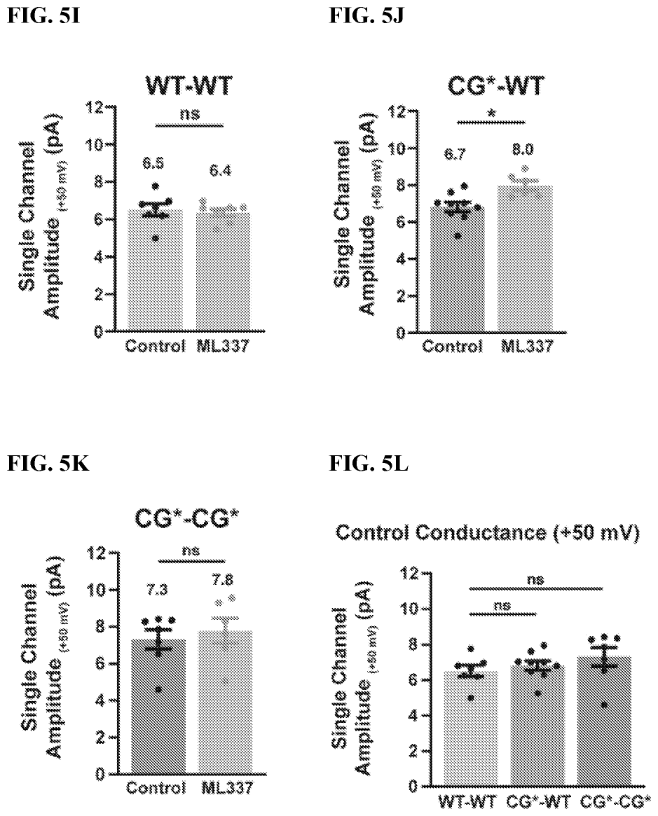

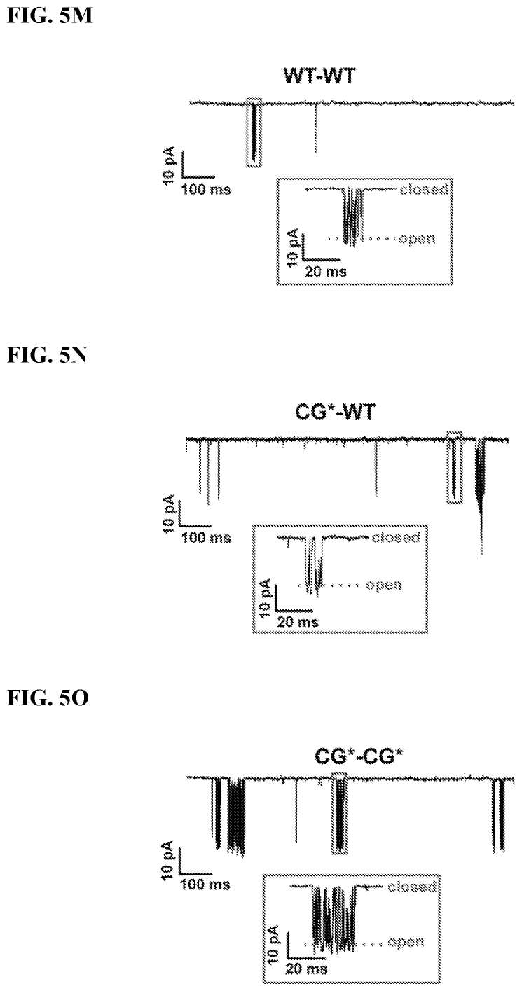

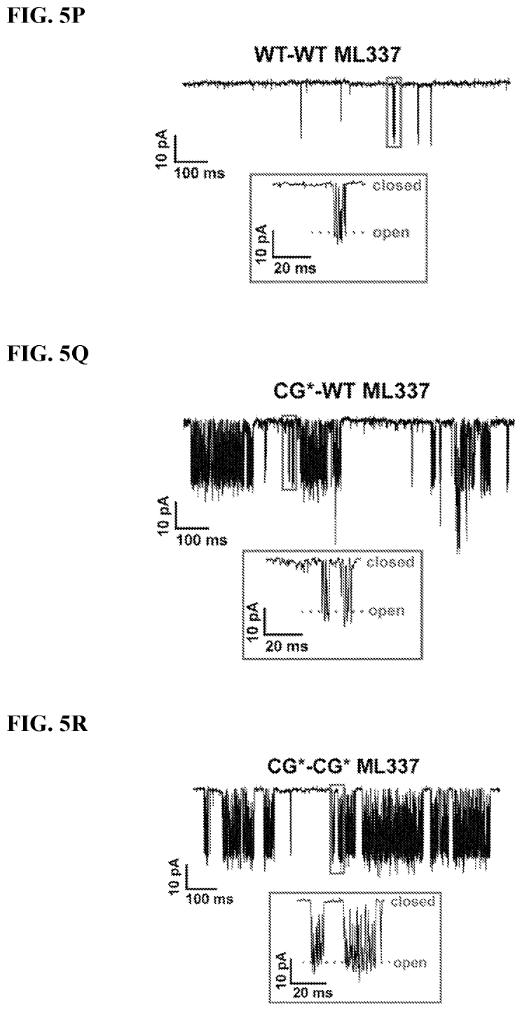

K_{2P}2.1 (TREK-1) tandem channels were further investigated by single channel recordings from HEK293 cells in cell attached mode. Application of 20 μM CAT335 to WT-WT had no impact on open probability (Pₒ), but resulted in large increases in Pₒ when applied to CG*-WT (P_{o CAT335}/P_{o control}=6.3) and CG*-CG* (P_{o CAT335}/P_{o control}=7.8) tandems (FIGS. 5A-5C, 5M-5R). In contrast, application of 20 μM BL-1249 resulted in large increases in Pₒ for all three tandem channels (FIGS. 5A-5C). We also found that the basal Pₒ of the CG*-CG* (P_{o control}=0.058) was higher than WT-WT (P_{o control}=0.020) and CG*-WT (P_{o control}=0.027) (FIG. 5D). While CAT335 elicited a large increase in Pₒ, it had minimal to no effect on single channel conductance at both −100 mV and +50 mV for all tandem channels (FIGS. 5E-5L), indicating CAT335 activation is largely a result of increasing Pₒ, in agreement with the mechanism of action of other K_{2P} modulator pocket ligands ML335 and ML402.

### Example 4: Attenuated TREK-1^{CG}* Double Mutants Increase Dynamic Response to CAT335

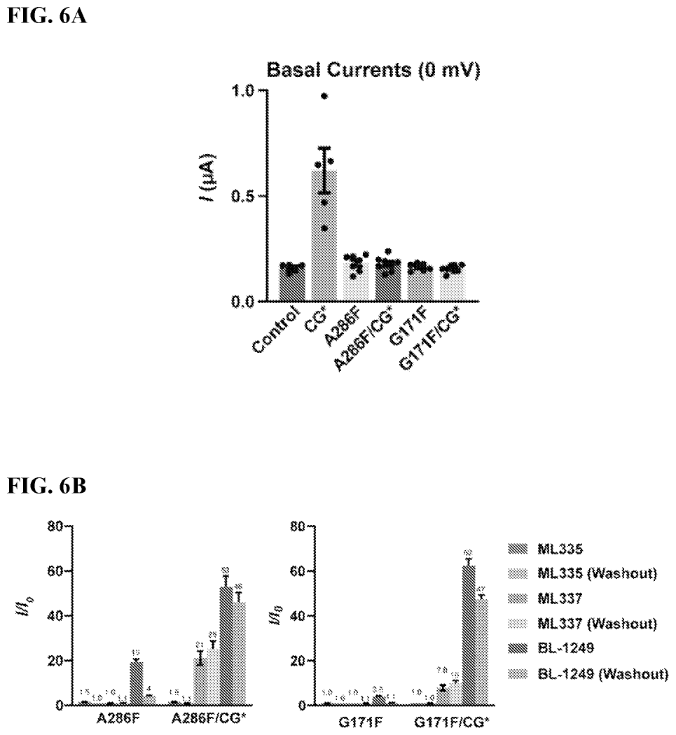

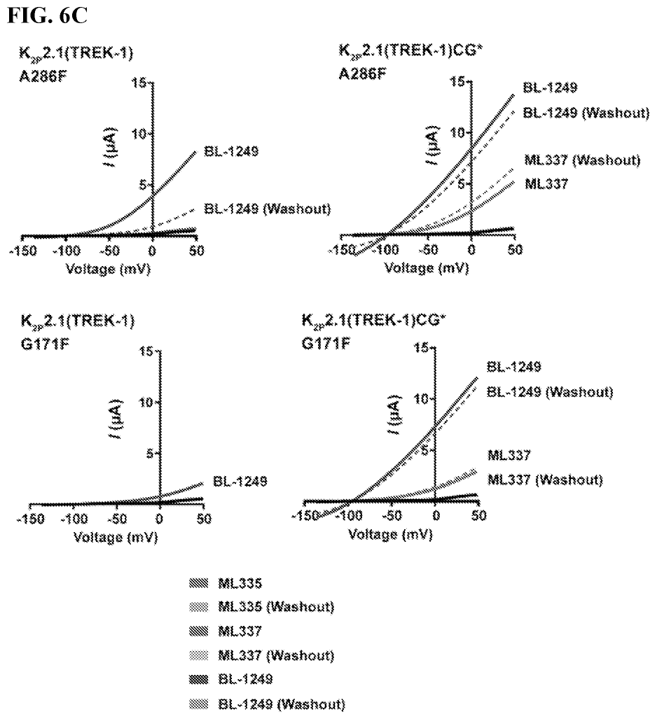

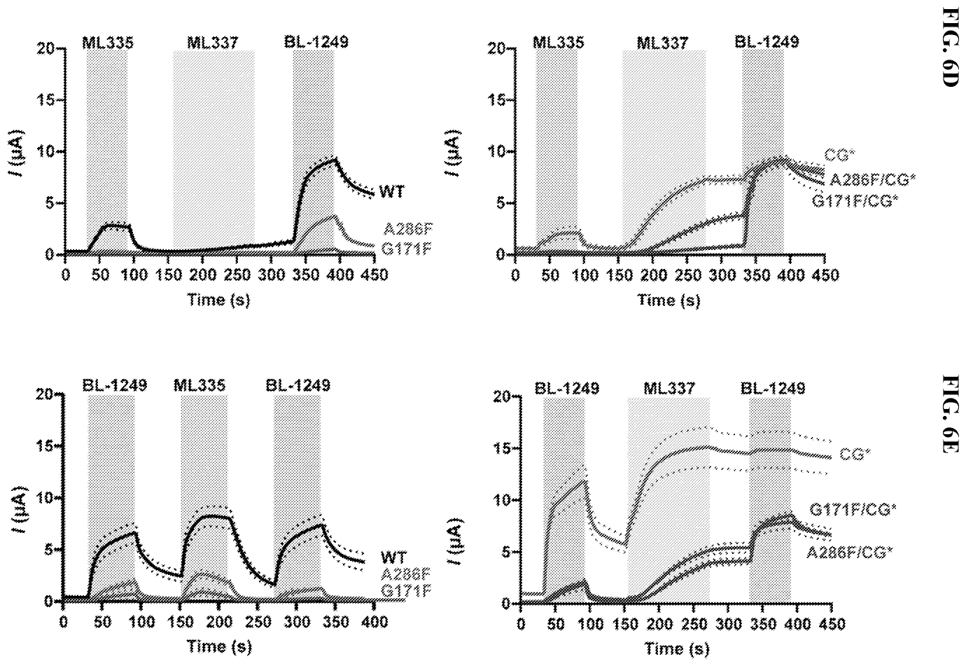

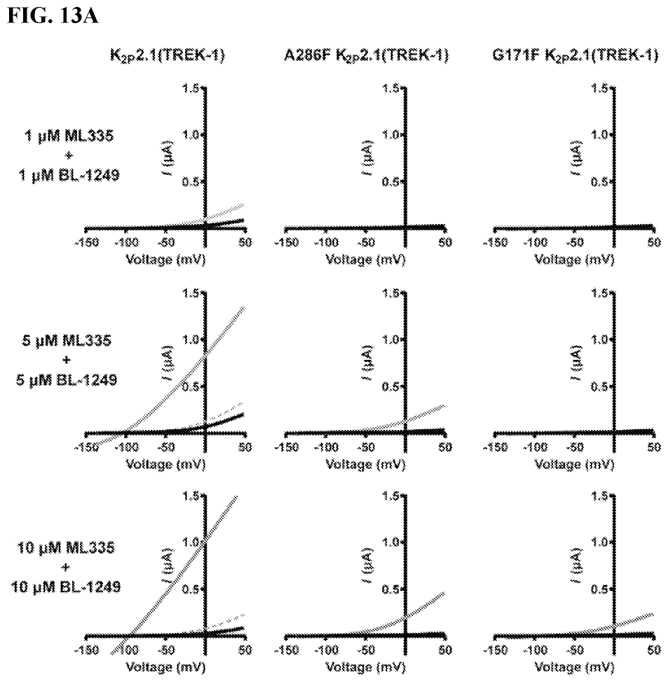

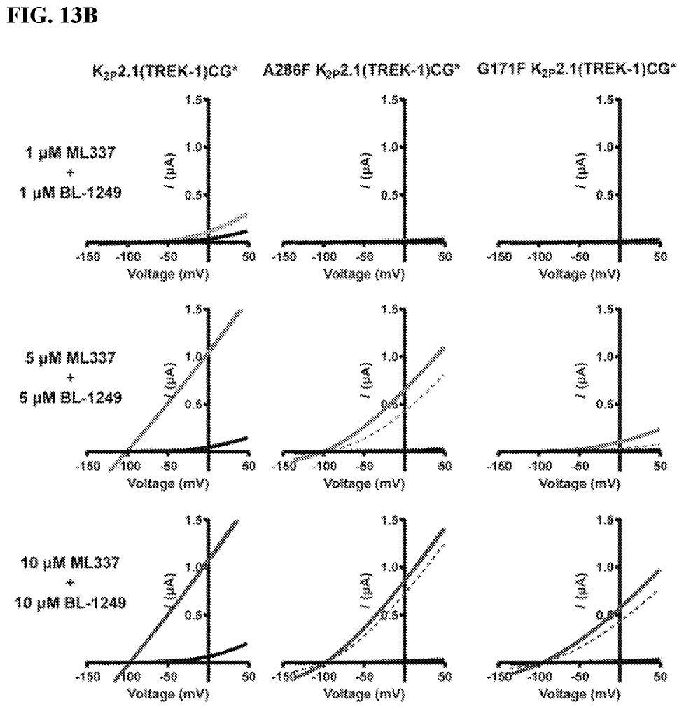

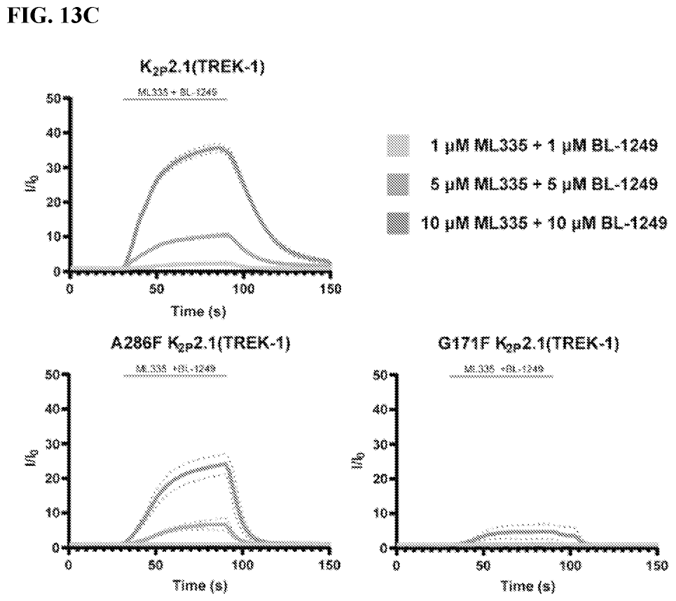

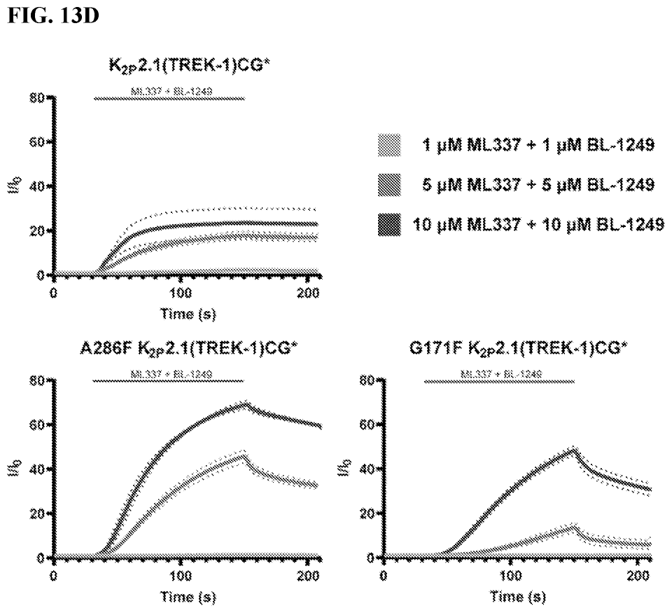

To exploit the orthogonal pharmacology of TREK-1^{CG}* and wild-type K_{2P}2.1 (TREK-1) in response to CAT335, we hypothesized TREK-1^{CG}* could serve as a useful tool for modulating membrane potential and intracellular [K⁺ ]. Chemogenetic ion channels based on modified nicotinic acetylcholine receptor (nAChR) ligand binding domains (LBDs) coupled with ion pore domains from GlyR (Cl⁻) and 5HT3 (Na⁺/K⁺/Ca²⁺) channels have been reported (36,42). While advantageous due to their modular design and potent ligands (low nanomolar), changes in membrane potential result from the influx of ions into the cell: Cl⁻ to electrically shunt or Na⁺/K⁺ to depolarize the plasma membrane. In contrast, TREK-1^{CG}* hyperpolarizes cells by the outflux of K⁺ and the decrease of input resistance. One limitation of TREK-1^{CG}* is that the high basal K⁺ leak causes significant depolarization of cells when exogenously expressed, even in the absence of activator CAT335 (FIG. 6A). To address this limitation, we incorporated a recently reported K_{2P}2.1 (TREK-1) attenuating mutations (G171F and A286F) which place bulky phenyl groups near the intracellular cavity of the channel (38). These mutations greatly reduce the basal current of K_{2P}2.1 (TREK-1), however can be pharmacologically overcome by activators such as flufenamic acid (FFA) or by the activating mutation G137I. A286F and G171F TREK-1^{CG}* double mutant channels were expressed in Xenopus oocytes and their sensitivity to activators ML335, CAT335 and BL-1249 were examined. Channels possessing the A286F or G171F mutations had significantly reduced basal currents when compared to TREK-1^{CG}*, with basal currents nearly indistinguishable from the uninjected oocyte negative control (FIG. 6A). A286F and G171F mutations reduced K_{2P}2.1 (TREK-1) sensitivity to 20 μM ML335 (I_{ML335}/Control between 1-1.8) (FIGS. 6B-6E), suggesting these mutations favor the adoption of an inactive conformation that occludes the cryptic binding pocket. As expected, A286F and G171F K_{2P}2.1 (TREK-1) were also insensitive to 20 μM CAT335, however their CG* double mutant congeners were both activated by CAT335 (I_{CAT335}/I_{control}=21±3 for A286F/CG* and I_{CAT335}/I_{Control}=7.8±1.2 for G171F/CG*) (FIGS. 6B-6C). Activation with 20 μM CAT335 was slower and smaller than TREK-1^{CG}* activation over 2 minutes, likely reflecting the lower affinity of A286F and G171F K_{2P}2.1 (TREK-1) for cryptic modulator pocket ligands. Interestingly, subsequent addition of 20 μM BL-1249 induced very large currents from A286F TREK-1^{CG}* and G171F TREK-1^{CG}* (I_{BL-1249}/I_{control}=53+5 and I_{BL-1249}/I_{Control}=62+3, respectively), but had a smaller activating effect on the A286F and G171F K_{2P}2.1 (TREK-1) channels (I_{BL-1249}/I_{control}=19.4±1.3 and 3.8±0.5, respectively). This result suggests cooperativity between the two distal binding sites, where activation with one activator increases the affinity for the other. This was also supported by the fact that activating A286F and G171 TREK-1^{CG}* with BL-1249 before the addition of 20 μM CAT335 increased the rate and extent of CAT335 activation. Further, testing the simultaneous application of K_{2P} modulator pocket activators and BL-1249 at 1, 5 and 10 M resulted in strong activation, including channels bearing the attenuating mutations A286F and G171F (FIGS. 13A-13D).

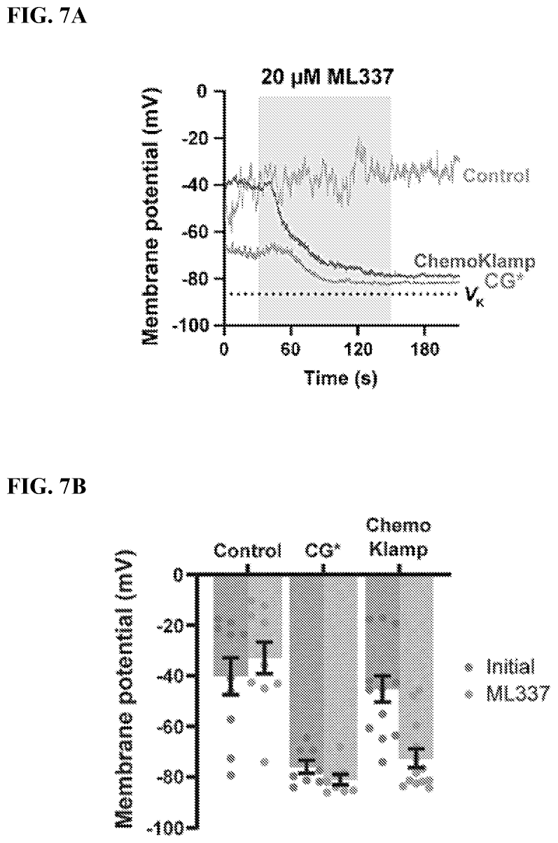

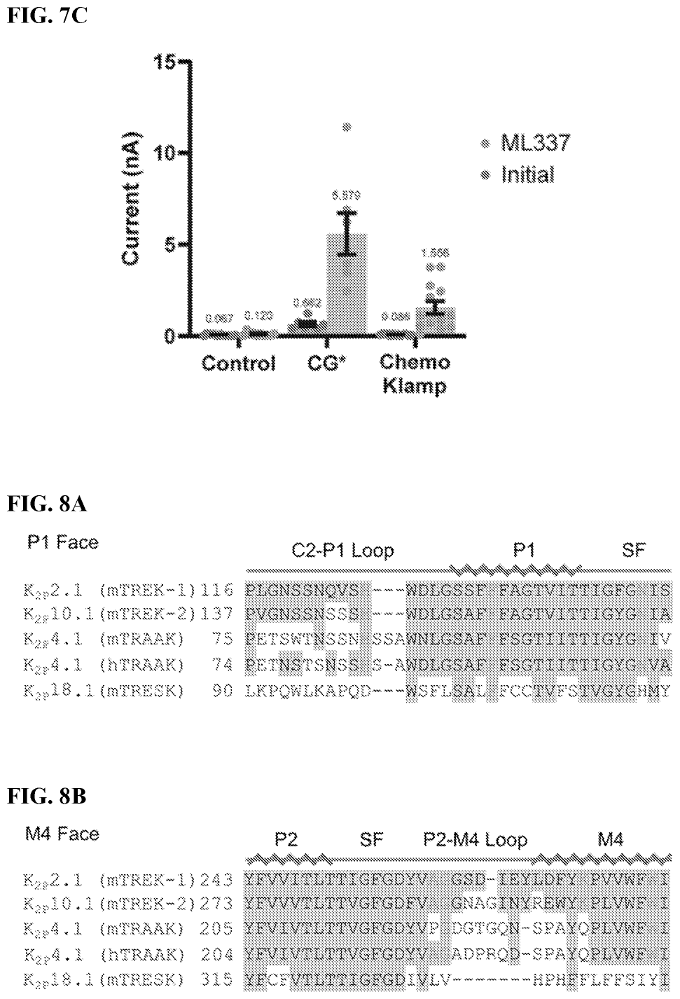

We next explored the potential of TREK-1^{CG}* and A286F TREK-1^{CG}* channels to modulate resting membrane potential (RMP) in HEK293 cells. Similar to what was observed in oocytes, expression of TREK-1^{CG}* resulted in large HEK293 basal currents (660±110 pA at 0 mV), while A286F TREK-1^{CG}* currents (90 i 10 pA at 0 mV) were similar to control cells (70±20 pA at 0 mV) (FIG. 7A). These increased basal currents were reflected in the RMP of HEK293 cells, with TREK-1^{CG}* expressing cells being significantly hyperpolarized (RMP=−76±3 mV) relative to A286F TREK-1^{CG}* (RMP=−45±5 mV) and control cells (RMP=−40±7 mV) (FIG. 7B). Application of CAT335 resulted in a rapid, irreversible hyperpolarization of TREK-1^{CG}* (ΔRMP=−6 mV) and A286F TREK-1^{CG}* (ΔRMP=−27 mV) while resulting in a small depolarization of control cells (ΔRMP=+7 mV) (FIG. 7C). Having validated the potential of A286F TREK-1^{CG}* in HEK cells, we applied this chemogenetic system to dissociated primary mouse hippocampal neurons.

### Example 5: K_{2P}10.1 (TREK-2)^{CG}* and K_{2P}4.1 (TRAAK)^{CG}*

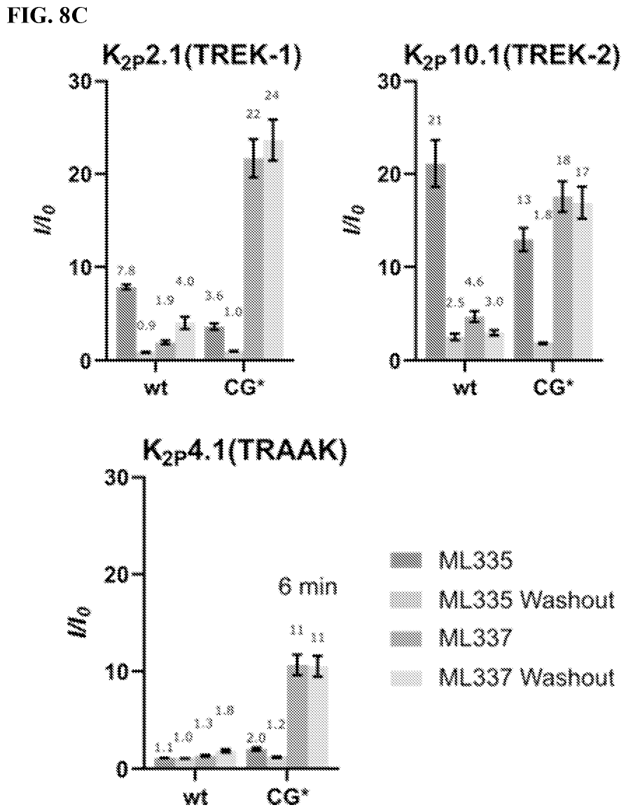

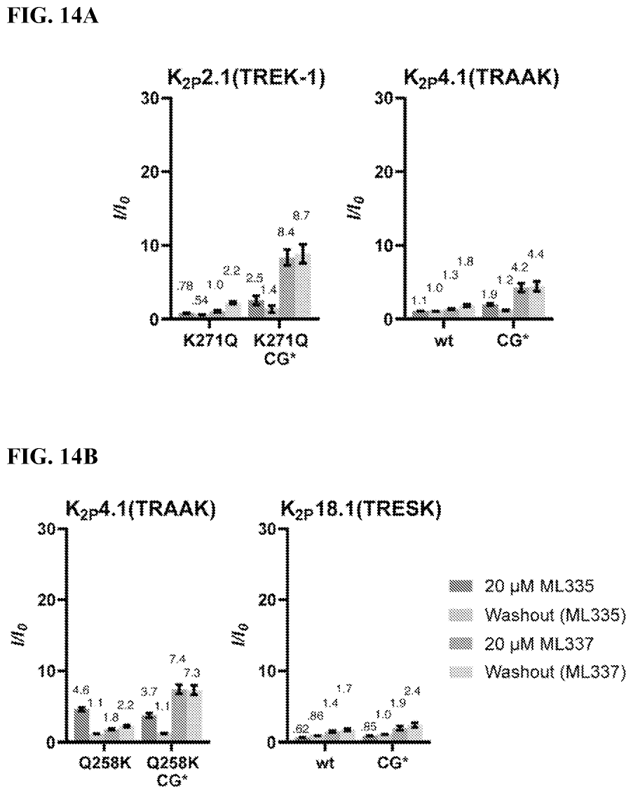

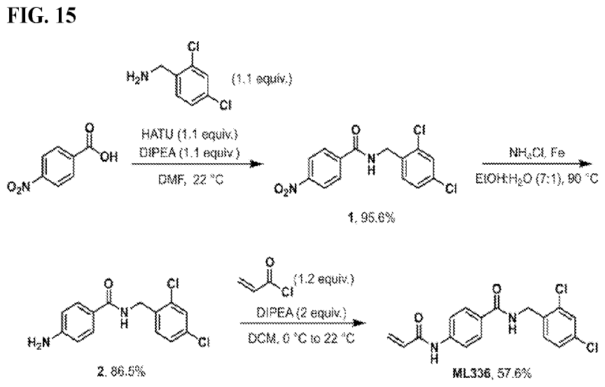

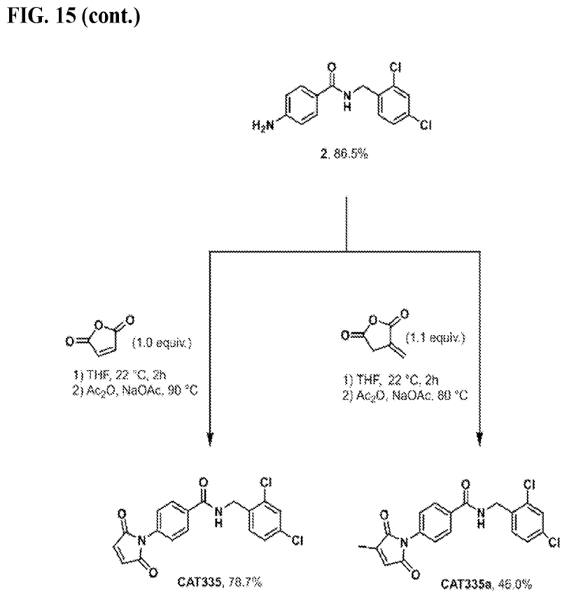

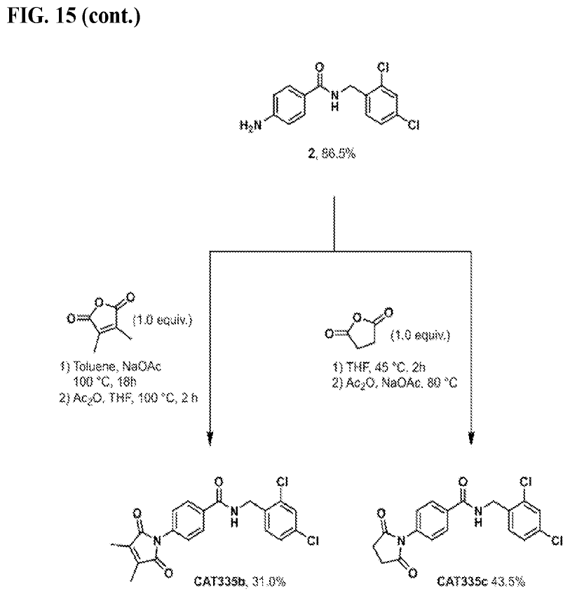

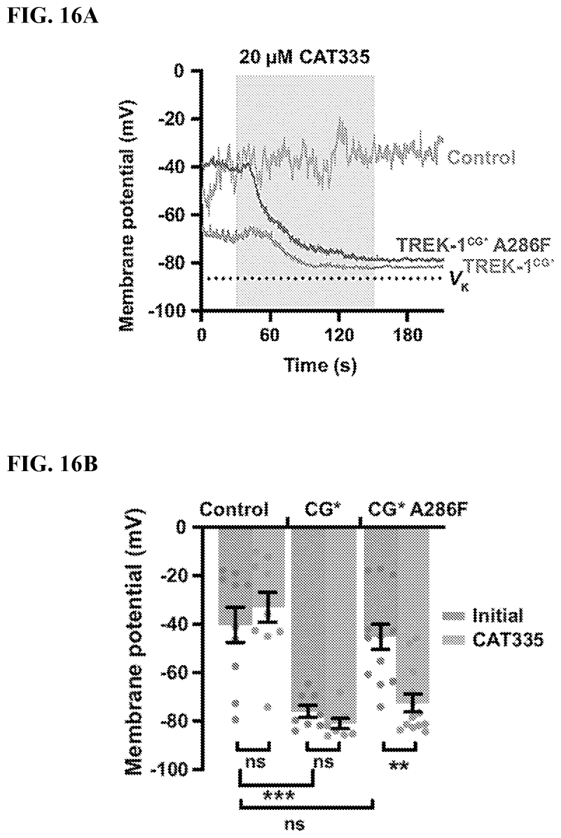

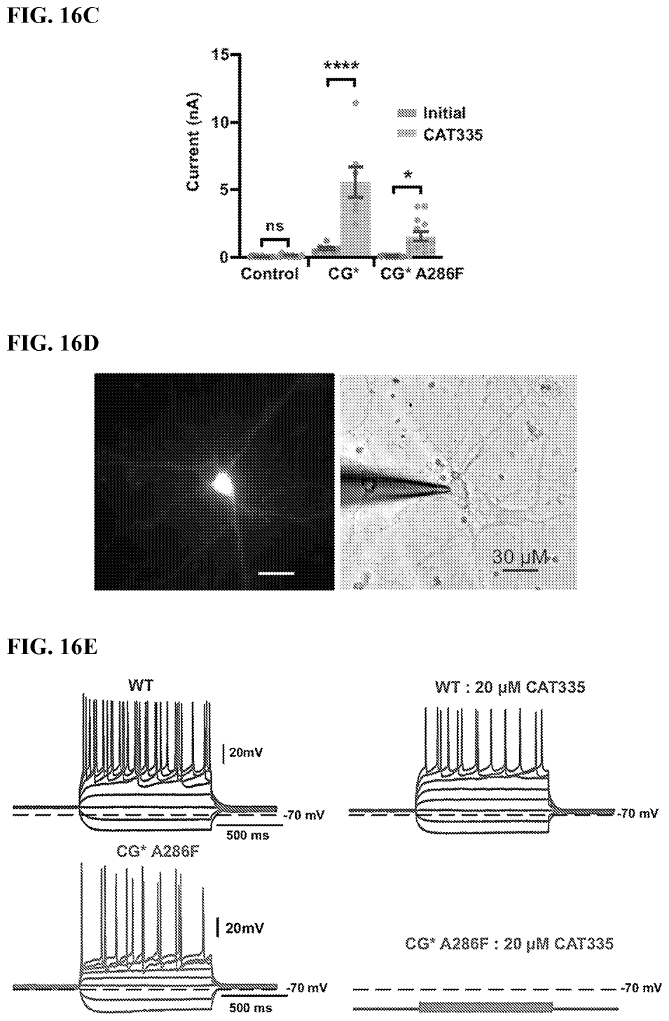

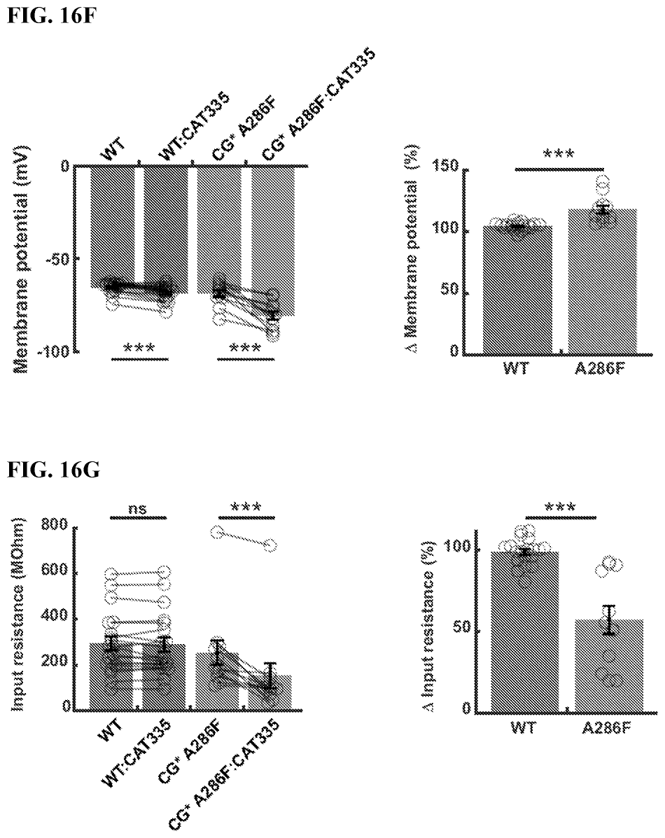

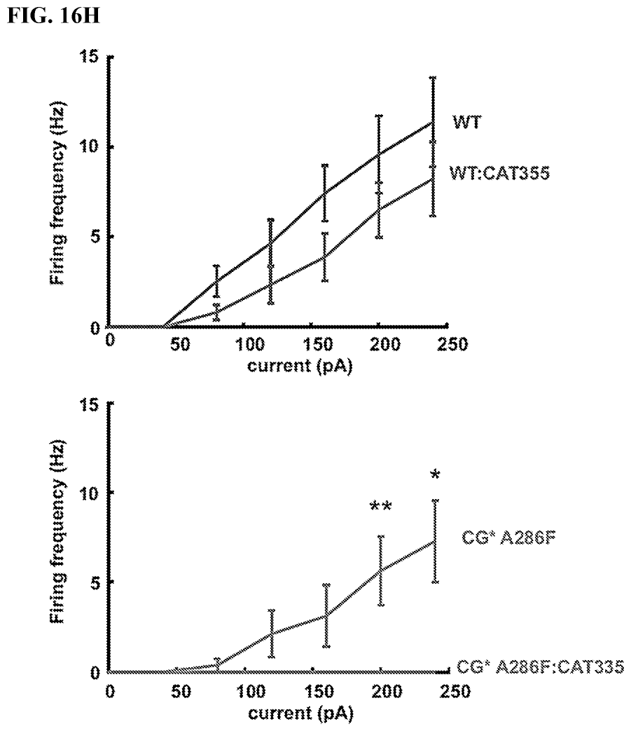

Having shown that CAT335 can be used to selectively and irreversibly activate K_{2P}2.1 (TREK-1)^{CG}*, we wondered if the strategy could be extended to the other members of the TREK subfamily. Sequence alignment of K_{2P}2.1(TREK-1), K_{2P}10.1(TREK-2) and K_{2P}4.1 (TRAAK) shows the channels are highly conserved at the P1 face of the modulator pocket near the CG* mutation (FIG. 8A). The M4 face of the pocket is highly conserved in K₂P10.1(TREK-2), however diverges with K_{2P}4.1(TRAAK) (FIG. 8B). In particular, K_{2P}4.1(TRAAK) lacks a key lysine residue that forms a cation-π interaction with K_{2P} modulator pocket ligands ML402 and ML335, explaining the channel's relative insensitivity to these activators (37). We found that 20 μM CAT335 strongly and irreversibly activated both K_{2P}10.1 (TREK-2)^{CG}* and K_{2P}4.1(TRAAK)^{CG}* channels (FIG. 8C). While K_{2P}10.1 (TREK-2)^{CG}* activation was similar to K₂P2.1(TREK-1)^{CG}* (I_{CAT335}/I_{control}=18±2), K₂P4.1 (TRAAK)^{CG}* required a longer application of CAT335 (6 min) to approach maximal activation (I_{CAT335}/I_{control}=11±1). The more distantly related K₂P18.1 (TRESK)^{CG}*channel, however, was not activated by 20 μM CAT335 (FIGS. 14A-14B). We next generated Q258K K₂P4.1 (TRAAK)^{CG}* double mutant that has an analogous lysine to the cation-π forming Lys271 of K_{2P}2.1 (TREK-1). Q258K K₂P4.1 (TRAAK) was more sensitive to 20 μM ML335 and CAT335 (I_{ML335}/I_{control}=3.7±0.3, I_{CAT335}/I_{control}=7.4+0.6 after 2 min) than K₂P4.1(TRAAK)^{CG}* (I_{ML}335/I_{control}=1.9±0.1, I_{CAT335}/I_{control}=4.2±0.6 after 2 min) (FIGS. 14A-14B).

### DISCUSSION

We report the development of chemogenetic tools capable of selectively activating TREK channels through the selective engagement of an engineered cysteine residue at the K_{2P} modulator pocket. The efficacy of this system was measured functionally by TEVC, whole-cell and single channel patch clamp electrophysiology, and demonstrated that CAT335 can activate K₂P2.1 (TREK-1)^{CG}* channels up to 22-fold. X-ray crystallography, confirming CAT335 activates K_{2P}2.1 (TREK-1) through a similar molecular wedging mechanism as ML335, and that this binding places the maleimide in proximity to the nucleophilic Cys131 of K_{2P}2.1 (TREK-1)^{CG}*. Unlike typical chemogenetic systems which rely on bump-hole strategies to achieve selectivity, we believe our system relies on the chemoselectivity of maleimide-cysteine labeling to irreversibly activate channels. This strategy has the advantage of improving target engagement over non-covalent ligands, which was especially important for low-affinity interactions like those found in K_{2P}4.1(TRAAK)^{CG}*.

We also showed that chemogenetic tools can be used in ways not possible with ligands targeting wt channels. We demonstrated that concatenated tandems bearing 0, 1 or 2 CG* mutations possess non-linear activation profiles, implying that the activation of a single subunit of a K_{2P} dimer is sufficient for activation of the C-type gate. Further, activation of a single subunit seemed to provide >50% activation, implying cooperativity between the two monomers. This was supported by single channel experiments, which showed that fold-changes in Pₒ were >50% for channels possessing a single CG* site. We hypothesize similar systems can be engineered to better understand the behavior of heterodimeric K_{2P} channels.

We hypothesize the utility of chemogenetic K_{2P} channels could expand beyond studying the mechanisms of K_{2P} activation. By combining inactivating K_{2P}2.1(TREK-1) mutations G171F or A286F mutations with the CG* mutation, we generated an ‘off-on’ K_{2P}2.1(TREK-1) that possessed greatly reduced leak currents while still being sensitive to activation by CAT335. Currently, a limited number of optogenetic and chemogenetic tools are available for modulating [K⁺], and those that exist possess limitations such as poor temporal control and small amplitudes of depolarization (35, 43, 44, 45). Chemogenetic K2P channels can thus provide a novel method by which excitable cells membrane potential can be hyperpolarized in a complementary way to the favored channelrhodopsins and chloride-channel targeting PSAM/PSEM and DREADD systems.

Finally, we showed the our covalent, chemogenetic targeting system can activate other K2P channels such as K2P10.1 (TREK-2) and K2P4.1 (TRAAK). The activation of K2P4.1 (TRAAK) is of particular interest, as previous studies revealed K2P4.1 (TRAAK) was insensitive to K2P modulator pocket ligands ML335 and ML402 due to the lack of a key cation-π interaction in the modulator pocket. The fact that ML337 can activate K2P4.1 (TRAAK)CG* alludes to the fact that the powerful covalent engagement can improve target engagement of weak interactions. We hypothesize that this system can therefore be employed to explore the broader pharmacology of the K2P modulator pocket across other K2P channels and be utilized in cysteine-tethering screening to identify novel motifs capable of binding this cryptic binding pocket.

### Experimental

### Molecular Biololgy

K2P2.1_{cyst} and S131C K2P2.1_{cryst} bearing C-terminal green fluorescent protein (GFP) and His₁₀ tag were expressed from a previously described Pichia pastoris pPICZ vector (44). Plasmids were linearized with PmeI and transformed into.

Mouse K_{2P}2.1 (TREK-1) (Genbank accession number: NP_034737.2), mouse K₂P10.1 (TREK-2) (NM_001316665.1), mouse K₂P4.1 (TRAAK) (NM_008431.3), human K₂P4.1 (TRAAK) (NM_033310.2) and mouse K2P18.1(TRESK) (NM_207261.3) were used for this study. Point mutations were introduced by site-directed mutagenesis using custom primers and confirmed by sequencing before use. For studies using Xenopus oocytes, K_{2P} channels were subcloned into a previously reported pGEMHE/pMO vector (46). For expression in HEK293 cells, mouse K_{2P}2.1 (TREK-1) and mutants were expressed from a previously described pIRES2-EGFP vector in HEK293 cells (46,47). K_{2P}2.1 (TREK-1) tandems were constructed by connecting the open reading frames for the individual subunits with a linker encoding the AAAGSGGSGGSGGSSGSSGS (SEQ ID NO: 19) sequence. Tandems used for HEK293 cells included an N-terminal HA tag (YPYDVPDYA (SEQ ID NO: 20)) on the first subunit.

mRNA for oocyte injections was prepared from linearized plasmid DNA (linearized with AflII) using mMessage Machine T7 Transcription Kit (Thermo Fisher Scientific). RNA was purified using RNEasy kit (Qiagen) and stored as stocks and dilutions in RNAse-free water at −80° C.

### Protein Expression

K₂P2.1_{cryst} and S131C K2P2.1_{cryst} bearing C-terminal green fluorescent protein (GFP) and His₁₀ tag were expressed from a previously described Pichia pastoris pPICZ vector (44). Plasmids were linearized with PmeI and transformed into P. pastoris SMD 1163H by electroporation. Multi-integration recombinants were selected by plating transformants onto yeast extract peptone dextrose sorbitol (YPDS) plates having increasing concentrations of zeocin (1-4 mg ml⁻¹). Expression levels of individual transformants were evaluated by FSEC as previously described (45).

Large-scale expression was carried out in a 7L Bioreactor (Labfors5, Infors HT). First, a 250 ml starting culture was grown in buffered minimal medium (2×YNB, 1% glycerol, 0.4 mg l⁻¹ biotin, 100 mM potassium phosphate, pH 6.0) in shaker flasks for two days at 29° C. Cells were pelleted by centrifugation (3,000g, 10 minutes, 20° C.) and used to inoculate the bioreactor. Cells were grown in minimal medium (4% glycerol, 0.93 g l⁻¹ CaSO₄·2H₂O, 18.2 g l⁻¹ K₂SO₄, 14.9 g l⁻¹ MgSO₄·7H₂O, 9 g l⁻¹ (NH₄)₂SO₄, 25 g l⁻¹ Na⁺ hexametaphosphate, 4.25 ml l⁻¹ PTM₁ trace metals stock solution prepared accordingly to standard Invitrogen protocol) until the glycerol in the fermenter was completely metabolized marked by a spike in pO₂ (around 24 h). Fed-batch phase was then initiated by adding a solution of 50% glycerol and 12 ml l⁻¹ of trace metals at 15-30% of full pump speed until the wet cell mass reached approximately 250 g l⁻¹ (around 24 h). pO₂ was measured continuously and kept at a minimum of 30%. Feed rate was automatically regulated accordingly. pH was maintained at 5.0 by the addition of a 30% ammonium hydroxide solution.

After the fed-batch phase was completed, cells were then starved to deplete glycerol by stopping the feeder pump until a pO₂ spike appeared. After starvation, the temperature was set to 27° C., and the induction was initiated with addition of methanol in three steps: (1) initially, the methanol concentration was kept at 0.1% for 2 h in order to adapt the cells; (2) methanol concentration was then increased to 0.3% for 3 h; and (3) methanol was then increased to 0.5% and expression continued for 48-60 h. Cells were then pelleted by centrifugation (6,000g, 1 h, 4° C.), snap frozen in liquid nitrogen, and stored at −80° C.

### Protein Purification

In a typical preparation, 10 g of cells were broken by cryo-milling (Retsch model MM400) in liquid nitrogen (5×3 min, 25 Hz). All subsequent purification was carried out at 4° C. Cell powder was added at a ratio of 1 g cell powder to 3 ml lysis buffer (200 mM KCl, 21 mM OGNG (octyl glucose neopentyl glycol, Anatrace), 30 mM HTG (n-heptyl-P-D-thioglucopyranoside, Anatrace), 0.1% CHS, 0.1 mg ml⁻¹ DNase 1 mM PMSF, 100 mM Tris-Cl, pH 8.2). Membranes were extracted for 3 h with gentle stirring followed by centrifugation (100,000g, 45 minutes at 4° C.).

Solubilized proteins were purified by affinity chromatography using batch purification. Anti-GFP nanobodies were conjugated with CNBr Sepharose beads (GE Healthcare, #17-0430-02). The resin was added to the cleared supernatant at a ratio of 1 ml of resin per 10 g of cell powder and incubated at 4° C. for 3 h with gentle shaking. Resin was collected into a column and washed with ten column volumes (CV) of buffer A (200 mM KCl, 10 mM OGNG, 15 mM HTG, 0.018% CHS, 50 mM Tris-Cl, pH 8.0) followed by a second wash step using 10 CV of buffer B containing (200 mM KCl, 5 mM OGNG, 15 mM HTG, 0.018% CHS, 50 mM Tris-Cl, pH 8.0). The resin was then washed with additional ten CV of buffer C (200 mM KCl, 3.85 mM OGNG, 15 mM HTG, 0.0156% CHS, 50 mM Tris-Cl, pH 8.0). On column cleavage of the affinity tag was achieved by incubating the resin with buffer C supplemented to contain 350 mM KCl, 1 mM EDTA, and 3C protease at ratio of 50:1 resin volume:protease volume (46). The resin was incubated overnight at 4° C. Cleaved sample was collected and the resin washed with two CV of SEC buffer (200 mM KCl, 2.1 mM OGNG, 15 mM HTG, 0.012% CHS, 20 mM Tris-Cl, pH 8.0). Purified sample was concentrated and applied to a Superdex 200 column pre-equilibrated with SEC buffer. Peak fractions were evaluated by SDS-PAGE (15% acrylamide) for purity, pooled and concentrated.

### Crystallization and Refinement

Purified K_{2P}2.1_{cryst} was concentrated to 6 mg ml⁻¹ by centrifugation (Amicon Ultra-15, 50 kDa molecular mass cut-off, Millipore) and crystallized by hanging-drop vapor diffusion at 4° C. using a mixture of 0.2 μl of protein and 0.1 μl of precipitant over 100 μl of reservoir containing 20-25% PEG400, 200 mM KCl, 100 mM HEPES pH 8.0, 1 mM CdCl₂. Crystals appeared in 12 h and grew to full size (200-300 μM) in about a week. Crystals were cryoprotected with buffer D (200 mM KCl, 0.2% OGNG, 15 mM HTG, 0.02% CHS, 100 mM HEPES pH 8.0.1 mM CdCl₂) with 5% step increase of PEG400 up to a final concentration of 38% and flash-frozen in liquid nitrogen.

K_{2P}2.1_{cryst} ML335 and ML402 complex crystals grew in the same conditions as K₂P2.1_{cyst}, but the protein was incubated for at least 1 h with 2.5 mM of activator before setting the crystal plates. ML335 and ML402 are insoluble in aqueous solutions, so they were dissolved in 100% DMSO at a concentration of 500 mM. Then each compound was diluted 1:100 in SEC buffer to 5 mM concentration, giving a milky solution. This solution was mixed 1:1 to K₂P2.1_{cryst} previously concentrated to 12 mg ml⁻¹. The K_{2P}2.1_{crys}/ML402 mixture resulted in a clear solution, while the mixture with ML335 was slightly milky. The samples were briefly centrifuged in a table-top centrifuge (10,000 g) to remove any insoluble material before setting the crystal plates.

Datasets for K_{2P}2.1, K_{2P}2.1-ML335, and K_{2P}2.1-ML402 were collected at 100 K using synchrotron radiation at ALS Beamline 8.3.1 Berkeley, California and APS GM/CAT beamline 23-IDB/D Chicago, Illinois using wavelengths of 1.1159 Å and 1.0332 Å, respectively, processed with XDS39, scaled and merged with Aimless40. Final resolution cut-off was 3.1 Å, 3.0 Å and 2.8 Å for K_{2P}2.1_{cryst}, K_{2P}2.1_{cryst}-ML335 and K_{2P}2.1_{cryst}-ML402, respectively, using the CC_{1/2} criterion^{41,42}. Structures were solved by molecular replacement using the K_{2P}4.1(G124I) structure (PDB: 4RUE)¹⁵ as search model. Several cycles of manual rebuilding, using COOT⁴³, and refinement using REFMAC5⁴⁴ and PHENIX⁴⁵ were carried out to improve the electron density map. Twofold local medium NCS restraints were employed during refinement for residues 28-103, 110-158 and 194-260. K_{2P}2.1, K_{2P}2.1-ML335 and K_{2P}2.1-ML402 structures have, respectively, 95.2%/0.4%, 92.0%/0.5% and 95.7%/0.4% residues in favoured regions/outliers of the Ramachandran plot as assessed by Molprobity⁴⁶.

### Patch-Clamp Electrophysiology

50% confluent cells were co-transfected (in 35-mm diameter wells) with 10-100 ng of the K_{2P}2.1 plasmid and 400 ng of an eGFP plasmid (for visualization) using LipofectAMINE 2000 (Invitrogen) for 24 h, after which the cells were either plated onto coverslips or the media exchanged with fresh media. Cells were plated onto Matrigel coated coverslips (BD Biosciences) 1-2 hours before experiments.

Whole-cell patch-clamp experiments with K_{2P}2.1(TREK-1) and mutants were carried out 24-48 h after transfection. Acquisition and analysis for voltage-clamp experiments were performed using pCLAMP10 and an Axopatch 200B amplifier (Molecular Devices). Current-clamp experiments were acquired using an Axopatch 700B amplifier (Molecular Devices). Pipettes were pulled using a Flaming/Brown micropipette puller (P-97, Sutter Instruments) and flame-polished using a MF-830 microforge (Narishige). Electrode resistance after filling with pipette internal solution ranged from 2 to 5 MΩ. Currents were low-pass filtered at 2 kHz and sampled at 10 kHz. Pipette solution contained the following: 145 mM KCl, 3 mM MgCl₂, 5 mM EGTA and 20 mM HEPES (pH 7.2 with KOH). Bath solution contained the following: 145 mM NaCl, 5 mM KCl, 1 mM CaCl₂, 3 mM MgCl₂ and 20 mM HEPES (pH 7.4 with NaOH). K_{2P}2.1 currents were elicited by a 1 s ramp from −100 to +50 mV from a −80 mV holding potential. Current-clamp recordings were taken with no holding current (I=0). Only K_{2P}2.1(TREK-1)^{CG}* whole-cell patches with basal currents between 500-2000 nA at 0 mV were used Criteria for A286F K_{2P}2.1(TREK-1)^{CG}*. ML335 or CAT335 were applied at 20 μM in bath solution and were perfused at 3 ml min⁻¹ until potentiation stabilized (1-3 min). Data were analyzed using Clampfit 11 and GraphPad Prism 9.

Single-channel recordings of K_{2P}2.1 (TREK-1) tandem mutants were recorded in cell-attached mode using HEK293 cells 48 h after transfection. Acquisition and analysis was preformed using pCLAMP9 and an Axopatch 200B amplifier. Pipettes were pulled using a laser-based micropipette puller (P-2000, Sutter Instruments). Electrode resistance after filling with pipette internal solution ranged from 6 to 10 MΩ. Pipette solution contained the following: 150 mM KCl, 3.6 mM CaCl₂, 10 mM HEPES (pH 7.4 with KOH). The bath solution was identical to the pipette solution. Pₒ was measured from 30-120 s recordings of K_{2P}2.1 (TREK-1) activity holding at −100 mV. Channel conductance of K_{2P}2.1 (TREK-1) tandems were measured from 30-60s recordings at either −100 mV or +50 mV holding potential. Recordings of CAT335 activated channels were obtained by bath applying 20 μM CAT335 for 2 min to the coverslip before transferring to the recording chamber. This was necessary as application of CAT335 via perfusion or through the pipette solution activated slowly (>5 min), making it difficult to measure Pₒ from fully activated channels. BL-1249 was applied at 20 μM in the bath solution via perfusion at 3 mL min⁻¹ (2-4 min). Data were analyzed using Clampfit 11 and GraphPad Prism 9.

Two-Electrode Voltage-Clamp (tevc) Electrophysiology

Xenopus laevis oocytes were harvested according to UCSF IACUC Protocol AN129690 and digested using collagenase (Worthington Biochemical Corporation, #LS004183, 0.8-1.0 mg/mL) in Ca²¹-free ND96 (96 mM NaCl, 2 mM KCl, 3.8 mM MgCl₂, 5 mM HEPES pH 7.4) immediately post-harvest, as previously reported (31,33). Oocytes were maintained at 18° C. in ND96 (96 mM NaCl, 2 mM KCl, 1.8 mM CaCl₂, 2 mM MgCl₂, 5 mM HEPES pH 7.4) supplemented with antibiotics (100 units mL⁻¹ penicillin, 100 μg mL⁻¹ streptomycin, 50 μg mL⁻¹ gentimycin) and used for experiments within one week of harvest.

Defolliculated stage V-VI oocytes were microinjected with 0.5-36 ng mRNA and currents were recorded within 16-48 h hours of injection.

For recordings, oocytes were impaled with borosilicate recording microelectrodes (0.4-2.0 MΩ resistance) backfilled with 3 M KCl and were subjected to constant perfusion of ND96 at a rate of 3 ml min⁻¹. Recording solutions containing K_{2P} activators were prepared immediately prior to use from DMSO stocks (20-100 mM), with final DMSO concentrations of 0.1%. Currents were evoked from a −80 mV holding potential followed by a 300 ms ramp from −150 mV to +50 mV. Data were recorded using a GeneClamp 500B amplifier (MDS Analytical Technologies) controlled by pCLAMP8 software (Molecular Devices), and digitized at 1 kHz using Digidata 1332 Å digitizer (MDS Analytical Technologies). For each recording, control solution (ND96) was perfused over a single oocyte until current was stable before switching to solutions containing the test compounds at various concentrations. Representative traces and dose response plots were generated in GraphPad Prism 9.

### Chemical Synthesis—Materials and Instrumentation

Chemical reagents and solvents (dry) were purchased from commercial suppliers and used without further purification. Synthesis of ML335 was carried out as previously reported (31). Thin layer chromatography (TLC) (Silicycle, F254, 250 μm) was performed on glass backed plates pre-coated with silica gel and were visualized by fluorescence quenching under UV light. Column chromatography was performed on Silicycle Sili-prep cartridges using a Biotage Isolera Four automated flash chromatography system. NMR spectra were measured using a Varian INOVA 400 MHz spectrometer (with 5 mm QuadNuclear Z-Grad probe). Chemical shifts are expressed in parts per million (ppm) and are referenced to CDCl₃ (7.26 ppm, 77.0 ppm) or DMSO (2.50 ppm, 40 ppm). Coupling constants are reported as Hertz (Hz). Splitting patterns are indicated as follows: s, singlet; d, doublet; t, triplet; q, quartet, dd, doublet of doublet; m, multiplet. LC-MS was carried out using a Waters Micromass ZQTM, equipped with Waters 2795 Separation Module and Waters 2996 Photodiode Array Detector and an XTerra MS C18, 5 μm, 4.6×50 mm column at ambient temperature. The mobile phases were MQ-H₂O with 0.1% formic acid (eluent A) and HPLC grade methanol with 0.1% formic acid (eluent B). Signals were monitored at 254 over 15 min with a gradient of 10-100% eluent B.

### Chemical Syntheses

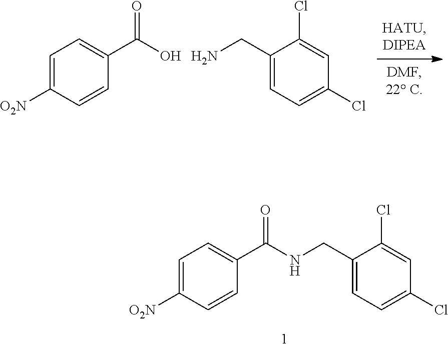 [SMILES: O=C(O)c1ccc([N+](=O)[O-])cc1 ; NCc1ccc(Cl)cc1Cl ; O=C(NCc1ccc(Cl)cc1Cl)c1ccc([N+](=O)[O-])cc1]

### Synthesis of N-[(2,4-dichlorophenyl)methyl]-4-nitrobenzamide, 1

A round-bottom flask was charged with 4-nitrobenzoic acid (2.00 g, 12.0 mmol), 2,4-dichlorobenzylamine (1.77 mL, 13.2 mmol), and HATU (5.00 g, 13.2 mmol). Anhydrous DMF (20 mL) and anhydrous diisopropylethylamine (4.17 mL, 24.0 mmol, 2.0 equiv.) were added and the flask was flushed with nitrogen, sealed, and stirred at 22° C. for 18 h. The reaction was diluted with ethyl acetate (150 mL) and washed with saturated sodium bicarbonate (2×100 mL), 10% citric acid in water (2×100 mL) and brine (1×100 mL).

The combined organics were dried with anhydrous magnesium sulfate, filtered and the solvent removed in vacuo. The crude solid was recrystallized from ethanol affording N-[(2,4-dichlorophenyl)methyl]-4-nitrobenzamide (1) as pale yellow needles (3.72 g, 11.4 mmol, 95.6%). 1H NMR (400 MHz, DMSO-d₆) δ 9.39 (t, J=5.7 Hz, 1H), 8.37-8.31 (m, 2H), 8.17-8.11 (m, 2H), 7.64 (t, J=1.2 Hz, 1H), 7.43 (d, J=1.3 Hz, 2H), 4.55 (d, J=5.7 Hz, 2H). ¹³C NMR (100 MHz, DMSO-d₆) δ 165.32, 149.62, 140.00, 135.57, 133.51, 132.83, 130.83, 129.33, 129.11, 127.84, 124.06, 40.96. LC/MS (ESI) exact mass for C₁₄H₉³⁵Cl₂N₂O₃⁻ [M−H]⁻ calc. 323.00, found 323.02.

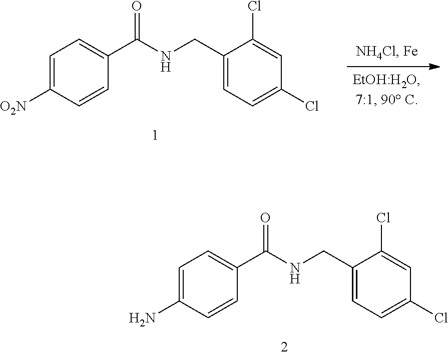 [SMILES: O=C(NCc1ccc(Cl)cc1Cl)c1ccc([N+](=O)[O-])cc1 ; Nc1ccc(C(=O)NCc2ccc(Cl)cc2Cl)cc1]

### Synthesis of 4-amino-N-(2,4-dichlorobenzyl)benzamide, 2

A round-bottom flask was charged with 1 (2.11 g, 6.49 mmol), iron powder (1.09 g, 19.5 mmol) and ammonium chloride (3.21 g, 64.9 mmol). Ethanol (100 mL) and water (15 mL) were added and the reaction was sealed and heated to 90° C. for 18 h. The crude reaction was filtered through diatomaceous earth, washing with ethyl acetate (25 mL). The combined filtrate was diluted with ethyl acetate (75 mL) and washed with dilute sodium hydroxide (2×100 mL) and brine (1×100 mL). The combined organics were dried with anhydrous magnesium sulfate, filtered and the solvent removed in vacuo. The crude solid was triturated with 25% ethyl acetate in hexanes and the remaining solid collected by vacuum filtration affording 2 as a pale tan solid (1.66 g, 5.61 mmol, 86.5% yield). 1H NMR (400 MHz, DMSO-d₆) δ 8.60 (t, J=5.9 Hz, 1H), 7.68-7.53 (m, 3H), 7.49-7.24 (m, 2H), 6.57 (d, J=8.6 Hz, 2H), 5.67 (s, 2H), 4.45 (d, J=5.8 Hz, 2H). ¹³C NMR (100 MHz, DMSO) δ 166.90, 152.36, 136.74, 133.13, 132.35, 130.31, 129.34, 128.89, 127.71, 120.96, 113.01. LCMS (ESI) exact mass for C₁₄H₁₃³⁵Cl₂N₂O₁⁺ [M+H]⁺ calcd: 295.04, found 294.91.

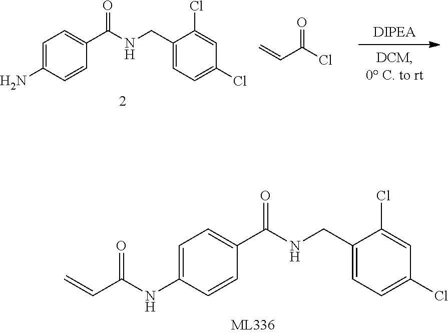

### Synthesis of ML336

A round-bottom flask was charged with 2 (295 mg, 999 mol, 1.00 equiv.), dichloromethane (5 mL) and diisopropylethylamine (348 μL, 2.00 mmol, 2.00 equiv.). The reaction was cooled to 0° C. and acryloyl chloride (109 mg, 1.20 mmol, 1.20 equiv) was added dropwise and the reaction allowed to warm to rt overnight. The crude reaction was concentrated in vacuo, then purified by column chromatography (0-100% EOAc in hexanes) affording ML336 as a white solid (201 mg, 576 mol, 57.6%). ¹H NMR (400 MHz, DMSO-d₆) δ ppm 10.38 (s, 1H), 8.97 (t, J=5.8 Hz, 1H), 7.91 (d, J=8.8 Hz, 2H), 7.78 (d, J=8.8 Hz, 2H), 7.62 (d, J=2.1 Hz, 1H), 7.43 (dd, J=8.4, 2.1 Hz, 1H), 7.37 (d, J=8.4 Hz, 1H), 6.47 (m, 1H), 6.31 (dd, J=16.9, 2.0 Hz, 1H), 5.81 (dd, J=10.0, 2.0 Hz, 1H), 4.51 (d, J=5.7 Hz, 2H). ¹³C NMR (100 MHz, DMSO) δ ppm 166.39, 163.90, 142.30, 136.23, 133.32, 132.58, 132.12, 130.53, 129.15, 129.01, 128.74, 127.98, 127.80, 119.08, 40.65, 40.44, 40.23, 40.02, 39.81, 39.60, 39.39. LCMS (ESI) exact mass for C₁₇H₁₅³⁵Cl₂N₂O₂⁺ [M+H]⁺ calcd: 349.05, found 348.99.

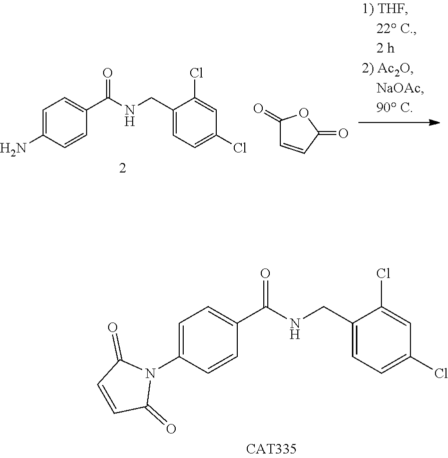 [SMILES: Nc1ccc(C(=O)NCc2ccc(Cl)cc2Cl)cc1 ; O=C1C=CC(=O)O1 ; O=C(NCc1ccc(Cl)cc1Cl)c1ccc(N2C(=O)C=CC2=O)cc1]

### Synthesis of CAT335

A vial was charged with 2 (200 mg, 678 mol, 1.00 equiv.), maleic anhydride (66.44 mg, 678 mol, 1.00 equiv.) and anhydrous THF (1.5 mL). The reaction was flushed with nitrogen, sealed, and stirred at 22° C. After 2 h, acetic anhydride (1.5 mL) and sodium acetate (167 mg, 2.03 mmol, 3.00 equiv.) were added and the reaction heated to 90° C. for 2 h. The reaction was cooled to rt, then diluted with water (10 mL). The resulting white precipitate was collected by vacuum filtration and washed with water (3×5 mL) and diethyl ether (3×5 mL), affording CAT335 as a white solid (200 mg, 533 mol, 78.7%). ¹H NMR (400 MHz, CDCl₃): δ ppm 7.90 (d, J=7.7 Hz, 2H), 7.48-7.54 (m, 2H), 7.42-7.47 (m, 2H), 7.25-7.28 (m, 1H), 6.91 (s, 2H), 6.62 (br s, 1H), 4.72 ppm (d, J=6.1 Hz, 2H). ¹³C NMR (100 MHz, CDCl₃) δ ppm 168.99, 134.41, 134.37, 134.33 (br s, 1 C) 134.27, 134.08, 133.10, 131.36, 129.46, 127.87, 127.50, 125.61, 41.65. LCMS (ESI) exact mass for C₁₄H₉³⁵Cl₂N₂O₃⁻ [M−H]⁻ calcd: 375.03, found 374.92.

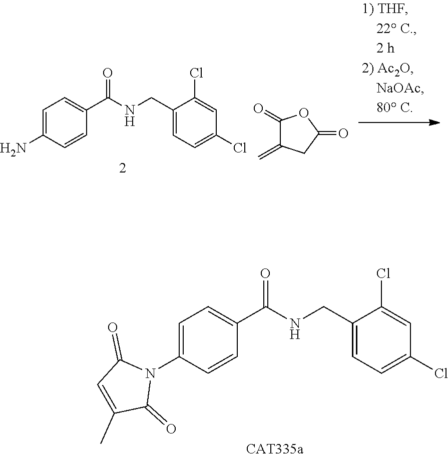

### Synthesis of CAT335a

A vial was charged with 2 (30 mg, 102 mol, 1.0 equiv.) and anhydrous THF (0.2 mL). Itaconic anhydride (13 mg, 116 mol, 1.1 equiv.) was added and the reaction stirred for 30 min, after which a yellow precipitate formed. 0.2 mL acetic anhydride and sodium acetate (25 mg. The reaction was flushed with nitrogen, sealed, and stirred at 22° C. After 2 h, acetic anhydride (1.5 mL) and sodium acetate (25 mg, 3.0 mmol, 3.0 equiv.) were added and the reaction heated to 80° C. for 3 h. The reaction was cooled to rt, then quenched with sat. aq. NaHCO₃ and extracted with EtOAc. The combined organics were dried with anhydrous sodium sulfate, filtered and the solvent removed in vacuo. The crude product was purified by column chromatography (0-100% EtOAc in hexanes) affording CAT335a as a white solid (18 mg, 46 mol, 46%). 1H NMR (400 MHz, CDCl₃) δ ppm 7.87 (d, J=8.64 Hz, 2H) 7.48 (d, J=8.64 Hz, 2H) 7.37-7.43 (m, 2H) 7.23 (dd, J=8.28, 2.07 Hz, 1H) 6.81 (br t, J=5.72 Hz, 1H) 6.51 (q, J=1.70 Hz, 1H) 4.68 (d, J=6.09 Hz, 2H) 2.19 (d, J=1.70 Hz, 3H). ¹³C NMR (100 MHz, CDCl₃) δ ppm 170.18, 169.05, 166.58, 146.09, 134.77, 134.24, 134.16, 134.13, 132.76, 131.12, 129.39, 127.83, 127.68, 127.43, 125.36, 41.54, 11.20. LCMS (ESI) exact mass for C₁₉H₁₅³⁵Cl₂N₂O₃+[M+H]⁺ calcd: 389.05, found 389.14.

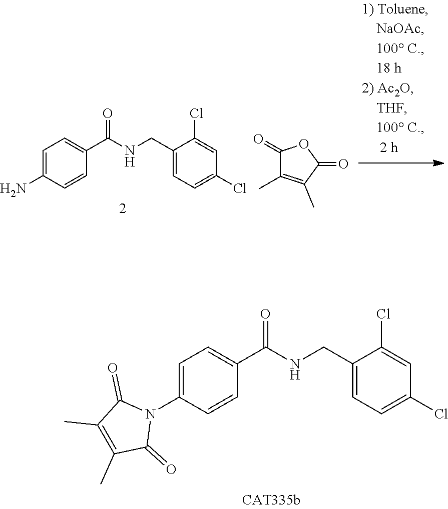

### Synthesis of CAT335b

A vial was charged with 2 (59 mg, 200 mol, 1.0 equiv.) and toluene (0.4 mL). 2,3-dimethyl maleic anhydride (25 mg, 200 mol, 1.0 equiv.) and sodium acetate (49 mg, 600 μmol, 3.0 equiv.) were added and the reaction sealed, heated to 100° C. and stirred for 18 h. Acetic anhydride (0.4 mL) and THF (0.4 mL) were added and the reaction stirred for an additional 2 h. The reaction was cooled to rt, then quenched with sat. aq. NaHCO₃ and extracted with EtOAc. The combined organics were dried with anhydrous sodium sulfate, filtered and the solvent removed in vacuo. The crude product was purified by column chromatography (0-100% EtOAc in hexanes) affording CAT335b as a white solid (25 mg, 62 μmol, 31%). 1H NMR (400 MHz, CDCl₃) δ ppm 7.84-7.89 (m, 2H) 7.49-7.53 (m, 2H) 7.38-7.44 (m, 2H) 7.24 (dd, J=8.28, 2.07 Hz, 1H) 6.71 (br t, J=5.66 Hz, 1H) 4.69 (d, J=5.97 Hz, 2H) 2.08 (s, 6H). ¹³C NMR (100 MHz, CDCl₃) δ ppm 170.34, 166.10, 137.51, 135.73, 135.39, 133.53, 132.86, 132.78, 132.52, 132.63, 130.50, 128.71 127.77, 127.22, 125.31, 40.64, 7.87. LCMS (ESI) exact mass for C₂₀H₁₇³⁵Cl₂N₂O₃⁺ [M+H]⁺ calcd: 403.06, found 403.17.

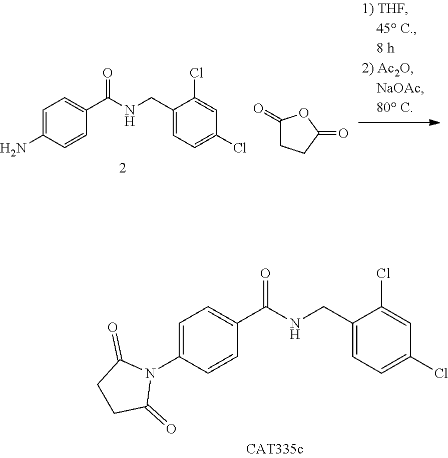 [SMILES: Nc1ccc(C(=O)NCc2ccc(Cl)cc2Cl)cc1 ; O=C1CCC(=O)O1 ; O=C(NCc1ccc(Cl)cc1Cl)c1ccc(N2C(=O)CCC2=O)cc1]

### Synthesis of CAT335c

A vial equipped with a magnetic stirbar was charged with 2 (148 mg, 500 μmol, 1 equiv.), succinic anhydride (50 mg, 500 μmol, 1 equiv.) and anhydrous THF (2 mL) and heated to 45° C. for 8 h. Acetic anhydride (2 mL) and sodium acetate (123 mg, 1.50 mmol, 3 equiv.) were then added and the reaction heated to 80° C. for 16 h. The reaction was cooled to rt and diluted with water (5 mL). The resulting white solid was collected by vacuum filtration, then recrystallized from 3:1 Ethyl acetate:hexanes to afford CAT335c as a white solid (82.0 mg, 217 μmol, 43.5%). 1H NMR (400 MHz, DMSO-d₆) δ=9.16 (br t, J=5.7 Hz, 1H), 8.02 (d, J=8.0 Hz, 2H), 7.64 (s, 1H), 7.47-7.36 (m, 4H), 4.54 (d, J=5.6 Hz, 2H), 2.81 (s, 4H). ¹³C NMR (100 MHz, DMSO-d₆) δ=177.2, 166.3, 136.0, 135.8, 134.0, 133.4, 132.7, 130.6, 129.1, 128.4, 127.8, 127.4, 40.8, 29.0. LCMS (ESI) exact mass for C₁₈H₁₅³⁵Cl₂N₂O₃⁺ [M+H]⁺ calcd: 377.05, found 376.91.

### General Method for Synthesis of Sultam Analogs

Most of chemicals were purchased from Sinopharm Chemical Reagent Co. (SCRC), Sigma-Aldrich, or Alfa. ¹H NMR or ¹⁹F NMR spectra were recorded on Bruker AVIII 400 or Bruker AVIII 500. LCMS measurement was run on Agilent 1200 HPLC/6100 SQ System using the follow conditions:

Method A: Mobile Phase: A: Water (10 mmol NH₄HCO₃) B: ACN; Gradient Phase: 5% B increase to 95% B within 1.4 min, 95% B with 1.6 min (total runtime:3 min); Flow Rate: 1.8 mL/min; Column: Xbridge C18, 3.0*30 mm, 2.5 μm; Column Temperature: 50° C. Detectors: UV (214, 4 nm) and MS (ESI, POS mode, 110 to 1000 amu), ES-API.

Method B: Mobile Phase: A: Water (10 mM NH₄HCO₃) B: Acetonitrile; Gradient Phase: 5% to 95% B within 1.4 min, 95% B with 1.4 min, back to 5% B within 0.01 min; Flow Rate: 1.8 mL/min; Column: XBridge C18, 4.6*50 mm, 3.5 um; Column Temperature: 50° C. Detectors: ADC ELSD, DAD (214 nm and 254 nm), MSD (ES-API).

Method C: Mobile Phase: A: Water (0.05% TFA) B: ACN (0.05% TFA); Gradient Phase: 5% B increase to 100% B within 1.3 min, 100% B with 1.7 min (total runtime: 3 min); Flow Rate: 2.0 mL/min; Column: SunFire C18, 4.6*50 mm, 3.5 μm; Column Temperature: 50° C. Detectors: UV (214 nm and 254 nm) and MS (ESI, Pos mode, 110 to 1000 amu), ES-API.

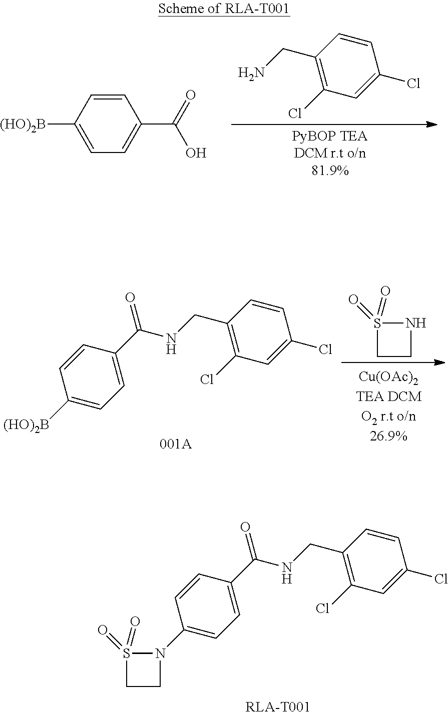 [SMILES: O=C(O)c1ccc(B(O)O)cc1 ; NCc1ccc(Cl)cc1Cl ; O=C(NCc1ccc(Cl)cc1Cl)c1ccc(B(O)O)cc1 ; O=S1(=O)CCN1 ; O=C(NCc1ccc(Cl)cc1Cl)c1ccc(N2CCS2(=O)=O)cc1]

### Step 1: (4-((2,4-Dichlorobenzyl)carbamoyl)phenyl)boronic acid

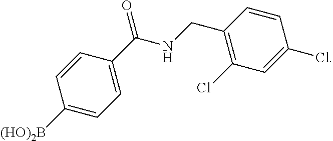 [SMILES: O=C(NCc1ccc(Cl)cc1Cl)c1ccc(B(O)O)cc1]

A mixture of 4-boronobenzoic acid (500 mg, 3.01 mmol), (2,4-dichlorophenyl) methanamine (637 mg, 3.62 mmol), benzotriazole-1-yl-oxytripyrrolidino phosphonium hexafluorophosphate (1.90 g, 3.62 mmol), triethylamine (913 mg, 9.04 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (methanol/dichloromethane=0-5%) to obtain (4-((2,4-dichlorobenzyl)carbamoyl) phenyl)boronic acid (800 mg, 81.9% yield) as a colorless oil. LC-MS: m/z=324.0 (M+H)⁺, retention time: 1.66 min (Method B).

### Step 2: N-(2,4-dichlorobenzyl)-4-(1,1-dioxido-1,2-thiazetidin-2-yl)benzamide

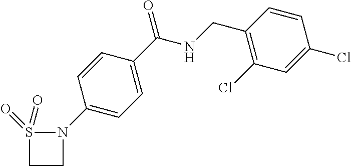 [SMILES: O=C(NCc1ccc(Cl)cc1Cl)c1ccc(N2CCS2(=O)=O)cc1]

A mixture of (4-((2,4-dichlorobenzyl)carbamoyl)phenyl)boronic acid (500 mg, 1.54 mmol), 1,2-thiazetidine 1,1-dioxide (165 mg, 1.54 mmol), cupric acetate (841 mg, 4.63 mmol) and triethylamine (574 mg, 4.63 mmol) in dichloromethane (10.0 mL) was stirred at room temperature under oxygen overnight. The reaction was concentrated under reduced pressure. The crude product was purified by flash chromatography (methanol/dichloromethane=0-5%) to obtain N-(2,4-dichlorobenzyl)-4-(1,1-dioxido-1,2-thiazetidin-2-yl)benzamide (160 mg, 26.9% yield) as a white solid. LC-MS: m/z=384.9 (M+H)⁺, retention time: 4.70 min (Method C).

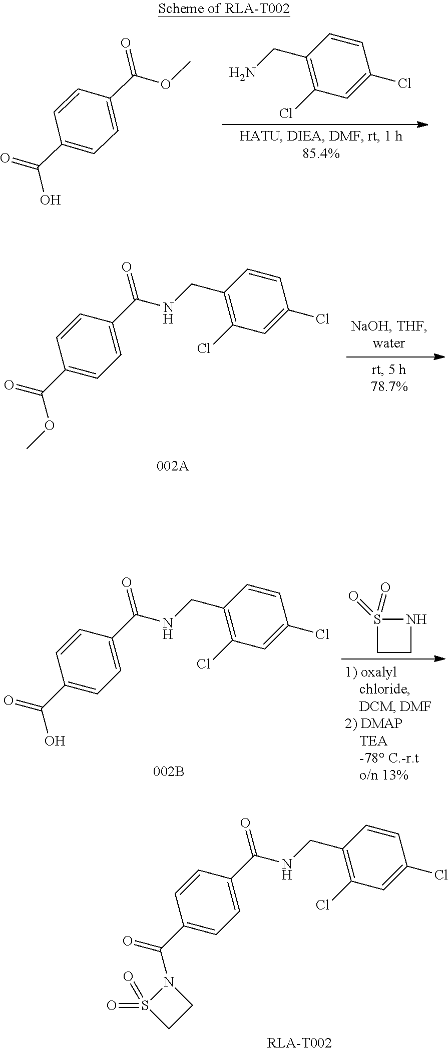 [SMILES: COC(=O)c1ccc(C(=O)O)cc1 ; NCc1ccc(Cl)cc1Cl ; COC(=O)c1ccc(C(=O)NCc2ccc(Cl)cc2Cl)cc1 ; O=C(O)c1ccc(C(=O)NCc2ccc(Cl)cc2Cl)cc1 ; O=S1(=O)CCN1 ; O=C(NCc1ccc(Cl)cc1Cl)c1ccc(C(=O)N2CCS2(=O)=O)cc1]

### Step 1: Methyl 4-((2,4-dichlorobenzyl)carbamoyl)benzoate

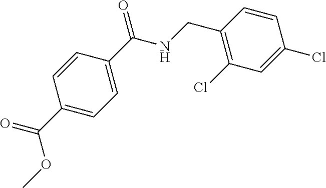 [SMILES: COC(=O)c1ccc(C(=O)NCc2ccc(Cl)cc2Cl)cc1]

A mixture of (2,4-dichlorophenyl)methanamine (2.4 g, 13.9 mmol), 4-(methoxycarbonyl)benzoic acid (2.5 g, 13.9 mmol), 2-(7-azabenzotriazol-1-yl)-N,N,N′,N′-tetramethyluronium hexafluorophosphate (6.3 g, 16.7 mmol) and N,N-diisopropylethylamine (3.6 g, 27.8 mmol) in N,N-dimethylformamide (20 mL) was stirred at room temperature for 2 hours. The reaction was quenched with water (150 mL) and extracted with ethyl acetate (100 mL*3). The combined organic phases were washed with brine, dried over sodium sulfate, filtered, concentrated and purified by column chromatography (25% ethyl acetate in petroleum ether) to give methyl 4-((2,4-dichlorobenzyl)carbamoyl)benzoate (4.0 g, 11.8 mmol, 85.4%) as white solid. LC-MS: m/z=338.1 (M+H)⁺, retention time: 2.09 min (Method C).

### Step 2: 4-((2,4-Dichlorobenzyl)carbamoyl)benzoic acid

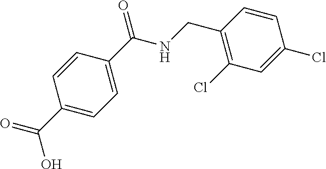 [SMILES: O=C(O)c1ccc(C(=O)NCc2ccc(Cl)cc2Cl)cc1]

A mixture of 4-((2,4-dichlorobenzyl)carbamoyl)benzoate (4.0 g, 11.8 mmol) and sodium hydroxide (944 mg, 23.6 mmol) in tetrohydrofuran (25 mL) and water (2.5 mol) was stirred at room temperature for 5 hours. The reaction mixture was concentrated and dissolved in water (30 mL). The solution was acidified with hydrochloric acid till pH=5. Then the mixture was filtered, and the residue was washed with water and dried to give 4-((2,4-dichlorobenzyl)carbamoyl) benzoic acid (3.0 g, 9.3 mol, 78.7%) as white solid. LC-MS: m/z=323.9 (M+H)⁺, retention time: 1.83 min (Method C).

### Step 3: N-(2,4-dichlorobenzyl)-4-(1,1-dioxido-1,2-thiazetidine-2-carbonyl)benzamide

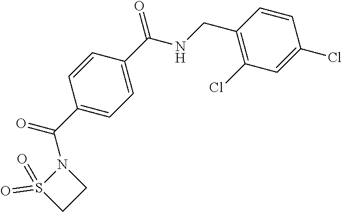 [SMILES: O=C(NCc1ccc(Cl)cc1Cl)c1ccc(C(=O)N2CCS2(=O)=O)cc1]

To a mixture of 4-((2,4-dichlorobenzyl)carbamoyl)benzoic acid (200 mg, 0.62 mmol) in dichloromethane was added oxalyl chloride (313 mg, 2.47 mmol) and N,N-dimethylformamide (1 drop) at 0° C. The mixture was stirred at 0° C. for 2 h and the resulting mixture was concentrated under reduced pressure to give 4-((2,4-dichlorobenzyl)carbamoyl)benzoyl chloride as a white solid. Then the aroyl chloride was added to a solution of 1,2-thiazetidine-1,1-dioxide (99 mg, 0.93 mmol) and 4-(dimethyl amino)pyri dine (5 mg, 0.04 mmol) in anhydrous dichloromethane (10 mL) at −78° C. The reaction mixture was stirred for 30 min before triethylamine (62 mg, 0.62 mmol) was added dropwise over 10 min at −78° C. forming a white precipitate. The mixture was then allowed to stir at room temperature for 12 h. The reaction mixture was filtered and the filtrate was concentrated under reduced pressure at 30° C. The pale-yellow oil was purified by prep-HPLC to give N-(2,4-dichlorobenzyl)-4-(1,1-dioxido-1,2-thiazetidine-2-carbon-yl)benzamide (33 mg, 1300 yield) as a white solid. LC-MS: m/z=412.9 (M+H)+, retention time: 1.95 min (Method C).

TABLE 2
Structures of the compounds and their TREK activity.

| | | | TREK-1 | TREK- | TREK-2 |
|---|---|---|---|---|---|
| Entry | Structure | TREK-1 | GC | 2 | GC |
| 1 | 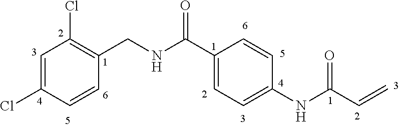 [SMILES: C=CC(=O)Nc1ccc(C(=O)NCc2ccc(Cl)cc2Cl)cc1] | +++ | +++ | | |
| 2 | 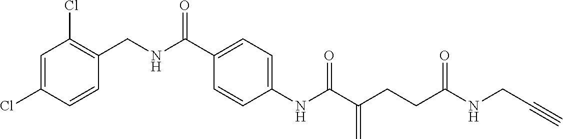 [SMILES: C#CCNC(=O)CCC(=C)C(=O)Nc1ccc(C(=O)NCc2ccc(Cl)cc2Cl)cc1] | +++ | +++ | | |
| 3 | 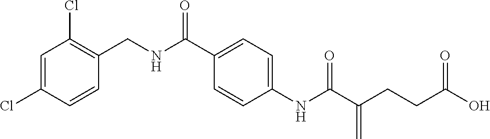 [SMILES: C=C(CCC(=O)O)C(=O)Nc1ccc(C(=O)NCc2ccc(Cl)cc2Cl)cc1] | ++ | ++ | | |
| 4 | 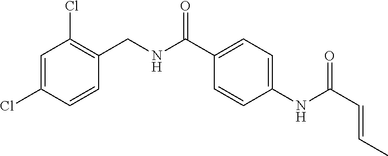 [SMILES: C/C=C/C(=O)Nc1ccc(C(=O)NCc2ccc(Cl)cc2Cl)cc1] | 1.2-fold − | | | |
| 5 | 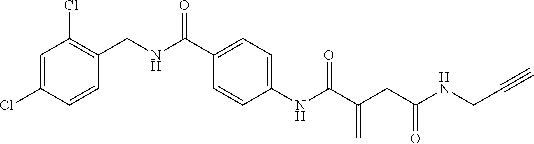 [SMILES: C#CCNC(=O)CC(=C)C(=O)Nc1ccc(C(=O)NCc2ccc(Cl)cc2Cl)cc1] | 7.0-fold +++ | | | |
| 6 | 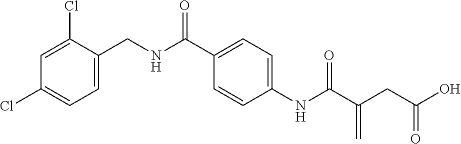 [SMILES: C=C(CC(=O)O)C(=O)Nc1ccc(C(=O)NCc2ccc(Cl)cc2Cl)cc1] | 1.7 to 2.5-fold + | | | |
| 7 | 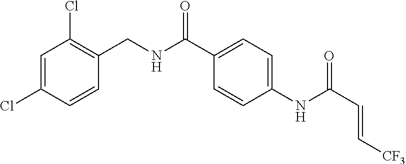 [SMILES: O=C(/C=C/C(F)(F)F)Nc1ccc(C(=O)NCc2ccc(Cl)cc2Cl)cc1] | 1.0-fold − | | | |
| 8 | 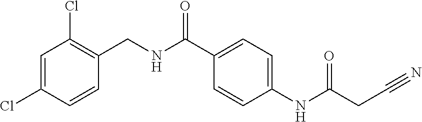 [SMILES: N#CCC(=O)Nc1ccc(C(=O)NCc2ccc(Cl)cc2Cl)cc1] | 3.6-fold ++ | | | |
| 9 | 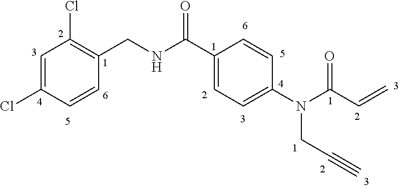 [SMILES: C#CCN(C(=O)C=C)c1ccc(C(=O)NCc2ccc(Cl)cc2Cl)cc1] | 1.0-fold − | | | |
| 10 | 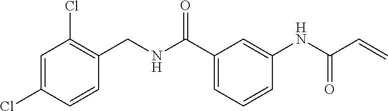 [SMILES: C=CC(=O)Nc1cccc(C(=O)NCc2ccc(Cl)cc2Cl)c1] | 1.2-fold − | | | |
| 11 | 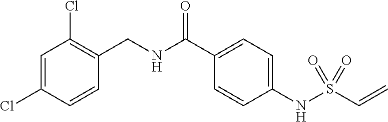 [SMILES: C=CS(=O)(=O)Nc1ccc(C(=O)NCc2ccc(Cl)cc2Cl)cc1] | 1.3-fold − | | | |
| 12 | 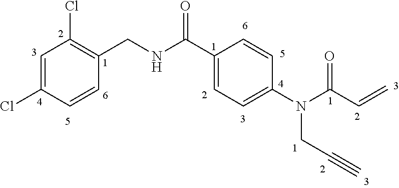 [SMILES: C#CCN(C(=O)C=C)c1ccc(C(=O)NCc2ccc(Cl)cc2Cl)cc1] | | | | |
| 13 | 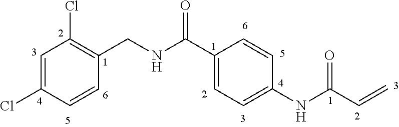 [SMILES: C=CC(=O)Nc1ccc(C(=O)NCc2ccc(Cl)cc2Cl)cc1] | | | | |
| 14 | 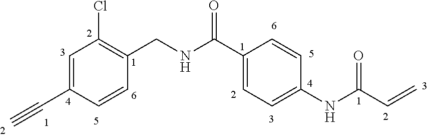 [SMILES: C#Cc1ccc(CNC(=O)c2ccc(NC(=O)C=C)cc2)c(Cl)c1] | | | | |
| 15 | 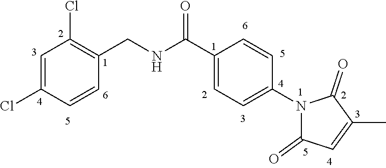 [SMILES: CC1=CC(=O)N(c2ccc(C(=O)NCc3ccc(Cl)cc3Cl)cc2)C1=O] | + | +++ | + | +++ |
| 16 | 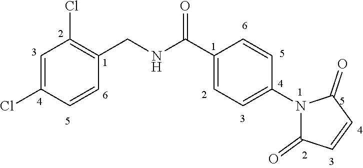 [SMILES: O=C(NCc1ccc(Cl)cc1Cl)c1ccc(N2C(=O)C=CC2=O)cc1] | + | +++ | + | +++ |
| 17 | 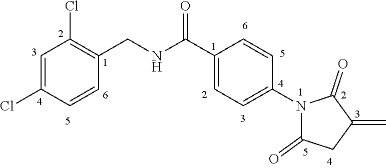 [SMILES: C=C1CC(=O)N(c2ccc(C(=O)NCc3ccc(Cl)cc3Cl)cc2)C1=O] | + | ++ | | |
| 18 | 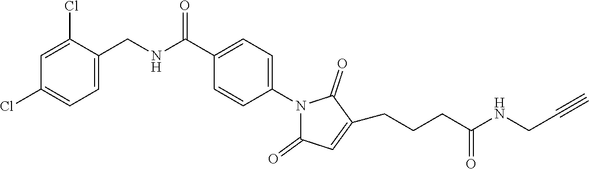 [SMILES: C#CCNC(=O)CCCC1=CC(=O)N(c2ccc(C(=O)NCc3ccc(Cl)cc3Cl)cc2)C1=O] | − | ++ | | |
| 19 | 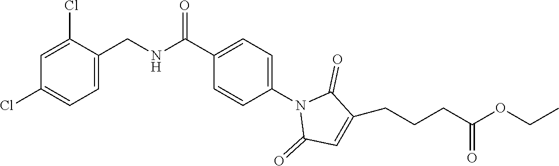 [SMILES: CCOC(=O)CCCC1=CC(=O)N(c2ccc(C(=O)NCc3ccc(Cl)cc3Cl)cc2)C1=O] | − | ++ | | |
| 20 | 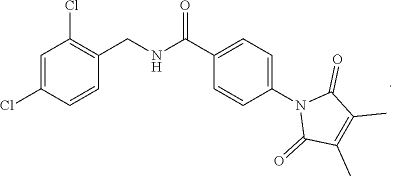 [SMILES: CC1=C(C)C(=O)N(c2ccc(C(=O)NCc3ccc(Cl)cc3Cl)cc2)C1=O] | − | ++ | − | + |
| 21 | 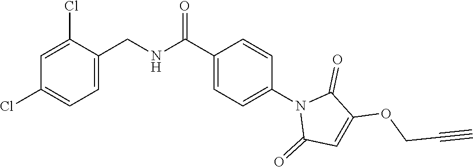 [SMILES: C#CCOC1=CC(=O)N(c2ccc(C(=O)NCc3ccc(Cl)cc3Cl)cc2)C1=O] | − | − | | |
| 22 | 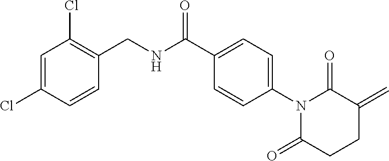 [SMILES: C=C1CCC(=O)N(c2ccc(C(=O)NCc3ccc(Cl)cc3Cl)cc2)C1=O] | − | − | | |
| 23 | 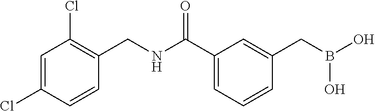 [SMILES: O=C(NCc1ccc(Cl)cc1Cl)c1cccc(CB(O)O)c1] | 1.0-fold − | | | |
| 24 | 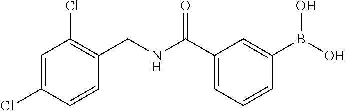 [SMILES: O=C(NCc1ccc(Cl)cc1Cl)c1cccc(B(O)O)c1] | 1.3-fold + | | | |
| 25 | 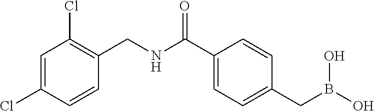 [SMILES: O=C(NCc1ccc(Cl)cc1Cl)c1ccc(CB(O)O)cc1] | 1.5-fold + | | | |
| 26 | 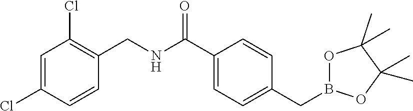 [SMILES: CC1(C)OB(Cc2ccc(C(=O)NCc3ccc(Cl)cc3Cl)cc2)OC1(C)C] | 1.6-fold + | | | |
| 27 | 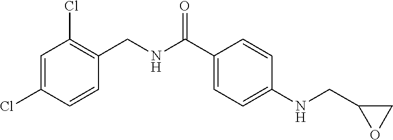 [SMILES: O=C(NCc1ccc(Cl)cc1Cl)c1ccc(NCC2CO2)cc1] | 2.5-fold ++ | | | |
| 28 | 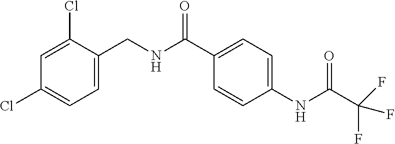 [SMILES: O=C(NCc1ccc(Cl)cc1Cl)c1ccc(NC(=O)C(F)(F)F)cc1] | 3.3-fold ++ | | | |
| 29 | 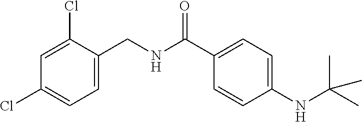 [SMILES: CC(C)(C)Nc1ccc(C(=O)NCc2ccc(Cl)cc2Cl)cc1] | | | | |
| 30 | 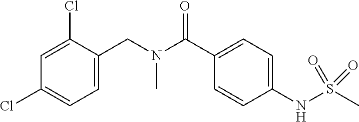 [SMILES: CN(Cc1ccc(Cl)cc1Cl)C(=O)c1ccc(NS(C)(=O)=O)cc1] | 1.0-fold − | | | |
| 31 | 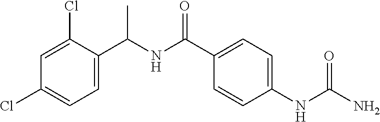 [SMILES: CC(NC(=O)c1ccc(NC(N)=O)cc1)c1ccc(Cl)cc1Cl] | 1.3-fold − | | | |
| 32 | 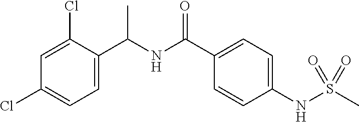 [SMILES: CC(NC(=O)c1ccc(NS(C)(=O)=O)cc1)c1ccc(Cl)cc1Cl] | 1.2-fold − | | | |
| 33 | 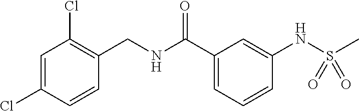 [SMILES: CS(=O)(=O)Nc1cccc(C(=O)NCc2ccc(Cl)cc2Cl)c1] | 2.2-fold ++ | | | |
| 34 | 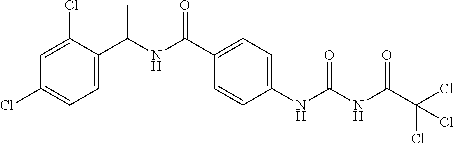 [SMILES: CC(NC(=O)c1ccc(NC(=O)NC(=O)C(Cl)(Cl)Cl)cc1)c1ccc(Cl)cc1Cl] | 1.2-fold − | | | |
| 35 | 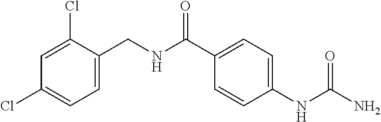 [SMILES: NC(=O)Nc1ccc(C(=O)NCc2ccc(Cl)cc2Cl)cc1] | 6.4-fold +++ | | | |
| 36 | 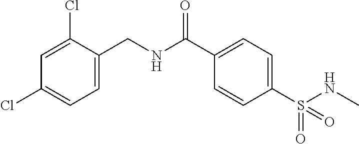 [SMILES: CNS(=O)(=O)c1ccc(C(=O)NCc2ccc(Cl)cc2Cl)cc1] | 3-fold ++ | | | |
| 37 | 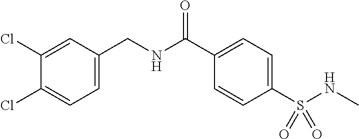 [SMILES: CNS(=O)(=O)c1ccc(C(=O)NCc2ccc(Cl)c(Cl)c2)cc1] | 1.5-fold + | | | |
| 38 | 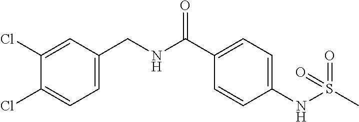 [SMILES: CS(=O)(=O)Nc1ccc(C(=O)NCc2ccc(Cl)c(Cl)c2)cc1] | 4.8-fold +++ | | | |
| 39 | 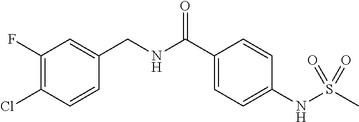 [SMILES: CS(=O)(=O)Nc1ccc(C(=O)NCc2ccc(Cl)c(F)c2)cc1] | 1.5-fold + | | | |
| 40 | 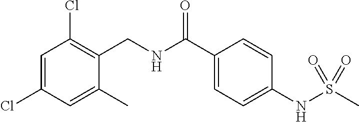 [SMILES: Cc1cc(Cl)cc(Cl)c1CNC(=O)c1ccc(NS(C)(=O)=O)cc1] | 1.1-fold − | | | |
| 41 | 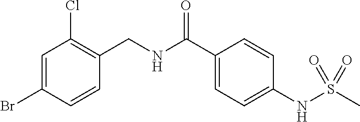 [SMILES: CS(=O)(=O)Nc1ccc(C(=O)NCc2ccc(Br)cc2Cl)cc1] | 3.3-fold ++ | | | |
| 42 | 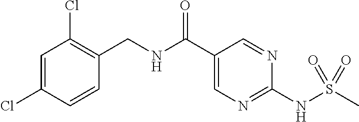 [SMILES: CS(=O)(=O)Nc1ncc(C(=O)NCc2ccc(Cl)cc2Cl)cn1] | 1.4-fold + | | | |
| 43 | 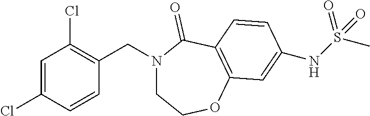 [SMILES: CS(=O)(=O)Nc1ccc2c(c1)OCCN(Cc1ccc(Cl)cc1Cl)C2=O] | 1.6-fold + | | | |
| 44 | 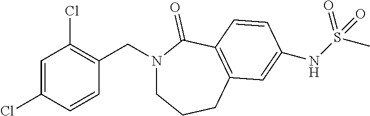 [SMILES: CS(=O)(=O)Nc1ccc2c(c1)CCCN(Cc1ccc(Cl)cc1Cl)C2=O] | 1.0-fold − | | | |
| 45 | 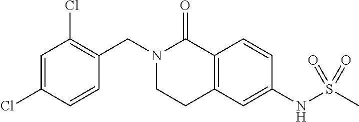 [SMILES: CS(=O)(=O)Nc1ccc2c(c1)CCN(Cc1ccc(Cl)cc1Cl)C2=O] | 1.1-fold − | | | |
| 46 | 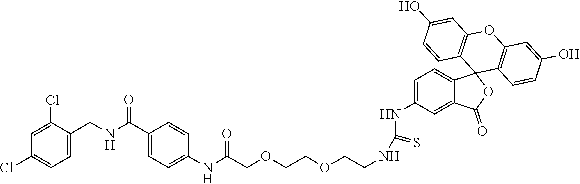 [SMILES: O=C(COCCOCCNC(=S)Nc1ccc2c(c1)C(=O)OC21c2ccc(O)cc2Oc2cc(O)ccc21)Nc1ccc(C(=O)NCc2ccc(Cl)cc2Cl)cc1] | 4-fold +++ | | | |
| 47 | 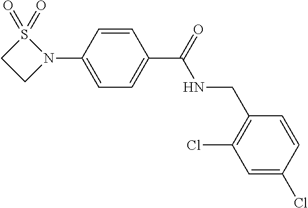 [SMILES: O=C(NCc1ccc(Cl)cc1Cl)c1ccc(N2CCS2(=O)=O)cc1] | − | + | | |
| 48 | 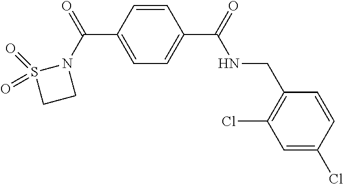 [SMILES: O=C(NCc1ccc(Cl)cc1Cl)c1ccc(C(=O)N2CCS2(=O)=O)cc1] | + | + | | |

Fold-activation at 20 uM in oocytes expressing TREK1 (or in select cases other channels).
+++>5 fold
++2.5-5 fold
+1.5-2.5 fold

General procedure for synthesis of cyanoacrylamide examples RLA-5769, 5770, 5771, 5798.

To a solution of 4-amino-N-(2,4-dichlorobenzyl)benzamide (50 mg, 0.17 mmol) in CH₂Cl₂ (2 mL), was added the corresponding cyanoacrylic acid (2 eq, 0.34 mmol), DMAP (1.33 eq, 0.23 mmol), and EDCI·HCl (2.66 eq, 0.45 mmol). The reaction mixture was then stirred at 70° C. until conversion was judged to be complete by UPLC/MS analysis. After cooling, water was added to the reaction mixture and the crude product collected as a solid by filtration, washed with CHCl₃ and dried in vacuo.

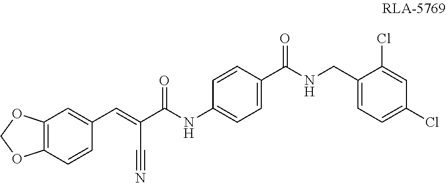 [SMILES: N#C/C(=C\c1ccc2c(c1)OCO2)C(=O)Nc1ccc(C(=O)NCc2ccc(Cl)cc2Cl)cc1]

(E)-4-(3-(benzo[d][1,3]dioxol-5-yl)-2-cyanoacrylamido)-N-(2,4-dichlorobenzyl)benzamide (37 mg, 44%) was obtained as a white solid following the general procedure. ¹H NMR (400 MHz, DMSO-d₆) δ ppm 10.57 (s, 1H), 9.04 (t, 1H), 8.24 (s, 1H), 7.95 (d, 2H), 7.79 (d, 2H), 7.66 (d, 2H), 7.57 (d, 1H), 7.44-7.33 (dd, 2H), 7.19 (d, 1H), 6.21 (s, 1H), 4.51 (d, 2H). LCMS (ESI) exact mass for C₂₅H₁₇Cl₂N₃O₄ [M+H]⁺ calcd: 493.06, found 494.27.

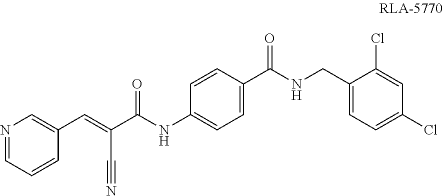 [SMILES: N#C/C(=C\c1cccnc1)C(=O)Nc1ccc(C(=O)NCc2ccc(Cl)cc2Cl)cc1]

(E)-4-(2-cyano-3-(pyridin-3-yl)acrylamido)-N-(2,4-dichlorobenzyl)benzamide (34 mg, 44%), was obtained as a white solid following the general procedure. ¹H NMR (400 MHz, DMSO-d₆) δ ppm 10.73 (s, 1H), 9.05 (t, 1H), 8.77 (s, 1H), 8.45 (d, 1H), 8.40 (s, 1H), 7.95 (d, 2H), 7.80 (m, 2H), 7.69-7.64 (m, 2H), 7.43-7.38 (dd, 2H), 4.52 (d, 2H). LCMS (ESI) exact mass for C₂₃H₁₆Cl₂N₄O₂ [M+H]⁺ calcd: 450.06, found 451.29.

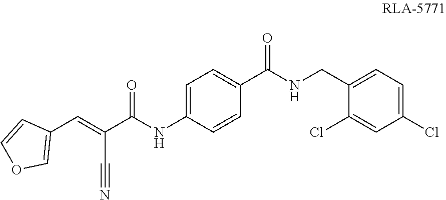 [SMILES: N#C/C(=C\c1ccoc1)C(=O)Nc1ccc(C(=O)NCc2ccc(Cl)cc2Cl)cc1]

(E)-4-(2-cyano-3-(furan-3-yl)acrylamido)-N-(2,4-dichlorobenzyl)benzamide (12 mg, 16%) was obtained as a white solid following the general procedure. 1H NMR (400 MHz, DMSO-d₆) δ ppm 10.54 (s, 1H), 9.03 (t, 1H), 8.52 (s, 1H), 8.28 (s, 1H), 7.99 (s, 1H), 7.93 (d, 2H), 7.82 (d, 2H), 7.64 (s, 1H), 7.45-7.33 (dd, 2H), 7.22 (s, 1H), 4.51 (d, 2H). LCMS (ESI) exact mass for C₂₂H₁₅Cl₂N₃O₃ [M+H]⁺ calcd: 439.04, found 440.29.

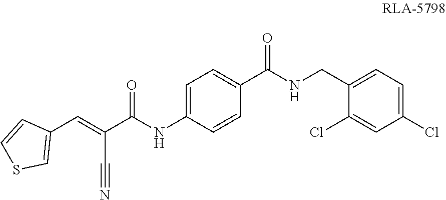 [SMILES: N#C/C(=C\c1ccsc1)C(=O)Nc1ccc(C(=O)NCc2ccc(Cl)cc2Cl)cc1]

(E)-4-(2-cyano-3-(thiophen-3-yl)acrylamido)-N-(2,4-dichlorobenzyl)benzamide (40 mg, 52%) was obtained as a yellow solid following the general procedure. ¹H NMR (400 MHz, DMSO-d₆) δ ppm 10.55 (s, 1H), 9.02 (t, 1H), 8.48 (s, 1H), 8.35 (s, 1H), 7.95-7.78 (m, 5H), 7.63 (d, J=2.1 Hz, 1H), 7.43-7.33 (m, 3H), 4.51 (d, 2H). LCMS (ESI) exact mass for C₂₂H₁₅Cl₂N₃O₂S [M+H]⁺ calcd: 455.02, found 456.34.

### Synthesis of (E)-4-(2-cyano-3-cyclopropylacrylamido)-N-(2,4-dichlorobenzyl)benzamide (RLA-5797)

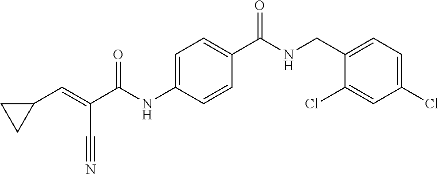 [SMILES: N#C/C(=C\C1CC1)C(=O)Nc1ccc(C(=O)NCc2ccc(Cl)cc2Cl)cc1]

To a solution of 4-amino-N-(2,4-dichlorobenzyl)benzamide (50 mg, 0.17 mmol) in CH₂Cl₂ (2 mL), was added (E)-2-cyano-3-cyclopropylacrylic acid (2 eq, 0.34 mmol), DMAP (1.33 eq, 0.23 mmol), and EDCI·HCl (2.66 eq, 0.45 mmol). The reaction mixture was then stirred at 70° C. until conversion was judged to be complete by UPLC/MS analysis. After cooling, water was added to the reaction mixture. The solution was extracted with EtOAc three times. The combined organic phase was then washed with NH₄Cl sat. aq. and brine, dried over MgSO₄, filtered, and concentrated to afford the crude product. Purification was performed by automated flash chromatography on a Silicycle 25g silica gel cartridge, eluting over 15 column volumes with a gradient of 0 to 100% EtOAc in n-hexane (35 mL/min) to obtain the desired compound as a white solid (18 mg, 26%). ¹H NMR (400 MHz, (CD₃)₂CO) 6 ppm 9.32 (s, 1H), 8.25 (t, 1H), 7.96 (d, 2H), 7.82 (d, 2H), 7.52 (d, 2H), 7.37 (d, 1H), 7.14 (d, 1H), 4.68 (d, 2H), 2.10 (m, 1H) 1.36 (m, 2H), 1.07 (m, 2H). LCMS (ESI) exact mass for C₂₁H₁₇Cl₂N₃O₂ [M+H]⁺ calcd: 413.07, found 414.37.

### INFORMAL SEQUENCE LISTING

### Chemogenetic Sequences Modified to Include a Cysteine Residue at an Amino Position Corresponding to Position 131 of TREK-1

SEQ ID NO: 1:
Modified TREK-1 from Mus musculus
MAAPDLLDPKSAAQNSKPRLSFSSKPTVLASRVESDSAINVMKWK
TVSTIFLVVVLYLIIGATVFKALEQPQEISQRTTIVIQKQTFIAQ
HACVNSTELDELIQQIVAAINAGIIPLGNSSNQVSHWDLGCSFFF
AGTVITTIGFGNISPRTEGGKIFCIIYALLGIPLFGFLLAGVGDQ
LGTIFGKGIAKVEDTFIKWNVSQTKIRIISTIIFILFGCVLFVAL
PAVIFKHIEGWSALDAIYFVVITLTTIGFGDYVAGGSDIEYLDFY
KPVVWFWILVGLAYFAAVLSMIGDWLRVISKKTKEEVGEFRAHAA
EWTANVTAEFKETRRRLSVEIYDKFQRATSVKRKLSAELAGNHNQ
ELTPCRRTLSVNHLTSEREVLPPLLKAESIYLNGLTPHCAGEDIA
VIENMK* 131
SEQ ID NO: 2:
Modified TREK-1 from Mus musculus
SFSSKPTVLASRVESDSAINVMKWKTVSTIFLVVVLYLIIGATVF
KALEQPQEISQRTTIVIQKQTFIAQHACVNSTELDELIQQIVAAI
NAGIIPLGNSSNQVSHWDLGCSFFFAGTVITTIGFGNISPRTEGG
KIFCIIYALLGIPLFGFLLAGVGDQLGTIFGKGIAKVEDTFIKWN
VSQTKIRIISTIIFILFGCVLFVALPAVIFKHIEGWSALDAIYFV
VITLTTIGFGDYVAGGSDIEYLDFYKPVVWFWILVGLAYFAAVLS
MIGDWLRVISKKTKEEVGEFRAHAAEWTANVT 111
SEQ ID NO: 3:
Modified TREK-1 from Mus musculus
VSTIFLVVVLYLIIGATVFKALEQPQEISQRTTIVIQKQTFIAQH
ACVNSTELDELIQQIVAAINAGIIPLGNSSNQVSHWDLGCSFFFA
GTVITTIGFGNISPRTEGGKIFCIIYALLGIPLFGFLLAGVGDQL
GTIFGKGIAKVEDTFIKWNVSQTKIRIISTIIFILFGCVLFVALP
AVIFKHIEGWSALDAIYFVVITLTTIGFGDYVAGGSDIEYLDFYK
PVVWFWILVGLAYFAAVLSMIGDWLRVISKKTKEEVGEFRA 85
SEQ ID NO: 4:
Modified TREK-1 from Homo sapiens
MMNPRAKRDFYLAAPDLLDPKSAAQNSKPRLSFSTKPTVLASRVE
SDTTINVMKWKTVSTIFLVVVLYLIIGATVFKALEQPHEISQRTT
IVIQKQTFISQHSCVNSTELDELIQQIVAAINAGIIPLGNTSNQI
SHWDLGCSFFFAGTVITTIGFGNISPRTEGGKIFCIIYALLGIPL
FGFLLAGVGDQLGTIFGKGIAKVEDTFIKWNVSQTKIRIISTIIF
ILFGCVLFVALPAIIFKHIEGWSALDAIYFVVITLTTIGFGDYVA
GGSDIEYLDFYKPVVWFWILVGLAYFAAVLSMIGDWLRVISKKTK
EEVGEFRAHAAEWTANVTAEFKETRRRLSVEIYDKFQRATSIKRK
LSAELAGNHNQELTPCRRTLSVNHLTSERDVLPPLLKTESIYLNG
LTPHCAGEEIAVIENIK* 142
SEQ ID NO: 5:
Modified TREK-2 from Mus musculus
MKFPIETPRKQVNWDPKVAVPAAAPPVCQPKSATNGHHPVPRLSI
SSRATVVARMEGASQGGLQTVMKWKTVVAIFVVVVVYLVTGGLVF
RALEQPFESSQKNTIALEKAEFLRDHICVSPQELETLIQHALDAD
NAGVSPVGNSSNSSSHWDLGCAFFFAGTVITTIGYGNIAPSTEGG
KIFCILYAIFGIPLFGFLLAGIGDQLGTIFGKSIARVEKVFRKKQ
VSQTKIRVISTILFILAGCIVFVTIPAVIFKYIEGWTALESIYFV
VVTLTTVGFGDFVAGGNAGINYREWYKPLVWFWILVGLAYFAAVL
SMIGDWLRVLSKKTKEEVGEIKAHAAEWKANVTAEFRETRRRLSV
EIHDKLQRAATIRSMERRRLGLDQRAHSLDMLSPEKRSVFAALDT
GRFKASSQESINNRPNNLRLKGPEQLTKHGQGASEDNIINKFGST
SKLTKRKNKDLKKTLPEDVQKIYKTFRNYSLDEEKKEDETEKMCN
SDNSSTAMLTECIQQQAEMENGMVPTDTKDQGLENNSLLEDR
N* 156
SEQ ID NO: 6:
Modified TREK-2 from Mus musculus
QGGLQTVMKWKTVVAIFVVVVVYLVTGGLVFRALEQPFESSQKNT
IALEKAEFLRDHICVSPQELETLIQHALDADNAGVSPVGNSSNSS
SHWDLGCAFFFAGTVITTIGYGNIAPSTEGGKIFCILYAIFGIPL
FGFLLAGIGDQLGTIFGKSIARVEKVFRKKQVSQTKIRVISTILF
ILAGCIVFVTIPAVIFKYIEGWTALESIYFVVVTLTTVGFGDFVA
GGNAGINYREWYKPLVWFWILVGLAYFAAVLSMIGDWLRVLSKKT
KE 97
SEQ ID NO: 7:
Modified TREK-2 from Homo sapiens
MKFPIETPRKQVNWDPKVAVPAAAPVCQPKSATNGQPPAPAPTPT
PRLSISSRATVVARMEGTSQGGLQTVMKWKTVVAIFVVVVVYLVT
GGLVFRALEQPFESSQKNTIALEKAEFLRDHVCVSPQELETLIQH
ALDADNAGVSPIGNSSNNSSHWDLGCAFFFAGTVITTIGYGNIAP
STEGGKIFCILYAIFGIPLFGFLLAGIGDQLGTIFGKSIARVEKV
FRKKQVSQTKIRVISTILFILAGCIVFVTIPAVIFKYIEGWTALE
SIYFVVVTLTTVGFGDFVAGGNAGINYREWYKPLVWFWILVGLAY
FAAVLSMIGDWLRVLSKKTKEEVGEIKAHAAEWKANVTAEFRETR
RRLSVEIHDKLQRAATIRSMERRRLGLDQRAHSLDMLSPEKRSVF
AALDTGRFKASSQESINNRPNNLRLKGPEQLNKHGQGASEDNIIN
KFGSTSRLTKRKNKDLKKTLPEDVQKIYKTFRNYSLDEEKKEEET
EKMCNSDNSSTAMLTDCIQQHAELENGMIPTDTKDREPENNSLLE
DRN* 161
SEQ ID NO: 8:
Modified TREK-2 from Homo sapiens
GLQTVMKWKTVVAIFVVVVVYLVTGGLVFRALEQPFESSQKNTIA
LEKAEFLRDHVCVSPQELETLIQHALDADNAGVSPIGNSSNNSSH
WDLGCAFFFAGTVITTIGYGNIAPSTEGGKIFCILYAIFGIPLFG
FLLAGIGDQLGTIFGKSIARVEKVFRKKQVSQTKIRVISTILFIL
AGCIVFVTIPAVIFKYIEGWTALESIYFVVVTLTTVGFGDFVAGG
NAGINYREWYKPLVWFWILVGLAYFAAVLSMIGDWLRVLSKKTKE
EVGE 95
SEQ ID NO: 9:
Modified TRAAK from Mus musculus
MRSTTLLALLALVLLYLVSGALVFQALEQPHEQQAQKKMDHGRDQ
FLRDHPCVSQKSLEDFIKLLVEALGGGANPETSWTNSSNHSSAWN
LGCAFFFSGTIITTIGYGNIVLHTDAGRLFCIFYALVGIPLFGML
LAGVGDRLGSSLRRGIGHIEAIFLKWHVPPGLVRSLSAVLFLLIG
CLLFVLTPTFVFSYMESWSKLEAIYFVIVTLTTVGFGDYVPGDGT
GQNSPAYQPLVWFWILFGLAYFASVLTTIGNWLRAVSRRTRAEMG
GLTAQAASWTGTVTARVTQRTGPSAPPPEKEQPLLPSSLPAPPAV
VEPAGRPGSPAPAEKVETPSPPTASALDYPSENLAFIDESSDTQS
ERGCALPRAPRGRRRPNPSKKPSRPRGPGRLRDKAVPV* 93
SEQ ID NO: 10:
Modified TRAAK from Mus musculus
MRSTTLLALLALVLLYLVSGALVFQALEQPHEQQAQKKMDHGRDQ
FLRDHPCVSQKSLEDFIKLLVEALGGGANPETSWTNSSNHSSAWN
LGCAFFFSGTIITTIGYGNIVLHTDAGRLFCIFYALVGIPLFGML
LAGVGDRLGSSLRRGIGHIEAIFLKWHVPPGLVRSLSAVLFLLIG
CLLFVLTPTFVFSYMESWSKLEAIYFVIVTLTTVGFGDYVPGDGT
GQNSPAYQPLVWFWILFGLAYFASVLTTIGNWLRAVSRRTRAEMG
GLTAQ 93
SEQ ID NO: 11:
Modified TRAAK from Homo sapiens
MRSTTLLALLALVLLYLVSGALVFRALEQPHEQQAQRELGEVREK
FLRAHPCVSDQELGLLIKEVADALGGGADPETNSTSNSSHSAWDL
GCAFFFSGTIITTIGYGNVALRTDAGRLFCIFYALVGIPLFGILL
AGVGDRLGSSLRHGIGHIEAIFLKWHVPPELVRVLSAMLFLLIGC
LLFVLTPTFVFCYMEDWSKLEAIYFVIVTLTTVGFGDYVAGADPR
QDSPAYQPLVWFWILLGLAYFASVLTTIGNWLRVVSRRTRAEMGG
LTAQAASWTGTVTARVTQRAGPAAPPPEKEQPLLPPPPCPAQPLG
RPRSPSPPEKAQPPSPPTASALDYPSENLAFIDESSDTQSERGCP
LPRAPRGRRRPNPPRKPVRPRGPGRPRDKGVPV* 92
SEQ ID NO: 12:
Modified TRAAK from Homo sapiens
MRSTTLLALLALVLLYLVSGALVFRALEQPHEQQAQRELGEVREK
FLRAHPCVSDQELGLLIKEVADALGGGADPETNSTSNSSHSAWDL
GCAFFFSGTIITTIGYGNVALRTDAGRLFCIFYALVGIPLFGILL
AGVGDRLGSSLRHGIGHIEAIFLKWHVPPELVRVLSAMLFLLIGC
LLFVLTPTFVFCYMEDWSKLEAIYFVIVTLTTVGFGDYVAGADPR
QDSPAYQPLVWFWILLGLAYFASVLTTIGNWLRVVSRRTRAEMGG
LTAQAASWTGTVTARVTQRAGPAAPPPEKEQ 92

### REFERENCES

1. Enyedi, P. & Czirjak, G. Molecular background of leak K⁺ currents: two-pore domain potassium channels. Physiol Rev 90, 559-605 (2010).
2. Feliciangeli, S., Chatelain, F.C., Bichet, D. & Lesage, F. The family of K2P channels: salient structural and functional properties. J Physiol (2014).
3. Lesage, F. & Barhanin, J. Molecular physiology of pH-sensitive background K(2P) channels. Physiology (Bethesda) 26, 424-37 (2011).
4. Kanda, H. et al. TREK-1 and TRAAK Are Principal K(+) Channels at the Nodes of Ranvier for Rapid Action Potential Conduction on Mammalian Myelinated Afferent Nerves. Neuron (2019).
5. Brohawn, S. G. et al. The mechanosensitive ion channel TRAAK is localized to the mammalian node of Ranvier. Elife 8(2019).
6. Alloui, A. et al. TREK-1, a K⁺ channel involved in polymodal pain perception. Embo J 25, 2368-76 (2006).
7. Devilliers, M. et al. Activation of TREK-1 by morphine results in analgesia without adverse side effects. Nat Commun 4, 2941 (2013).
8. Vivier, D. et al. Development of the first Two-Pore Domain Potassium Channel TREK-1 (TWIK-Related K+ Channel 1)-selective agonist possessing in vivo anti-nociceptive activity. J Med Chem (2017).
9. Yoshida, K. et al. Leak potassium channels regulate sleep duration. Proc Natl Acad Sci USA 115, E9459-E9468 (2018).
10. Steinberg, E. A., Wafford, K. A., Brickley, S. G., Franks, N. P. & Wisden, W. The role of K2p channels in anaesthesia and sleep. Pflugers Arch 467, 907-16 (2015).
11. Yarishkin, O. et al. TREK-1 channels regulate pressure sensitivity and calcium signaling in trabecular meshwork cells. J Gen Physiol 150, 1660-1675 (2018).
12. Royal, P. et al. Migraine-Associated TRESK Mutations Increase Neuronal Excitability through Alternative Translation Initiation and Inhibition of TREK. Neuron 101, 232-245 e6 (2019).
13. Heurteaux, C. et al. Deletion of the background potassium channel TREK-1 results in a depression-resistant phenotype. Nat Neurosci 9, 1134-41 (2006).
14. Lambert, M. et al. Loss of KCNK3 is a hallmark of RV hypertrophy/dysfunction associated with pulmonary hypertension. Cardiovasc Res 114, 880-893 (2018).
15. Zyrianova, T. et al. K2P2.1 (TREK-1) potassium channel activation protects against hyperoxia-induced lung injury. Sci Rep 10, 22011 (2020).
16. Honore, E., Maingret, F., Lazdunski, M. & Patel, A.J. An intracellular proton sensor commands lipid- and mechano-gating of the K(+) channel TREK-1. EMBO J 21, 2968-76 (2002).
17. Sandoz, G., Douguet, D., Chatelain, F., Lazdunski, M. & Lesage, F. Extracellular acidification exerts opposite actions on TREK1 and TREK2 potassium channels via a single conserved histidine residue. Proc Natl Acad Sci USA 106, 14628-33 (2009).
18. Brohawn, S. G., Campbell, E. B. & MacKinnon, R. Physical mechanism for gating and mechanosensitivity of the human TRAAK K⁺ channel. Nature 516, 126-30 (2014).
19. Maingret, F. et al. TREK-1 is a heat-activated background K(+) channel. Embo J 19, 2483-91 (2000).
20. Natale, A. M., Deal, P. E. & Minor, D. L. Structural Insights into the Mechanisms and Pharmacology of K. J Mol Biol 433, 166995 (2021).
21. Mathie, A., Veale, E. L., Cunningham, K. P., Holden, R. G. & Wright, P. D. Two-Pore Domain Potassium Channels as Drug Targets: Anesthesia and Beyond. Annu Rev Pharmacol Toxicol (2020).
22. Decher, N., Rinné, S., Bedoya, M., Gonzalez, W. & Kiper, A. K. Molecular Pharmacology of K 2P Potassium Channels. Cell Physiol Biochem 55, 87-107 (2021).
23. Sterbuleac, D. Molecular determinants of chemical modulation of two-pore domain potassium channels. Chem Biol Drug Des 94, 1596-1614 (2019).
24. Levitz, J. et al. Heterodimerization within the TREK channel subfamily produces a diverse family of highly regulated potassium channels. Proc Natl Acad Sci USA 113, 4194-9 (2016).
25. Hwang, E. M. et al. A disulphide-linked heterodimer of TWIK-1 and TREK-1 mediates passive conductance in astrocytes. Nat Commun 5, 3227 (2014).
26. Czirjak, G. & Enyedi, P. Formation of functional heterodimers between the TASK-1 and TASK-3 two-pore domain potassium channel subunits. J Biol Chem 277, 5426-32 (2002).
27. Lengyel, M., Czirják, G., Jacobson, D. A. & Enyedi, P. TRESK and TREK-2 two-pore-domain potassium channel subunits form functional heterodimers in primary somatosensory neurons. J Biol Chem 295, 12408-12425 (2020).
28. Sternson, S. M. & Roth, B. L. Chemogenetic tools to interrogate brain functions. Annu Rev Neurosci 37, 387-407 (2014).
29. Atasoy, D. & Sternson, S. M. Chemogenetic Tools for Causal Cellular and Neuronal Biology. Physiol Rev 98, 391-418 (2018).
30. Hertz, N. T. et al. Chemical genetic approach for kinase-substrate mapping by covalent capture of thiophosphopeptides and analysis by mass spectrometry. Curr Protoc Chem Biol 2, 15-36 (2010).
31. Shah, K., Liu, Y., Deirmengian, C. & Shokat, K. M. Engineering unnatural nucleotide specificity for Rous sarcoma virus tyrosine kinase to uniquely label its direct substrates. Proc Natl Acad Sci USA 94, 3565-70 (1997).
32. Runcie, A. C., Chan, K. H., Zengerle, M. & Ciulli, A. Chemical genetics approaches for selective intervention in epigenetics. Curr Opin Chem Biol 33, 186-94 (2016).
33. Baud, M. G. J. et al. Chemical biology. A bump-and-hole approach to engineer controlled selectivity of BET bromodomain chemical probes. Science 346, 638-641 (2014).
34. Alexander, G. M. et al. Remote control of neuronal activity in transgenic mice expressing evolved G protein-coupled receptors. Neuron 63, 27-39 (2009).
35. Armbruster, B.N., Li, X., Pausch, M.H., Herlitze, S. & Roth, B.L. Evolving the lock to fit the key to create a family of G protein-coupled receptors potently activated by an inert ligand. Proc Natl Acad Sci USA 104, 5163-8 (2007).
36. Magnus, C. J. et al. Ultrapotent chemogenetics for research and potential clinical applications. Science 364(2019).
37. Lolicato, M. et al. K2P2.1 (TREK-1)-activator complexes reveal a cryptic selectivity filter binding site. Nature 547, 364-368 (2017).
38. Al-Moubarak, E., Veale, E. L. & Mathie, A. Pharmacologically reversible, loss of function mutations in the TM2 and TM4 inner pore helices of TREK-1 K2P channels. Sci Rep 9, 12394 (2019).
39. Bagriantsev, S. N., Clark, K. A. & Minor, D. L., Jr. Metabolic and thermal stimuli control K(2P)2.1 (TREK-1) through modular sensory and gating domains. EMBO J 31, 3297-308 (2012).
40. Pope, L. et al. Protein and Chemical Determinants of BL-1249 Action and Selectivity for K2P Channels. ACS Chem Neurosci 9, 3153-3165 (2018).
41. Schewe, M. et al. A pharmacological master key mechanism that unlocks the selectivity filter gate in K(+) channels. Science 363, 875-880 (2019).
42. Magnus, C. J. et al. Chemical and genetic engineering of selective ion channel-ligand interactions. Science 333, 1292-6 (2011).
43. Auffenberg, E. et al. Remote and reversible inhibition of neurons and circuits by small molecule induced potassium channel stabilization. Sci Rep 6, 19293 (2016).
44. Fortin, D. L. et al. Optogenetic photochemical control of designer K+ channels in mammalian neurons. J Neurophysiol 106, 488-96 (2011).
45. Sandoz, G., Levitz, J., Kramer, R. H. & Isacoff, E. Y. Optical control of endogenous proteins with a photoswitchable conditional subunit reveals a role for TREK1 in GABA(B) signaling. Neuron 74, 1005-14 (2012).
46. Bagriantsev, S. N., Peyronnet, R., Clark, K. A., Honore, E. & Minor, D. L., Jr. Multiple modalities converge on a common gate to control K2P channel function. EMBO J 30, 3594-606 (2011).
47. Bagriantsev, S. N. et al. A high-throughput functional screen identifies small molecule regulators of temperature- and mechano-sensitive K2P channels. ACS Chem Biol 8, 1841-51 (2013).

What is claimed is:

1. A compound, or a pharmaceutically acceptable salt thereof, having the formula (I): 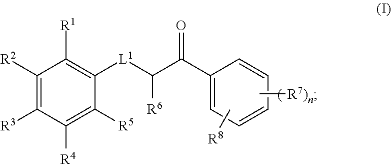
wherein:
L¹ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCHX¹₂, —OCH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCHX²₂, —OCH₂X², —CN, —N₃, —SOₙ₂R^{2A}, —SOᵥ₂NR^{2B}R^{2C}, —NHR^{2B}R^{2C}, —ONR^{2B}R^{2C}, —NHC(O)NHNR^{2B}R^{2C}, —NHC(O)NR^{2B}R^{2C}, —N(O)ₘ₂, NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCHX³₂, —OCH₂X³, —CN, —N₃, —SOₙ₃R^{3A}, —SOᵥ₃NR^{3B}R^{3C}, —N—R^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D} —NR^{3B}OR^{3D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCHX⁴₂, —OCH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCHX⁵₂, —OCH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, substituted or unsubstituted alkyl, or substituted or unsubstituted heteroalkyl; or R⁶ is optionally joined with L¹ to form a substituted or unsubstituted heterocycloalkyl;
R⁷ is independently halogen, —CX⁷₃, —CHX⁷₂, —CH₂X⁷, —OCX⁷₃, —OCHX⁷₂, —OCH₂X⁷, —CN, —N₃, —SOₙ₇R^{7A}, —SOᵥ₇NR^{7B}R^{7C}, —NR^{7B}R^{7C}, —ONR^{7B}R^{7C}, —NHC(O)NHNR^{7B}R^{7C}, NHC(O)NR^{7B}R^{7C}, —N(O)ₘ₇, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, —NR^{7B}OR^{7D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{7A}, R^{7B}, R^{7C}, and R^{7D} are independently hydrogen, halogen, —CF₃, —Cl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each X¹, X², X³, X⁴, X⁵, and X⁷ is independently halogen;
n is an integer from 0 to 3;
n1, n2, n3, n4, n5, and n7 are independently an integer from 0 to 4;
m1, m2, m3, m4, m5, m7, v1, v2, v3, v4, v5, and v7 are independently 1 or 2; and
R⁸ is a cysteine binding moiety or a serine binding moiety.

2. The compound of claim 1, wherein the cysteine binding moiety is: 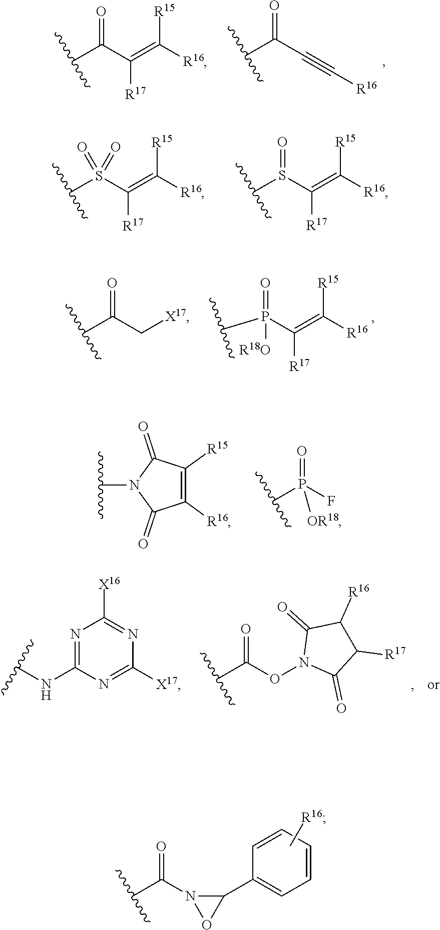
wherein:
R¹⁵ is hydrogen, halogen, —CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁶ is hydrogen, halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B} ONR^{16A}R^{16B}, —NHC═(O)NHNR^{16A}R^{16B}, —NHC(O)NR^{16A}R^{16B}, —N(O)ₘ₁₆, —NR^{16A}R^{16B} C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁷ is hydrogen, halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁸ is hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, and R^{18C} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;
X, X¹⁵, X¹⁶, X¹⁷, and X¹⁸ are independently —F, —Cl, —Br, or —I;
n15, n16, and n17 are independently an integer from 0 to 4; and
m15, m16, m17, v15, v16, and v17 are independently 1 or 2.

3. The compound of claim 2, wherein R¹⁵, R¹⁶, R¹⁷, and R¹⁸ are independently hydrogen, substituted or unsubstituted C₁-C₄ alkyl, or substituted or unsubstituted 2 to 4 membered heteroalkyl.

4. The compound of claim 2, wherein R¹⁵, R¹⁶, R¹⁷, and R¹⁸ are independently hydrogen or unsubstituted C₁-C₂ alkyl and X¹⁷ is —Cl.

5. The compound of claim 1, wherein the cysteine binding moiety is:

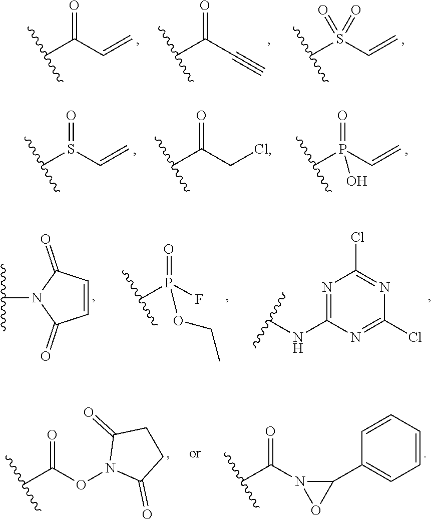

6. The compound of claim 1, wherein the serine binding moiety is: 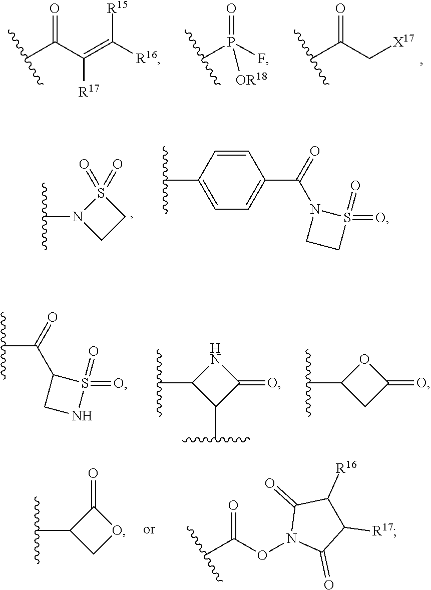
wherein:
R¹⁵, R¹⁶, R¹⁶, and R¹⁸ are independently hydrogen, substituted or unsubstituted C₁-C₄ alkyl, or substituted or unsubstituted 2 to 4 membered heteroalkyl; and
X¹⁷ is halogen.

7. The compound of claim 6, wherein R¹⁵, R¹⁶, R¹⁷, and R¹⁸ are independently hydrogen or unsubstituted C₁-C₂ alkyl and X¹⁷ is —Cl.

8. The compound of claim 6, wherein the serine binding moiety is:

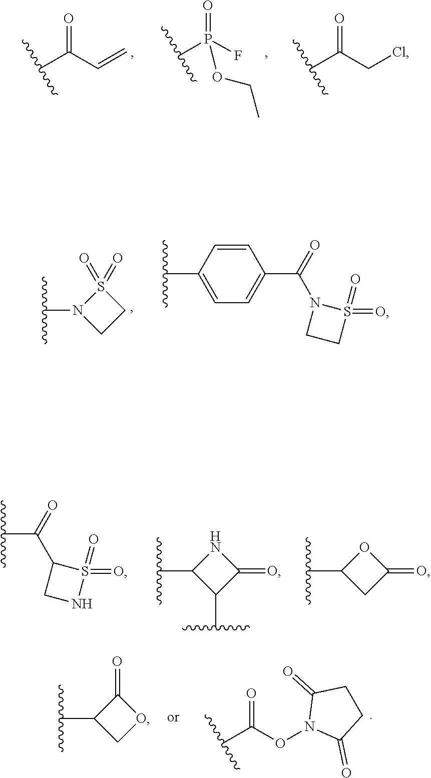

9. The compound of claim 1, wherein R¹ is hydrogen or halogen.

10. The compound of claim 1, wherein R¹ is —Cl.

11. The compound of claim 1, wherein R² is hydrogen or halogen.

12. The compound of claim 1, wherein R² is hydrogen.

13. The compound of claim 1, wherein R² is —F or —Cl.

14. The compound of claim 1, wherein R³ is halogen or C₁-C₄ substituted or unsubstituted alkynyl.

15. The compound of claim 1, wherein R³ is halogen.

16. The compound of claim 1, wherein R³ is —Cl, —Br, or —I.

17. The compound of claim 1, wherein R³ is unsubstituted 1λ³,2λ³-ethyne.

18. The compound of claim 1, wherein R⁴ and R⁵ are hydrogen.

19. The compound of claim 1, wherein R⁶ is hydrogen, substituted or unsubstituted C₁-C₄ alkyl; or R⁶ and L¹ are joined together to form a substituted or unsubstituted 5 to 7 membered heterocycloalkyl.

20. The compound of claim 1, wherein R⁶ is hydrogen.

21. The compound of claim 1, wherein R⁶ is unsubstituted methyl.

22. The compound of claim 1, wherein R⁶ and L¹ are joined together to form a substituted or unsubstituted 5 to 7 membered heterocycloalkyl.

23. The compound of claim 1, wherein R⁶ and L¹ are joined together to form a substituted or unsubstituted 1λ²-azepan-2-one, substituted or unsubstituted 1,4λ²-oxazepan-5-one, or substituted or unsubstituted 1λ²-piperidin-2-one.

24. The compound of claim 1, wherein R⁷ is independently a substituted or unsubstituted C₁-C₄ alkyl, substituted or unsubstituted 2 to 4 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

25. The compound of claim 1, wherein R⁷ is an unsubstituted C₁-C₄ alkyl, unsubstituted 2 to 4 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl.

26. A TREK family protein or homolog thereof comprising a cysteine residue at an amino position corresponding to position 131 of TREK-1.

27. The TREK family protein or homolog thereof of claim 26, wherein the TREK family protein is a TREK-1 protein, a TREK-2 protein, or a TRAKK protein.

28. The TREK family protein or homolog thereof of claim 26, wherein the TREK family protein comprises any one of SEQ ID NOS: 1-12.

29. The TREK family protein or homolog thereof of claim 26, wherein the TREK family protein comprises SEQ ID NO: 1.

30. The TREK family protein or homolog thereof of claim 26, wherein the TREK family protein comprises SEQ ID NO: 2.

31. The TREK family protein or homolog thereof of claim 26, wherein the TREK family protein comprises SEQ ID NO: 3.

32. The TREK family protein or homolog thereof of claim 26, wherein the TREK family protein comprises SEQ ID NO: 4.

33. The TREK family protein or homolog thereof of claim 26, wherein the TREK family protein comprises SEQ ID NO: 5.

34. The TREK family protein or homolog thereof of claim 26, wherein the TREK family protein comprises SEQ ID NO: 6.

35. The TREK family protein or homolog thereof of claim 26, wherein the TREK family protein comprises SEQ ID NO: 7.

36. The TREK family protein or homolog thereof of claim 26, wherein the TREK family protein comprises SEQ ID NO: 8.

37. The TREK family protein or homolog thereof of claim 26, wherein the TREK family protein comprises SEQ ID NO: 9.

38. The TREK family protein or homolog thereof of claim 26, wherein the TREK family protein comprises SEQ ID NO: 10.

39. The TREK family protein or homolog thereof of claim 26, wherein the TREK family protein comprises SEQ ID NO: 11.

40. The TREK family protein or homolog thereof of claim 26, wherein the TREK family protein comprises SEQ ID NO: 12.

41. A nucleic acid encoding the TREK family protein or homolog thereof of one of claims 26 to 40.

42. A viral particle comprising the nucleic acid of claim 41.

43. A method of treating a disease or adverse condition related to low TREK family protein activity in a subject in need thereof, the method comprising administering to the subject an effective amount of a nucleic acid encoding the TREK family protein of claim 26 and a therapeutically effective amount of a TREK family protein agonist, wherein said TREK family protein agonist comprises a cysteine binding moiety capable of covalently binding said TREK family protein at said cysteine residue.

44. The method of claim 43, wherein the TREK family protein is a TREK-1 protein, a TREK-2 protein, or a TRAAK protein.

45. The method of claim 43, wherein said nucleic acid is within a viral particle.

46. The method of claim 45, wherein the viral particle is an inactivated or genetically modified human papillomavirus, rhinovirus, hepatitis B virus, or herpesvirus.

47. The method of claim 43, wherein said TREK family protein agonist is a short-hairpin RNA (shRNA), a small interference RNA (siRNA), a piwi-interacting RNA (piRNA), a microRNA (miRNA), an antisense oligonucleotide, a morpholinooligonucleotide, a CRISPR Cas guide RNA (gRNA), a small molecule compound, an antibody, or a nucleic acid.

48. The method of claim 47, wherein said TREK family protein agonist is a short-hairpin RNA (shRNA).

49. The method of claim 47, wherein said TREK family protein agonist is a small interference RNA (siRNA).

50. The method of claim 47, wherein said TREK family protein agonist is a piwi-interacting RNA (piRNA).

51. The method of claim 47, wherein said TREK family protein agonist is a microRNA (miRNA).

52. The method of claim 47, wherein said TREK family protein agonist is an antisense oligonucleotide.

53. The method of claim 47, wherein said TREK family protein agonist is a GapmeR.

54. The method of claim 47, wherein said TREK family protein agonist is a morpholinooligonucleotide.

55. The method of claim 47, wherein said TREK family protein agonist is a CRISPR Cas guide RNA (gRNA).

56. The method of claim 55, wherein said CRISPR Cas guide RNA is CRISPR Cas 12 guide RNA.

57. The method of claim 55, wherein said CRISPR Cas guide RNA is CRISPR Cas 9 guide RNA.

58. The method of claim 47, wherein said TREK family protein agonist is aTREK family protein antibody.

59. The method of claim 47, wherein said TREK family protein agonist is a nucleic acid binding said cysteine residue.

60. The method of claim 47, wherein said TREK family protein agonist is the compound of any one of claims 1 to 25.

61. The method of claim 43, wherein the cysteine residue is at an amino position corresponding to position 131 of TREK-1.

62. The method of claim 43, wherein said disease or adverse condition is chronic pain, nerve injury, lack of sleep, high intraocular pressure, headache, depression, pulmonary hypertension, lung injury, or decompression sickness.

63. The method of claim 62, wherein the nerve injury is an injury of the dorsal ganglion nerve.

64. A method of treating a disease or adverse condition related to low TREK family protein activity in a subject in need thereof, the method comprising administering to said subject a gene editing system capable of mutating a TREK family protein to comprise a cysteine residue at an amino position corresponding to position 131 of TREK-1, and a TREK family protein agonist, wherein said TREK family protein agonist comprises a cysteine binding moiety that is capable of covalently binding said TREK family protein at said cysteine residue.

65. The method of claim 64, wherein the TREK family protein is a TREK-1 protein, a TREK-2 protein, or a TRAAK protein.

66. The method of claim 64, wherein said gene editing system is a CRISPR Cas guide RNA (gRNA), a transcription activator-like effector nuclease (TALEN), or a zinc-finger nuclease (ZFN).

67. The method of claim 66, wherein said CRISPR Cas guide RNA is CRISPR Cas 12 guide RNA.

68. The method of claim 66, wherein said CRISPR Cas guide RNA is CRISPR Cas 9 guide RNA.

69. The method of claim 64, wherein said TREK family protein agonist is a nucleic acid binding said cysteine residue.

70. The method of claim 64, wherein said TREK family protein agonist is the compound of any one of claims 1 to 25.

71. The method of claim 64, wherein the cysteine residue is at an amino position corresponding to position 131 of TREK-1.

72. A method of increasing TREK family protein activity in a tissue, said method comprising administering to said tissue a nucleic acid encoding the TREK family protein of one of claims 26 to 40 and a TREK family protein agonist, wherein said TREK family protein agonist comprises a cysteine binding moiety capable of covalently binding said TREK family protein at said cysteine residue.

73. The method of claim 72, wherein the TREK family protein is a TREK-1 protein, a TREK-2 protein, or a TRAAK protein.

74. The method of claim 72, wherein said TREK family protein agonist is a short-hairpin RNA (shRNA), a small interference RNA (siRNA), a piwi-interacting RNA (piRNA), a microRNA (miRNA), an antisense oligonucleotide, a morpholinooligonucleotide, a CRISPR Cas guide RNA (gRNA), a small molecule compound, an antibody, or a nucleic acid.

75. The method of claim 74, wherein said TREK family protein agonist is a short-hairpin RNA (shRNA).

76. The method of claim 74, wherein said TREK family protein agonist is a small interference RNA (siRNA).

77. The method of claim 74, wherein said TREK family protein agonist is a piwi-interacting RNA (piRNA).

78. The method of claim 74, wherein said TREK family protein agonist is a microRNA (miRNA).

79. The method of claim 74, wherein said TREK family protein agonist is an antisense oligonucleotide.

80. The method of claim 74, wherein said TREK family protein agonist is a GapmeR.

81. The method of claim 74, wherein said TREK family protein agonist is a morpholinooligonucleotide.

82. The method of claim 74, wherein said TREK family protein agonist is a CRISPR Cas guide RNA (gRNA).

83. The method of claim 82, wherein said CRISPR Cas guide RNA is CRISPR Cas 12 guide RNA.

84. The method of claim 82, wherein said CRISPR Cas guide RNA is CRISPR Cas 9 guide RNA.

85. The method of claim 74, wherein said TREK family protein agonist is aTREK family protein antibody.

86. The method of claim 72, wherein said TREK family protein agonist is a nucleic acid binding said cysteine residue.

87. The method of claim 72, wherein said TREK family protein agonist is the compound of any one of claims 1 to 25.

88. The method of claim 72, wherein the cysteine residue is at an amino position corresponding to position 131 of TREK-1.

89. The method of claim 72, wherein said tissue is a brain, heart, nerve, nerve ganglia, eye, smooth muscle, endocrine, pancreas, prostate, or sensory organ tissue.
